(19) United States
(12) Patent Application Publication
(10) Pub. No.: US 2020/0306209 A1
(43) Pub. Date: Oct. 1, 2020

(54) METHOD OF TREATING ACID-BASE DISORDERS

(71) Applicant: Tricida, Inc., South San Francisco, CA (US)

(72) Inventors: Gerrit KLAERNER, Hillsborough, CA (US); Dawn Parsell OTTO, Cedar Park, TX (US); Yuri STASIV, Mountain View, CA (US); Vandana MATHUR, Woodside, CA (US); Claire LOCKEY, Belmont, CA (US)

(21) Appl. No.: 16/829,607

(22) Filed: Mar. 25, 2020

Related U.S. Application Data
(60) Provisional application No. 62/825,006, filed on Mar. 27, 2019.

(51) Int. Cl. A61K 31/132; A61K 9/00; A61P 3/12; A61K 31/785

(52) U.S. Cl. CPC A61K 31/132; A61K 31/785; A61P 3/12; A61K 9/0053

(57) ABSTRACT

The present disclosure provides, inter alia, pharmaceutical compositions for and methods of treating an animal, including a human, and methods of preparing such compositions. In certain embodiments, the pharmaceutical compositions contain nonabsorbable compositions and may be used, for example, to treat diseases or other metabolic conditions in which removal of protons, the conjugate base of a strong acid and/or a strong acid from the gastrointestinal tract would provide physiological benefits such as normalizing serum bicarbonate concentrations and the blood pH in an animal, including a human. In certain embodiments, compositions for and methods of improving the quality of life and/or physical function score of such patients are also provided. In certain embodiments, compositions for and methods of slowing the progression of kidney disease in such patients are also provided.

### CROSS REFERENCE TO RELATED APPLICATIONS

The present application claims priority to provisional application No. 62/825,006, filed Mar. 27, 2019, which is hereby incorporated by reference in its entirety herein.

The present invention generally relates to methods of treating acid-base disorders that may be used, for example, in the treatment of metabolic acidosis.

Metabolic acidosis is the result of metabolic and dietary processes that in various disease states create a condition in which non-volatile acids accumulate in the body, causing a net addition of protons (H⁺) or the loss of bicarbonate (HCO₃⁻). Metabolic acidosis occurs when the body accumulates acid from metabolic and dietary processes and the excess acid is not completely removed from the body by the kidneys. Chronic kidney disease is often accompanied by metabolic acidosis due to the reduced capacity of the kidney to excrete hydrogen ions secondary to an inability to reclaim filtered bicarbonate (HCO₃⁻), synthesize ammonia (ammoniagenesis), and excrete titratable acids. Clinical practice guidelines recommend initiation of alkali therapy in patients with non-dialysis-dependent chronic kidney disease (CKD) when the serum bicarbonate level is <22 mEq/L to prevent or treat complications of metabolic acidosis. (Clinical practice guidelines for nutrition in chronic renal failure, K/DOQI, National Kidney Foundation, Am. J. Kidney Dis. 2000; 35:S1-140; Raphael, K L, Zhang, Y, Wei, G, et al. 2013, Serum bicarbonate and mortality in adults in NHANES III, Nephrol. Dial. Transplant 28: 1207-1213). These complications include malnutrition and growth retardation in children, exacerbation of bone disease, increased muscle degradation, reduced albumin synthesis, and increased inflammation. (Leman, J, Litzow, J R, Lennon, E J. 1966. The effects of chronic acid loads in normal man: further evidence for the participation of bone mineral in the defense against chronic metabolic acidosis, J. Clin. Invest. 45: 1608-1614; Franch H A, Mitch W E, 1998, Catabolism in uremia: the impact of metabolic acidosis, J. Am. Soc. Nephrol. 9: S78-81; Ballmer, P E, McNurlan, M A, Hulter, H N, et al., 1995, Chronic metabolic acidosis decreases albumin synthesis and induces negative nitrogen balance in humans, J. Clin. Invest. 95: 39-45; Farwell, W R, Taylor, E N, 2010, Serum anion gap, bicarbonate and biomarkers of inflammation in healthy individuals in a national survey, CMAJ 182:137-141). Overt metabolic acidosis is present in a large proportion of patients when the estimated glomerular filtration rate is below 30 ml/min/1.73 m². (KDOQI bone guidelines: American Journal of Kidney Diseases (2003) 42:S1-S201. (suppl); Widmer B, Gerhardt R E, Harrington J T, Cohen J J, Serum electrolyte and acid base composition: The influence of graded degrees of chronic renal failure, Arch Intern Med 139:1099-1102, 1979; Dobre M, Yang, W, Chen J, et. al., Association of serum bicarbonate with risk of renal and cardiovascular outcomes in CKD: a report from the chronic renal insufficiency cohort (CRIC) study. Am. J. Kidney Dis. 62: 670-678, 2013; Yaqoob, M M. Acidosis and progression of chronic kidney disease. Curr. Opin. Nephrol. Hypertens. 19: 489-492, 2010).

Metabolic acidosis, regardless of etiology, lowers extracellular fluid bicarbonate and, thus, decreases extracellular pH. The relationship between serum pH and serum bicarbonate is described by the Henderson-Hasselbalch equation

pH=pK′+log[HCO₃⁻]/[(0.03×PₐCO₂)]

where 0.03 is the physical solubility coefficient for CO₂, [HCO₃⁻] and PₐCO₂ are the concentrations of bicarbonate and the partial pressure of carbon dioxide, respectively.

There are several laboratory tests that can be used to define metabolic acidosis. The tests fundamentally measure either bicarbonate (HCO₃⁻) or proton (H⁺) concentration in various biological samples, including venous or arterial blood. These tests can measure either bicarbonate (HCO₃⁻) or proton (H⁺) concentration by enzymatic methodology, by ion selective electrodes or by blood gas analysis. In both the enzymatic and ion selective electrode methods, bicarbonate is “measured.” Using blood gas analysis, bicarbonate level can be calculated using the Henderson-Hasselbalch equation.

Arterial blood gas (ABG) analysis is commonly performed for clinical evaluation, but the procedure has certain limitations in the form of reduced patient acceptability because of painful procedure and the potential to cause complications such as arterial injury, thrombosis with distal ischaemia, haemorrhage, aneurysm formation, median nerve damage and reflex sympathetic dystrophy. Venous blood gas (VBG) analysis is a relatively safer procedure as fewer punctures are required thus reducing the risk of needle stick injury to the health care workers. Therefore, as set out below, when the invention requires assessment of metabolic acidosis, it is preferred to complete this assessment using VBG analysis. Any measurements specified herein are preferably achieved by VBG analysis where possible, for example measurements of blood or serum bicarbonate levels.

The most useful measurements for the determination of acidosis rely on a measurement of the venous plasma bicarbonate (or total carbon dioxide [tCO₂]), or arterial plasma bicarbonate (or total carbon dioxide [tCO₂]), serum electrolytes Cl⁻, K⁺, and Na⁺, and a determination of the anion gap. In the clinical laboratory, measurement of venous plasma or serum electrolytes includes an estimation of the tCO₂. This measurement reflects the sum of circulating CO₂ [i.e., the total CO₂ represented by bicarbonate (HCO₃⁻), carbonic acid, (H₂CO₃) and dissolved CO₂ (0.03×PCO₂)]. tCO₂ can also be related to HCO₃⁻ by using a simplified and standardized form of the Henderson-Hasselbalch equation: tCO₂=HCO₃⁻+0.03PCO₂, where PCO₂ is the measured partial pressure of CO₂ Since HCO₃⁻ concentration is greater than 90% of the tCO₂, and there are small amounts of H₂CO₃, then venous tCO₂ is often used as a reasonable approximation of the venous HCO₃⁻ concentration in the blood. Especially during chronic kidney disease, an abnormal plasma HCO₃ value<22 mEq/L generally indicates metabolic acidosis.

Changes in serum Cl⁻ concentration can provide additional insights into possible acid-base disorders, particularly when they are disproportionate to changes in serum Na⁺ concentration. When this occurs, the changes in serum Cl⁻ concentration are typically associated with reciprocal changes in serum bicarbonate. Thus, in metabolic acidosis with normal anion gap, serum Cl⁻ increases >105 mEq/L as serum bicarbonate decreases <22 mEq/L.

Calculation of the anion gap [defined as the serum Na⁺—(Cl⁻+HCO₃⁻)] is an important aspect of the diagnosis of metabolic acidosis. Metabolic acidosis may be present with a normal or an elevated anion gap. However, an elevated anion gap commonly signifies the presence of metabolic acidosis, regardless of the change in serum HCO₃⁻. An anion gap greater than 20 mEq/L (normal anion gap is 8 to 12 mEq/L) is a typical feature of metabolic acidosis.

Arterial blood gases are used to identify the type of an acid-base disorder and to determine if there are mixed disturbances. In general, the result of arterial blood gas measures should be coordinated with history, physical exam and the routine laboratory data listed above. An arterial blood gas measures the arterial carbon dioxide tension (PₐCO₂), acidity (pH), and the oxygen tension (PₐO₂). The HCO₃⁻ concentration is calculated from the pH and the PₐCO₂. Hallmarks of metabolic acidosis are a pH<7.35, PₐCO₂<35 mm Hg and HCO₃⁻<22 mEq/L. The value of PₐO₂ (normal 80-95 mmHg) is not used in making the diagnosis of metabolic acidosis but may be helpful in determining the cause. Acid-base disturbance are first classified as respiratory or metabolic. Respiratory disturbances are those caused by abnormal pulmonary elimination of CO₂, producing an excess (acidosis) or deficit (alkalosis) of CO₂ (carbon dioxide) in the extracellular fluid. In respiratory acid-base disorders, changes in serum bicarbonate (HCO₃⁻) are initially a direct consequence of the change in PCO₂ with a greater increase in PCO₂ resulting in an increase in HCO₃⁻. (Adrogue H J, Madias N E, 2003, Respiratory acidosis, respiratory alkalosis, and mixed disorders, in Johnson R J, Feehally J (eds): Comprehensive Clinical Nephrology. London, CV Mosby, pp. 167-182). Metabolic disturbances are those caused by excessive intake of, or metabolic production or losses of, nonvolatile acids or bases in the extracellular fluid. These changes are reflected by changes in the concentration of bicarbonate anion (HCO₃⁻) in the blood; adaptation in this case involves both buffering (immediate), respiratory (hours to days) and renal (days) mechanisms. (DuBose T D, MacDonald G A: renal tubular acidosis, 2002, in DuBose T D, Hamm L L (eds): Acid-base and electrolyte disorders: A companion to Brenners and Rector's the Kidney, Philadelphia, WB Saunders, pp. 189-206).

The overall hydrogen ion concentration in the blood is defined by the ratio of two quantities, the serum HCO₃⁻ content (regulated by the kidneys) and the PCO₂ content (regulated by the lungs) and is expressed as follows:

[H⁺]∝(PCO₂/[HCO₃⁻])

The consequence of an increase in the overall hydrogen ion concentration is a decline in the major extracellular buffer, bicarbonate. Normal blood pH is between 7.38 and 7.42, corresponding to a hydrogen ion (H⁺) concentration of 42 to 38 nmol/L (Goldberg M: Approach to Acid-Base Disorders. 2005. In Greenberg A, Cheung A K (eds) Primer on Kidney Diseases, National Kidney Foundation, Philadelphia, Elsevier-Saunders, pp. 104-109.). Bicarbonate (HCO₃⁻) is an anion that acts to buffer against pH disturbances in the body, and normal levels of plasma bicarbonate range from 22-26 mEq/L (Szerlip H M: Metabolic Acidosis, 2005, in Greenberg A, Cheung A K (eds) Primer on Kidney Diseases, National Kidney Foundation, Philadelphia, Elsevier-Saunders, pp. 74-89.). Acidosis is the process which causes a reduction in blood pH (acidemia) and reflects the accumulation of hydrogen ion (H⁺) and its consequent buffering by bicarbonate ion (HCO₃⁻) resulting in a decrease in serum bicarbonate. Metabolic acidosis can be represented as follows:

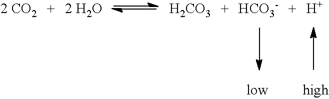

(Clinical practice guidelines for nutrition in chronic renal failure. K/DOQI, National Kidney Foundation. Am. J. Kidney Dis. 2000; 35:S1-140). Using this balance equation, the loss of one HCO₃⁻ is equivalent to the addition of one H⁺ and conversely, the gain of one HCO₃⁻ is equivalent to the loss of one H. Thus, changes in blood pH, particularly increases in H⁺ (lower pH, acidosis) can be corrected by increasing serum HCO₃⁻ or, equivalently, by decreasing serum H.

In order to maintain extracellular pH within the normal range, the daily production of acid must be excreted from the body. Acid production in the body results from the metabolism of dietary carbohydrates, fats and amino acids. Complete oxidation of these metabolic substrates produces water and CO₂. The carbon dioxide generated by this oxidation (˜20,000 mmol/day) is efficiently exhaled by the lungs, and represents the volatile acid component of acid-base balance.

In contrast, nonvolatile acids (˜50-100 mEq/day) are produced by the metabolism of sulfate- and phosphate-containing amino acids and nucleic acids. Additional nonvolatile acids (lactic acid, butyric acid, acetic acid, other organic acids) arise from the incomplete oxidation of fats and carbohydrates, and from carbohydrate metabolism in the colon, where bacteria residing in the colon lumen convert the substrates into small organic acids that are then absorbed into the bloodstream. The impact of short chain fatty acids on acidosis is somewhat minimized by anabolism, for example into long-chain fatty acids, or catabolism to water and CO₂.

The kidneys maintain pH balance in the blood through two mechanisms: reclaiming filtered HCO₃⁻ to prevent overall bicarbonate depletion and the elimination of nonvolatile acids in the urine. Both mechanisms are necessary to prevent bicarbonate depletion and acidosis.

In the first mechanism, the kidneys reclaim HCO₃⁻ that is filtered by the glomerulus. This reclamation occurs in the proximal tubule and accounts for ˜4500 mEq/day of reclaimed HCO₃⁻. This mechanism prevents HCO₃⁻ from being lost in the urine, thus preventing metabolic acidosis. In the second mechanism, the kidneys eliminate enough H⁺ to equal the daily nonvolatile acid production through metabolism and oxidation of protein, fats and carbohydrates. Elimination of this acid load is accomplished by two distinct routes in the kidney, comprising active secretion of H⁺ ion and ammoniagenesis. The net result of these two interconnected processes is the elimination of the 50-100 mEq/day of nonvolatile acid generated by normal metabolism.

Thus, normal renal function is needed to maintain acid-base balance. During chronic kidney disease, filtration and reclamation of HCO₃⁻ is impaired as is generation and secretion of ammonia. These deficits rapidly lead to chronic metabolic acidosis which is, itself, a potent antecedent to end-stage renal disease. With continued acid production from metabolism, a reduction in acid elimination will disturb the H⁺/HCO₃⁻ balance such that blood pH falls below the normal value of pH=7.38-7.42.

Treatment of metabolic acidosis by alkali therapy is usually indicated to raise and maintain the plasma pH to greater than 7.20. Sodium bicarbonate (NaHCO₃) is the agent most commonly used to correct metabolic acidosis. NaHCO₃ can be administered intravenously to raise the serum HCO₃⁻ level adequately to increase the pH to greater than 7.20. Further correction depends on the individual situation and may not be indicated if the underlying process is treatable or the patient is asymptomatic. This is especially true in certain forms of metabolic acidosis. For example, in high-anion gap (AG) acidosis secondary to accumulation of organic acids, lactic acid, and ketones, the cognate anions are eventually metabolized to HCO₃⁻. When the underlying disorder is treated, the serum pH corrects; thus, caution should be exercised in these patients when providing alkali to raise the pH much higher than 7.20, to prevent an increase in bicarbonate above the normal range (>26 mEq/L).

Citrate is an appropriate alkali therapy to be given orally or IV, either as the potassium or sodium salt, as it is metabolized by the liver and results in the formation of three moles of bicarbonate for each mole of citrate. Potassium citrate administered IV should be used cautiously in the presence of renal impairment and closely monitored to avoid hyperkalemia.

Intravenous sodium bicarbonate (NaHCO₃) solution can be administered if the metabolic acidosis is severe or if correction is unlikely to occur without exogenous alkali administration. Oral alkali administration is the preferred route of therapy in persons with chronic metabolic acidosis. The most common alkali forms for oral therapy include NaHCO₃ tablets where 1 g of NaHCO₃ is equal to 11.9 mEq of HCO₃⁻. However, the oral form of NaHCO₃ is not approved for medical use and the package insert of the intravenous sodium bicarbonate solution includes the following contraindications, warnings and precautions (Hospira label for NDC 0409-3486-16):
Contraindications: Sodium Bicarbonate Injection, USP is contraindicated in patients who are losing chloride by vomiting or from continuous gastrointestinal suction, and in patients receiving diuretics known to produce a hypochloremic alkalosis.
Warnings: Solutions containing sodium ions should be used with great care, if at all, in patients with congestive heart failure, severe renal insufficiency and in clinical states in which there exists edema with sodium retention. In patients with diminished renal function, administration of solutions containing sodium ions may result in sodium retention. The intravenous administration of these solutions can cause fluid and/or solute overloading resulting in dilution of serum electrolyte concentrations, overhydration, congested states or pulmonary edema.
Precautions: [ . . . ] The potentially large loads of sodium given with bicarbonate require that caution be exercise in the use of sodium bicarbonate in patients with congestive heart failure or other edematous or sodium-retaining states, as well as in patients with oliguria or anuria.

Acid-base disorders are common in chronic kidney disease and heart failure patients. Chronic kidney disease (CKD) progressively impairs renal excretion of the approximately 1 mmol/kg body weight of hydrogen ions generated in healthy adults (Yaqoob, M M. 2010, Acidosis and progression of chronic kidney disease, Curr. Opin. Nephrol. Hyperten. 19:489-492.). Metabolic acidosis, resulting from the accumulation of acid (H⁺) or depletion of base (HCO₃⁻) in the body, is a common complication of patients with CKD, particularly when the glomerular filtration rate (GFR, a measure of renal function) falls below 30 ml/min/1.73 m². Metabolic acidosis has profound long term effects on protein and muscle metabolism, bone turnover and the development of renal osteodystrophy. In addition, metabolic acidosis influences a variety of paracrine and endocrine functions, again with long term consequences such as increased inflammatory mediators, reduced leptin, insulin resistance, and increased corticosteroid and parathyroid hormone production (Mitch W E, 1997, Influence of metabolic acidosis on nutrition, Am. J. Kidney Dis. 29:46-48.). The net effect of sustained metabolic acidosis in the CKD patient is loss of bone and muscle mass, a negative nitrogen balance, and the acceleration of chronic renal failure due to hormonal and cellular abnormalities (De Brito-Ashurst I, Varagunam M, Raftery M J, et al, 2009, Bicarbonate supplementation slows progression of CKD and improves nutritional status, J. Am. Soc. Nephrol. 20: 2075-2084). Conversely, the potential concerns with alkali therapy in CKD patients include expansion of extracellular fluid volume associated with sodium ingestion, resulting in the development or aggravation of hypertension, facilitation of vascular calcification, and the decompensation of existing heart failure. CKD patients of moderate degree (GFR at 20-25% of normal) first develop hyperchloremic acidosis with a normal anion gap due to the inability to reclaim filtered bicarbonate and excrete proton and ammonium cations. As they progress toward the advanced stages of CKD the anion gap increases, reflective of the continuing degradation of the kidney's ability to excrete the anions that were associated with the unexcreted protons. Serum bicarbonate in these patients rarely goes below 15 mmol/L with a maximum elevated anion gap of approximately 20 mmol/L. The non-metabolizable anions that accumulate in CKD are buffered by alkaline salts from bone (Lemann J Jr, Bushinsky D A, Hamm L L Bone buffering of acid and base in humans. Am. J. Physiol Renal Physiol. 2003 November, 285(5):F811-32).

The majority of patients with chronic kidney disease have underlying diabetes (diabetic nephropathy) and hypertension, leading to deterioration of renal function. In almost all patients with hypertension a high sodium intake will worsen the hypertension. Accordingly, kidney, heart failure, diabetes and hypertensive guidelines strictly limit sodium intake in these patients to less than 1.5 g or 65 mEq per day (HFSA 2010 guidelines, Lindenfeld 2010, J Cardiac Failure V16 No 6 P475). Chronic anti-hypertensive therapies often induce sodium excretion (diuretics) or modify the kidney's ability to excrete sodium and water (such as, for example, Renin Angiotensin Aldosterone System inhibiting “RAASi” drugs). However, as kidney function deteriorates, diuretics become less effective due to an inability of the tubule to respond. The RAASi drugs induce life-threatening hyperkalemia as they inhibit renal potassium excretion. Given the additional sodium load, chronically treating metabolic acidosis patients with amounts of sodium-containing base that often exceed the total daily recommended sodium intake is not a reasonable practice. As a consequence, oral sodium bicarbonate is not commonly prescribed chronically in these diabetic nephropathy patients. Potassium bicarbonate is also not acceptable as patients with CKD are unable to readily excrete potassium, leading to severe hyperkalemia.

Despite these shortcomings, the role of oral sodium bicarbonate has been studied in the small subpopulation of non-hypertensive CKD patients. As part of the Kidney Research National Dialogue, alkali therapy was identified as having the potential to slow the progression of CKD, as well as to correct metabolic acidosis. The annual age-related decline in glomerular filtration rate (GFR) after the age of 40 is 0.75-1.0 ml/min/1.73 m² in normal individuals. In CKD patients with fast progression, a steeper decline of >4 ml/min/1.73 m² annually can be seen. Glomerular filtration rate or estimated glomerular filtration rate is typically used to characterize kidney function and the stage of chronic kidney disease. The five stages of chronic kidney disease and the GFR for each stage is as follows:

Stage 1 with normal or high GFR (GFR>90 mL/min/1.73 m²)

Stage 2 Mild CKD (GFR=60-89 mL/min/1.73 m²)

Stage 3A Moderate CKD (GFR=45-59 mL/min/1.73 m²)

Stage 3B Moderate CKD (GFR=30-44 mL/min/1.73 m²)

Stage 4 Severe CKD (GFR=15-29 mL/min/1.73 m²)

Stage 5 End Stage CKD (GFR<15 mL/min/1.73 m²).

In one outcome study, De Brito-Ashurst et al showed that bicarbonate supplementation preserves renal function in CKD (De Brito-Ashurst I, Varagunam M, Raftery M J, et al, 2009, Bicarbonate supplementation slows progression of CKD and improves nutritional status, J. Am. Soc. Nephrol. 20: 2075-2084). The study randomly assigned 134 adult patients with CKD (creatinine clearance [CrCl] 15 to 30 ml/min per 1.73 m²) and serum bicarbonate 16 to 20 mmol/L to either supplementation with oral sodium bicarbonate or standard of care for 2 years. The average dose of bicarbonate in this study was 1.82 g/day, which provides 22 mEq of bicarbonate per day. The primary end points were rate of CrCl decline, the proportion of patients with rapid decline of CrCl (>3 ml/min per 1.73 m²/yr), and end-stage renal disease (“ESRD”) (CrCl<10 ml/min). Compared with the control group, decline in CrCl was slower with bicarbonate supplementation (decrease of 1.88 ml/min per 1.73 m² for patients receiving bicarbonate versus a decrease of 5.93 ml/min per 1.73 m² for control group; P<0.0001). Patients supplemented with bicarbonate were significantly less likely to experience rapid progression (9% versus 45%; relative risk 0.15; 95% confidence interval 0.06 to 0.40; P<0.0001). Similarly, fewer patients supplemented with bicarbonate developed ESRD (6.5% versus 33%; relative risk 0.13; 95% confidence interval 0.04 to 0.40; P<0.001).

Hyperphosphatemia is a common co-morbidity in patients with CKD, particularly in those with advanced or end-stage renal disease. Sevelamer hydrochloride is a commonly used ion-exchange resin that reduces serum phosphate concentration. However, reported drawbacks of this agent include metabolic acidosis apparently due to the net absorption of HCl in the process of binding phosphate in the small intestine. Several studies in patients with CKD and hyperphosphatemia who received hemodialysis or peritoneal dialysis found decreases in serum bicarbonate concentrations with the use of Sevelamer hydrochloride (Brezina, 2004 Kidney Int. V66 S90 (2004) S39-S45; Fan, 2009 Nephrol Dial Transplant (2009) 24:3794).

Among the various aspects of the present disclosure, the following is a useful guide for one method for treating metabolic acidosis (without wishing to be bound by theory). When an H⁺ is pumped into the stomach a HCO₃⁻ enters the systemic circulation and raises the serum bicarbonate concentration. The initial binding of gastric H⁺ to a nonabsorbable composition as described herein results in HCO₃⁻ entering the systemic circulation and raising the serum bicarbonate concentration. The more H⁺ bound the greater the increase in systemic HCO₃⁻. The binding of Cl the nonabsorbable composition prevents subsequent exchange of luminal Cl for HCO₃⁻ which would counteract the initial rise in HCO₃⁻. The analogous clinical situation to administering the composition is vomiting. Administration of the composition is essentially causing the loss of gastric HCl as in vomiting. If a person vomits they lose gastric HCl and have an increase in serum bicarbonate. The increase in serum bicarbonate persists only if they are not given a lot of oral for example as NaCl, which would allow subsequent exchange of intestinal Cl for HCO₃⁻ and dissipate the increase in serum bicarbonate concentration. The disclosure is not limited by these requirements, and instead they are set out in full below.

Among the various aspects of the present disclosure may be noted a method of treating an individual afflicted with a chronic acid/base disorder characterized by a baseline serum bicarbonate value of less than 22 mEq/l. The method comprises oral administration of a pharmaceutical composition comprising a nonabsorbable composition having the capacity to bind a target species selected from the group consisting of protons, a conjugate base of a strong acid, and a strong acid as it transits the digestive system and increase the individual's serum bicarbonate value to at least 24 mEq/l but less than 30 mEq/l.

Among the various aspects of the present disclosure may be noted a method of treating an individual afflicted with a chronic acid/base disorder characterized by a baseline serum bicarbonate value of less than 22 mEq/l. The method comprises oral administration of a pharmaceutical composition comprising a nonabsorbable composition having the capacity to bind a target species selected from the group consisting of protons, a conjugate base of a strong acid, and a strong acid as it transits the digestive system and increase the individual's serum bicarbonate value to at least 24 mEq/l but not greater than 29 mEq/l.

Another aspect of the present disclosure is a method of treating an individual afflicted with an acid-base disorder characterized by a baseline serum bicarbonate value of less than 22 mEq/l, the method comprising oral administration of a daily dose of a pharmaceutical composition having the capacity to remove at least 5 meq of a target species as it transits the digestive system to increase the individual's serum bicarbonate value to at least 24 mEq/l but not greater than 29 mEq/l from baseline within a treatment period not greater than 1 month. The target species is selected from the group consisting of protons, strong acids, and conjugate bases of strong acids.

Another aspect of the present disclosure is a composition for use in a method of treating metabolic acidosis in an adult human patient by increasing that patient's serum bicarbonate value by at least 1 mEq/L over 15 days of treatment (i.e., within 15 days of treatment), said composition being a nonabsorbable composition having the capacity to remove protons from the patient. In this aspect, the composition may be administered orally, and so would be an orally administered nonabsorbable composition as defined herein.

In certain embodiments, the orally administered nonabsorbable composition comprises cations (such as Na⁺, K⁺, Mg²⁺, Ca²⁺ Li⁺, or a combination thereof) that are exchanged for protons as the nonabsorbable composition transits the digestive system, and the protons are then excreted from the body along with the nonabsorbable composition upon defecation. The net effect is reduction in protons in the body, in exchange for an increase in one or more cations. In this embodiment, the pharmaceutical composition may also optionally comprise a pharmaceutically acceptable carrier, diluent or excipient, or a combination thereof that does not significantly interfere with the proton-binding characteristics of the nonabsorbable composition in vivo. Optionally, the pharmaceutical composition may also comprise an additional therapeutic agent.

In certain embodiments, the orally administered nonabsorbable composition comprises anions that are exchanged for chloride ions and if the anion comprised by the orally administered nonabsorbable composition is a stronger base (e.g., OH⁻) than the removed base (e.g., Cl⁻, HSO₄⁻, or SO₄²⁻), the net effect is the removal of a strong acid from the body (e.g., HCl or H₂SO₄) in exchange for a weak acid (e.g., H₂O). In this embodiment, the pharmaceutical composition may also optionally comprise a pharmaceutically acceptable carrier, diluent or excipient, or a combination thereof that does not significantly interfere with the chloride-binding characteristics of the nonabsorbable composition in vivo. Optionally, the pharmaceutical composition may also comprise an additional therapeutic agent.

In certain embodiments, the orally administered nonabsorbable composition is a neutral composition having the capacity to bind and remove a strong acid, such as HCl or H₂SO₄, from the body upon oral administration. The nonabsorbable composition may, but does not necessarily, introduce (i.e., by ion exchange) counterbalancing cations or anions in the process of removing the acid. In this embodiment, binding of both ionic species of HCl (H⁺ and Cr) may be achieved through favorable surface energy of the bulk material, which can include hydrogen bonding and other interactions as well as ionic interactions. Complexation of HCl can occur on functional groups that are dehydrated and upon administration in an acidic aqueous medium, result in the hydrochloride salt of the functional group.

Among the various aspects of the present disclosure may further be noted a method of treating an individual afflicted with a chronic acid/base disorder comprising oral administration of a pharmaceutical composition containing a nonabsorbable composition having the capacity to bind protons and chloride ions as it transits the digestive system and remove the bound protons and chloride ions from the individual's digestive system via defecation. In each of these embodiments, the pharmaceutical composition may also optionally comprise a pharmaceutically acceptable carrier, diluent or excipient, or a combination thereof that does not significantly interfere with the chloride-binding characteristics of the nonabsorbable composition in vivo. Optionally, the pharmaceutical composition may also comprise an additional therapeutic agent.

In one embodiment, any of the methods of treating an individual afflicted with an acid-base disorder disclosed in this application comprise: i) the individual having a diet regimen, or ii) the method including, specifying, prescribing or recommending a diet regimen. In one embodiment, said diet regimen is an alkaline diet regimen. In one embodiment, said diet regimen is a conventional low-protein diet regimen (<0.6 g/kg per day). In one embodiment, said diet regimen is a very low-protein diet regimen (0.3-0.4 g/kg per day). In one embodiment, said diet regimen is a vegetarian diet regimen. In one embodiment, said diet regimen is a vegetarian diet regimen supplemented with either essential amino acids or a mixture of essential amino acids and nitrogen-free ketoanalogues (keto diet regimen). In one embodiment, said diet regimen is ketoanalogue-supplemented vegetarian very low-protein diet. In one embodiment, said diet regimen is a vegan diet regimen. In one embodiment, said diet regimen is a casein diet regimen. In one embodiment, said diet regimen is an adenine-containing diet regimen. In one embodiment, said diet regimen comprises one or more base-producing vegetables (e.g. carrots, cauliflower, eggplant, lettuce, potatoes, spinach, tomatoes, or zucchini, or a combination thereof). In one embodiment, said diet regimen comprises one or more base-producing fruits (e.g. apple, apricot, oranges, peaches, pears, raisins, or strawberries, or a combination thereof). In one embodiment, said diet regimen does not comprise acid-producing meat.

In one embodiment the diet commences one year before administering the nonabsorbable composition. In another embodiment the diet commences six months before administering the nonabsorbable composition. In another embodiment the diet commences one month before administering the nonabsorbable composition. In another embodiment the diet regimen commences when the administering of the nonabsorbable composition commences. In another embodiment the diet commences one month after administering the nonabsorbable composition. In another embodiment the diet commences six months after administering the nonabsorbable composition. In another embodiment the diet commences one year after administering the nonabsorbable composition.

Among the various aspects of the present disclosure may further be noted a method of improving the quality of life of a patient afflicted with chronic kidney disease and an acid-base disorder characterized by a baseline serum bicarbonate value of ≤22 mEq/L. The method comprises oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

Among the various aspects of the present disclosure may further be noted a method of treating an individual afflicted with chronic kidney disease, the method comprising administering a composition described herein.

In one embodiment, the rate of progression of the individual's chronic kidney disease is decreased. In this embodiment, the rate of progression may decrease for at least about 1 month, at least about 4 months, at least about 6 months, or at least about 12 months. As shown in FIG. 35, the rate of progression of chronic kidney disease is decreased to such an extent that the Death/Dialysis/≥50% eGFR decline (DD50) is reduced to 4% for populations treated with veverimer (TRC101) relative to 10.8% for populations treated with placebo.

Among the various aspects of the present disclosure may further be noted a method of decreasing the rate of progression of chronic kidney disease in an individual, the method comprising administering a composition described herein.

In one embodiment, the individual is afflicted with metabolic acidosis. The metabolic acidosis may be eubicarbonatemic metabolic acidosis. The metabolic acidosis may be characterized by a blood serum or blood plasma bicarbonate value not in excess of about 25 mEq/l, 24 mEq/l, or 23 mEq/l. The metabolic acidosis may be characterized by a blood serum or blood plasma bicarbonate value of less than about 22 mEq/l.

In another embodiment, the rate of decrease in the progression of chronic kidney disease is measurable by a decreased rate of change in eGFR.

In another embodiment, the decreased rate of change in eGFR occurs to the extent that eGFR stops decreasing.

In another embodiment, the decreased rate of change in eGFR occurs to the extent that there is an improvement in eGFR.

In another embodiment, the delay in the progression of chronic kidney disease includes the individual's stage of chronic kidney disease remaining constant. The patient may remain at stage 1, 2, 3A, 3B, 4 or 5 of chronic kidney disease. In this embodiment, the patient may remain at the claimed stage of chronic kidney disease for at least about 1 month, at least about 4 months, at least about 6 months, or at least about 12 months.

In any of the aspects disclosed herein, the blood pressure of the patient after treatment is unchanged relative to the blood pressure of the patient before treatment.

In any of the aspects disclosed herein, the blood pressure of the patient during treatment is unchanged relative to the blood pressure of the patient before treatment.

In any of the aspects disclosed herein, there is not a significant change in the blood pressure of the patient after treatment relative to the blood pressure of the patient before treatment.

In any of the aspects disclosed herein, there is not a significant change in the blood pressure of the patient during treatment relative to the blood pressure of the patient before treatment.

In any of the aspects disclosed herein the method or composition does not adversely affect blood pressure of treated patient or individual.

In another embodiment, a method of improving the quality of life of a patient afflicted with chronic kidney disease and an acid-base disorder is provided. This method comprises oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in quality of life is statistically significant compared to a placebo control group for a period of at least twelve weeks as assessed by a Quality of Life (QoL) questionnaire.

Another embodiment provides a method of improving quality of life of a patient afflicted with chronic kidney disease and an acid-base disorder, wherein the patient has a baseline serum bicarbonate value of ≤22 mEq/L. This method comprises orally administering to the patient an effective amount of TRC101 once daily for a period of time sufficient to statistically significantly increase the patient's quality of life compared to a placebo control.

A further embodiment provides a method of improving quality of life of a patient afflicted with metabolic acidosis disease. This method comprises administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the patient's quality of life compared to a placebo control group over the period, wherein the improvement in quality of life is statistically significant.

Another embodiment provides a pharmaceutical composition for improving the quality of life of a human patient afflicted with chronic kidney disease and an acid-base disorder, the patient having a baseline serum bicarbonate level of ≤22 mEq/L prior to treatment. This composition is a nonabsorbable composition having the capacity to: (a) remove a target species from the patient selected from the group consisting of protons, strong acids, and conjugate bases of strong acids; and (b) improve the patient's quality of life compared to a placebo control in a statistically significant manner over at least a twelve-week period.

A further embodiment is a pharmaceutical composition for improving the quality of life of a human patient suffering from a disease or disorder by increasing that patient's serum bicarbonate value by at least 1 mEq/L over at least twelve weeks of treatment. In this embodiment, the composition: (a) is a nonabsorbable composition having the capacity to remove a target species from the patient selected from the group consisting of protons, strong acids, and conjugate bases of strong acids; (b) is characterized by a target species binding capacity of at least 3 mEq/g in a Simulated Small Intestine Inorganic Buffer (SIB) assay; and (c) has the capacity to improve the patient's quality of life compared to a placebo control in a statistically significant manner over at least the twelve-week period.

Another embodiment is a pharmaceutical composition for improving the quality of life of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in quality of life compared to a placebo control is statistically significant over the twelve-week period.

A further embodiment is a method of improving the physical function of a patient afflicted with chronic kidney disease and an acid-base disorder characterized by a baseline serum bicarbonate value of ≤22 mEq/L. In this embodiment, the method comprises oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

A further embodiment is a method of improving the physical function of a patient afflicted with chronic kidney disease and an acid-base disorder. This method comprises oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in physical function is statistically significant compared to a placebo control group at least twelve weeks after initiation of treatment as assessed by the patient's answers to question 3 of the Kidney Disease Quality of Life Short Form (KDQOL-SF).

Another embodiment is a method of improving the physical function of a patient afflicted with chronic kidney disease and an acid-base disorder, wherein the patient has a baseline serum bicarbonate value of ≤22 mEq/L. This method comprises orally administering to the patient an effective amount of TRC101 once daily for a period of time sufficient to statistically significantly increase the patient's physical function score based on answers to question 3 of the Kidney Disease Quality of Life Short Form (KDQOL-SF) compared to the patient's baseline physical function score.

A further embodiment is a method of improving the physical function of a patient afflicted with metabolic acidosis disease. This method comprises administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the physical function score of the patient compared to a placebo control group at the end of the period, wherein the improvement in the physical function score is statistically significant.

Yet another embodiment is pharmaceutical composition for improving the physical function score of a human patient afflicted with chronic kidney disease and an acid-base disorder, the patient having a baseline serum bicarbonate level of ≤22 mEq/L prior to treatment. In this embodiment, the composition is a nonabsorbable composition having the capacity to: (a) remove a target species from the patient selected from the group consisting of protons, strong acids, and conjugate bases of strong acids; and (b) improve the patient's physical function score based on answers to question 3 of the Kidney Disease Quality of Life Short Form (KDQOL-SF) compared to a placebo control in a statistically significant manner at the end of at least a twelve-week period.

A further embodiment is a pharmaceutical composition for improving the physical function score of a human patient suffering from a disease or disorder by increasing that patient's serum bicarbonate value by at least 1 mEq/L over at least twelve weeks of treatment. In this embodiment, the composition: (a) is a nonabsorbable composition having the capacity to remove a target species from the patient selected from the group consisting of protons, strong acids, and conjugate bases of strong acids; (b) is characterized by a target species binding capacity of at least 3 mEq/g in a Simulated Small Intestine Inorganic Buffer (SIB) assay; and (c) has the capacity to improve the patient's physical function score based on answers to question 3 of the Kidney Disease Quality of Life Short Form (KDQOL-SF) compared to a placebo control in a statistically significant manner at the end of an at least the twelve-week period.

Another embodiment is a pharmaceutical composition for improving the physical function score of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in physical function score is a statistically significant improvement over a baseline physical function score based on answers to question 3 of the Kidney Disease Quality of Life Short Form (KDQOL-SF) compared to a placebo control at the end of the at least twelve-week period.

A further embodiment is a method of slowing the progression of kidney disease in a patient afflicted with chronic kidney disease and an acid-base disorder characterized by a baseline serum bicarbonate value of ≤22 mEq/L. In this embodiment, the method comprises oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

Another embodiment is a method of slowing the progression of kidney disease in a patient afflicted with chronic kidney disease and an acid-base disorder, wherein the patient has a baseline serum bicarbonate value of ≤22 mEq/L. This method comprises orally administering to the patient an effective amount of TRC101 once daily for a period of time sufficient to increase the patient's serum bicarbonate by at least 1 mEq/L.

Another embodiment is a method of slowing the progression of kidney disease in a patient afflicted with chronic kidney disease and metabolic acidosis disease. This method comprises administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to slow the progression of kidney disease.

A further embodiment is a pharmaceutical composition for slowing the progression of kidney disease in a human patient afflicted with chronic kidney disease and an acid-base disorder, the patient having a baseline serum bicarbonate level of ≤22 mEq/L prior to treatment. In this embodiment, the composition is a nonabsorbable composition having the capacity to: (a) remove a target species from the patient selected from the group consisting of protons, strong acids, and conjugate bases of strong acids; and (b) slow the progression of kidney disease in a human patient over at least a twelve-week period.

A further embodiment is a pharmaceutical composition for slowing the progression of kidney disease in a human patient afflicted with chronic kidney disease and an acid-base disorder by increasing that patient's serum bicarbonate value by at least 1 mEq/L over at least twelve weeks of treatment. In this embodiment, the composition: (a) is a nonabsorbable composition having the capacity to remove a target species from the patient selected from the group consisting of protons, strong acids, and conjugate bases of strong acids; (b) is characterized by a target species binding capacity of at least 3 mEq/g in a Simulated Small Intestine Inorganic Buffer (SIB) assay; and (c) has the capacity to slow the progression of kidney disease over at least the twelve-week period.

Another embodiment is a pharmaceutical composition for slowing the progression of kidney disease in a human patient also suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the progression of kidney disease in the patient is slowed over the twelve-week period compared to a placebo control group not receiving the pharmaceutical composition.

Another embodiment is a pharmaceutical composition for use in a method of treating an acid-base disorder in a patient, wherein the method of treatment improves the quality of life of the patient.

Yet another embodiment is a pharmaceutical composition for use in a method of treating an acid-base disorder in a patient, wherein the method of treatment improves the physical function of the patient.

De Brito-Ashurst et al. is one of six published prospective randomized, controlled clinical studies of alkali supplementation and dietary intervention, which demonstrate that increasing serum bicarbonate levels results in improved renal outcomes associated with chronic metabolic acidosis. The five other studies are: Garneata L, Stancu A, Dragomir D, et al., 2016, Ketoanalogue-Supplemented Vegetarian Very Low-Protein Diet and CKD Progression, J. Am. Soc. Nephrol. 27: 2164-2176; Phisitkul S, Khanna A, Simoni J, et al., 2010, Amelioration of metabolic acidosis in patients with low GFR reduced kidney endothelin production and kidney injury, and better preserved GFR, Kidney International 77: 617-623; Goraya N, Simoni J, Jo C, Wesson D, 2013, A comparison of treating metabolic acidosis in CKD stage 4 hypertensive kidney disease with fruits and vegetables or sodium bicarbonate, Clin. J. Am. Soc. Nephrol. 8: 371-381; Goraya N, Simoni J, Jo C, Wesson D, 2014, Treatment of metabolic acidosis in patients with stage 3 chronic kidney disease with fruits and vegetables or oral bicarbonate reduces urine angiotensinogen and preserves glomerular filtration rate, Kidney International 86: 1031-1038; and Mahajan A, Simoni J, Sheather S, et al., 2010, Daily oral sodium bicarbonate preserves glomerular filtration rate by slowing its decline in early hypertensive nephropathy, Kidney International 78: 303-309.

Garneata et al. assessed the effects of a ketoanalogue-supplemented vegetarian very low protein diet (0.3 g/kg/day) in diet-compliant patients to those of a usual mixed-source low protein diet (0.6 g/kg/day). Baseline serum bicarbonate was similar in the two treatment groups (16.7-16.8 mEq/L), however the end of study serum bicarbonate value was significantly higher in the vegetarian very low protein diet group than the usual mixed-source low protein diet group. Efficacy of the vegetarian very low protein diet to reduce incidence of renal events was most noted in patients with initial eGFR<20 mL/min.1.73 m².

In those embodiments in which the nonabsorbable composition binds chloride ions, it is generally preferred that the nonabsorbable composition selectively bind chloride ions relative to other physiologically significant competing anions such as bicarbonate equivalent anions, phosphate anions, and the conjugate bases of bile and fatty acids that are present in the GI tract. Stated differently, it is generally preferred that the nonabsorbable composition remove more chloride ions than any other competing anion in the GI tract.

In those embodiments in which the nonabsorbable composition binds protons, it is generally preferred that the nonabsorbable composition bind protons without delivering sodium, potassium, calcium, magnesium, and/or other electrolytes in exchange for the protons in an amount that is physiologically detrimental. As a result, treatment with the nonabsorbable composition will not significantly contribute to edema, hypertension, hyperkalemia, hypercalcemia or a similar disorder associated with an elevated load of sodium, potassium, calcium or other electrolyte. Similarly, in those embodiments in which the nonabsorbable composition binds protons, it is generally preferred that the nonabsorbable composition bind protons without removing an amount of sodium, potassium, calcium, magnesium and/or other electrolytes along with the protons. As a result, treatment with the nonabsorbable composition will not significantly contribute to hypotension, hypokalemia, hypocalcemia or other disorder associated with a depressed serum concentration of sodium, potassium, calcium, magnesium or other electrolyte.

In certain embodiments, the polymers preferably bind and maintain their ability to bind proton and anions at the physiological conditions found along the gastrointestinal (GI) lumen. These conditions can change according to dietary intake (see, for example, Fordtran J, Locklear T. Ionic constituents and osmolality of gastric and small-intestinal fluids after eating. Digest Dis Sci. 1966; 11(7):503-21) and location along the GI tract (Binder, H et al. Chapters 41-45 in “Medical Physiology”, 2nd Edition, Elsevier [2011]. Boron and Boulpaep [Ed.]). Rapid binding of proton and chloride in the stomach and small intestine is desirable. High binding levels and selectivity for chloride later in the GI tract (lower small intestine and large intestine) is also desirable. In general, the polymers also preferably have a pKₐ such that the majority of amines are protonated under the various pH and electrolyte conditions encountered along the GI tract and are thereby capable of removing proton, along with an appropriate counter anion (preferably chloride), from the body into the feces.

Since the stomach is an abundant source of HCl, and the stomach is the first site of potential HCl binding (after the mouth), and since residence time in the stomach is short (gastric residence half-life of approximately 90 minutes), compared to the rest of the GI tract (small intestine transit time of approximately 4 hours; whole gut transit time of 2-3 days; Read, N W et al. Gastroenterology [1980] 79:1276), it is desirable for the polymer of the present disclosure to demonstrate rapid kinetics of proton and chloride binding in the lumen of this organ, as well as in in vitro conditions designed to mimic the stomach lumen (e.g. SGF). Phosphate is a potential interfering anion for chloride binding in the stomach and small intestine, where phosphate is mostly absorbed (Cross, H S et al Miner Electrolyte Metab [1990] 16:115-24). Therefore rapid and preferential binding of chloride over phosphate is desirable in the small intestine and in in vitro conditions designed to mimic the small intestine lumen (e.g. SIB). Since the transit time of the colon is slow (2-3 days) relative to the small intestine, and since conditions in the colon will not be encountered by an orally administered polymer until after stomach and small intestine conditions have been encountered, kinetics of chloride binding by a polymer of the present disclosure do not have to be as rapid in the colon or in in vitro conditions designed to mimic the late small intestine/colon. It is, however, important that chloride binding and selectivity over other interfering anions is high, for example, at 24 and/or 48 hours or longer.

Other aspects and features will be in part apparent and in part pointed out hereinafter.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1A-1C is a flow chart schematically depicting the mechanism of action of the polymer when passing through the gastrointestinal tract of an individual from oral ingestion/stomach (FIG. 1A), to the upper GI tract (FIG. 1B) to the lower GI tract/colon (FIG. 1C).

FIG. 2 is a graph of the effect of TRC101 on serum bicarbonate in a rat model of adenine-induced nephropathy and metabolic acidosis in Part 1 of the study described in Example 1.

FIGS. 3A, 3B and 3C are graphs of the effect of TRC101 on fecal excretion of chloride (FIG. 3A), sulfate (FIG. 3B), and phosphate (FIG. 3C) in a rat model of adenine-induced nephropathy and metabolic acidosis in Part 1 of the study described in Example 1.

FIG. 4 is a graph of the effect of TRC101 on serum bicarbonate in a rat model of adenine-induced nephropathy and metabolic acidosis in Part 2 of the study described in Example 1.

FIGS. 5A, 5B and 5C are graphs of the effect of TRC101 on fecal excretion of chloride (FIG. 5A), sulfate (FIG. 5B), and phosphate (FIG. 5C) in a rat model of adenine-induced nephropathy and metabolic acidosis in Part 2 of the study described in Example 1.

FIGS. 6A, 6B and 6C are graphs of the in vivo chloride (FIG. 6A), sulfate (FIG. 6B) and phosphate (FIG. 6C) binding capacities of TRC101 and bixalomer in a pig with normal renal function in the study described in Example 2.

FIG. 7 is a line graph showing the mean change in serum bicarbonate (SBC) from baseline (BL) and standard error (SE) by treatment group over time in a human study as described more fully in Example 3 (Part 1).

FIG. 8 is a bar graph showing the least squares mean (LS Mean) change from baseline (CFB) to end of treatment in serum bicarbonate (SBC) by treatment group in a human study as described more fully in Example 3 (Part 1). Single asterisk (“*”) indicates statistically significant difference (p<0.5) and double asterisk (“**”) indicates highly statistically significant difference (p<0.0001).

FIG. 9 is a bar graph showing the effect on serum bicarbonate (SBC) levels and standard error (SE) at days 8 and 15 resulting from treatment (Tx=treatment) and upon withdrawal of TRC101 in a human study as described more fully in Example 3 (Part 1).

FIG. 10 is a line graph showing the mean change in serum bicarbonate (SBC) and standard error (SE) for the four TRC101 active arms and the two placebo arms (pooled) of the study described more fully in Example 3 (Parts 1 and 2).

FIG. 11 is a bar graph showing the least squares mean (LS Mean) change from baseline (CFB) in serum bicarbonate (SBC) by treatment group over time for the four TRC101 active arms and the two placebo arms (pooled) of the study described more fully in Example 3 (Parts 1 and 2). Single asterisk (“*”) indicates statistically significant difference (p<0.5) and double asterisk (“**”) indicates highly statistically significant difference (p<0.0001).

FIG. 12 is a bar graph showing the treatment effect on serum bicarbonate (SBC) levels and standard error (SE) at days 8 and 15 resulting from treatment (Tx=treatment) with and upon withdrawal of TRC101 in a human study as described more fully in Example 3 (Parts 1 and 2).

FIGS. 13A, 13B, 13C and 13D are graphs showing the changes in serum bicarbonate (FIG. 13A), serum chloride (FIG. 13B), serum sodium (FIG. 13C) and serum potassium (FIG. 13D) for the four TRC101 active arms (combined) vs the two placebo arms (pooled) over time for the study described more fully in Example 3 (Parts 1 and 2).

FIG. 14 is a graph showing the changes in the calculated anion gap for the four TRC101 active arms (combined) vs the two placebo arms (pooled) over time for the study described more fully in Example 3 (Parts 1 and 2).

FIG. 15 is a dataset analysis diagram and timeline, as described in greater detail in Example 4.

FIG. 16 is a population analysis flow chart, as described in greater detail in Example 4.

FIG. 17 is an illustration of the subpopulation used in the Cox Regression Analysis, as described in greater detail in Example 4.

FIG. 18 is an analysis diagram and timeline for the clinical trial as described in more detail in Example 5.

FIG. 19A is a graph showing the composite primary endpoint at the end of the treatment period for the clinical study described in more detail in Example 5.

FIG. 19B is a graph showing the achievement of serum bicarbonate thresholds at various time points for the clinical study described in more detail in Example 5.

FIG. 19C is a graph showing the change from baseline in serum bicarbonate over time at various time points for the clinical study described in more detail in Example 5.

FIGS. 20A-20B are graphs showing that TRC101-treated subjects experienced a statistically significant improvement in quality of life, particularly, in physical function, based on results from Question #3 of the KDQOL-SF survey for the clinical study described in more detail in Example 5.

FIG. 21 is a copy of Question #3 of the KDQOL-SF survey for the clinical study described in Example 5. The score conversion is as follows: 1 (limited a lot)=0; 2 (limited a little)=50; 3 (not limited)=100. Total score=sum of all 10, divided by 10.

FIG. 22A is a copy of the Single Chair Stand and Repeated Chair Stand protocols, including the scoring criteria (FIG. 22B), as described in more detail in Example 5.

FIG. 23 is table showing the analysis from baseline in total score in kidney disease and quality of life (Question 3) at week 12, as described in more detail in Example 5.

FIG. 24 is a table showing the analysis from baseline in time (seconds) of completing repeated chair stand at the end of week 12, as described in more detail in Example 5.

FIG. 25 is a diagram showing the overall design of the Retrospective Model, as described in more detail in Example 4.

FIG. 26 is a graph showing the time to first occurrence of DD40 endpoint, as described in more detail in Example 4.

FIG. 27 is a table showing the differences in outcome of TRC101 treated patients against placebo treated patients in the combined TRCA-301 and TRCA-301E 52 week study, as described in more detail in Example 6.

FIG. 28 is a graph showing SBC durability effect for TRC101-treated patients against placebo treated patients at the end of the TRCA-301 and TRCA-301E 52 week study, as described in more detail in Example 6.

FIG. 29 is a graph showing the mean change from baseline in serum bicarbonate level for TRC101 treated patients and placebo treated patients at various time points across the combined TRCA-301 and TRCA-301E 52 week study, as described in more detail in Example 6.

FIG. 30 is a graph showing the mean change from baseline in KDQOL-Physical Functioning Domain for TRC101 treated patients and placebo treated patients across the TRCA-301 and TRCA-301E 52 week study, as described in more detail in Example 6.

FIG. 31 is a graph showing the mean change from baseline in time to perform the repeated chair stand test for TRC101 treated patients and placebo treated patients at various time points in the TRCA-301 and TRCA-301E 52 week study, as described in more detail in Example 6.

FIG. 32 is a table showing the difference between active vs placebo for adverse effects occurring at ≥2% of the proportions of patients affected for TRC101-treated patients and placebo-treated patients in the TRCA-301 and TRCA-301E 52 week study, as described in more detail in Example 6.

FIG. 33 is a table showing the adverse events occurring at ≥5% of the study populations and the proportions of TRC101-treated patients and placebo-treated patients in the TRCA-301 and TRCA-301E 52 week study, as described in more detail in Example 6.

FIG. 34 is a table summarizing the number of withdrawals in both TRC101-treated patients and placebo-treated patients across the TRCA-301 and the TRCA-301E studies, as described in more detail in Example 6.

FIG. 35 is a table showing the incidences of death, dialysis, ≥50% eGFR decline and DD50 across the TRCA-301 and the TRCA-301E study for TRC101-treated patients and placebo-treated patients, without annualising the incidence rate.

FIG. 36 is a table showing the annualised incidence rate of death, death/dialysis and DD50 across the TRCA-301 and the TRCA-301E study for TRC101-treated patients and placebo-treated patients.

### DETAILED DESCRIPTION OF THE INVENTION

### Abbreviations and Definitions

The following definitions and methods are provided to better define the present invention and to guide those of ordinary skill in the art in the practice of the present invention. Unless otherwise noted, terms are to be understood according to conventional usage by those of ordinary skill in the relevant art.

The term “absorption capacity” as used herein in connection with a polymer and a swelling agent (or in the case of a mixture of swelling agents, the mixture of swelling agents) is the amount of the swelling agent (or such mixture) absorbed during a period of at least 16 hours at room temperature by a given amount of a dry polymer (e.g., in the form of a dry bead) immersed in an excess amount of the swelling agent (or such mixture).

The term “acrylamide” denotes a moiety having the structural formula H₂C═CH—C(O)NR—*, where * denotes the point of attachment of the moiety to the remainder of the molecule and R is hydrogen, hydrocarbyl, or substituted hydrocarbyl.

The term “acrylic” denotes a moiety having the structural formula H₂C═CH—C(O)O—*, where * denotes the point of attachment of the moiety to the remainder of the molecule.

The term “adult” refers to an individual over 18 years of age.

The term “alicyclic”, “alicyclo” or “alicyclyl” means a saturated monocyclic group of 3 to 8 carbon atoms and includes cyclopentyl, cyclohexyl, cycloheptyl, and the like.

The term “aliphatic” denotes saturated and non-aromatic unsaturated hydrocarbyl moieties having, for example, one to about twenty carbon atoms or, in specific embodiments, one to about twelve carbon atoms, one to about ten carbon atoms, one to about eight carbon atoms, or even one to about four carbon atoms. The aliphatic groups include, for example, alkyl moieties such as methyl, ethyl, n-propyl, isopropyl, n-butyl, isobutyl, sec-butyl, tert-butyl, pentyl, iso-amyl, hexyl and the like, and alkenyl moieties of comparable chain length.

The term “alkanol” denotes an alkyl moiety that has been substituted with at least one hydroxyl group. In some embodiments, alkanol groups are “lower alkanol” groups comprising one to six carbon atoms, one of which is attached to an oxygen atom. In other embodiments, lower alkanol groups comprise one to three carbon atoms.

The term “alkenyl group” encompasses linear or branched carbon radicals having at least one carbon-carbon double bond. The term “alkenyl group” can encompass conjugated and non-conjugated carbon-carbon double bonds or combinations thereof. An alkenyl group, for example and without being limited thereto, can encompass two to about twenty carbon atoms or, in a particular embodiment, two to about twelve carbon atoms. In certain embodiments, alkenyl groups are “lower alkenyl” groups having two to about four carbon atoms. Examples of alkenyl groups include, but are not limited thereto, ethenyl, propenyl, allyl, vinyl, butenyl and 4-methylbutenyl. The terms “alkenyl group” and “lower alkenyl group”, encompass groups having “cis” or “trans” orientations, or alternatively, “E” or “Z” orientations.

The term “alkyl group” as used, either alone or within other terms such as “haloalkyl group,” “aminoalkyl group” and “alkylamino group”, encompasses saturated linear or branched carbon radicals having, for example, one to about twenty carbon atoms or, in specific embodiments, one to about twelve carbon atoms. In other embodiments, alkyl groups are “lower alkyl” groups having one to about six carbon atoms. Examples of such groups include, but are not limited thereto, methyl, ethyl, n-propyl, isopropyl, n-butyl, isobutyl, sec-butyl, tert-butyl, pentyl, iso-amyl, hexyl and the like. In more specific embodiments, lower alkyl groups have one to four carbon atoms.

The term “alkylamino group” refers to amino groups directly attached to the remainder of the molecule via the nitrogen atom of the amino group and wherein the nitrogen atom of the alkylamino group is substituted by one or two alkyl groups. In some embodiments, alkylamino groups are “lower alkylamino” groups having one or two alkyl groups of one to six carbon atoms, attached to a nitrogen atom. In other embodiments, lower alkylamino groups have one to three carbon atoms. Suitable “alkylamino” groups may be mono or dialkylamino such as N-methylamino, N-ethylamino, N,N-dimethylamino, N,N-diethylamino, pentamethyleneamine and the like.

The term “allyl” denotes a moiety having the structural formula H₂C═CH—CH₂—*, where * denotes the point of attachment of the moiety to the remainder of the molecule and the point of attachment is to a heteroatom or an aromatic moiety.

The term “allylamine” denotes a moiety having the structural formula H₂C═CH—CH₂N(X₈)(X₉), wherein X₈ and X₉ are independently hydrogen, hydrocarbyl, or substituted hydrocarbyl, or X₈ and X₉ taken together form a substituted or unsubstituted alicyclic, aryl, or heterocyclic moiety, each as defined in connection with such term, typically having from 3 to 8 atoms in the ring.

The term “amine” or “amino” as used alone or as part of another group, represents a group of formula —N(X₈)(X₉), wherein X₈ and X₉ are independently hydrogen, hydrocarbyl, or substituted hydrocarbyl, heteroaryl, or heterocyclo, or X₈ and X₉ taken together form a substituted or unsubstituted alicyclic, aryl, or heterocyclic moiety, each as defined in connection with such term, typically having from 3 to 8 atoms in the ring.

The term “aminoalkyl group” encompasses linear or branched alkyl groups having one to about ten carbon atoms, any one of which may be substituted with one or more amino groups, directly attached to the remainder of the molecule via an atom other than a nitrogen atom of the amine group(s). In some embodiments, the aminoalkyl groups are “lower aminoalkyl” groups having one to six carbon atoms and one or more amino groups. Examples of such groups include aminomethyl, aminoethyl, aminopropyl, aminobutyl and aminohexyl.

The terms “anion exchange material” and “cation exchange material” take their normal meaning in the art. For example, the terms “anion exchange material” and “cation exchange material” refer to materials that exchange anions and cations, respectively. Anion and cation exchange materials are typically water-insoluble substances which can exchange some of their cations or anions, respectively, for similarly charged anions or cations contained in a medium with which they are in contact. Anion exchange materials may contain positively charged groups, which are fixed to the backbone materials and allow passage of anions but reject cations. A non-exhaustive list of such positively charged groups includes: amino group, alkyl substituted phosphine, and alkyl substituted sulphides. A non-exhaustive list of cation or anion exchange materials includes: clays (e.g., bentonite, kaolinite, and illite), vermiculite, zeolites (e.g., analcite, chabazite, sodalite, and clinoptilolite), synthetic zeolites, polybasic acid salts, hydrous oxides, metal ferrocyanides, and heteropolyacids. Cation exchange materials can contain negatively charged groups fixed to the backbone material, which allow the passage of cations but reject anions. A non-exhaustive list of such negatively charged groups includes: sulphate, carboxylate, phosphate, and benzoate.

The term “aromatic group” or “aryl group” means an aromatic group having one or more rings wherein such rings may be attached together in a pendent manner or may be fused. In particular embodiments, an aromatic group is one, two or three rings. Monocyclic aromatic groups may contain 5 to 10 carbon atoms, typically 5 to 7 carbon atoms, and more typically 5 to 6 carbon atoms in the ring. Typical polycyclic aromatic groups have two or three rings. Polycyclic aromatic groups having two rings typically have 8 to 12 carbon atoms, preferably 8 to 10 carbon atoms in the rings. Examples of aromatic groups include, but are not limited to, phenyl, naphthyl, tetrahydronaphthyl, indanyl, biphenyl, phenanthryl, anthryl or acenaphthyl.

The term “bead” is used to describe a crosslinked polymer that is substantially spherical in shape.

The term “bicarbonate equivalent” is used to describe an organic acid or anion that yields bicarbonate when metabolized. Citrate and succinate are exemplary bicarbonate equivalents.

The term “binds” as used herein in connection with a polymer and one or more ions, that is, a cation (e.g. “proton-binding” polymer) and an anion, is an “ion-binding” polymer and/or when it associates with the ion, generally though not necessarily in a non-covalent manner, with sufficient association strength that at least a portion of the ion remains bound under the in vitro or in vivo conditions in which the polymer is used for sufficient time to effect a removal of the ion from solution or from the body.

The term “ceramic material” takes its normal meaning in the art. In certain embodiments, the term “ceramic material” refers to an inorganic, nonmetallic, solid material comprising metal, nonmetal or metalloid atoms primarily held in ionic and covalent bonds. A non-exhaustive list of examples of ceramic materials includes: barium titanate, bismuth strontium calcium copper oxide, boron oxide, earthenware, ferrite, lanthanum carbonate, lead zirconate, titanate, magnesium diboride, porcelain, sialon, silicon carbide, silicon nitride, titanium carbide, yttrium barium copper oxide, zinc oxide, zirconium dioxide, and partially stabilised zirconia. In certain embodiments, the term “clinically significant increase” as used herein in connection with a treatment refers to a treatment that improves or provides a worthwhile change in an individual from a dysfunctional state back to a relatively normal functioning state, or moves the measurement of that state in the direction of normal functioning, or at least a marked improvement to untreated. A number of methods can be used to calculate clinical significance. A non-exhaustive list of methods for calculating clinical significance includes: Jacobson-Truax, Gulliksen-Lord-Novick, Edwards-Nunnally, Hageman-Arrindell, and Hierarchical Linear Modeling (HLM).

The term “crosslink density” denotes the average number of connections of the amine containing repeat unit to the rest of the polymer. The number of connections can be 2, 3, 4 and higher. Repeat units in linear, non-crosslinked polymers are incorporated via 2 connections. To form an insoluble gel, the number of connections should be greater than 2. Low crosslinking density materials such as Sevelamer have on average about 2.1 connections between repeat units. More crosslinked systems such as bixalomer have on average about 4.6 connections between the amine-containing repeat units. “Crosslinking density” represents a semi-quantitative measure based on the ratios of the starting materials used. Limitations include the fact that it does not account for different crosslinking and polymerization methods. For example, small molecule amine systems require higher amounts of crosslinker as the crosslinker also serves as the monomer to form the polymer backbone whereas for radical polymerizations the polymer chain is formed independent from the crosslinking reaction. This can lead to inherently higher crosslinking densities under this definition for the substitution polymerization/small molecule amines as compared to radical polymerization crosslinked materials.

The term “crosslinker” as used, either alone or within other terms, encompasses hydrocarbyl or substituted hydrocarbyl, linear or branched molecules capable of reacting with any of the described monomers, or the infinite polymer network, as described in Formula 1, more than one time. The reactive group in the crosslinker can include, but is not limited to alkyl halide, epoxide, phosgene, anhydride, carbamate, carbonate, isocyanate, thioisocyanate, esters, activated esters, carboxylic acids and derivatives, sulfonates and derivatives, acyl halides, aziridines, α,β-unsaturated carbonyls, ketones, aldehydes, pentafluoroaryl groups, vinyl, allyl, acrylate, methacrylate, acrylamide, methacrylamide, styrenic, acrylonitriles and combinations thereof. In one exemplary embodiment, the crosslinker's reactive group will include alkyl halide, epoxide, anhydrides, isocyanates, allyl, vinyl, acrylamide, and combinations thereof. In one such embodiment, the crosslinker's reactive group will be alkyl halide, epoxide, or allyl.

The term “diallylamine” denotes an amino moiety having two allyl groups.

The terms “dry bead” and “dry polymer” refer to beads or polymers that contain no more than 5% by weight of a non-polymer swelling agent or solvent. Often the swelling agent/solvent is water remaining at the end of a purification. This is generally removed by lyophilization or oven drying before storage or further crosslinking of a preformed amine polymer. The amount of swelling agent/solvent can be measured by heating (e.g., heating to 100-200° C.) and measuring the resulting change in weight. This is referred to a “loss on drying” or “LOD.”

The term “estimated glomerular filtration rate” or eGFR refers to an estimate of the glomerular filtration rate and is estimated from the serum level of an endogenous filtration marker. Creatinine is a commonly used endogenous filtration marker in clinical practice and several equations have been proposed for estimating the glomerular filtration rate. As used herein, all eGFR values may be determined according to the CKD-EPI equation (Levey et al., A New Equation to Estimate Glomerular Filtration Rate. Ann Intern Med. 2009; 150:604-612):

GFR=41*min(Scr/κ,1)^{α}*max(Scr/κ,1)^{-1.209}*0.993^{Age}*1.018 [if female]*1.159 [if black]

wherein Scr is serum creatinine (mg/dL), κ is 0.7 for females and 0.9 for males, a is −0.329 for females and −0.411 for males, min indicates the minimum of Scr/κ or 1, and max indicates the maximum of Scr/κ or 1.

The term “ethereal” denotes a moiety having an oxygen bound to two separate carbon atoms as depicted the structural formula *−HₓCO—CHₓ—*, where * denotes the point of attachment to the remainder of the moiety and x independently equals 0, 1, 2, or 3.

The term “gel” is used to describe a crosslinked polymer that has an irregular shape.

The term “glomerular filtration rate” or GFR is the volume of fluid filtered from the renal (kidney) glomerular capillaries into the Bowman's capsule per unit time. GFR cannot be measured directly; instead, it is measured indirectly (mGFR) as the clearance of an exogenous filtration marker (e.g., inulin, iothalamate, iohexol, etc.) or estimated (eGFR) using an endogenous filtration marker.

The term “halo” means halogens such as fluorine, chlorine, bromine or iodine atoms.

The term “haloalkyl group” encompasses groups wherein any one or more of the alkyl carbon atoms is substituted with halo as defined above. Specifically encompassed are monohaloalkyl, dihaloalkyl and polyhaloalkyl groups including perhaloalkyl. A monohaloalkyl group, for example, may have either an iodo, bromo, chloro or fluoro atom within the group. Dihalo and polyhaloalkyl groups may have two or more of the same halo atoms or a combination of different halo groups. “Lower haloalkyl group” encompasses groups having 1-6 carbon atoms. In some embodiments, lower haloalkyl groups have one to three carbon atoms. Examples of haloalkyl groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaliphatic” describes a chain of 1 to 25 carbon atoms, typically 1 to 12 carbon atoms, more typically 1 to 10 carbon atoms, and most typically 1 to 8 carbon atoms, and in some embodiments 1 to 4 carbon atoms that can be saturated or unsaturated (but not aromatic), containing one or more heteroatoms, such as halogen, oxygen, nitrogen, sulfur, phosphorus, or boron. A heteroatom atom may be a part of a pendant (or side) group attached to a chain of atoms (e.g., —CH(OH)—CH(NH₂)— where the carbon atom is a member of a chain of atoms) or it may be one of the chain atoms (e.g., —ROR— or —RNHR— where each R is aliphatic). Heteroaliphatic encompasses heteroalkyl and heterocyclo but does not encompass heteroaryl.

The term “heteroalkyl” describes a fully saturated heteroaliphatic moiety.

The term “heteroaryl” means a monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. As defined herein, the terms “heteroaryl” and “aryl” are mutually exclusive. “Heteroarylene” means a divalent heteroaryl radical.

The term “heteroatom” means an atom other than carbon and hydrogen. Typically, but not exclusively, heteroatoms are selected from the group consisting of halogen, sulfur, phosphorous, nitrogen, boron and oxygen atoms. Groups containing more than one heteroatom may contain different heteroatoms.

The term “heterocyclo,” “heterocyclic,” or heterocyclyl” means a saturated or unsaturated group of 4 to 8 ring atoms in which one or two ring atoms are heteroatom such as N, O, B, P and S(O)ₙ, where n is an integer from 0 to 2, the remaining ring atoms being carbon. Additionally, one or two ring carbon atoms in the heterocyclyl ring can optionally be replaced by a —C(O)— group. More specifically the term heterocyclyl includes, but is not limited to, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group.

The term “hydrocarbon group” or “hydrocarbyl group” means a chain of 1 to 25 carbon atoms, typically 1 to 12 carbon atoms, more typically 1 to 10 carbon atoms, and most typically 1 to 8 carbon atoms. Hydrocarbon groups may have a linear or branched chain structure. Typical hydrocarbon groups have one or two branches, typically one branch. Typically, hydrocarbon groups are saturated. Unsaturated hydrocarbon groups may have one or more double bonds, one or more triple bonds, or combinations thereof. Typical unsaturated hydrocarbon groups have one or two double bonds or one triple bond; more typically unsaturated hydrocarbon groups have one double bond.

“Initiator” is a term used to describe a reagent that initiates a polymerization.

The term “measured glomerular filtration rate” or “mGFR” refers to a measurement of the glomerular filtration rate using any chemical (e.g., inulin, iothalamate, iohexol, etc.) that has a steady level in the blood, and is freely filtered but neither reabsorbed nor secreted by the kidneys according to standard technique.

The term “Michael acceptor” takes its normal meaning in the art. In certain embodiments the term “Michael acceptor” refers to activated olefins, such as α,β-unsaturated carbonyl compounds. A Michael acceptor can be a conjugated system with an electron withdrawing group, such as cyano, keto or ester. A non-exhaustive list of examples of Michael acceptors includes: vinyl ketones, alkyl acrylates, acrylo nitrile, and fumarates.

The term “molecular weight per nitrogen” or “MW/N” represents the calculated molecular weight in the polymer per nitrogen atom. It represents the average molecular weight to present one amine function within the crosslinked polymer. It is calculated by dividing the mass of a polymer sample by the moles of nitrogen present in the sample. “MW/N” is the inverse of theoretical capacity, and the calculations are based upon the feed ratio, assuming full reaction of crosslinker and monomer. The lower the molecular weight per nitrogen the higher the theoretical capacity of the crosslinked polymer.

The term “nonabsorbable” as used herein takes its normal meaning in the art. Therefore, if something is nonabsorbable it is not absorbed during its passage through the human GI tract. This could be measured by any appropriate means. One option known to the skilled person would be to examine faeces to see if the nonabsorbable material is recovered after passing through the GI tract. As a practical matter, the amount of a nonabsorbable material recovered in this scenario will never be 100% of the material administered. For example, about 90-99% of the material might be recovered from the faeces. Another option known to the skilled person would be to look for the presence of the material in the lymph, blood, interstitial fluid, secretions from various organs (e.g., pancreas, liver, gut, etc.) or in the body of organs (e.g., liver, kidney, lungs, etc.) as oral administration of a nonabsorbable material would not result in an increase in the amount of that material in these matrices and tissues. Nonabsorbable compositions may be particulate compositions that are essentially insoluble in the human GI tract and have a particle size that is large enough to avoid passive or active absorption through the human GI tract. As an example, nonabsorbable compositions is meant to imply that the substance does not enter the lymph, blood, interstitial fluids or organs through the main entry points of the human GI tract, namely by paracellular entry between gut epithelial cells, by endocytic uptake through gut epithelial cells, or through entry via M cells comprising the gut epithelial antigen sampling and immune surveillance system (Jung, 2000), either through active or passive transport processes. There is a known size limit for a particulate to be absorbed in the human GI tract (Jung et al., European Journal of Pharmaceutics and Biopharmaceutics 50 (2000) 147-160; Jani et al., International Journal of Pharmaceutics, 84 (1992) 245-252; and Jani et al., J. Pharm. Pharmacol. 1989, 41:809-812), so the skilled person would know that materials that, when in the GI tract, have a size of at least 1 micrometers would be nonabsorbable.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes embodiments in which the heterocyclyl group is substituted with an alkyl group and embodiments in which the heterocyclyl group is not substituted with alkyl.

“Particle size” is measured by wet laser diffraction using Mie theory. Particles are dispersed in an appropriate solvent, such as water or methanol, and added to the sample chamber to achieve red channel obscuration of 10-20%. Sonication may be performed, and a dispersing agent, such as a surfactant (e.g. Tween-80), may be added in order to disrupt weak particle-particle interactions. The refractive index setting of the particles used for size distribution calculation is selected to minimize artifacts in the results and the R parameter value, determined by the laser diffraction software. The D(0.1), D(0.5), and D(0.9) values characterizing the particle size distribution by volume-basis are recorded.

“Pharmaceutically acceptable” as used in connection with a carrier, diluent or excipient means a carrier, diluent or an excipient, respectively, that is useful in preparing a pharmaceutical composition that is generally safe, non-toxic and neither biologically nor otherwise undesirable for veterinary use and/or human pharmaceutical use.

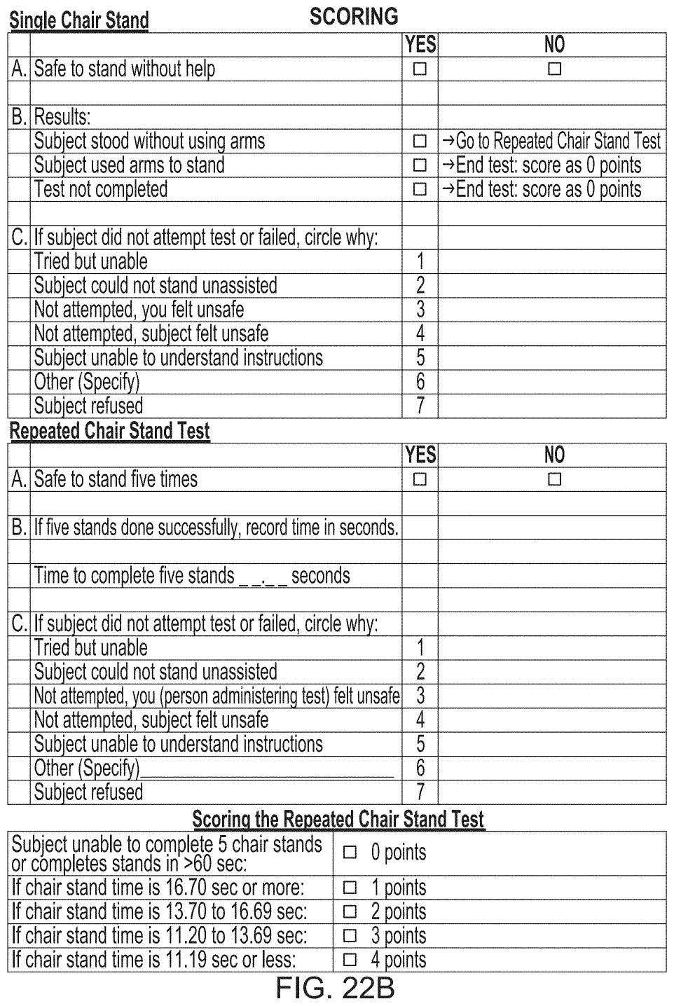

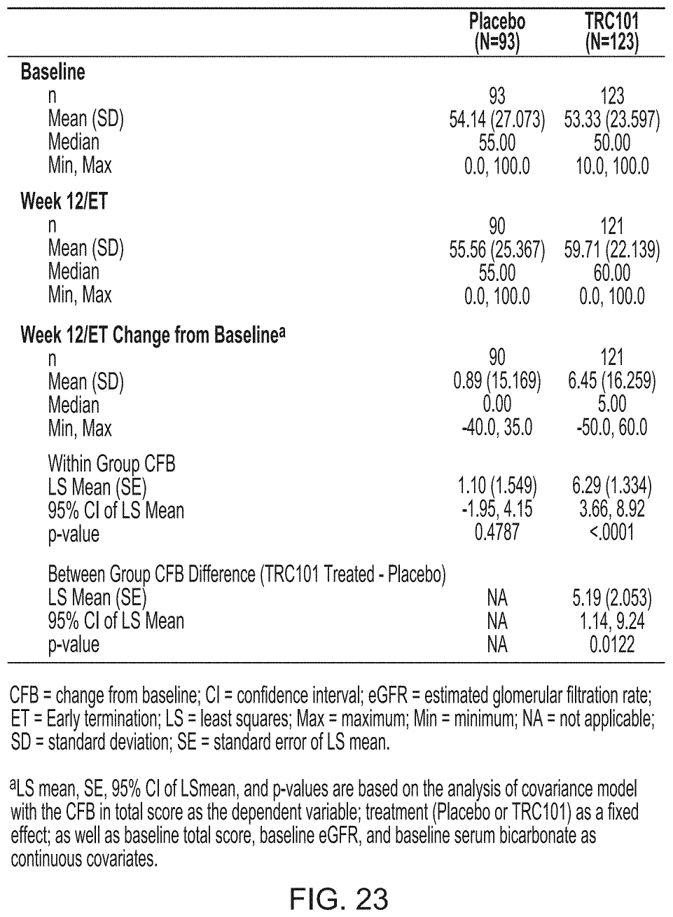

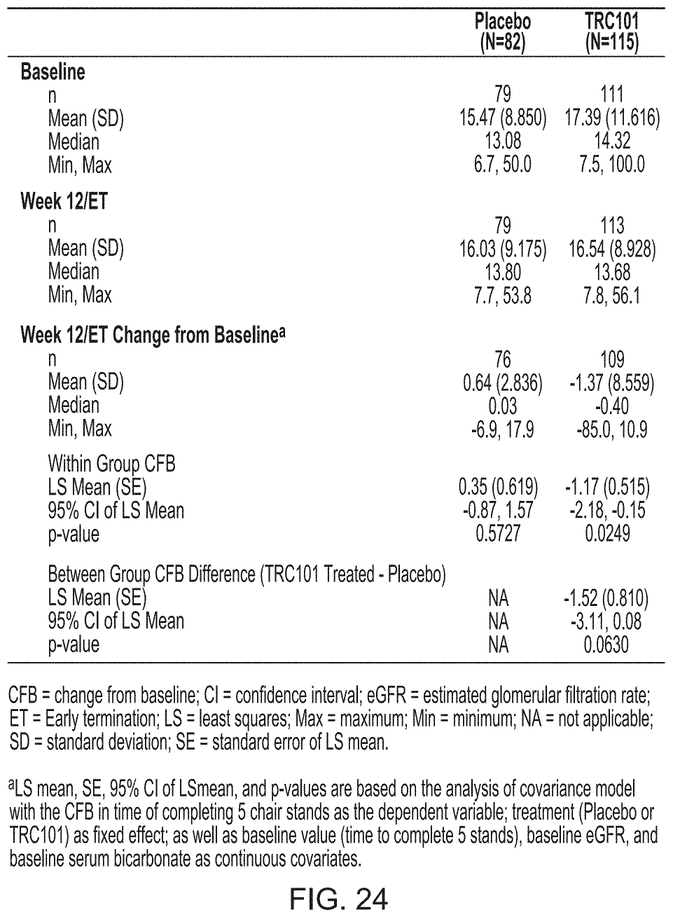

The term “physical function” as used herein in connection with a patient afflicted with chronic kidney disease and an acid-base disorder may be assessed using (i) the Kidney Disease and Quality of Life (KDQOL) Short Form-36, Question 3 (Physical Functioning Domain) as illustrated in FIG. 22A & 22B and Example 5, or (iii) both the KDQOL Short Form-36 Question 3 and the standardized repeated chair stand test (i.e., “i” and “ii” of this paragraph).

The term “post polymerization crosslinking” is a term that describes a reaction to an already formed bead or gel, where more crosslinking is introduced to the already formed bead or gel to create a bead or gel that has an increased amount of crosslinking.

The term “post polymerization modification” is a term that describes a modification to an already formed bead or gel, where a reaction or a treatment introduces an additional functionality. This functionality can be linked either covalently or non-covalently to the already formed bead.

The term “quaternized amine assay” (“QAA”) describes a method to estimate the amount of quaternary amines present in a given crosslinked polymer sample. This assay measures chloride binding of a crosslinked polymer at a pH of 11.5. At this pH, primary, secondary and tertiary amines are not substantially protonated and do not substantially contribute to chloride binding. Therefore, any binding observed under these conditions can be attributed to the presence of permanently charged quaternary amines. The test solution used for QAA assay is 100 mM sodium chloride at a pH of 11.5. The concentration of chloride ions is similar to that in the SGF assay which is used to assess total binding capacity of crosslinked polymers. Quaternary amine content as a percentage of total amines present is calculated as follows:

$$\% Quaternary amines = \frac{Chloride bound ( mmol / g ) in QAA}{Chloride bound ( mmol / g ) in S GF} \times 100$$

To perform the QAA assay, the free-amine polymer being tested is prepared at a concentration of 2.5 mg/ml (e.g. 25 mg dry mas) in 10 mL of QAA buffer. The mixture is incubated at 37° C. for ˜16 hours with agitation on a rotisserie mixer. After incubation and mixing, 600 microliters of supernatant is removed and filtered using a 800 microliter, 0.45 micrometer pore size, 96-well poly propylene filter plate. With the samples arrayed in the filter plate and the collection plate fitted on the bottom, the unit is centrifuged at 1000×g for 1 minute to filter the samples. After filtration into the collection plate, the respective filtrates are diluted appropriately before measuring for chloride content. The IC method (e.g. ICS-2100 Ion Chromatography, Thermo Fisher Scientific) used for the analysis of chloride content in the filtrates consists of a 15 mM KOH mobile phase, an injection volume of 5 microliters, with a run time of three minutes, a washing/rinse volume of 1000 microliters, and flow rate of 1.25 mL/min. To determine the chloride bound to the polymer, the following calculation is completed:

$$Binding capacity expressed as mmol chloride / g dry polymer = \frac{( C l start - C l eq )}{2.5}$$

where Cl start corresponds to the starting concentration of chloride in the QAA buffer, Cl eq corresponds to the equilibrium value of chloride in the measured filtrates after exposure to the test polymer, and 2.5 is the polymer concentration in mg/ml.

The terms “short chain carboxylic acid” or “short chain fatty acid” take their normal meaning in the art. In certain embodiments, the terms “short chain carboxylic acid” or “short chain fatty acid” refer to carboxylic acids having a chain length of 0, 1, 2, 3, 4, 5 or 6 carbon atoms long. A non-exhaustive list of examples of short chain carboxylic acids includes: formic acid, acetic acid, propionic acid, butyric acid, isobutyric acid, valeric acid, isovaleric acid, and lactic acid.

“Simulated Gastric Fluid” or “SGF” Assay describes a test to determine total chloride binding capacity for a test polymer using a defined buffer that simulates the contents of gastric fluid as follows: Simulated gastric fluid (SGF) consists of 35 mM NaCl, 63 mM HCl, pH 1.2. To perform the assay, the free-amine polymer being tested is prepared at a concentration of 2.5 mg/ml (25 mg dry mass) in 10 mL of SGF buffer. The mixture is incubated at 37° C. overnight for ˜12-16 hours with agitation on a rotisserie mixer. Unless another time period is otherwise stated, SGF binding data or binding capacities recited herein are determined in a time period of this duration. After incubation and mixing, the tubes containing the polymer are centrifuged for 2 minutes at 500-1000×g to pellet the test samples. Approximately 750 microliters of supernatant are removed and filtered using an appropriate filter, for example a 0.45 micrometer pore-size syringe filter or an 800 microliter, 1 micrometer pore-size, 96-well, glass filter plate that has been fitted over a 96-well 2 mL collection plate. With the latter arrangement, multiple samples tested in SGF buffer can be prepared for analysis, including the standard controls of free amine Sevelamer, free amine bixalomer and a control tube containing blank buffer that is processed through all of the assay steps. With the samples arrayed in the filter plate and the collection plate fitted on the bottom, the unit is centrifuged at 1000×g for 1 minute to filter the samples. In cases of small sample sets, a syringe filter may be used in lieu of the filter plate, to retrieve ˜2-4 mL of filtrate into a 15 mL container. After filtration, the respective filtrates are diluted 4× with water and the chloride content of the filtrate is measured via ion chromatography (IC). The IC method (e.g. Dionex ICS-2100, Thermo Scientific) consists of an AS11 column and a 15 mM KOH mobile phase, an injection volume of 5 microliters, with a run time of 3 minutes, a washing/rinse volume of 1000 microliters, and flow rate of 1.25 mL/min. To determine the chloride bound to the polymer, the following calculation is completed:

$$\frac{( C l start - C l eq ) \times 4}{2.5} .$$

Binding capacity expressed as mmol chloride/g polymer: where Cl start corresponds to the starting concentration of chloride in the SGF buffer, Cl eq corresponds to the equilibrium value of chloride in the diluted measured filtrates after exposure to the test polymer, 4 is the dilution factor and 2.5 is the polymer concentration in mg/ml.

“Simulated Small Intestine Inorganic Buffer” or “SIB” is a test to determine the chloride and phosphate binding capacity of free amine test polymers in a selective specific interfering buffer assay (SIB). The chloride and phosphate binding capacity of free amine test polymers, along with the chloride and phosphate binding capacity of free amine Sevelamer and bixalomer control polymers, was determined using the selective specific interfering buffer assay (SIB) as follows: The buffer used for the SIB assay comprises 36 mM NaCl, 20 mM NaH₂PO₄, 50 mM 2-(N-morpholino)ethanesulfonic acid (MES) buffered to pH 5.5. The SIB buffer contains concentrations of chloride, phosphate and pH that are present in the human duodenum and upper gastrointestinal tract (Stevens T, Conwell D L, Zuccaro G, Van Lente F, Khandwala F, Purich E, et al. Electrolyte composition of endoscopically collected duodenal drainage fluid after synthetic porcine secretin stimulation in healthy subjects. Gastrointestinal endoscopy. 2004; 60(3):351-5, Fordtran J, Locklear T. Ionic constituents and osmolality of gastric and small-intestinal fluids after eating. Digest Dis Sci. 1966; 11(7):503-21) and is an effective measure of the selectivity of chloride binding compared to phosphate binding by a polymer. To perform the assay, the free amine polymer being tested is prepared at a concentration of 2.5 mg/ml (25 mg dry mass) in 10 mL of SIB buffer. The mixture is incubated at 37° C. for 1 hour with agitation on a rotisserie mixer. Unless another time period is otherwise stated, SIB binding data or binding capacities recited herein are determined in a time period of this duration. After incubation and mixing, the tubes containing the polymer are centrifuged for 2 minutes at 1000λg to pellet the test samples. 750 microliter of supernatant is removed and filtered using an 800 microliter, 1 micrometer pore-size, 96-well, glass filter plate that has been fitted over a 96-well 2 mL collection plate; with this arrangement multiple samples tested in SIB buffer can be prepared for analysis, including the standard controls of free amine Sevelamer, free amine bixalomer and a control tube containing blank buffer that is processed through all of the assay steps. With the samples arrayed in the filter plate and the collection plate fitted on the bottom, the unit is centrifuged at 1000×g for 1 minute to filter the samples. In cases of small sample sets, a syringe filter (0.45 micrometer) may be used in lieu of the filter plate, to retrieve ˜2-4 mL of filtrate into a 15 mL vial. After filtration into the collection plate, the respective filtrates are diluted before measuring for chloride or phosphate content. For the measurement of chloride and phosphate, the filtrates under analysis are diluted 4× with water. The chloride and phosphate content of the filtrate is measured via ion chromatography (IC). The IC method (e.g. Dionex ICS-2100, Thermo Scientific) consists of an AS24A column, a 45 mM KOH mobile phase, an injection volume of 5 microliters, with a run time of about 10 minutes, a washing/rinse volume of 1000 microliter, and flow rate of 0.3 mL/min. To determine the chloride bound to the polymer, the following calculation is completed:

$$Binding capacity expressed as mmol chloride / g polymer = \frac{( {Cl}_{start} - {Cl}_{final} ) \times 4}{2.5}$$

where CIₛₜₐᵣₜ corresponds to the starting concentration of chloride in the SIB buffer, CI_{final} corresponds to the final value of chloride in the measured diluted filtrates after exposure to the test polymer, 4 is the dilution factor and 2.5 is the polymer concentration in mg/ml. To determine the phosphate bound to the polymer, the following calculation is completed:

$$Binding capacity expressed as mmol phosphate / g polymer = \frac{( {P }_{start} - {P}_{final} ) \times 4}{2.5}$$

where Pₛₜₐᵣₜ corresponds to the starting concentration of phosphate in the SIB buffer, P_{final} corresponds to the final value of phosphate in the measured diluted filtrates after exposure to the test polymer, 4 is the dilution factor and 2.5 is the polymer concentration in mg/ml.

In certain embodiments, the term “statistically significant” refers to the likelihood that a relationship between two or more variables is caused by something other than random chance. More precisely, the significance level, α, defined for a study is the probability of the study rejecting the null hypothesis, given that it were true, and the p-value, p, of a result is the probability of obtaining a result at least as extreme, given that the null hypothesis were true. The result is statistically significant, by the standards of the study, when p<α. The significance level for a study is chosen before data collection, and typically set to 5%.

The term “substituted hydrocarbyl,” “substituted alkyl,” “substituted alkenyl,” “substituted aryl,” “substituted heterocyclo,” or “substituted heteroaryl” as used herein denotes hydrocarbyl, alkyl, alkenyl, aryl, heterocyclo, or heteroaryl moieties which are substituted with at least one atom other than carbon and hydrogen, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, keto, acyl, acyloxy, nitro, amino, amido, nitro, cyano, thiol, ketals, acetals, esters and ethers.

“Swelling Ratio” or simply “Swelling” describes the amount of water absorbed by a given amount of polymer divided by the weight of the polymer aliquot. The Swelling Ratio is expressed as: swelling=(g swollen polymer−g dry polymer)/g dry polymer. The method used to determine the Swelling Ratio for any given polymer comprised the following:
a. 50-100 mg of dry (less than 5 wt % water content) polymer is placed into an 11 mL sealable test tube (with screw cap) of known weight (weight of tube=Weight A).
b. Deionized water (10 mL) is added to the tube containing the polymer. The tube is sealed and tumbled for 16 hours (overnight) at room temperature. After incubation, the tube is centrifuged at 3000×g for 3 minutes and the supernatant is carefully removed by vacuum suction. For polymers that form a very loose sediment, another step of centrifugation is performed.
c. After step (b), the weight of swollen polymer plus tube (Weight B) is recorded.
d. Freeze at −40° C. for 30 minutes. Lyophilize for 48 h. Weigh dried polymer and test tube (recorded as Weight C).
e. Calculate g water absorbed per g of polymer, defined as: [(Weight B−Weight A)−(Weight C−Weight A)]/(Weight C−Weight A).

A “target ion” is an ion to which the polymer binds, and usually refers to the major ions bound by the polymer, or the ions whose binding to the polymer is thought to produce the therapeutic effect of the polymer (e.g., proton and chloride binding which leads to net removal of HCl).

The term “theoretical capacity” represents the calculated, expected binding of hydrochloric acid in an “SGF” assay, expressed in mmol/g. The theoretical capacity is based on the assumption that 100% of the amines from the monomer(s) and crosslinker(s) are incorporated in the crosslinked polymer based on their respective feed ratios. Theoretical capacity is thus equal to the concentration of amine functionalities in the polymer (mmol/g). The theoretical capacity assumes that each amine is available to bind the respective anions and cations and is not adjusted for the type of amine formed (e.g. it does not subtract capacity of quaternary amines that are not available to bind proton).

“Therapeutically effective amount” means the amount of a proton-binding crosslinked polymer that, when administered to a patient for treating a disease, is sufficient to effect such treatment for the disease. The amount constituting a “therapeutically effective amount” will vary depending on the polymer, the severity of the disease and the age, weight, etc., of the mammal to be treated.

“Treating” or “treatment” of a disease includes (i) inhibiting the disease, i.e., arresting or reducing the development of the disease or its clinical symptoms; or (ii) relieving the disease, i.e., causing regression of the disease or its clinical symptoms. Inhibiting the disease, for example, would include prophylaxis.

The term “triallylamine” denotes an amino moiety having three allyl groups.

The term “vinyl” denotes a moiety having the structural formula RₓH_{y}C═CH—*, where * denotes the point of attachment of the moiety to the remainder of the molecule wherein the point of attachment is a heteroatom or aryl, X and Y are independently 0, 1 or 2, such that X+Y=2, and R is hydrocarbyl or substituted hydrocarbyl.

The term “weight percent crosslinker” represents the calculated percentage, by mass, of a polymer sample that is derived from the crosslinker. Weight percent crosslinker is calculated using the feed ratio of the polymerization, and assumes full conversion of the monomer and crosslinker(s). The mass attributed to the crosslinker is equal to the expected increase of molecular weight in the infinite polymer network after reaction (e.g., 1,3-dichloropropane is 113 amu, but only 42 amu are added to a polymer network after crosslinking with DCP because the chlorine atoms, as leaving groups, are not incorporated into the polymer network).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and not exclusive (i.e., there may be other elements in addition to the recited elements).

### EMBODIMENTS

In accordance with the present disclosure, acid-base disorders may be treated using pharmaceutical compositions comprising a nonabsorbable composition having the capacity to remove clinically significant quantities of protons, the conjugate base of one or more strong acids, and/or one or more strong acids. An individual afflicted with a an acute or chronic acid/base disorder characterized by a baseline serum bicarbonate value of less than 22 mEq/l may thus be treated by oral administration of a pharmaceutical composition comprising the nonabsorbable composition which then transits the individual's digestive system, binds a target species (protons, one or more conjugate base(s) of a strong acid and/or one or more strong acid(s)) as it transits the digestive system, and removes the bound target species by normal biological function (defecation).

In general, the individual afflicted with an acute or chronic acid/base disorder may be at any stage of chronic kidney disease. For example, in one embodiment the afflicted individual has not yet reached end stage renal disease (“ESRD”) sometimes also referred to as end stage chronic kidney disease and is not yet on dialysis (i.e., the individual has a mGFR (or eGFR) of at least 15 mL/min/1.73 m²). In some embodiments, the afflicted individual will be Stage 3B CKD (i.e., the individual has a mGFR (or eGFR) in the range of 30-44 mL/min/1.73 m² for at least three months). In some embodiments, the afflicted individual will be Stage 3A CKD (i.e., the individual has a mGFR (or eGFR) in the range of 45-59 mL/min/1.73 m² for at least three months). Thus, for example, in some embodiments the afflicted individual has a mGFR or an eGFR of less than 60 mL/min/1.73 m² for at least three months. By way of further example, in some embodiments the afflicted individual has a mGFR or an eGFR of less than 45 mL/min/1.73 m² for at least three months. By way of further example, in some embodiments the afflicted individual has a mGFR or an eGFR of less than 30 mL/min/1.73 m² for at least three months. By way of further example, in some embodiments the afflicted individual has a mGFR or an eGFR of 15-30, 15-45, 15-60, 30-45 or even 30-60 mL/min/1.73 m² for at least three months.

The baseline serum bicarbonate value may be the serum bicarbonate concentration determined at a single time point or may be the mean or median value of two or more serum bicarbonate concentrations determined at two or more time-points. For example, in one embodiment the baseline serum bicarbonate value may be the value of the serum bicarbonate concentration determined at a single time point and the baseline serum bicarbonate value is used as a basis to determine an acute acidic condition requiring immediate treatment. In another embodiment, the baseline serum bicarbonate treatment value is the mean value of the serum bicarbonate concentration for serum samples drawn at different time points (e.g., different days). By way of further example, in one such embodiment the baseline serum bicarbonate treatment value is the mean value of the serum bicarbonate concentration for serum samples drawn on different days (e.g., at least 2, 3, 4, 5 or more days, that may be consecutive or separated by one or more days or even weeks). By way of further example, in one such embodiment the baseline serum bicarbonate treatment value is the mean value of the serum bicarbonate concentration for serum samples drawn on two consecutive days preceding the initiation of treatment.

In one embodiment, the acid-base disorder being treated is characterized by a baseline serum bicarbonate value of less than 21 mEq/l. For example, in one such embodiment the acid-base disorder being treated is characterized by a baseline serum bicarbonate value of less than 20 mEq/l. By way of further example, in one such embodiment the acid-base disorder being treated is characterized by a baseline serum bicarbonate value of less than 19 mEq/l. By way of further example, in one such embodiment the acid-base disorder being treated is characterized by a baseline serum bicarbonate value of less than 18 mEq/l. By way of further example, in one such embodiment the acid-base disorder being treated is characterized by a baseline serum bicarbonate value of less than 17 mEq/l. By way of further example, in one such embodiment the acid-base disorder being treated is characterized by a baseline serum bicarbonate value of less than 16 mEq/l. By way of further example, in one such embodiment the acid-base disorder being treated is characterized by a baseline serum bicarbonate value of less than 15 mEq/l. By way of further example, in one such embodiment the acid-base disorder being treated is characterized by a baseline serum bicarbonate value of less than 14 mEq/l. By way of further example, in one such embodiment the acid-base disorder being treated is characterized by a baseline serum bicarbonate value of less than 13 mEq/l. By way of further example, in one such embodiment the acid-base disorder being treated is characterized by a baseline serum bicarbonate value of less than 12 mEq/l. By way of further example, in one such embodiment the acid-base disorder being treated is characterized by a baseline serum bicarbonate value of less than 11 mEq/l. By way of further example, in one such embodiment the acid-base disorder being treated is characterized by a baseline serum bicarbonate value of less than 10 mEq/l. By way of further example, in one such embodiment the acid-base disorder being treated is characterized by a baseline serum bicarbonate value of less than 9 mEq/l.

In general, however, the acid-base disorder being treated is characterized by a baseline serum bicarbonate value of at least 9 mEq/l. For example, in one such embodiment, the acid-base disorder is characterized by a baseline serum bicarbonate value of at least 10 mEq/l. By way of further example, in one such embodiment, the acid-base disorder is characterized by a baseline serum bicarbonate value of at least 11 mEq/l. By way of further example, in one such embodiment, the acid-base disorder is characterized by a baseline serum bicarbonate value of at least 12 mEq/l. By way of further example, in one such embodiment, the acid-base disorder is characterized by a baseline serum bicarbonate value of at least 13 mEq/l. By way of further example, in one such embodiment, the acid-base disorder is characterized by a baseline serum bicarbonate value of at least 14 mEq/l. By way of further example, in one such embodiment, the acid-base disorder is characterized by a baseline serum bicarbonate value of at least 15 mEq/l. By way of further example, in one such embodiment, the acid-base disorder is characterized by a baseline serum bicarbonate value of at least 16 mEq/l. By way of further example, in one such embodiment, the acid-base disorder is characterized by a baseline serum bicarbonate value of at least 17 mEq/l. By way of further example, in one such embodiment, the acid-base disorder is characterized by a baseline serum bicarbonate value of at least 18 mEq/l. By way of further example, in one such embodiment, the acid-base disorder is characterized by a baseline serum bicarbonate value of at least 19 mEq/l. By way of further example, in one such embodiment, the acid-base disorder is characterized by a baseline serum bicarbonate value of at least 20 mEq/l. By way of further example, in one such embodiment, the acid-base disorder is characterized by a baseline serum bicarbonate value of at least 21 mEq/l.

In certain embodiments, the acid-base disorder being treated is characterized by a baseline serum bicarbonate value in the range of 9 to 21 mEq/l. For example, in one such embodiment the acid-base disorder is characterized by a baseline serum bicarbonate value in the range of 12 to 20 mEq/l. By way of further example, in one such embodiment the acid-base disorder is characterized by a baseline serum bicarbonate value in the range of 12 to 19 mEq/l. By way of further example, in one such embodiment the acid-base disorder is characterized by a baseline serum bicarbonate value in the range of 12 to 18 mEq/l. By way of further example, in one such embodiment the acid-base disorder is characterized by a baseline serum bicarbonate value in the range of 12 to 17 mEq/l. By way of further example, in one such embodiment the acid-base disorder is characterized by a baseline serum bicarbonate value in the range of 12 to 16 mEq/l. By way of further example, in one such embodiment the acid-base disorder is characterized by a baseline serum bicarbonate value in the range of 9 to 11 mEq/l. By way of further example, in one such embodiment the acid-base disorder is characterized by a baseline serum bicarbonate value in the range of 12-14. By way of further example, in one such embodiment the acid-base disorder is characterized by a baseline serum bicarbonate value in the range of 15-17. By way of further example, in one such embodiment the acid-base disorder is characterized by a baseline serum bicarbonate value in the range of 18-21.

In certain embodiments, oral administration of a pharmaceutical composition containing a nonabsorbable composition increases the individual's serum bicarbonate value from baseline to an increased serum bicarbonate value that exceeds the baseline serum bicarbonate value by at least 1 mEq/l. For example, in one such embodiment the treatment increases the individual's serum bicarbonate value to an increased serum bicarbonate value that exceeds the baseline serum bicarbonate value by at least 1.5 mEq/l. By way of further example in one such embodiment the treatment increases the individual's serum bicarbonate value to an increased serum bicarbonate value that exceeds the baseline serum bicarbonate value by at least 2 mEq/l. By way of further example in one such embodiment the treatment the individual's serum bicarbonate value to an increased serum bicarbonate value that exceeds the baseline serum bicarbonate value by at least 2.5 mEq/l. By way of further example in one such embodiment the treatment increases the individual's serum bicarbonate value to an increased serum bicarbonate value that exceeds the baseline serum bicarbonate value by at least at least 3 mEq/l. By way of further example in one such embodiment the treatment increases the baseline serum bicarbonate value to an increased serum bicarbonate value that exceeds the baseline serum bicarbonate value by at least 3.5 mEq/l. By way of further example in one such embodiment the treatment increases the individual's serum bicarbonate value to an increased serum bicarbonate value that exceeds the baseline serum bicarbonate value by at least 4 mEq/l. By way of further example in one such embodiment the treatment increases the individual's serum bicarbonate value to an increased serum bicarbonate value that exceeds the baseline serum bicarbonate value by at least 5 mEq/l but does not exceed 29 mEq/l. By way of further example in one such embodiment the treatment increases the individual's serum bicarbonate value to an increased serum bicarbonate value that exceeds the baseline serum bicarbonate value by at least 5 mEq/l but does not exceed 28 mEq/l. By way of further example in one such embodiment the treatment increases the individual's serum bicarbonate value to an increased serum bicarbonate value that exceeds the baseline serum bicarbonate value by at least 5 mEq/l but does not exceed 27 mEq/l. By way of further example in one such embodiment the treatment increases the individual's serum bicarbonate value to an increased serum bicarbonate value that exceeds the baseline serum bicarbonate value by at least 5 mEq/l but does not exceed 26 mEq/l. By way of further example in one such embodiment the treatment increases the individual's serum bicarbonate value to an increased serum bicarbonate value that exceeds the baseline serum bicarbonate value by at least 6 mEq/l but does not exceed 29 mEq/l. By way of further example in one such embodiment the treatment increases the individual's serum bicarbonate value to an increased serum bicarbonate value that exceeds the baseline serum bicarbonate value by at least 6 mEq/l but does not exceed 28 mEq/l. By way of further example in one such embodiment the treatment increases the individual's serum bicarbonate value to an increased serum bicarbonate value that exceeds the baseline serum bicarbonate value by at least 6 mEq/l but does not exceed 27 mEq/l. By way of further example in one such embodiment the treatment increases the individual's serum bicarbonate value to an increased serum bicarbonate value that exceeds the baseline serum bicarbonate value by at least 6 mEq/l but does not exceed 26 mEq/l. By way of further example in one such embodiment the treatment increases the individual's serum bicarbonate value to an increased serum bicarbonate value that exceeds the baseline serum bicarbonate value by at least 7 mEq/l but does not exceed 29 mEq/l. By way of further example in one such embodiment the treatment increases the individual's serum bicarbonate value to an increased serum bicarbonate value that exceeds the baseline serum bicarbonate value by at least 7 mEq/l but does not exceed 28 mEq/l. By way of further example in one such embodiment the treatment increases the individual's serum bicarbonate value to an increased serum bicarbonate value that exceeds the baseline serum bicarbonate value by at least 7 mEq/l but does not exceed 27 mEq/l. By way of further example in one such embodiment the treatment increases the individual's serum bicarbonate value to an increased serum bicarbonate value that exceeds the baseline serum bicarbonate value by at least 7 mEq/l but does not exceed 26 mEq/l. By way of further example in one such embodiment the treatment increases the individual's serum bicarbonate value to an increased serum bicarbonate value that exceeds the baseline serum bicarbonate value by at least 8 mEq/l but does not exceed 29 mEq/l. By way of further example in one such embodiment the treatment increases the individual's serum bicarbonate value to an increased serum bicarbonate value that exceeds the baseline serum bicarbonate value by at least 8 mEq/l but does not exceed 28 mEq/l. By way of further example in one such embodiment the treatment increases the individual's serum bicarbonate value to an increased serum bicarbonate value that exceeds the baseline serum bicarbonate value by at least 8 mEq/l but does not exceed 27 mEq/l. By way of further example in one such embodiment the treatment increases the individual's serum bicarbonate value to an increased serum bicarbonate value that exceeds the baseline serum bicarbonate value by at least 8 mEq/l but does not exceed 26 mEq/l. By way of further example in one such embodiment the treatment increases the individual's serum bicarbonate value to an increased serum bicarbonate value that exceeds the baseline serum bicarbonate value by at least 9 mEq/l but does not exceed 29 mEq/l. By way of further example in one such embodiment the treatment increases the individual's serum bicarbonate value to an increased serum bicarbonate value that exceeds the baseline serum bicarbonate value by at least 9 mEq/l but does not exceed 28 mEq/l. By way of further example in one such embodiment the treatment increases the individual's serum bicarbonate value to an increased serum bicarbonate value that exceeds the baseline serum bicarbonate value by at least 9 mEq/l but does not exceed 27 mEq/l. By way of further example in one such embodiment the treatment increases the individual's serum bicarbonate value to an increased serum bicarbonate value that exceeds the baseline serum bicarbonate value by at least 9 mEq/l but does not exceed 26 mEq/l. In each of the foregoing exemplary embodiments recited in this paragraph, the treatment enables the increased serum bicarbonate value to be sustained over a prolonged period of at least one week, at least one month, at least two months, at least three months, at least six months, or even at least one year.

In certain embodiments, the treatment increases the individual's serum bicarbonate value from a baseline serum bicarbonate value in the range of 12 to 21 mEq/l to an increased value in the range of 24 mEq/l to 29 mEq/l. For example, in one such embodiment the treatment increases the individual's serum bicarbonate value from a baseline serum bicarbonate value in the range of 12 to 17 mEq/l to an increased value in the range of 24 mEq/l to 29 mEq/l. By way of further example, in one such embodiment the treatment increases the individual's serum bicarbonate value from a baseline serum bicarbonate value in the range of 12 to 14 mEq/l to an increased value in the range of 24 mEq/l to 29 mEq/l. By way of further example, in one such embodiment the treatment increases the individual's serum bicarbonate value from a baseline serum bicarbonate value in the range of 15 to 17 mEq/l to an increased value in the range of 24 mEq/l to 29 mEq/l. By way of further example, in one such embodiment the treatment increases the individual's serum bicarbonate value from a baseline serum bicarbonate value in the range of 18 to 21 mEq/l to an increased value in the range of 24 mEq/l to 29 mEq/l. In each of the foregoing embodiments recited in this paragraph, the treatment enables the increased serum bicarbonate value to be sustained over a prolonged period of at least one week, at least one month, at least two months, at least three months, at least six months, or even at least one year.

In certain embodiments, the treatment achieves a clinically significant increase is achieved within a treatment period of less than one month. For example, in one such embodiment, the treatment achieves a clinically significant increase within a treatment period of 25 days. By way of further example, in one such embodiment the treatment achieves the clinically significant increase is achieved within a treatment period of 3 weeks. By way of further example, in one such embodiment the treatment achieves the clinically significant increase is achieved within a treatment period of 15 days. By way of further example, in one such embodiment the treatment achieves the clinically significant increase is achieved within a treatment period of 2 weeks. By way of further example, in one such embodiment the treatment achieves the clinically significant increase is achieved within a treatment period of 10 days. By way of further example, in one such embodiment the treatment achieves the clinically significant increase is achieved within a treatment period of 1 week. By way of further example, in one such embodiment the treatment achieves the clinically significant increase is achieved within a treatment period of 6 days. By way of further example, in one such embodiment the treatment achieves the clinically significant increase is achieved within a treatment period of 5 days. By way of further example, in one such embodiment the treatment achieves the clinically significant increase is achieved within a treatment period of 4 days. By way of further example, in one such embodiment the treatment achieves the clinically significant increase is achieved within a treatment period of 3 days. By way of further example, in one such embodiment the treatment achieves the clinically significant increase is achieved within a treatment period of 2 days. By way of further example, in one such embodiment the treatment achieves the clinically significant increase is achieved within a treatment period of 1 day. By way of further example, in one such embodiment the treatment achieves the clinically significant increase is achieved within a treatment period of 12 hours.

In certain embodiments, the treatment achieves a clinically significant increase is achieved without any change in the individual's diet or dietary habits relative to the period immediately preceding the initiation of treatment. For example, in one such embodiment the clinically significant increase is achieved independent of the individual's diet or dietary habits.

In certain embodiments, the individual's serum bicarbonate value returns to the baseline value±2.5 mEq/l within 1 month of the cessation of treatment. For example, in one such embodiment the individual's serum bicarbonate value returns to the baseline value±2.5 mEq/l within 3 weeks of the cessation of treatment. By way of further example, in one such embodiment the individual's serum bicarbonate value returns to the baseline value±2.5 mEq/l within 2 weeks of the cessation of treatment. By way of further example, in one such embodiment the individual's serum bicarbonate value returns to the baseline value±2.5 mEq/l within 10 days of the cessation of treatment. By way of further example, in one such embodiment the individual's serum bicarbonate value returns to the baseline value±2.5 mEq/l within 9 days of the cessation of treatment. By way of further example, in one such embodiment the individual's serum bicarbonate value returns to the baseline value±2.5 mEq/l within 8 days of the cessation of treatment. By way of further example, in one such embodiment the individual's serum bicarbonate value returns to the baseline value±2.5 mEq/l within 7 days of the cessation of treatment. By way of further example, in one such embodiment the individual's serum bicarbonate value returns to the baseline value±2.5 mEq/l within 6 days of the cessation of treatment. By way of further example, in one such embodiment the individual's serum bicarbonate value returns to the baseline value±2.5 mEq/l within 5 days of the cessation of treatment. By way of further example, in one such embodiment the individual's serum bicarbonate value returns to the baseline value±2.5 mEq/l within 4 days of the cessation of treatment. By way of further example, in one such embodiment the individual's serum bicarbonate value returns to the baseline value±2.5 mEq/l within 3 days of the cessation of treatment. By way of further example, in one such embodiment the individual's serum bicarbonate value returns to the baseline value±2.5 mEq/l within 2 days of the cessation of treatment. By way of further example, in one such embodiment the individual's serum bicarbonate value returns to the baseline value±2.5 mEq/l within 1 day of the cessation of treatment.

In certain embodiments, the individual's serum bicarbonate value returns to the baseline value±2 mEq/l within 1 month of the cessation of treatment. For example, in one such embodiment the individual's serum bicarbonate value returns to the baseline value±2 mEq/l within 3 weeks of the cessation of treatment. By way of further example, in one such embodiment the individual's serum bicarbonate value returns to the baseline value±2 mEq/l within 2 weeks of the cessation of treatment. By way of further example, in one such embodiment the individual's serum bicarbonate value returns to the baseline value±2 mEq/l within 10 days of the cessation of treatment. By way of further example, in one such embodiment the individual's serum bicarbonate value returns to the baseline value±2 mEq/l within 9 days of the cessation of treatment. By way of further example, in one such embodiment the individual's serum bicarbonate value returns to the baseline value±2 mEq/l within 8 days of the cessation of treatment. By way of further example, in one such embodiment the individual's serum bicarbonate value returns to the baseline value±2 mEq/l within 7 days of the cessation of treatment. By way of further example, in one such embodiment the individual's serum bicarbonate value returns to the baseline value±2 mEq/l within 6 days of the cessation of treatment. By way of further example, in one such embodiment the individual's serum bicarbonate value returns to the baseline value±2 mEq/l within 5 days of the cessation of treatment. By way of further example, in one such embodiment the individual's serum bicarbonate value returns to the baseline value±2 mEq/l within 4 days of the cessation of treatment. By way of further example, in one such embodiment the individual's serum bicarbonate value returns to the baseline value±2 mEq/l within 3 days of the cessation of treatment. By way of further example, in one such embodiment the individual's serum bicarbonate value returns to the baseline value±2 mEq/l within 2 days of the cessation of treatment. By way of further example, in one such embodiment the individual's serum bicarbonate value returns to the baseline value±2 mEq/l within 1 day of the cessation of treatment.

In certain embodiments, the individual's serum bicarbonate value returns to the baseline value±1.5 mEq/l within 1 month of the cessation of treatment. For example, in one such embodiment the individual's serum bicarbonate value returns to the baseline value±1.5 mEq/l within 3 weeks of the cessation of treatment. By way of further example, in one such embodiment the individual's serum bicarbonate value returns to the baseline value±1.5 mEq/l within 2 weeks of the cessation of treatment. By way of further example, in one such embodiment the individual's serum bicarbonate value returns to the baseline value±1.5 mEq/l within 10 days of the cessation of treatment. By way of further example, in one such embodiment the individual's serum bicarbonate value returns to the baseline value±1.5 mEq/l within 9 days of the cessation of treatment. By way of further example, in one such embodiment the individual's serum bicarbonate value returns to the baseline value±1.5 mEq/l within 8 days of the cessation of treatment. By way of further example, in one such embodiment the individual's serum bicarbonate value returns to the baseline value±1.5 mEq/l within 7 days of the cessation of treatment. By way of further example, in one such embodiment the individual's serum bicarbonate value returns to the baseline value±1.5 mEq/l within 6 days of the cessation of treatment. By way of further example, in one such embodiment the individual's serum bicarbonate value returns to the baseline value±1.5 mEq/l within 5 days of the cessation of treatment. By way of further example, in one such embodiment the individual's serum bicarbonate value returns to the baseline value±1.5 mEq/l within 4 days of the cessation of treatment. By way of further example, in one such embodiment the individual's serum bicarbonate value returns to the baseline value±1.5 mEq/l within 3 days of the cessation of treatment. By way of further example, in one such embodiment the individual's serum bicarbonate value returns to the baseline value±1.5 mEq/l within 2 days of the cessation of treatment. By way of further example, in one such embodiment the individual's serum bicarbonate value returns to the baseline value±1.5 mEq/l within 1 day of the cessation of treatment.

In certain embodiments, the individual's serum bicarbonate value returns to the baseline value±1 mEq/l within 1 month of the cessation of treatment. For example, in one such embodiment the individual's serum bicarbonate value returns to the baseline value±1 mEq/l within 3 weeks of the cessation of treatment. By way of further example, in one such embodiment the individual's serum bicarbonate value returns to the baseline value±1 mEq/l within 2 weeks of the cessation of treatment. By way of further example, in one such embodiment the individual's serum bicarbonate value returns to the baseline value±1 mEq/l within 10 days of the cessation of treatment. By way of further example, in one such embodiment the individual's serum bicarbonate value returns to the baseline value±1 mEq/l within 9 days of the cessation of treatment. By way of further example, in one such embodiment the individual's serum bicarbonate value returns to the baseline value±1 mEq/l within 8 days of the cessation of treatment. By way of further example, in one such embodiment the individual's serum bicarbonate value returns to the baseline value±1 mEq/l within 7 days of the cessation of treatment. By way of further example, in one such embodiment the individual's serum bicarbonate value returns to the baseline value±1 mEq/l within 6 days of the cessation of treatment. By way of further example, in one such embodiment the individual's serum bicarbonate value returns to the baseline value±1 mEq/l within 5 days of the cessation of treatment. By way of further example, in one such embodiment the individual's serum bicarbonate value returns to the baseline value±1 mEq/l within 4 days of the cessation of treatment. By way of further example, in one such embodiment the individual's serum bicarbonate value returns to the baseline value±1 mEq/l within 3 days of the cessation of treatment. By way of further example, in one such embodiment the individual's serum bicarbonate value returns to the baseline value±1 mEq/l within 2 days of the cessation of treatment. By way of further example, in one such embodiment the individual's serum bicarbonate value returns to the baseline value±1 mEq/l within 1 day of the cessation of treatment.

In certain embodiments, upon the cessation of treatment the individual's serum bicarbonate value decreases by at least 1 mEq/l within 1 month of the cessation of treatment. For example, in one such embodiment. For example, in one such embodiment the individual's serum bicarbonate value decreases by at least 1 mEq/l within 3 weeks of the cessation of treatment. By way of further example, in one such embodiment the individual's serum bicarbonate value decreases by at least 1 mEq/l within 2 weeks of the cessation of treatment. By way of further example, in one such embodiment the individual's serum bicarbonate value decreases by at least 1 mEq/l within 10 days of the cessation of treatment. By way of further example, in one such embodiment the individual's serum bicarbonate value decreases by at least 1 mEq/l within 9 days of the cessation of treatment. By way of further example, in one such embodiment the individual's serum bicarbonate value decreases by at least 1 mEq/l within 8 days of the cessation of treatment. By way of further example, in one such embodiment the individual's serum bicarbonate value decreases by at least 1 mEq/l within 7 days of the cessation of treatment. By way of further example, in one such embodiment the individual's serum bicarbonate value decreases by at least 1 mEq/l within 6 days of the cessation of treatment. By way of further example, in one such embodiment the individual's serum bicarbonate value decreases by at least 1 mEq/l within 5 days of the cessation of treatment. By way of further example, in one such embodiment the individual's serum bicarbonate value decreases by at least 1 mEq/l within 4 days of the cessation of treatment. By way of further example, in one such embodiment the individual's serum bicarbonate value decreases by at least 1 mEq/l within 3 days of the cessation of treatment. By way of further example, in one such embodiment the individual's serum bicarbonate value decreases by at least 1 mEq/l within 2 days of the cessation of treatment. By way of further example, in one such embodiment the individual's serum bicarbonate value decreases by at least 1 mEq/l within 1 day of the cessation of treatment. By way of further example, in one such embodiment the individual's serum bicarbonate value decreases by at least 1 mEq/l within 12 hours of the cessation of treatment.

In certain embodiments, upon the cessation of treatment the individual's serum bicarbonate value decreases by at least 1.5 mEq/l within 1 month of the cessation of treatment. For example, in one such embodiment. For example, in one such embodiment the individual's serum bicarbonate value decreases by at least 1.5 mEq/l within 3 weeks of the cessation of treatment. By way of further example, in one such embodiment the individual's serum bicarbonate value decreases by at least 1.5 mEq/l within 2 weeks of the cessation of treatment. By way of further example, in one such embodiment the individual's serum bicarbonate value decreases by at least 1.5 mEq/l within 10 days of the cessation of treatment. By way of further example, in one such embodiment the individual's serum bicarbonate value decreases by at least 1.5 mEq/l within 9 days of the cessation of treatment. By way of further example, in one such embodiment the individual's serum bicarbonate value decreases by at least 1.5 mEq/l within 8 days of the cessation of treatment. By way of further example, in one such embodiment the individual's serum bicarbonate value decreases by at least 1.5 mEq/l within 7 days of the cessation of treatment. By way of further example, in one such embodiment the individual's serum bicarbonate value decreases by at least 1.5 mEq/l within 6 days of the cessation of treatment. By way of further example, in one such embodiment the individual's serum bicarbonate value decreases by at least 1.5 mEq/l within 5 days of the cessation of treatment. By way of further example, in one such embodiment the individual's serum bicarbonate value decreases by at least 1.5 mEq/l within 4 days of the cessation of treatment. By way of further example, in one such embodiment the individual's serum bicarbonate value decreases by at least 1.5 mEq/l within 3 days of the cessation of treatment. By way of further example, in one such embodiment the individual's serum bicarbonate value decreases by at least 1.5 mEq/l within 2 days of the cessation of treatment. By way of further example, in one such embodiment the individual's serum bicarbonate value decreases by at least 1.5 mEq/l within 1 day of the cessation of treatment. By way of further example, in one such embodiment the individual's serum bicarbonate value decreases by at least 1.5 mEq/l within 12 hours of the cessation of treatment.

In certain embodiments, upon the cessation of treatment the individual's serum bicarbonate value decreases by at least 2 mEq/l within 1 month of the cessation of treatment. For example, in one such embodiment. For example, in one such embodiment the individual's serum bicarbonate value decreases by at least 2 mEq/l within 3 weeks of the cessation of treatment. By way of further example, in one such embodiment the individual's serum bicarbonate value decreases by at least 2 mEq/l within 2 weeks of the cessation of treatment. By way of further example, in one such embodiment the individual's serum bicarbonate value decreases by at least 2 mEq/l within 10 days of the cessation of treatment. By way of further example, in one such embodiment the individual's serum bicarbonate value decreases by at least 2 mEq/l within 9 days of the cessation of treatment. By way of further example, in one such embodiment the individual's serum bicarbonate value decreases by at least 2 mEq/l within 8 days of the cessation of treatment. By way of further example, in one such embodiment the individual's serum bicarbonate value decreases by at least 2 mEq/l within 7 days of the cessation of treatment. By way of further example, in one such embodiment the individual's serum bicarbonate value decreases by at least 2 mEq/l within 6 days of the cessation of treatment. By way of further example, in one such embodiment the individual's serum bicarbonate value decreases by at least 2 mEq/l within 5 days of the cessation of treatment. By way of further example, in one such embodiment the individual's serum bicarbonate value decreases by at least 2 mEq/l within 4 days of the cessation of treatment. By way of further example, in one such embodiment the individual's serum bicarbonate value decreases by at least 2 mEq/l within 3 days of the cessation of treatment. By way of further example, in one such embodiment the individual's serum bicarbonate value decreases by at least 2 mEq/l within 2 days of the cessation of treatment. By way of further example, in one such embodiment the individual's serum bicarbonate value decreases by at least 2 mEq/l within 1 day of the cessation of treatment. By way of further example, in one such embodiment the individual's serum bicarbonate value decreases by at least 2 mEq/l within 12 hours of the cessation of treatment.

In certain embodiments, upon the cessation of treatment the individual's serum bicarbonate value decreases by at least 2.5 mEq/l within 1 month of the cessation of treatment. For example, in one such embodiment. For example, in one such embodiment the individual's serum bicarbonate value decreases by at least 2.5 mEq/l within 3 weeks of the cessation of treatment. By way of further example, in one such embodiment the individual's serum bicarbonate value decreases by at least 2.5 mEq/l within 2 weeks of the cessation of treatment. By way of further example, in one such embodiment the individual's serum bicarbonate value decreases by at least 2.5 mEq/l within 10 days of the cessation of treatment. By way of further example, in one such embodiment the individual's serum bicarbonate value decreases by at least 2.5 mEq/l within 9 days of the cessation of treatment. By way of further example, in one such embodiment the individual's serum bicarbonate value decreases by at least 2.5 mEq/l within 8 days of the cessation of treatment. By way of further example, in one such embodiment the individual's serum bicarbonate value decreases by at least 2.5 mEq/l within 7 days of the cessation of treatment. By way of further example, in one such embodiment the individual's serum bicarbonate value decreases by at least 2.5 mEq/l within 6 days of the cessation of treatment. By way of further example, in one such embodiment the individual's serum bicarbonate value decreases by at least 2.5 mEq/l within 5 days of the cessation of treatment. By way of further example, in one such embodiment the individual's serum bicarbonate value decreases by at least 2.5 mEq/l within 4 days of the cessation of treatment. By way of further example, in one such embodiment the individual's serum bicarbonate value decreases by at least 2.5 mEq/l within 3 days of the cessation of treatment. By way of further example, in one such embodiment the individual's serum bicarbonate value decreases by at least 2.5 mEq/l within 2 days of the cessation of treatment. By way of further example, in one such embodiment the individual's serum bicarbonate value decreases by at least 2.5 mEq/l within 1 day of the cessation of treatment. By way of further example, in one such embodiment the individual's serum bicarbonate value decreases by at least 2.5 mEq/l within 12 hours of the cessation of treatment.

In certain embodiments, upon the cessation of treatment the individual's serum bicarbonate value decreases by at least 3 mEq/l within 1 month of the cessation of treatment. For example, in one such embodiment. For example, in one such embodiment the individual's serum bicarbonate value decreases by at least 3 mEq/l within 3 weeks of the cessation of treatment. By way of further example, in one such embodiment the individual's serum bicarbonate value decreases by at least 3 mEq/l within 2 weeks of the cessation of treatment. By way of further example, in one such embodiment the individual's serum bicarbonate value decreases by at least 3 mEq/l within 10 days of the cessation of treatment. By way of further example, in one such embodiment the individual's serum bicarbonate value decreases by at least 3 mEq/l within 9 days of the cessation of treatment. By way of further example, in one such embodiment the individual's serum bicarbonate value decreases by at least 3 mEq/l within 8 days of the cessation of treatment. By way of further example, in one such embodiment the individual's serum bicarbonate value decreases by at least 3 mEq/l within 7 days of the cessation of treatment. By way of further example, in one such embodiment the individual's serum bicarbonate value decreases by at least 3 mEq/l within 6 days of the cessation of treatment. By way of further example, in one such embodiment the individual's serum bicarbonate value decreases by at least 3 mEq/l within 5 days of the cessation of treatment. By way of further example, in one such embodiment the individual's serum bicarbonate value decreases by at least 3 mEq/l within 4 days of the cessation of treatment. By way of further example, in one such embodiment the individual's serum bicarbonate value decreases by at least 3 mEq/l within 3 days of the cessation of treatment. By way of further example, in one such embodiment the individual's serum bicarbonate value decreases by at least 3 mEq/l within 2 days of the cessation of treatment. By way of further example, in one such embodiment the individual's serum bicarbonate value decreases by at least 3 mEq/l within 1 day of the cessation of treatment. By way of further example, in one such embodiment the individual's serum bicarbonate value decreases by at least 3 mEq/l within 12 hours of the cessation of treatment.

In certain embodiments, upon the cessation of treatment the individual's serum bicarbonate value decreases by at least 3.5 mEq/l within 1 month of the cessation of treatment. For example, in one such embodiment. For example, in one such embodiment the individual's serum bicarbonate value decreases by at least 3.5 mEq/l within 3 weeks of the cessation of treatment. By way of further example, in one such embodiment the individual's serum bicarbonate value decreases by at least 3.5 mEq/l within 2 weeks of the cessation of treatment. By way of further example, in one such embodiment the individual's serum bicarbonate value decreases by at least 3.5 mEq/l within 10 days of the cessation of treatment. By way of further example, in one such embodiment the individual's serum bicarbonate value decreases by at least 3.5 mEq/l within 9 days of the cessation of treatment. By way of further example, in one such embodiment the individual's serum bicarbonate value decreases by at least 3.5 mEq/l within 8 days of the cessation of treatment. By way of further example, in one such embodiment the individual's serum bicarbonate value decreases by at least 3.5 mEq/l within 7 days of the cessation of treatment. By way of further example, in one such embodiment the individual's serum bicarbonate value decreases by at least 3.5 mEq/l within 6 days of the cessation of treatment. By way of further example, in one such embodiment the individual's serum bicarbonate value decreases by at least 3.5 mEq/l within 5 days of the cessation of treatment. By way of further example, in one such embodiment the individual's serum bicarbonate value decreases by at least 3.5 mEq/l within 4 days of the cessation of treatment. By way of further example, in one such embodiment the individual's serum bicarbonate value decreases by at least 3.5 mEq/l within 3 days of the cessation of treatment. By way of further example, in one such embodiment the individual's serum bicarbonate value decreases by at least 3.5 mEq/l within 2 days of the cessation of treatment. By way of further example, in one such embodiment the individual's serum bicarbonate value decreases by at least 3.5 mEq/l within 1 day of the cessation of treatment. By way of further example, in one such embodiment the individual's serum bicarbonate value decreases by at least 3.5 mEq/l within 12 hours of the cessation of treatment.

In certain embodiments, upon the cessation of treatment the individual's serum bicarbonate value decreases by at least 4 mEq/l within 1 month of the cessation of treatment. For example, in one such embodiment. For example, in one such embodiment the individual's serum bicarbonate value decreases by at least 4 mEq/l within 3 weeks of the cessation of treatment. By way of further example, in one such embodiment the individual's serum bicarbonate value decreases by at least 4 mEq/l within 2 weeks of the cessation of treatment. By way of further example, in one such embodiment the individual's serum bicarbonate value decreases by at least 4 mEq/l within 10 days of the cessation of treatment. By way of further example, in one such embodiment the individual's serum bicarbonate value decreases by at least 4 mEq/l within 9 days of the cessation of treatment. By way of further example, in one such embodiment the individual's serum bicarbonate value decreases by at least 4 mEq/l within 8 days of the cessation of treatment. By way of further example, in one such embodiment the individual's serum bicarbonate value decreases by at least 4 mEq/l within 7 days of the cessation of treatment. By way of further example, in one such embodiment the individual's serum bicarbonate value decreases by at least 4 mEq/l within 6 days of the cessation of treatment. By way of further example, in one such embodiment the individual's serum bicarbonate value decreases by at least 4 mEq/l within 5 days of the cessation of treatment. By way of further example, in one such embodiment the individual's serum bicarbonate value decreases by at least 4 mEq/l within 4 days of the cessation of treatment. By way of further example, in one such embodiment the individual's serum bicarbonate value decreases by at least 4 mEq/l within 3 days of the cessation of treatment. By way of further example, in one such embodiment the individual's serum bicarbonate value decreases by at least 4 mEq/l within 2 days of the cessation of treatment. By way of further example, in one such embodiment the individual's serum bicarbonate value decreases by at least 4 mEq/l within 1 day of the cessation of treatment. By way of further example, in one such embodiment the individual's serum bicarbonate value decreases by at least 4 mEq/l within 12 hours of the cessation of treatment.

In certain embodiments, upon the cessation of treatment the individual's serum bicarbonate value decreases by at least 4.5 mEq/l within 1 month of the cessation of treatment. For example, in one such embodiment. For example, in one such embodiment the individual's serum bicarbonate value decreases by at least 4.5 mEq/l within 3 weeks of the cessation of treatment. By way of further example, in one such embodiment the individual's serum bicarbonate value decreases by at least 4.5 mEq/l within 2 weeks of the cessation of treatment. By way of further example, in one such embodiment the individual's serum bicarbonate value decreases by at least 4.5 mEq/l within 10 days of the cessation of treatment. By way of further example, in one such embodiment the individual's serum bicarbonate value decreases by at least 4.5 mEq/l within 9 days of the cessation of treatment. By way of further example, in one such embodiment the individual's serum bicarbonate value decreases by at least 4.5 mEq/l within 8 days of the cessation of treatment. By way of further example, in one such embodiment the individual's serum bicarbonate value decreases by at least 4.5 mEq/l within 7 days of the cessation of treatment. By way of further example, in one such embodiment the individual's serum bicarbonate value decreases by at least 4.5 mEq/l within 6 days of the cessation of treatment. By way of further example, in one such embodiment the individual's serum bicarbonate value decreases by at least 4.5 mEq/l within 5 days of the cessation of treatment. By way of further example, in one such embodiment the individual's serum bicarbonate value decreases by at least 4.5 mEq/l within 4 days of the cessation of treatment. By way of further example, in one such embodiment the individual's serum bicarbonate value decreases by at least 4.5 mEq/l within 3 days of the cessation of treatment. By way of further example, in one such embodiment the individual's serum bicarbonate value decreases by at least 4.5 mEq/l within 2 days of the cessation of treatment. By way of further example, in one such embodiment the individual's serum bicarbonate value decreases by at least 4.5 mEq/l within 1 day of the cessation of treatment. By way of further example, in one such embodiment the individual's serum bicarbonate value decreases by at least 4.5 mEq/l within 12 hours of the cessation of treatment.

In certain embodiments, upon the cessation of treatment the individual's serum bicarbonate value decreases by at least 5 mEq/l within 1 month of the cessation of treatment. For example, in one such embodiment. For example, in one such embodiment the individual's serum bicarbonate value decreases by at least 5 mEq/l within 3 weeks of the cessation of treatment. By way of further example, in one such embodiment the individual's serum bicarbonate value decreases by at least 5 mEq/l within 2 weeks of the cessation of treatment. By way of further example, in one such embodiment the individual's serum bicarbonate value decreases by at least 5 mEq/l within 10 days of the cessation of treatment. By way of further example, in one such embodiment the individual's serum bicarbonate value decreases by at least 5 mEq/l within 9 days of the cessation of treatment. By way of further example, in one such embodiment the individual's serum bicarbonate value decreases by at least 5 mEq/l within 8 days of the cessation of treatment. By way of further example, in one such embodiment the individual's serum bicarbonate value decreases by at least 5 mEq/l within 7 days of the cessation of treatment. By way of further example, in one such embodiment the individual's serum bicarbonate value decreases by at least 5 mEq/l within 6 days of the cessation of treatment. By way of further example, in one such embodiment the individual's serum bicarbonate value decreases by at least 5 mEq/l within 5 days of the cessation of treatment. By way of further example, in one such embodiment the individual's serum bicarbonate value decreases by at least 5 mEq/l within 4 days of the cessation of treatment. By way of further example, in one such embodiment the individual's serum bicarbonate value decreases by at least 5 mEq/l within 3 days of the cessation of treatment. By way of further example, in one such embodiment the individual's serum bicarbonate value decreases by at least 5 mEq/l within 2 days of the cessation of treatment. By way of further example, in one such embodiment the individual's serum bicarbonate value decreases by at least 5 mEq/l within 1 day of the cessation of treatment. By way of further example, in one such embodiment the individual's serum bicarbonate value decreases by at least 5 mEq/l within 12 hours of the cessation of treatment.

In one embodiment, the baseline serum bicarbonate value is the value of the serum bicarbonate concentration determined at a single time point. In another embodiment, the baseline serum bicarbonate value is the mean value of at least two serum bicarbonate concentrations determined at different time-points. For example, in one such embodiment the baseline serum bicarbonate value is the mean value of at least two serum bicarbonate concentrations for serum samples drawn on different days. By way of further example, the baseline serum bicarbonate value is the mean or median value of at least two serum bicarbonate concentrations for serum samples drawn on non-consecutive days. By way of further example, in one such method the non-consecutive days are separated by at least two days. By way of further example, in one such method the non-consecutive days are separated by at least one week. By way of further example, in one such method the non-consecutive days are separated by at least two weeks. By way of further example, in one such method the non-consecutive days are separated by at least three weeks.

In certain embodiments, the daily dose is no more than 100 g/day of the nonabsorbable composition. For example, in one such embodiment the daily dose is no more than 90 g/day of the nonabsorbable composition. By way of further example, in one such embodiment the daily dose is no more than 75 g/day of the nonabsorbable composition. By way of further example, in one such embodiment the daily dose is no more than 65 g/day of the nonabsorbable composition. By way of further example, in one such embodiment the daily dose is no more than 50 g/day of the nonabsorbable composition. By way of further example, in one such embodiment the daily dose is no more than 40 g/day of the nonabsorbable composition. By way of further example, in one such embodiment the daily dose is no more than 30 g/day of the nonabsorbable composition. By way of further example, in one such embodiment the daily dose is no more than 25 g/day of the nonabsorbable composition. By way of further example, in one such embodiment the daily dose is no more than 20 g/day of the nonabsorbable composition. By way of further example, in one such embodiment the daily dose is no more than 15 g/day of the nonabsorbable composition. By way of further example, in one such embodiment the daily dose is no more than 10 g/day of the nonabsorbable composition. By way of further example, in one such embodiment the daily dose is no more than 5 g/day of the nonabsorbable composition.

In certain embodiments, the individual is treated with the daily dose for a period of at least one day. For example, in one such embodiment the individual is treated with the daily dose for a period of at least one week. By way of further example, in one such embodiment the individual is treated with the daily dose for a period of at least one month. By way of further example, in one such embodiment the individual is treated with the daily dose for a period of at least two months. By way of further example, in one such embodiment the individual is treated with the daily dose for a period of at least three months. By way of further example, in one such embodiment the individual is treated with the daily dose for a period of at least several months. By way of further example, in one such embodiment the individual is treated with the daily dose for a period of at least six months. By way of further example, in one such embodiment the individual is treated with the daily dose for a period of at least one year.

In certain embodiments of the method of the present disclosure, the daily dose of the nonabsorbable composition has the capacity to remove at least about 5 mEq/day of the target species. For example, in one such embodiment the daily dose of the nonabsorbable composition has the capacity to remove at least about 6 mEq/day of the target species. By way of further example, in one such embodiment the daily dose of the nonabsorbable composition has the capacity to remove at least about 7 mEq/day of the target species. By way of further example, in one such embodiment the daily dose of the nonabsorbable composition has the capacity to remove at least about 8 mEq/day of the target species. By way of further example, in one such embodiment the daily dose of the nonabsorbable composition has the capacity to remove at least about 9 mEq/day of the target species. By way of further example, in one such embodiment the daily dose of the nonabsorbable composition has the capacity to remove at least about 10 mEq/day of the target species. By way of further example, in one such embodiment the daily dose of the nonabsorbable composition has the capacity to remove at least about 11 mEq/day of the target species. By way of further example, in one such embodiment the daily dose of the nonabsorbable composition has the capacity to remove at least about 12 mEq/day of the target species. By way of further example, in one such embodiment the daily dose of the nonabsorbable composition has the capacity to remove at least about 13 mEq/day of the target species. By way of further example, in one such embodiment the daily dose of the nonabsorbable composition has the capacity to remove at least about 14 mEq/day of the target species. By way of further example, in one such embodiment the daily dose of the nonabsorbable composition has the capacity to remove at least about 15 mEq/day of the target species. By way of further example, in one such embodiment the daily dose of the nonabsorbable composition has the capacity to remove at least about 16 mEq/day of the target species. By way of further example, in one such embodiment the daily dose of the nonabsorbable composition has the capacity to remove at least about 17 mEq/day of the target species. By way of further example, in one such embodiment the daily dose of the nonabsorbable composition has the capacity to remove at least about 18 mEq/day of the target species. By way of further example, in one such embodiment the daily dose of the nonabsorbable composition has the capacity to remove at least about 19 mEq/day of the target species. By way of further example, in one such embodiment the daily dose of the nonabsorbable composition has the capacity to remove at least about 20 mEq/day of the target species. By way of further example, in one such embodiment the daily dose of the nonabsorbable composition has the capacity to remove at least about 21 mEq/day of the target species. By way of further example, in one such embodiment the daily dose of the nonabsorbable composition has the capacity to remove at least about 22 mEq/day of the target species. By way of further example, in one such embodiment the daily dose of the nonabsorbable composition has the capacity to remove at least about 23 mEq/day of the target species. By way of further example, in one such embodiment the daily dose of the nonabsorbable composition has the capacity to remove at least about 24 mEq/day of the target species. By way of further example, in one such embodiment the daily dose of the nonabsorbable composition has the capacity to remove at least about 25 mEq/day of the target species. By way of further example, in one such embodiment the daily dose of the nonabsorbable composition has the capacity to remove at least about 26 mEq/day of the target species. By way of further example, in one such embodiment the daily dose of the nonabsorbable composition has the capacity to remove at least about 27 mEq/day of the target species. By way of further example, in one such embodiment the daily dose of the nonabsorbable composition has the capacity to remove at least about 28 mEq/day of the target species. By way of further example, in one such embodiment the daily dose of the nonabsorbable composition has the capacity to remove at least about 29 mEq/day of the target species. By way of further example, in one such embodiment the daily dose of the nonabsorbable composition has the capacity to remove at least about 30 mEq/day of the target species. By way of further example, in one such embodiment the daily dose of the nonabsorbable composition has the capacity to remove at least about 31 mEq/day of the target species. By way of further example, in one such embodiment the daily dose of the nonabsorbable composition has the capacity to remove at least about 32 mEq/day of the target species. By way of further example, in one such embodiment the daily dose of the nonabsorbable composition has the capacity to remove at least about 33 mEq/day of the target species. By way of further example, in one such embodiment the daily dose of the nonabsorbable composition has the capacity to remove at least about 34 mEq/day of the target species. By way of further example, in one such embodiment the daily dose of the nonabsorbable composition has the capacity to remove at least about 35 mEq/day of the target species. By way of further example, in one such embodiment the daily dose of the nonabsorbable composition has the capacity to remove at least about 36 mEq/day of the target species. By way of further example, in one such embodiment the daily dose of the nonabsorbable composition has the capacity to remove at least about 37 mEq/day of the target species. By way of further example, in one such embodiment the daily dose of the nonabsorbable composition has the capacity to remove at least about 38 mEq/day of the target species. By way of further example, in one such embodiment the daily dose of the nonabsorbable composition has the capacity to remove at least about 39 mEq/day of the target species. By way of further example, in one such embodiment the daily dose of the nonabsorbable composition has the capacity to remove at least about 40 mEq/day of the target species. By way of further example, in one such embodiment the daily dose of the nonabsorbable composition has the capacity to remove at least about 41 mEq/day of the target species. By way of further example, in one such embodiment the daily dose of the nonabsorbable composition has the capacity to remove at least about 42 mEq/day of the target species. By way of further example, in one such embodiment the daily dose of the nonabsorbable composition has the capacity to remove at least about 43 mEq/day of the target species. By way of further example, in one such embodiment the daily dose of the nonabsorbable composition has the capacity to remove at least about 44 mEq/day of the target species. By way of further example, in one such embodiment the daily dose of the nonabsorbable composition has the capacity to remove at least about 45 mEq/day of the target species. By way of further example, in one such embodiment the daily dose of the nonabsorbable composition has the capacity to remove at least about 46 mEq/day of the target species. By way of further example, in one such embodiment the daily dose of the nonabsorbable composition has the capacity to remove at least about 47 mEq/day of the target species. By way of further example, in one such embodiment the daily dose of the nonabsorbable composition has the capacity to remove at least about 48 mEq/day of the target species. By way of further example, in one such embodiment the daily dose of the nonabsorbable composition has the capacity to remove at least about 49 mEq/day of the target species. By way of further example, in one such embodiment the daily dose of the nonabsorbable composition has the capacity to remove at least about 50 mEq/day of the target species.

In certain embodiments of the method of the present disclosure, the daily dose of the nonabsorbable composition removes at least about 5 mEq/day of the target species. For example, in one such embodiment the daily dose of the nonabsorbable composition removes at least about 6 mEq/day of the target species. By way of further example, in one such embodiment the daily dose of the nonabsorbable composition removes at least about 7 mEq/day of the target species. By way of further example, in one such embodiment the daily dose of the nonabsorbable composition removes at least about 8 mEq/day of the target species. By way of further example, in one such embodiment the daily dose of the nonabsorbable composition removes at least about 9 mEq/day of the target species. By way of further example, in one such embodiment the daily dose of the nonabsorbable composition removes at least about 10 mEq/day of the target species. By way of further example, in one such embodiment the daily dose of the nonabsorbable composition removes at least about 11 mEq/day of the target species. By way of further example, in one such embodiment the daily dose of the nonabsorbable composition removes at least about 12 mEq/day of the target species. By way of further example, in one such embodiment the daily dose of the nonabsorbable composition removes at least about 13 mEq/day of the target species. By way of further example, in one such embodiment the daily dose of the nonabsorbable composition removes at least about 14 mEq/day of the target species. By way of further example, in one such embodiment the daily dose of the nonabsorbable composition removes at least about 15 mEq/day of the target species. By way of further example, in one such embodiment the daily dose of the nonabsorbable composition removes at least about 16 mEq/day of the target species. By way of further example, in one such embodiment the daily dose of the nonabsorbable composition removes at least about 17 mEq/day of the target species. By way of further example, in one such embodiment the daily dose of the nonabsorbable composition removes at least about 18 mEq/day of the target species. By way of further example, in one such embodiment the daily dose of the nonabsorbable composition removes at least about 19 mEq/day of the target species. By way of further example, in one such embodiment the daily dose of the nonabsorbable composition removes at least about 20 mEq/day of the target species. By way of further example, in one such embodiment the daily dose of the nonabsorbable composition removes at least about 21 mEq/day of the target species. By way of further example, in one such embodiment the daily dose of the nonabsorbable composition removes at least about 22 mEq/day of the target species. By way of further example, in one such embodiment the daily dose of the nonabsorbable composition removes at least about 23 mEq/day of the target species. By way of further example, in one such embodiment the daily dose of the nonabsorbable composition removes at least about 24 mEq/day of the target species. By way of further example, in one such embodiment the daily dose of the nonabsorbable composition removes at least about 25 mEq/day of the target species. By way of further example, in one such embodiment the daily dose of the nonabsorbable composition removes at least about 26 mEq/day of the target species. By way of further example, in one such embodiment the daily dose of the nonabsorbable composition removes at least about 27 mEq/day of the target species. By way of further example, in one such embodiment the daily dose of the nonabsorbable composition removes at least about 28 mEq/day of the target species. By way of further example, in one such embodiment the daily dose of the nonabsorbable composition removes at least about 29 mEq/day of the target species. By way of further example, in one such embodiment the daily dose of the nonabsorbable composition removes at least about 30 mEq/day of the target species. By way of further example, in one such embodiment the daily dose of the nonabsorbable composition removes at least about 31 mEq/day of the target species. By way of further example, in one such embodiment the daily dose of the nonabsorbable composition removes at least about 32 mEq/day of the target species. By way of further example, in one such embodiment the daily dose of the nonabsorbable composition removes at least about 33 mEq/day of the target species. By way of further example, in one such embodiment the daily dose of the nonabsorbable composition removes at least about 34 mEq/day of the target species. By way of further example, in one such embodiment the daily dose of the nonabsorbable composition removes at least about 35 mEq/day of the target species. By way of further example, in one such embodiment the daily dose of the nonabsorbable composition removes at least about 36 mEq/day of the target species. By way of further example, in one such embodiment the daily dose of the nonabsorbable composition removes at least about 37 mEq/day of the target species. By way of further example, in one such embodiment the daily dose of the nonabsorbable composition removes at least about 38 mEq/day of the target species. By way of further example, in one such embodiment the daily dose of the nonabsorbable composition removes at least about 39 mEq/day of the target species. By way of further example, in one such embodiment the daily dose of the nonabsorbable composition removes at least about 40 mEq/day of the target species. By way of further example, in one such embodiment the daily dose of the nonabsorbable composition removes at least about 41 mEq/day of the target species. By way of further example, in one such embodiment the daily dose of the nonabsorbable composition removes at least about 42 mEq/day of the target species. By way of further example, in one such embodiment the daily dose of the nonabsorbable composition removes at least about 43 mEq/day of the target species. By way of further example, in one such embodiment the daily dose of the nonabsorbable composition removes at least about 44 mEq/day of the target species. By way of further example, in one such embodiment the daily dose of the nonabsorbable composition removes at least about 45 mEq/day of the target species. By way of further example, in one such embodiment the daily dose of the nonabsorbable composition removes at least about 46 mEq/day of the target species. By way of further example, in one such embodiment the daily dose of the nonabsorbable composition removes at least about 47 mEq/day of the target species. By way of further example, in one such embodiment the daily dose of the nonabsorbable composition removes at least about 48 mEq/day of the target species. By way of further example, in one such embodiment the daily dose of the nonabsorbable composition removes at least about 49 mEq/day of the target species. By way of further example, in one such embodiment the daily dose of the nonabsorbable composition removes at least about 50 mEq/day of the target species.

In certain embodiments of the method of the present disclosure, the daily dose of the nonabsorbable composition removes less than 60 mEq/day of the target species. For example, in one such method the daily dose removes less than 55 mEq/day of the target species. By way of further example, in one such embodiment the daily dose removes less than 50 mEq/day of the target species. By way of further example, in one such embodiment the daily dose removes less than 45 mEq/day of the target species. By way of further example, in one such embodiment the daily dose removes less than 40 mEq/day of the target species. By way of further example, in one such embodiment the daily dose removes less than 35 mEq/day of the target species. By way of further example, in one such embodiment the daily dose removes less than 34 mEq/day of the target species. By way of further example, in one such embodiment the daily dose removes less than 33 mEq/day of the target species. By way of further example, in one such embodiment the daily dose removes less than 32 mEq/day of the target species. By way of further example, in one such embodiment the daily dose removes less than 31 mEq/day of the target species. By way of further example, in one such embodiment the daily dose removes less than 30 mEq/day of the target species. By way of further example, in one such embodiment the daily dose removes less than 29 mEq/day of the target species. By way of further example, in one such embodiment the daily dose removes less than 28 mEq/day of the target species. By way of further example, in one such embodiment the daily dose removes less than 27 mEq/day of the target species. By way of further example, in one such embodiment the daily dose removes less than 26 mEq/day of the target species. By way of further example, in one such embodiment the daily dose removes less than 25 mEq/day of the target species. By way of further example, in one such embodiment the daily dose removes less than 24 mEq/day of the target species. By way of further example, in one such embodiment the daily dose removes less than 23 mEq/day of the target species. By way of further example, in one such embodiment the daily dose removes less than 22 mEq/day of the target species. By way of further example, in one such embodiment the daily dose removes less than 21 mEq/day of the target species. By way of further example, in one such embodiment the daily dose removes less than 20 mEq/day of the target species. By way of further example, in one such embodiment the daily dose removes less than 19 mEq/day of the target species. By way of further example, in one such embodiment the daily dose removes less than 18 mEq/day of the target species. By way of further example, in one such embodiment the daily dose removes less than 17 mEq/day of the target species. By way of further example, in one such embodiment the daily dose removes less than 16 mEq/day of the target species. By way of further example, in one such embodiment the daily dose removes less than 15 mEq/day of the target species. By way of further example, in one such embodiment the daily dose removes less than 14 mEq/day of the target species. By way of further example, in one such embodiment the daily dose removes less than 13 mEq/day of the target species. By way of further example, in one such embodiment the daily dose removes less than 12 mEq/day of the target species. By way of further example, in one such embodiment the daily dose removes less than 11 mEq/day of the target species. By way of further example, in one such embodiment the daily dose removes less than 10 mEq/day of the target species. By way of further example, in one such embodiment the daily dose removes less than 9 mEq/day of the target species. By way of further example, in one such embodiment the daily dose removes less than 8 mEq/day of the target species. By way of further example, in one such embodiment the daily dose removes less than 7 mEq/day of the target species. By way of further example, in one such embodiment the daily dose removes less than 6 mEq/day of the target species.

In certain embodiments of the method of the present disclosure, the daily dose of the nonabsorbable composition has insufficient capacity to remove more than 60 mEq/day of the target species. For example, in one such method the daily dose has insufficient capacity to remove more than 55 mEq/day of the target species. By way of further example, in one such embodiment the daily dose has insufficient capacity to remove more than 50 mEq/day of the target species. By way of further example, in one such embodiment the daily dose has insufficient capacity to remove more than 45 mEq/day of the target species. By way of further example, in one such embodiment the daily dose has insufficient capacity to remove more than 40 mEq/day of the target species. By way of further example, in one such embodiment the daily dose has insufficient capacity to remove more than 35 mEq/day of the target species. By way of further example, in one such embodiment the daily dose has insufficient capacity to remove more than 34 mEq/day of the target species. By way of further example, in one such embodiment the daily dose has insufficient capacity to remove more than 33 mEq/day of the target species. By way of further example, in one such embodiment the daily dose has insufficient capacity to remove more than 32 mEq/day of the target species. By way of further example, in one such embodiment the daily dose has insufficient capacity to remove more than 31 mEq/day of the target species. By way of further example, in one such embodiment the daily dose has insufficient capacity to remove more than 30 mEq/day of the target species. By way of further example, in one such embodiment the daily dose has insufficient capacity to remove more than 29 mEq/day of the target species. By way of further example, in one such embodiment the daily dose has insufficient capacity to remove more than 28 mEq/day of the target species. By way of further example, in one such embodiment the daily dose has insufficient capacity to remove more than 27 mEq/day of the target species. By way of further example, in one such embodiment the daily dose has insufficient capacity to remove more than 26 mEq/day of the target species. By way of further example, in one such embodiment the daily dose has insufficient capacity to remove more than 25 mEq/day of the target species. By way of further example, in one such embodiment the daily dose has insufficient capacity to remove more than 24 mEq/day of the target species. By way of further example, in one such embodiment the daily dose has insufficient capacity to remove more than 23 mEq/day of the target species. By way of further example, in one such embodiment the daily dose has insufficient capacity to remove more than 22 mEq/day of the target species. By way of further example, in one such embodiment the daily dose has insufficient capacity to remove more than 21 mEq/day of the target species. By way of further example, in one such embodiment the daily dose has insufficient capacity to remove more than 20 mEq/day of the target species. By way of further example, in one such embodiment the daily dose has insufficient capacity to remove more than 19 mEq/day of the target species. By way of further example, in one such embodiment the daily dose has insufficient capacity to remove more than 18 mEq/day of the target species. By way of further example, in one such embodiment the daily dose has insufficient capacity to remove more than 17 mEq/day of the target species. By way of further example, in one such embodiment the daily dose has insufficient capacity to remove more than 16 mEq/day of the target species. By way of further example, in one such embodiment the daily dose has insufficient capacity to remove more than 15 mEq/day of the target species. By way of further example, in one such embodiment the daily dose has insufficient capacity to remove more than 14 mEq/day of the target species. By way of further example, in one such embodiment the daily dose has insufficient capacity to remove more than 13 mEq/day of the target species. By way of further example, in one such embodiment the daily dose has insufficient capacity to remove more than 12 mEq/day of the target species. By way of further example, in one such embodiment the daily dose has insufficient capacity to remove more than 11 mEq/day of the target species. By way of further example, in one such embodiment the daily dose has insufficient capacity to remove more than 10 mEq/day of the target species. By way of further example, in one such embodiment the daily dose has insufficient capacity to remove more than 9 mEq/day of the target species. By way of further example, in one such embodiment the daily dose has insufficient capacity to remove more than 8 mEq/day of the target species. By way of further example, in one such embodiment the daily dose has insufficient capacity to remove more than 7 mEq/day of the target species. By way of further example, in one such embodiment the daily dose has insufficient capacity to remove more than 6 mEq/day of the target species.

In certain embodiments of the method of the present disclosure, the method comprises oral administration of a pharmaceutical composition to increase the individual's serum bicarbonate levels wherein: (i) the pharmaceutical composition binds a target species in the individual's digestive system when given orally, the target species being selected from the group consisting of protons, strong acids, and conjugate bases of strong acids; and (ii) the pharmaceutical composition increases the serum bicarbonate level by at least 1 mEq/l in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise at least 25 subjects, each cohort is prescribed the same diet during the study and the study lasts at least two weeks. In one embodiment, the first cohort receives a daily dose of the pharmaceutical composition that does not exceed 100 g/day. In one embodiment, the first cohort receives a daily dose of the pharmaceutical composition that does not exceed 50 g/day. In one embodiment, the first cohort receives a daily dose of the pharmaceutical composition that does not exceed 30 g/day. In one embodiment, the first cohort receives a daily dose of the pharmaceutical composition that does not exceed 25 g/day. In one embodiment, the first cohort receives a daily dose of the pharmaceutical composition that does not exceed 20 g/day. In one embodiment, the first cohort receives a daily dose of the pharmaceutical composition that does not exceed 15 g/day. In one embodiment, the first cohort receives a daily dose of the pharmaceutical composition that does not exceed 10 g/day. In one embodiment, the first cohort receives a daily dose of the pharmaceutical composition that does not exceed 5 g/day. In one embodiment, the target species is protons. In one embodiment, the target species is chloride ions. In one embodiment, the target species is a strong acid. In one embodiment, the target species is HCl. In one embodiment, the pharmaceutical composition is not absorbed when ingested.

In one embodiment, the individual or adult human patient has chronic kidney disease (CKD Stage 3-4; eGFR 20-<60 mL/min/1.73 m²) and a baseline serum bicarbonate value at the start of the study between 12 and 20 mEq/L. In one embodiment, the pharmaceutical composition increases the serum bicarbonate level of the individual or adult human patient by at least 2 mEq/l in the placebo controlled study. In one embodiment, the pharmaceutical composition increases the serum bicarbonate level of the individual or adult human patient by at least 3 mEq/l in the placebo controlled study. In one embodiment, the individual or adult human patient is not yet in need for kidney replacement therapy (dialysis or transplant). In one embodiment, the individual or adult human patient has not yet reached end stage renal disease (“ESRD”).

In one embodiment, the individual or adult human patient has a mGFR of at least 15 mL/min/1.73 m². In one embodiment, the individual or adult human patient has an eGFR of at least 15 mL/min/1.73 m². In one embodiment, the individual or adult human patient has a mGFR of at least 30 mL/min/1.73 m². In one embodiment, the individual or adult human patient has an eGFR of at least 30 mL/min/1.73 m². In one embodiment, the individual or adult human patient has a mGFR of less than 45 mL/min/1.73 m² for at least three months. In one embodiment, the individual or adult human patient has an eGFR of less than 45 mL/min/1.73 m² for at least three months. In one embodiment, the individual or adult human patient has a mGFR of less than 60 mL/min/1.73 m² for at least three months. In one embodiment, the individual or adult human patient has an eGFR of less than 60 mL/min/1.73 m² for at least three months. In one embodiment, the individual or adult human patient has Stage 3A CKD, Stage 3B CKD, or Stage 4 CKD.

While the methods described above refer to daily dose, a further aspect of the disclosure include the methods disclosed herein in which the dose is administered less frequently than once per day (while still being administered on a regular basis). In any of the disclosure, the daily dose specified may, instead, be administrated on a less frequent basis. For example, the doses disclosed here may be administered once every two or three days. Or the doses disclosed here may be administered once, twice or three times a week.

In addition to (or as a surrogate for) serum bicarbonate, other biomarkers of acid-base imbalance may be used as a measure of acid-base status. For example, blood (serum or plasma) pH, total CO₂, anion gap, and/or the concentration of other electrolytes (e.g., sodium, potassium, calcium, magnesium, chloride and/or sulfate) may be used as an indicator of acid-base imbalance. Similarly, net acid excretion (“NAE”), urine pH, urine ammonium concentration, and/or the concentration of other electrolytes in the urine (e.g., sodium, potassium, calcium, magnesium, chloride and/or sulfate) may be used as an indicator of acid-base imbalance.

| | Biomarker | | |
|---|---|---|---|
| Fluid | of interest | Normal/Target Value | Analytical Technique |
| Blood | Total CO₂ | 23-29 mmol/L | Blood gas analyzer; |
| (serum | | | enzymatic assay; ion |
| or | | | selective electrode |
| plasma) | Anion gap | 3-11 mEq/L | Obtained from standard |
| | | | chemistry electrolyte |
| | | | panel |
| | pH | 7.36 to 7.44 | Blood gas analyzer; |
| | | | enzymatic assay; ion |
| | | | selective electrode |
| | Electrolytes | Na = 135-145 mEq/L; | Obtained from standard |
| | | K = 3.5-5 mEq/L; | chemistry electrolyte |
| | | Total Ca = 8-10.5 mEq/L, | panel; |
| | | depending on age and sex; | ion selective electrodes |
| | | Mg = 1.5-2.5 mEq/L, | can be used for Na, Cl |
| | | depending on age; | and K |
| | | Cl = 95-105 mEq/L; | |
| | | phosphate = 2.5-4.5 mEq/L; | |
| | | sulfate = 1 mEq/L | |
| urine | pH | 4.5-8.0 | pH meter |
| | ammonium | 3-65 mmol/L | Enzymatic |
| | citrate | 150-1,191 mg/24-hour urine | Enzymatic |
| | | collection; ranges for 20 to 60 | |
| | | years of age | |
| | sodium | 20 mEq/L in spot samples, | Ion-selective |
| | | 41-227 mEq/L per day | electrode |
| | | (depending upon salt and fluid | |
| | | intake) | |
| | potassium | 17-77 mmol/24 hours; spot | Ion-selective |
| | | sample is ~45 mmol/L | electrode |
| | calcium | Urinary calcium is <250 mg/24 | Enzymatic |
| | | hours in males, <200 mg/24 | |
| | | hours in females | |
| | magnesium | Urinary magnesium is 51- | Enzymatic |
| | | 269 mg/24 hours; spot values | |
| | | are usually reported as a ratio | |
| | | with creatinine and are >0.035 | |
| | | mg Mg/mg creatinine | |
| | chloride | Urinary chloride is 40-224 | Ion-selective |
| | | mmol/24 hours | electrode |
| | Urine Anion | UAG = 0-10 mEq/L; | UAG = (Na⁺ + K⁺) − Cl⁻ in |
| | Gap | Metabolic acidosis indicated | urine. It is a measure |
| | (“UAG”) | when UAG > 20 mEq/L | of ammonium excretion, the |
| | | | primary mechanism for |
| | | | acid excretion. |
| | Net Acid | Urinary net acid excretion is | 24-hour urine collection |
| | Excretion | the total amount of acid | required; Direct NAE |
| | | excreted by the kidney per | measurement (mEq/day) = |
| | | day; the NAE value depends | [NH₄⁺] + [TA] − [HCO₃⁻], |
| | | on the age of the subject, | where TA is concentration |
| | | gender, and protein intake; | of titratable acids |
| | | typical NAE values range from | Indirect NAE |
| | | 9 mEq/day to 38 mEq/day | measurement (mEq/day) = |
| | | | (Cl + P + SO₄ + organic |
| | | | anions) − (Na + K + Ca + |
| | | | Mg). |

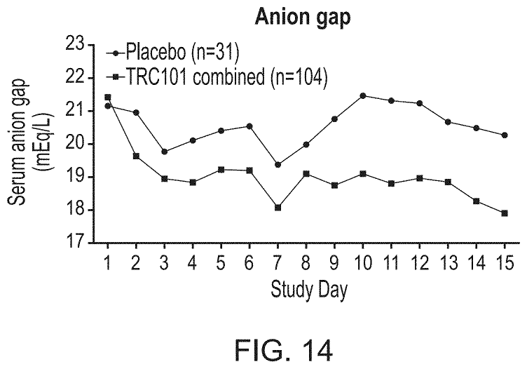

In one embodiment, treatment of an individual as described herein may improve an individuals' serum anion gap. For example, treating an acid base imbalance with a neutral composition having the capacity to bind both protons and anions (unaccompanied by the delivery of sodium or potassium ions) can increase serum bicarbonate without an accompanying increase in sodium or potassium (see Example 3 and FIGS. 13A, 13C and 13D). Consequently, the serum anion gap may be improved (decreased) by at least 1 mEq/l or more (e.g., at least 2 mEq/l) within a period as short as 2 weeks (see Example 3).

The various aspects and embodiments may have a range of advantages, such as improved or successful treatment of metabolic acidosis. Such improvements may also include reduced side effects, increased patient compliance, reduced drug loads, increased speed of treatment, increased magnitude of treatment, avoiding unwanted changes to other electrolytes and/or reduced drug-drug interactions. A further improvement may include reducing a patient's anion gap (as defined above) as part of the methods and other aspects disclosed herein. Further useful features of the disclosed aspects can be found in the examples.

Certain Specific Compositions for Use in Treatment

As previously noted, one aspect disclosed here is a composition for use in a method of treating metabolic acidosis in an adult human patient wherein in said treatment 0.1-12 g of said composition is administered to the patient per day, said composition being a nonabsorbable composition having the capacity to remove protons from the patient, wherein the nonabsorbable composition is characterized by a chloride ion binding capacity of at least 2.5 mEq/g in a Simulated Small Intestine Inorganic (“SIB”) assay. This aspect is based on the data in the examples showing the absorption and removal of HCl to successfully treat patients, allowing the amount of the composition to be set based on its capacity to bind chloride in the SIB assay. As shown in the examples, a composition with this specified level of chloride binding in the “SIB” assay can be used in the specified dose range to successfully treat metabolic acidosis in adult humans. In this aspect, the composition may be administered orally, and so would be an orally administered nonabsorbable composition as defined herein.

This aspect is based on the data in the examples showing the absorption and removal of HCl to successfully treat patients using a composition according to this aspect, allowing the amount of the composition to be set based on its capacity to bind chloride in the SIB assay. Surprisingly, the amounts required for successful treatment were relatively low.

Another aspect of the present disclosure is a composition for use in a method of treating metabolic acidosis in an adult human patient by increasing that patient's serum bicarbonate value by at least 1 mEq/L over 15 days of treatment, said composition being a nonabsorbable composition having the capacity to remove protons from the patient. In this aspect, the composition may be administered orally, and so would be an orally administered nonabsorbable composition as defined herein.

This aspect is based on the data in the examples showing the absorption and removal of HCl to successfully treat patients using a composition according to this aspect which provides new detail regarding the reductions possible using a composition of the disclosure. This aspect includes surprisingly rapid increases in the patient's serum bicarbonate level, for example in the first few days, as well as surprisingly large increases in serum bicarbonate level.

Another aspect of the present disclosure is a composition for use in a method of treating metabolic acidosis in an adult human patient, said patient having a serum bicarbonate level of less than 20 mEq/L prior to treatment, said composition being a nonabsorbable composition having the capacity to remove protons from the patient. In this aspect, the composition may be administered orally, and so would be an orally administered nonabsorbable composition as defined herein.

This aspect is based on the data in the examples showing, for the first time, the successful treatment of patients with a low serum bicarbonate level, for example levels that have not been shown to be so readily treated previously. The patients with lower serum bicarbonate levels responded particularly well to the treatment and this improvement for this subgroup is one advantage of this aspect.

Another aspect of the present disclosure is a composition for use in a method of treating metabolic acidosis in an adult human patient by increasing that patient's serum bicarbonate value by at least 1 mEq/L over 15 days of treatment, wherein in said treatment>12-100 g of said polymer is administered to the patient per day, said composition being a nonabsorbable composition having the capacity to remove protons from the patient, wherein the nonabsorbable composition is characterized by a chloride ion binding capacity of at least 2.5 mEq/g in a Simulated Small Intestine Inorganic Buffer (“SIB”) assay. In this aspect, the composition may be administered orally, and so would be an orally administered nonabsorbable composition as defined herein.

Another aspect of the present disclosure is a composition for use in a method of treating metabolic acidosis in an adult human patient wherein in said treatment>12-100 g of said composition is administered to the patient per day, said composition being a nonabsorbable composition having the capacity to remove protons from the patient, wherein the nonabsorbable composition is characterized by a chloride ion binding capacity of less than 2.5 mEq/g in a Simulated Small Intestine Inorganic Buffer (“SIB”) assay. In this aspect, the composition may be administered orally, and so would be an orally administered nonabsorbable composition as defined herein.

The chloride ion binding capacity in the SIB assay is affected by both the composition's selectivity for binding chloride and the total space available for chloride binding. The term “composition” refers to the active pharmaceutical ingredient, including any counter ions, but not to excipients. So, the “amount” of the composition is the amount of active pharmaceutical ingredient without including other parts of any unit dose form.

More specifically in these aspects, the amount of composition may be any amount disclosed herein in other sections within the range 0.1 g-12 g. For example, 1-11 g, 2-10 g, 3-9 g, 3-8 g, 3-7 g, 3-6 g, 3.5-5.5 g, 4-5 g, or 4.5-5 g of said polymer is administered to the patient per day, or 0.5 g, 1 g, 1.5 g, 2 g, 2.5 g, 3 g, 3.5 g, 4.0 g, 4.5 g or 5.0 g of the composition is administered to the patient per day.

More specifically in these aspects, the chloride ion binding capacity in a Simulated Small Intestine Inorganic Buffer (“SIB”) assay may be greater than 3, 3.5, 4, or 4.5 mEq/g. One upper limit for the chloride ion binding capacity in a SIB assay is 10 mEq/g. Other the upper limits may be 5, 5.5, 6, 6.5, 7, 7.5, 8, 8.5, 9, 9.5 or 10 mEq/g, or there may be no upper limit specified.

All combinations of the amount of composition and the chloride ion binding capacity mentioned here are also disclosed. For example, in one embodiment, the composition has a chloride ion binding capacity in a SIB assay is of at least 4.5 mEq/g and only 0.1-6 gs of composition is administered in the method of treating metabolic acidosis.

The composition in these aspects can additionally have any of the properties or features specified elsewhere herein. For example, the composition may be a nonabsorbable composition as described in the following section. In a similar fashion, the methods of treatment specified in these aspects may include any of the features disclosed in the preceding section regarding certain methods of treatment.

### Nonabsorbable Compositions

As previously noted, the nonabsorbable compositions having the medical uses described herein possess the capacity to remove clinically significant quantities of one or more target species: (i) protons, (ii) the conjugate base(s) of one or more strong acids (e.g., chloride, bisulfate (HSO₄⁻) and/or sulfate (SO₄⁻) ions) and/or (iii) one or more strong acids (e.g., HCl and/or H₂SO₄). To bind such target species, the nonabsorbable compositions may be selected from the group consisting of cation exchange compositions, anion exchange compositions, amphoteric ion exchange compositions, neutral compositions having the capacity to bind both protons and anions, composites thereof and mixtures thereof.

In general, the nonabsorbable composition has a preferred particle size range that is (i) large enough to avoid passive or active absorption through the GI tract and (ii) small enough to not cause grittiness or unpleasant mouth feel when ingested as a powder, sachet and/or chewable tablet/dosage form with a mean particle size of at least 3 microns. For example, in one such embodiment the nonabsorbable composition comprises a population of particles having a mean particle size (volume distribution) in the range of 5 to 1,000 microns. By way of further example, in one such embodiment the nonabsorbable composition comprises a population of particles having a mean particle size (volume distribution) in the range of 5 to 500 microns. By way of further example, in one such embodiment the nonabsorbable composition comprises a population of particles having a mean particle size (volume distribution) in the range of 10 to 400 microns. By way of further example, in one such embodiment the nonabsorbable composition comprises a population of particles having a mean particle size (volume distribution) in the range of 10 to 300 microns. By way of further example, in one such embodiment the nonabsorbable composition comprises a population of particles having a mean particle size (volume distribution) in the range of 20 to 250 microns. By way of further example, in one such embodiment the nonabsorbable composition comprises a population of particles having a mean particle size (volume distribution) in the range of 30 to 250 microns. By way of further example, in one such embodiment the nonabsorbable composition comprises a population of particles having a mean particle size (volume distribution) in the range of 40 to 180 microns. In certain embodiments, less than 7% of the particles in the population (volume distribution) have a diameter less than 10 microns. For example, in such embodiments less than 5% of the particles in the particles in the population (volume distribution) have a diameter less than 10 microns. By way of further example, in such embodiments less than 2.5% of the particles in the particles in the population (volume distribution) have a diameter less than 10 microns. By way of further example, in such embodiments less than 1% of the particles in the particles in the population (volume distribution) have a diameter less than 10 microns. In all embodiments, the particle size may be measured using the protocol set out in the abbreviations and definitions section (above).

To minimize GI side effects in patients that are often related to a large volume polymer gel moving through the GI tract, a low Swelling Ratio of the nonabsorbable composition is preferred (0.5 to 10 times its own weight in water). For example, in one such embodiment the nonabsorbable composition has a Swelling Ratio of less than 9. By way of further example, in one such embodiment the nonabsorbable composition has a Swelling Ratio of less than 8. By way of further example, in one such embodiment the nonabsorbable composition has a Swelling Ratio of less than 7. By way of further example, in one such embodiment the nonabsorbable composition has a Swelling Ratio of less than 6. By way of further example, in one such embodiment the nonabsorbable composition has a Swelling Ratio of less than 5. By way of further example, in one such embodiment the nonabsorbable composition has a Swelling Ratio of less than 4. By way of further example, in one such embodiment the nonabsorbable composition has a Swelling Ratio of less than 3. By way of further example, in one such embodiment the nonabsorbable composition has a Swelling Ratio of less than 2.

The amount of the target species (proton, conjugate base of a strong acid and/or strong acid) that is bound as the nonabsorbable composition transits the GI tract is largely a function of the binding capacity of the composition for the target species (protons, the conjugate base of a strong acid, and/or a strong acid) and the quantity of the nonabsorbable composition administered per day as a daily dose. In general, the theoretical binding capacity for a target species may be determined using a SGF assay and determining the amount of a species that appeared in or disappeared from the SGF buffer during the SGF assay. For example, the theoretical proton binding capacity of a cation exchange resin may be determined by measuring the increase in the amount of cations (other than protons) in the buffer during a SGF assay. Similarly, the theoretical anion binding capacity of an anion exchange resin (in a form other than the chloride form) may be determined by measuring the increase in the amount of anions (other than chloride ions) in the buffer during a SGF assay. Additionally, the theoretical anion binding capacity of a neutral composition for protons and the conjugate base of a strong acid may be determined by measuring the decrease in chloride concentration in the buffer during a SGF assay.

In general, the nonabsorbable composition will have a theoretical binding capacity for the target species of at least about 0.5 mEq/g (as determined in an SGF assay). For example, in some embodiments the nonabsorbable composition will have a theoretical binding capacity for the target species of at least about 1 mEq/g. By way of further example, in some embodiments the nonabsorbable composition will have a theoretical binding capacity for the target species of at least about 2 mEq/g. By way of further example, in some embodiments the nonabsorbable composition will have a theoretical binding capacity for the target species of at least about 3 mEq/g. By way of further example, in some embodiments the nonabsorbable composition will have a theoretical binding capacity for the target species of at least about 4 mEq/g. By way of further example, in some embodiments the nonabsorbable composition will have a theoretical binding capacity for the target species of at least about 5 mEq/g. By way of further example, in some embodiments the nonabsorbable composition will have a theoretical binding capacity for the target species of at least about 7.5 mEq/g. By way of further example, in some embodiments the nonabsorbable composition will have a theoretical binding capacity for the target species of at least about 10 mEq/g. By way of further example, in some embodiments the nonabsorbable composition will have a theoretical binding capacity for the target species of at least about 12.5 mEq/g. By way of further example, in some embodiments the nonabsorbable composition will have a theoretical binding capacity for the target species of at least about 15 mEq/g. By way of further example, in some embodiments the nonabsorbable composition will have a theoretical binding capacity for the target species of at least about 20 mEq/g. In general, the nonabsorbable composition will typically have a theoretical binding capacity for the target species that is not in excess of about 35 mEq/g. For example, in some embodiments, the theoretical binding capacity of the nonabsorbable compositions for the target species that is not be excess of 30 mEq/g. Thus, for example, the theoretical binding capacity of the nonabsorbable compositions for the target species may range from 2 to 25 mEq/g, 3 to 25 mEq/g, 5 to 25 mEq/g, 10 to 25 mEq/g, 5 to 20 mEq/g, 6 to 20 mEq/g, 7.5 to 20 mEq/g, or even 10 to 20 mEq/g. In those embodiments in which the target species comprises protons and at least one conjugate base, the binding capacities recited in this paragraph are the theoretical binding capacities for protons and the theoretical binding capacities for the conjugate base(s), independently and individually, and not the sum thereof.

In general, the nonabsorbable composition will have a theoretical binding capacity for protons of at least about 0.5 mEq/g (as determined in an SGF assay). For example, in some embodiments the nonabsorbable composition will have a theoretical binding capacity for protons of at least about 1 mEq/g. By way of further example, in some embodiments the nonabsorbable composition will have a theoretical binding capacity for protons of at least about 2 mEq/g. By way of further example, in some embodiments the nonabsorbable composition will have a theoretical binding capacity for protons of at least about 3 mEq/g. By way of further example, in some embodiments the nonabsorbable composition will have a theoretical binding capacity for protons of at least about 4 mEq/g. By way of further example, in some embodiments the nonabsorbable composition will have a theoretical binding capacity for protons of at least about 5 mEq/g. By way of further example, in some embodiments the nonabsorbable composition will have a theoretical binding capacity for protons of at least about 7.5 mEq/g. By way of further example, in some embodiments the nonabsorbable composition will have a theoretical binding capacity for protons of at least about 10 mEq/g. By way of further example, in some embodiments the nonabsorbable composition will have a theoretical binding capacity for protons of at least about 12.5 mEq/g. By way of further example, in some embodiments the nonabsorbable composition will have a theoretical binding capacity for protons of at least about 15 mEq/g. By way of further example, in some embodiments the nonabsorbable composition will have a theoretical binding capacity for protons of at least about 20 mEq/g. In general, the nonabsorbable composition will typically have a theoretical binding capacity for protons that is not in excess of about 35 mEq/g. For example, in some embodiments, the theoretical binding capacity of the nonabsorbable compositions for protons that is not be excess of 30 mEq/g. Thus, for example, the theoretical binding capacity of the nonabsorbable compositions for protons may range from 2 to 25 mEq/g, 3 to 25 mEq/g, 5 to 25 mEq/g, 10 to 25 mEq/g, 5 to 20 mEq/g, 6 to 20 mEq/g, 7.5 to 20 mEq/g, or even 10 to 20 mEq/g. In those embodiments in which the target species comprises protons and at least one conjugate base, the binding capacities recited in this paragraph are the theoretical binding capacities for protons and the theoretical binding capacities for the conjugate base(s), independently and individually, and not the sum thereof.

Phosphate, bicarbonate, bicarbonate equivalents, the conjugate bases of bile and fatty acids are potential interfering anions for chloride or other conjugate bases of strong acids (e.g., HSO₄⁻ and SO₄²⁻) in the stomach and small intestine. Therefore, rapid and preferential binding of chloride over phosphate, bicarbonate equivalents, and the conjugate bases of bile and fatty acids in the small intestine is desirable and the SIB assay may be used to determine kinetics and preferential binding. Since the transit time of the colon is slow (2-3 days) relative to the small intestine, and since conditions in the colon will not be encountered by an orally administered nonabsorbable composition until after stomach and small intestine conditions have been encountered, kinetics of chloride binding by a nonabsorbable composition do not need to be as rapid in the colon or under in vitro conditions designed to mimic the late small intestine/colon. It is, however, desirable that chloride binding and selectivity over other interfering anions is high, for example, at 24 and/or 48 hours or longer.

In one embodiment, the nonabsorbable composition is characterized by a chloride ion binding capacity of at least 1 mEq/g in a Simulated Small Intestine Inorganic Buffer (“SIB”) assay. For example, in one such embodiment the nonabsorbable composition is characterized by a chloride ion binding capacity of at least 1.5 mEq/g in a SIB assay. By way of further example, in one such embodiment the nonabsorbable composition is characterized by a chloride ion binding capacity of at least 2 mEq/g in a SIB assay. By way of further example, in one such embodiment the nonabsorbable composition is characterized by a chloride ion binding capacity of at least 2.5 mEq/g in a SIB assay. By way of further example, in one such embodiment the nonabsorbable composition is characterized by a chloride ion binding capacity of at least 3 mEq/g in a SIB assay. By way of further example, in one such embodiment the nonabsorbable composition is characterized by a chloride ion binding capacity of at least 3.5 mEq/g in a SIB assay. By way of further example, in one such embodiment the nonabsorbable composition is characterized by a chloride ion binding capacity of at least 4 mEq/g in a SIB assay. By way of further example, in one such embodiment the nonabsorbable composition is characterized by a chloride ion binding capacity of at least 4.5 mEq/g in a SIB assay. By way of further example, in one such embodiment the nonabsorbable composition is characterized by a chloride ion binding capacity of at least 5 mEq/g in a SIB assay. By way of further example, in one such embodiment the nonabsorbable composition is characterized by a chloride ion binding capacity of at least 5.5 mEq/g in a SIB assay. By way of further example, in one such embodiment the nonabsorbable composition is characterized by a chloride ion binding capacity of at least 6 mEq/g in a SIB assay.

In one embodiment, the nonabsorbable composition binds a significant amount of chloride relative to phosphate as exhibited, for example, in a SIB assay. For example, in one embodiment the ratio of the amount of bound chloride to bound phosphate in a SIB assay is at least 0.1:1, respectively. By way of further example, in one such embodiment the ratio of the amount of bound chloride to bound phosphate in a SIB assay is at least 0.2:1, respectively. By way of further example, in one such embodiment the ratio of the amount of bound chloride to bound phosphate in a SIB assay is at least 0.25:1, respectively. By way of further example, in one such embodiment the ratio of the amount of bound chloride to bound phosphate in a SIB assay is at least 0.3:1, respectively. By way of further example, in one such embodiment the ratio of the amount of bound chloride to bound phosphate in a SIB assay is at least 0.35:1, respectively. By way of further example, in one such embodiment the ratio of the amount of bound chloride to bound phosphate in a SIB assay is at least 0.4:1, respectively. By way of further example, in one such embodiment the ratio of the amount of bound chloride to bound phosphate in a SIB assay is at least 0.45:1, respectively. By way of further example, in one such embodiment the ratio of the amount of bound chloride to bound phosphate in a SIB assay is at least 0.5:1, respectively. By way of further example, in one such embodiment the ratio of the amount of bound chloride to bound phosphate in a SIB assay is at least 2:3, respectively. By way of further example, in one such embodiment the ratio of the amount of bound chloride to bound phosphate in a SIB assay is at least 0.75:1, respectively. By way of further example, in one such embodiment the ratio of the amount of bound chloride to bound phosphate in a SIB assay is at least 0.9:1, respectively. By way of further example, in one such embodiment the ratio of the amount of bound chloride to bound phosphate in a SIB assay is at least 1:1, respectively. By way of further example, in one such embodiment the ratio of the amount of bound chloride to bound phosphate in a SIB assay is at least 1.25:1, respectively. By way of further example, in one such embodiment the ratio of the amount of bound chloride to bound phosphate in a SIB assay is at least 1.5:1, respectively. By way of further example, in one such embodiment the ratio of the amount of bound chloride to bound phosphate in a SIB assay is at least 1.75:1, respectively. By way of further example, in one such embodiment the ratio of the amount of bound chloride to bound phosphate in a SIB assay is at least 2:1, respectively. By way of further example, in one such embodiment the ratio of the amount of bound chloride to bound phosphate in a SIB assay is at least 2.25:1, respectively. By way of further example, in one such embodiment the ratio of the amount of bound chloride to bound phosphate in a SIB assay is at least 2.5:1, respectively. By way of further example, in one such embodiment the ratio of the amount of bound chloride to bound phosphate in a SIB assay is at least 2.75:1, respectively. By way of further example, in one such embodiment the ratio of the amount of bound chloride to bound phosphate in a SIB assay is at least 3:1, respectively. By way of further example, in one such embodiment the ratio of the amount of bound chloride to bound phosphate in a SIB assay is at least 4:1, respectively. By way of further example, in one such embodiment the ratio of the amount of bound chloride to bound phosphate in a SIB assay is at least 5:1, respectively.

In one embodiment, the orally administered nonabsorbable composition is characterized by a proton-binding capacity and a chloride binding capacity in Simulated Gastric Fluid of at least 1 mEq/g in a SGF assay. For example, in one such embodiment the nonabsorbable composition is characterized by a proton-binding capacity and a chloride binding capacity in a SGF assay of at least 2 mEq/g. By way of further example, in one such embodiment the nonabsorbable composition is characterized by a proton-binding capacity and a chloride binding capacity in a SGF assay of at least 3 mEq/g. By way of further example, in one such embodiment the nonabsorbable composition is characterized by a proton-binding capacity and a chloride binding capacity in a SGF assay of at least 4 mEq/g. By way of further example, in one such embodiment the nonabsorbable composition is characterized by a proton-binding capacity and a chloride binding capacity in a SGF assay of at least 5 mEq/g. By way of further example, in one such embodiment the nonabsorbable composition is characterized by a proton-binding capacity and a chloride binding capacity in a SGF assay of at least 6 mEq/g. By way of further example, in one such embodiment the nonabsorbable composition is characterized by a proton-binding capacity and a chloride binding capacity in a SGF assay of at least 7 mEq/g. By way of further example, in one such embodiment the nonabsorbable composition is characterized by a proton-binding capacity and a chloride binding capacity in a SGF assay of at least 8 mEq/g. By way of further example, in one such embodiment the nonabsorbable composition is characterized by a proton-binding capacity and a chloride binding capacity in a SGF assay of at least 9 mEq/g. By way of further example, in one such embodiment the nonabsorbable composition is characterized by a proton-binding capacity and a chloride binding capacity in a SGF assay of at least 10 mEq/g. By way of further example, in one such embodiment the nonabsorbable composition is characterized by a proton-binding capacity and a chloride binding capacity in a SGF assay of at least 11 mEq/g. By way of further example, in one such embodiment the nonabsorbable composition is characterized by a proton-binding capacity and a chloride binding capacity in a SGF assay of at least 12 mEq/g. By way of further example, in one such embodiment the nonabsorbable composition is characterized by a proton-binding capacity and a chloride binding capacity in a SGF assay of at least 13 mEq/g. By way of further example, in one such embodiment the nonabsorbable composition is characterized by a proton-binding capacity and a chloride binding capacity in a SGF assay of at least 14 mEq/g. By way of further example, in one such embodiment the nonabsorbable composition is characterized by a proton-binding capacity and a chloride binding capacity after 1 hour in SGF that is at least 50% of the proton-binding capacity and the chloride binding capacity, respectively, of the nonabsorbable composition at 24 hours in SGF. By way of further example, in one such embodiment the nonabsorbable composition is characterized by a proton-binding capacity and a chloride binding capacity after 1 hour in SGF that is at least 60% of the proton-binding capacity and the chloride binding capacity, respectively, of the nonabsorbable composition at 24 hours in SGF. By way of further example, in one such embodiment the nonabsorbable composition is characterized by a proton-binding capacity and a chloride binding capacity after 1 hour in SGF that is at least 70% of the proton-binding capacity and the chloride binding capacity, respectively, of the nonabsorbable composition at 24 hours in SGF. By way of further example, in one such embodiment the nonabsorbable composition is characterized by a proton-binding capacity and a chloride binding capacity after 1 hour in SGF that is at least 80% of the proton-binding capacity and the chloride binding capacity, respectively, of the nonabsorbable composition at 24 hours in SGF. By way of further example, in one such embodiment the nonabsorbable composition is characterized by a proton-binding capacity and a chloride binding capacity after 1 hour in SGF that is at least 90% of the proton-binding capacity and the chloride binding capacity, respectively, of the nonabsorbable composition at 24 hours in SGF.

In one embodiment, the nonabsorbable composition is a cation exchange material comprising an insoluble (in the gastric environment) support structure and exchangeable cations. The cation exchange material may be organic (e.g., polymeric), inorganic (e.g., a zeolite) or a composite thereof. The exchangeable cations may be selected, for example, from the group consisting of lithium, sodium, potassium, calcium, magnesium, iron and combinations thereof, and more preferably from the group consisting of sodium, potassium, calcium, magnesium, and combinations thereof. In such embodiments it is generally preferred that the nonabsorbable composition contain a combination of exchangeable cations that establish or maintain electrolyte homeostasis. For example, in one such embodiment the nonabsorbable composition optionally contains exchangeable sodium ions, but when included, the amount of the sodium ions in a daily dose is insufficient to increase the patient's serum sodium ion concentration to a value outside the range of 135 to 145 mEq/l. By way of further example, in one such embodiment the nonabsorbable composition optionally contains exchangeable potassium ions, but when included, the amount of the potassium ions in a daily dose is insufficient to increase the patient's serum potassium ion concentration to a value outside the range of 3.7 to 5.2 mEq/L. By way of further example, in one such embodiment the nonabsorbable composition optionally contains exchangeable magnesium ions, but when included, the amount of the magnesium ions in a daily dose is insufficient to increase the patient's serum magnesium ion concentration to a value outside the range of 1.7 to 2.2 mg/dL. By way of further example, in one such embodiment the nonabsorbable composition optionally contains exchangeable calcium ions, but when included, the amount of the calcium ions in a daily dose is insufficient to increase the patient's serum calcium ion concentration to a value outside the range of 8.5 to 10.2 mg/dL. By way of further example, in one such embodiment the nonabsorbable composition contains a combination of exchangeable cations selected from the group consisting of sodium, potassium, calcium, magnesium, and combinations thereof, designed to maintain serum Na⁺ levels within the range of 135 to 145 mEq/l, serum K⁺ levels within the range of 3.7 to 5.2 mEq/L, serum Mg²⁺ levels within the range of 1.7 to 2.2 mg/dL and serum Ca²⁺ levels within the range of 8.5 to 10.2 mg/dL.

In one embodiment, the nonabsorbable composition is a cation exchange material comprising an insoluble (in the gastric environment) support structure, optionally containing exchangeable sodium ions cations. The cation exchange material may be organic (e.g., polymeric), inorganic (e.g., a molecular sieve) or a composite thereof. In one such embodiment, the nonabsorbable composition contains less than 12% by weight sodium. For example, in one such embodiment the nonabsorbable composition contains less than 9% by weight sodium. By way of further example, in one such embodiment the nonabsorbable composition contains less than 6% by weight sodium. By way of further example, in one such embodiment the nonabsorbable composition contains less than 3% by weight sodium. By way of further example, in one such embodiment the nonabsorbable composition contains less than 1% by weight sodium. By way of further example, in one such embodiment the nonabsorbable composition contains less than 0.1% by weight sodium. By way of further example, in one such embodiment the nonabsorbable composition contains less than 0.01% by weight sodium. By way of further example, in one such embodiment the nonabsorbable composition contains between 0.05 and 3% by weight sodium.

In one exemplary embodiment, the nonabsorbable composition is a resin comprising any of a wide range of crosslinked polymeric materials that are able to bind protons in aqueous solutions. Exemplary crosslinked polymeric material comprises a polyanion crosslinked material selected from poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof. In one embodiment, the polyanion is coordinated to exchangeable monovalent cations, divalent cations, or a combination thereof. Exemplary monovalent cations include lithium, sodium, and potassium, or any combination thereof. Exemplary divalent cations include magnesium and calcium or combinations thereof.

In one exemplary embodiment, the nonabsorbable composition is a cation exchange resin comprising a polyanion backbone that exchanges cations for protons and has an average pKa of at least 4. For example, in one embodiment, the polyanion backbone has an average pKa of 4-5. By way of further example, in one such embodiment the polyanion backbone has an average pKa of 5-6. By way of further example, in one such embodiment the polyanion backbone has an average pKa of 6-7. By way of further example, in one such embodiment the polyanion backbone has an average pKa of greater than 7. Exemplary cation exchange resins include poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof. In one embodiment, these polyanion backbones are further functionalized with functional groups to affect the pKa. These functional groups can increase pKa when electron donating, or decrease pKa when electron withdrawing. Exemplary electron donating groups include amino, hydroxyl, methyl ether, ether, phenyl, and amido. Exemplary electron withdrawing groups include flouro, chloro, halo, sulphonyl, nitroxyl, trifluoromethyl, and cyano. Further exemplary cation exchange resins include resins modified with protonable functional groups including carboxylic acids and functionalized alcohols.

Polymeric cation exchanger resins may be prepared using a range of chemistries, including for example, (i) substitution polymerization of polyfunctional reagents at least one of which comprises basic anionic or conjugate-acid moieties, (2) radical polymerization of a monomer comprising at least one acid or conjugate-acid containing moiety, and (3) crosslinking of a basic anionic or conjugate-acid containing intermediate with a polyfunctional crosslinker, optionally containing basic anionic or conjugate-acid moieties. The resulting crosslinked polymers may thus, for example, be crosslinked homopolymers or crosslinked copolymers. By way of further example, the resulting crosslinked polymers will typically possess repeat units comprising basic anionic or conjugate-acid, separated by the same or varying lengths of repeating linker (or intervening) units. In some embodiments, the polymers comprise repeat units comprising a basic anionic or conjugate-acid moiety and an intervening linker unit. In other embodiments, multiple basic anionic or conjugate-acid containing repeat units are separated by one or more linker units. Additionally, the polyfunctional crosslinkers may comprise proton binding functional groups, e.g. basic anionic, (“active crosslinkers”) or may lack proton binding functional groups such as acrylates (“passive crosslinkers”).

In some embodiments, a basic anion or conjugate-acid monomer is polymerized and the polymer is concurrently crosslinked in a substitution polymerization reaction. The basic anion or conjugate-acid reactant (monomer) in the concurrent polymerization and crosslinking reaction can react more than one time for the substitution polymerization. In one such embodiment, the basic anion or conjugate-acid monomer is a branched basic anion or conjugate-acid possessing at least two reactive moieties to participate in the substitution polymerization reaction.

In one embodiment, the nonabsorbable composition comprises a cation exchange ceramic material. Porous inorganic binders exhibit a range of properties. Functionally, they are able to sequester materials on the basis of their size and polarity, as they exhibit a framework charge with porous structure. They are structurally diverse and can be crystalline or non-crystalline crystalline (amorphous). Classes of porous materials that fall under the class of inorganic binders include hydrous oxides (e.g., aluminum oxide) and metal alum ino-silicate compounds where the metal can be an alkali or alkali earth metal such sodium, potassium, lithium, magnesium or calcium. Many of these compounds have well-defined crystalline structures. This class of compounds has been used for various biopharmaceutical applications.

The pore diameters of inorganic microporous and mesoporous materials are measured in angstroms (Å) or nanometers (nm). According to IUPAC notation, microporous materials have pore diameters of less than 2 nm (20 Å) and macroporous materials have pore diameters of greater than 50 nm (500 Å); the mesoporous category thus lies in the middle with pore diameters between 2 and 50 nm (20-500 Å). The porosity of inorganic porous materials can be tuned or designed, by the appropriate use of poragen or “co-monomer metals” within the lattices of the porous material. By the appropriate choice of elements, the pore size has been seen to range in size from 3 Å to 8 Å. These compositions have a porous system allowing solute together with other dissolved species to enter the porous framework of the material, resulting in absorption of the dissolved species. Tuning the cavities and pore size of the materials, can allow adsorption of molecules of particular dimensions, while rejecting those of larger dimensions. From a binding perspective using size as a selectivity mechanism the chloride ion has the advantage of its small size (the radius of chloride anion is 1.8 Å, and the molecular weight of chloride anion is 35.5) compared to the other species present in the digestive tract.

| Molecules absorbed (small, polar | Molecules excluded (large, non |
|---|---|
| organic or inorganic) | polar and high molecular weight) |
| Water (Solubility in water; miscible, | Bile acids (Solubility in water; |
| Mw 18) | 0.24%, Mw 392.5) |
| HCl (Soluble in water (38%), Mw | Phosphoric acid (Solubility in |
| 36.5) | water; miscible, Mw 98.0) |
| Acetic acid (Solubility in water; | Fatty acids (Solubility in water, |
| miscible, Mw 60.0) | non miscible, Mw > 200 |

Exemplary cation exchange ceramic materials include any of a wide range of microporous or mesoporous ceramic materials. In one embodiment, the nonabsorbable composition comprises a molecular sieve, such as a molecular sieve selected from the group consisting of silica, titanosilicate, metalloaluminate, aluminophosphate and gallogerminate molecular sieves. In one embodiment, the nonabsorbable composition comprises a zeolite, a borosilicate, a gallosilicate, a ferrisilicate or a chromosilicate molecular sieve.

Inorganic porous materials exhibit the property of sequestering substances from an external environment. The mechanism to bind proton or chloride or HCl can be either an adsorptive or absorptive mechanism, where the ions are bound via the specific porosity of the matrix, or an ion exchange mechanism. The strong adsorptive force in zeolite molecular sieves are due to the polarity of the surface (hydroxyl metalloid) and cations that are exposed within the crystal lattice. The cations on the surface act as a site of strong localized positive charge that electrostatically attract the partial negative charges of polar molecules (for example, the chloride of HCl). A basic formula for zeolite can be represented by, M_{2/n}O.Al₂O₃.xSiO₂.yH₂O where M is a cation of n valence. The fundamental building block of the molecular sieve structure is tetrahedral with 4 oxygen anions surrounding a silicon or alumina cation. Sodium ions or other cations (e.g. potassium, calcium) make up the positive charge deficit of the alumina tetrahedron to extend the crystal lattice. In many molecular sieve types the sodium can be exchanged or the sodium can function as a permanent positive charge within the crystal lattice thus providing the electrostatic interaction. Given these mechanisms, hydrochloric acid can be sequestered from solution via a cation exchange mechanism (sodium for proton), anion exchange mechanism (hydroxide for chloride), or via electrostatic interaction of the hydrochloric acid ionic species.

The methods used to bind HCl are well known in the art and involve contacting the molecular sieve with a solution containing the desired HCl concentration in water. Exchange conditions include a temperature of about 25° C. to about 100° C., and a time of about 20 minutes to about 2 hours. These conditions include conditions and exposure times encountered in the gastrointestinal tract.

In one embodiment, the nonabsorbable composition is an anion exchange material comprising an insoluble (in the gastric environment) support structure and exchangeable anions. The anion exchange material may be organic (e.g., polymeric), inorganic (e.g., an apatite, hydrotalcite or a hydrated gel of aluminum, iron(III) or zirconium hydroxide) or a composite thereof.

In one embodiment, the nonabsorbable composition comprises an anion exchange material. Exemplary anion exchange materials include strongly and weakly basic anion exchange materials. For example, the anion exchange material may include any of a wide range of polymers comprising quaternary amine moieties, phosphonium salts, N-heteroaromatic salts, or combinations thereof. Other exemplary anion exchange materials include poly(ionic liquids), wherein the side chain is selected from the group consisting of salts of tetraalkyl ammonium, imidazolium, pyridinium, pyrrolidonium, guanidinium, piperidinium, and tetraalkyl phosphonium cations and combinations thereof. By way of further example, in one such embodiment the anion exchange material is a halide responsive polymer such that a conformational change occurs when about 1 mEq/g to about 35 mEq/g of chloride is initially bound to the polymer and subsequently retained for the duration of the GI transit time. In certain embodiments, the halide response conformational change occurs when 2 mEq/g to about 25 mEq/g chloride is bound, and in certain more specific embodiments, the halide response conformational change occurs when 3 to 25 mEq/g, 5 to 25 mEq/g, 10 to 25 mEq/g, 5 to 20 mEq/g, 6 to 20 mEq/g, 7.5 to 20 mEq/g, or even 10 to 20 mEq/g chloride is bound. The polymeric backbone of any of the aforementioned polymers can derive from vinyl, allyl, styrenic, acrylamide, meth(acrylamide), or copolymers thereof. By way of further example, the anion exchange functionality may be incorporated into the backbone of the polymer. Examples include poly(tetraalkyl ammonium), poly(imidazolium), poly(pyridinium), poly(pyrrolidonium), poly(piperidinium), and poly(tetraalkyl phosphonium) cations or combinations thereof. The exchangeable anion can consist of hydroxide, bicarbonate, acetate, nitrate or any pharmaceutically and biologically acceptable base or combination thereof.

In one embodiment, the nonabsorbable composition is an anion exchange material comprising at least 1 mEq/g of an anion selected from the group consisting of hydroxide, carbonate, citrate or other bicarbonate equivalent anion, or a combination thereof. In this embodiment, the nonabsorbable composition has the capacity to induce an increase in the individual's serum bicarbonate value, at least in part, by delivering a physiologically significant amount of hydroxide, carbonate, citrate or other bicarbonate equivalent, or a combination thereof. Exemplary bicarbonate equivalent anions include acetate, lactate and the conjugate bases of other short chain carboxylic acids. In one such embodiment, the nonabsorbable composition comprises at least 2 mEq/g of an anion selected from the group consisting of hydroxide, carbonate, citrate or other bicarbonate equivalent anion. By way of further example, in one such embodiment the nonabsorbable composition comprises at least 3 mEq/g of an anion selected from the group consisting of hydroxide, carbonate, citrate or other bicarbonate equivalent anion. By way of further example, in one such embodiment the nonabsorbable composition comprises at least 4 mEq/g of an anion selected from the group consisting of hydroxide, carbonate, citrate or other bicarbonate equivalent anion. By way of further example, in one such embodiment the nonabsorbable composition comprises at least 5 mEq/g of an anion selected from the group consisting of hydroxide, carbonate, citrate or other bicarbonate equivalent anion.

In one embodiment, the nonabsorbable composition is an anion exchange material comprising less than 10 mEq/g of an anion selected from the group consisting of hydroxide, carbonate, citrate or other bicarbonate equivalent anion, or a combination thereof. In one such embodiment, the nonabsorbable composition comprises less than 7.5 mEq/g of an anion selected from the group consisting of hydroxide, carbonate, citrate or other bicarbonate equivalent anion. By way of further example, in one such embodiment the nonabsorbable composition comprises less than 5 mEq/g of an anion selected from the group consisting of hydroxide, carbonate, citrate or other bicarbonate equivalent anion. By way of further example, in one such embodiment the nonabsorbable composition comprises less than 2.5 mEq/g of an anion selected from the group consisting of hydroxide, carbonate, citrate or other bicarbonate equivalent anion. By way of further example, in one such embodiment the nonabsorbable composition comprises less than 1 mEq/g of an anion selected from the group consisting of hydroxide, carbonate, citrate or other bicarbonate equivalent anion. By way of further example, in one such embodiment the nonabsorbable composition comprises less than 0.1 mEq/g of an anion selected from the group consisting of hydroxide, carbonate, citrate or other bicarbonate equivalent anion.

In one embodiment, the nonabsorbable composition comprises an amphoteric ion exchange resin. Exemplary amphoteric ion-exchange resins include crosslinked polystyrene, polyethylene or the like as a base material and quaternary ammonium group, carboxylic acid group and the like in (i) the same pendant groups (e.g., betaine-containing pendant groups) such as the amphoteric resin sold under the trade designation DIAION AMPO3 (Mitsubishi Chemical Corporation) or (ii) different pendant groups (e.g., mixed charged copolymers containing the residues of at least two different monomers, one containing ammonium groups and one containing carboxylic acid groups), to provide a function of ion-exchanging the both of cations and negative ions. Exemplary amphoteric ion-exchange resins containing a mixture of cation and anion exchange sites also include resins in which a linear polymer is trapped inside a crosslinked ion exchange resin, such as the amphoteric resin sold under the trade designation DOWEX™ Retardion 11A8 (Dow Chemical Company).

In one embodiment, the nonabsorbable composition comprises a neutral composition having the capacity to bind both protons and anions. Exemplary neutral nonabsorbable compositions that bind both protons and anions include polymers functionalized with propylene oxide, polymers functionalized with Michael acceptors, expanded porphyrins, covalent organic frameworks, and polymers containing amine and/or phosphine functional groups.

In those embodiments in which the nonabsorbable composition binds chloride ions, it is generally preferred that the nonabsorbable composition selectively bind chloride ions relative to other counter ions such as bicarbonate equivalent anions, phosphate anions, and the conjugate bases of bile and fatty acids. Stated differently, it is generally preferred in these embodiments that the nonabsorbable composition (i) remove more chloride ions than bicarbonate equivalent anions (ii) remove more chloride ions than phosphate anions, and (iii) remove more chloride ions than the conjugate bases of bile and fatty acids. Advantageously, therefore, treatment with the nonabsorbable composition does not induce or exacerbate hypophosphatemia (i.e., a serum phosphorous concentration of less than about 2.4 mg/dL, does not significantly elevate low density lipoproteins (“LDL”), or otherwise negatively impact serum or colon levels of metabolically relevant anions.

In some embodiments, the pharmaceutical composition comprises a crosslinked polymer containing the residue of an amine corresponding to Formula 1.

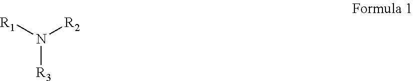

wherein R₁, R₂ and R₃ are independently hydrogen, hydrocarbyl, substituted hydrocarbyl provided, however, at least one of R₁, R₂ and R₃ is other than hydrogen. Stated differently, at least one of R₁, R₂ and R₃ is hydrocarbyl or substituted hydrocarbyl, and the others of R₁, R₂ and R₃ are independently hydrogen, hydrocarbyl, or substituted hydrocarbyl. In one embodiment, for example, R₁, R₂ and R₃ are independently hydrogen, aryl, aliphatic, heteroaryl, or heteroaliphatic provided, however, each of R₁, R₂ and R₃ are not hydrogen. By way of further example, in one such embodiment R₁, R₂ and R₃ are independently hydrogen, saturated hydrocarbons, unsaturated aliphatic, unsaturated heteroaliphatic, heteroalkyl, heterocyclic, aryl or heteroaryl, provided, however, each of R₁, R₂ and R₃ are not hydrogen. By way of further example, in one such embodiment R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ are not hydrogen. By way of further example, in one such embodiment R₁, R₂ and R₃ are independently hydrogen, alkyl, aminoalkyl, alkanol, aryl, haloalkyl, hydroxyalkyl, ethereal, heteroaryl or heterocyclic provided, however, each of R₁, R₂ and R₃ are not hydrogen. By way of further example, in one such embodiment R₁ and R₂ (in combination with the nitrogen atom to which they are attached) together constitute part of a ring structure, so that the monomer as described by Formula 1 is a nitrogen-containing heterocycle (e.g., piperidine) and R₃ is hydrogen, or heteroaliphatic. By way of further example, in one embodiment R₁, R₂ and R₃ are independently hydrogen, aliphatic or heteroaliphatic provided, however, at least one of R₁, R₂ and R₃ is other than hydrogen. By way of further example, in one embodiment R₁, R₂ and R₃ are independently hydrogen, allyl, or aminoalkyl.

In one embodiment, the crosslinked polymer comprises the residue of an amine corresponding to Formula 1 wherein R₁, R₂, and R₃ are independently hydrogen, heteroaryl, aryl, aliphatic or heteroaliphatic provided, however, at least one of R₁, R₂, and R₃ is aryl or heteroaryl. For example, in this embodiment R₁ and R₂, in combination with the nitrogen atom to which they are attached, may form a saturated or unsaturated nitrogen-containing heterocyclic ring. By way of further example, R₁ and R₂, in combination with the nitrogen atom to which they are attached may constitute part of a pyrrolidino, pyrrole, pyrazolidine, pyrazole, imidazolidine, imidazole, piperidine, pyridine, piperazine, diazine, or triazine ring structure. By way of further example, R₁ and R₂, in combination with the nitrogen atom to which they are attached may constitute part of a piperidine ring structure.

In one embodiment, the crosslinked polymer comprises the residue of an amine corresponding to Formula 1 wherein R₁, R₂, and R₃ are independently hydrogen, aliphatic, or heteroaliphatic provided, however, at least one of R₁, R₂, and R₃ is other than hydrogen. For example, in this embodiment R₁, R₂, and R₃ may independently be hydrogen, alkyl, alkenyl, allyl, vinyl, aminoalkyl, alkanol, haloalkyl, hydroxyalkyl, ethereal, or heterocyclic provided, however, at least one of R₁, R₂, and R₃ is other than hydrogen. By way of further example, in one such embodiment R₁ and R₂, in combination with the nitrogen atom to which they are attached, may form a saturated or unsaturated nitrogen-containing heterocyclic ring. By way of further example, in one such embodiment R₁ and R₂, in combination with the nitrogen atom to which they are attached may constitute part of a pyrrolidino, pyrrole, pyrazolidine, pyrazole, imidazolidine, imidazole, piperidine, piperazine, or diazine ring structure. By way of further example, in one such embodiment R₁ and R₂, in combination with the nitrogen atom to which they are attached may constitute part of a piperidine ring structure. By way of further example, in one such embodiment the amine corresponding to Formula 1 is acyclic and at least one of R₁, R₂, and R₃ is aliphatic or heteroaliphatic. By way of further example, in one such embodiment R₁, R₂, and R₃ are independently hydrogen, alkyl, allyl, vinyl, alicyclic, aminoalkyl, alkanol, or heterocyclic, provided at least one of R₁, R₂, and R₃ is other than hydrogen.

In one embodiment, the crosslinked polymer comprises the residue of an amine corresponding to Formula 1 and the crosslinked polymer is prepared by substitution polymerization of the amine corresponding to Formula 1 with a polyfunctional crosslinker (optionally also comprising amine moieties) wherein R₁, R₂, and R₃ are independently hydrogen, alkyl, aminoalkyl, or alkanol, provided at least one of R₁, R₂, and R₃ is other than hydrogen.

In some embodiments, the molecular weight per nitrogen of the polymers of the present disclosure may range from about 40 to about 1000 Daltons. In one embodiment, the molecular weight per nitrogen of the polymer is from about 40 to about 500 Daltons. In another embodiment, the molecular weight per nitrogen of the polymer is from about 50 to about 170 Daltons. In another embodiment, the molecular weight per nitrogen of the polymer is from about 60 to about 110 Daltons.

In some embodiments, an amine-containing monomer is polymerized and the polymer is concurrently crosslinked in a substitution polymerization reaction in the first reaction step. The amine reactant (monomer) in the concurrent polymerization and crosslinking reaction can react more than one time for the substitution polymerization. In one such embodiment, the amine monomer is a linear amine possessing at least two reactive amine moieties to participate in the substitution polymerization reaction. In another embodiment, the amine monomer is a branched amine possessing at least two reactive amine moieties to participate in the substitution polymerization reaction. Crosslinkers for the concurrent substitution polymerization and crosslinking typically have at least two amine-reactive moieties such as alkyl-chlorides, and alkyl-epoxides. In order to be incorporated into the polymer, primary amines react at least once and potentially may react up to three times with the crosslinker, secondary amines can react up to twice with the crosslinkers, and tertiary amines can only react once with the crosslinker. In general, however, the formation of a significant number of quaternary nitrogens/amines is generally not preferred because quaternary amines cannot bind protons.

Exemplary amines that may be used in substitution polymerization reactions described herein include 1,3-Bis[bis(2-aminoethyl)amino]propane, 3-Amino-1-{[2-(bis{2-[bis(3-aminopropyl)amino]ethyl}amino)ethyl](3-aminopropyl)amino}propane, 2-[Bis(2-aminoethyl)amino]ethanamine, Tris(3-aminopropyl)amine, 1,4-Bis[bis(3-aminopropyl)amino]butane, 1,2-Ethanediamine, 2-Amino-1-(2-aminoethylamino)ethane, 1,2-Bis(2-aminoethylamino)ethane, 1,3-Propanediamine, 3,3′-Diaminodipropylamine, 2,2-dimethyl-1,3-propanediamine, 2-methyl-1,3-propanediamine, N,N′-dimethyl-1,3-propanediamine, N-methyl-1,3-diaminopropane, 3,3′-diamino-N-methyldipropylamine, 1,3-diaminopentane, 1,2-diamino-2-methylpropane, 2-methyl-1,5-diaminopentane, 1,2-diaminopropane, 1,10-diaminodecane, 1,8-diaminooctane, 1,9-diaminooctane, 1,7-diaminoheptane, 1,6-diaminohexane, 1,5-diaminopentane, 3-bromopropylamine hydrobromide, N,2-dimethyl-1,3-propanediamine, N-isopropyl-1,3-diaminopropane, N,N′-bis(2-aminoethyl)-1,3-propanediamine, N,N′-bis(3-aminopropyl)ethylenediamine, N,N′-bis(3-aminopropyl)-1,4-butanediamine tetrahydrochloride, 1,3-diamino-2-propanol, N-ethylethylenediamine, 2,2′-diamino-N-methyldiethylamine, N,N′-diethylethylenediamine, N-isopropylethylenediamine, N-methylethylenediamine, N,N′-di-tert-butylethylenediamine, N,N′-diisopropylethylenediamine, N,N′-dimethylethylenediamine, N-butylethylenediamine, 2-(2-aminoethylamino)ethanol, 1,4,7,10,13,16-hexaazacyclooctadecane, 1,4,7,10-tetraazacyclododecane, 1,4,7-triazacyclononane, N,N′-bis(2-hydroxyethyl)ethylenediamine, piperazine, bis(hexamethylene)triamine, N-(3-hydroxypropyl)ethylenediamine, N-(2-Aminoethyl)piperazine, 2-Methylpiperazine, Hornopiperazine, 1,4,8,11-Tetraazacyclotetradecane, 1,4,8,12-Tetraazacyclopentadecane, 2-(Aminomethyl)piperidine, 3-(Methylamino)pyrrolidine

Exemplary crosslinking agents that may be used in substitution polymerization reactions and post-polymerization crosslinking reactions include, but are not limited to, one or more multifunctional crosslinking agents such as: dihaloalkanes, haloalkyloxiranes, alkyloxirane sulfonates, di(haloalkyl)amines, tri(haloalkyl) amines, diepoxides, triepoxides, tetraepoxides, bis (halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, I-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy) diphenylmethane, bisphenol F diglycidyl ether, 1,4-bis(2′,3′epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4-h-chromene-2-carboxylate, bis[4-(2,3-epoxy-propylthio)phenyl]-sulfide, 1,3-bis(3-glycidoxypropyl) tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy) propyl]dimethylsilyloxy]-1,3,5,7,9,11,14-heptacyclopentyltricyclo [7,3,3,15, 11]heptasiloxane, 4,4′methylenebis(N, N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate, 3-chloro-1-(3-chloropropylamino-2-propanol, 1,2-bis(3-chloropropylamino)ethane, Bis(3-chloropropyl)amine, 1,3-Dichloro-2-propanol, 1,3-Dichloropropane, 1-chloro-2,3-epoxypropane, tris[(2-oxiranyl)methyl]amine.

In some embodiments, the carbon to nitrogen ratio of the polymers of the present disclosure may range from about 2:1 to about 6:1, respectively. For example, in one such embodiment, the carbon to nitrogen ratio of the polymers of the present disclosure may range from about 2.5:1 to about 5:1, respectively. By way of further example, in one such embodiment, the carbon to nitrogen ratio of the polymers of the present disclosure may range from about 3:1 to about 4.5:1, respectively. By way of further example, in one such embodiment, the carbon to nitrogen ratio of the polymers of the present disclosure may range from about 3.25:1 to about 4.25:1, respectively. By way of further example, in one such embodiment, the carbon to nitrogen ratio of the polymers of the present disclosure may range from about 3.4:1 to about 4:1, respectively. In another embodiment, the molecular weight per nitrogen of the polymer is from about 60 to about 110 Daltons.

In some embodiments, the crosslinked polymer comprises the residue of an amine corresponding to Formula 1a and the crosslinked polymer is prepared by radical polymerization of an amine corresponding to Formula 1a:

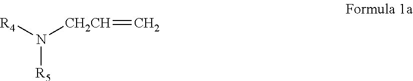

wherein R₄ and R₅ are independently hydrogen, hydrocarbyl, or substituted hydrocarbyl. In one embodiment, for example, R₄ and R₅ are independently hydrogen, saturated hydrocarbon, unsaturated aliphatic, aryl, heteroaryl, unsaturated heteroaliphatic, heterocyclic, or heteroalkyl. By way of further example, in one such embodiment R₄ and R₅ are independently hydrogen, aliphatic, heteroaliphatic, aryl, or heteroaryl. By way of further example, in one such embodiment R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic. By way of further example, in one such embodiment R₄ and R₅ are independently hydrogen, alkyl, allyl, aminoalkyl, alkanol, aryl, haloalkyl, hydroxyalkyl, ethereal, or heterocyclic. By way of further example, in one such embodiment R₄ and R₅ (in combination with the nitrogen atom to which they are attached) together constitute part of a ring structure, so that the monomer as described by Formula 1a is a nitrogen-containing heterocycle (e.g., piperidine). By way of further example, in one embodiment R₄ and R₅ are independently hydrogen, aliphatic or heteroaliphatic. By way of further example, in one embodiment R₄ and R₅ are independently hydrogen, allyl, or aminoalkyl.

In some embodiments, the crosslinked polymer comprises the residue of an amine corresponding to Formula 1b and the crosslinked polymer is prepared by substitution polymerization of the amine corresponding to Formula 1b with a polyfunctional crosslinker (optionally also comprising amine moieties):

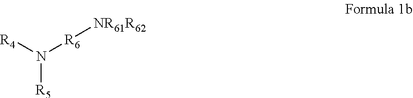

wherein R₄ and R₅ are independently hydrogen, hydrocarbyl, or substituted hydrocarbyl, R₆ is aliphatic and R₆₁ and R₆₂ are independently hydrogen, aliphatic, or heteroaliphatic. In one embodiment, for example, R₄ and R₅ are independently hydrogen, saturated hydrocarbon, unsaturated aliphatic, aryl, heteroaryl, heteroalkyl, or unsaturated heteroaliphatic. By way of further example, in one such embodiment R₄ and R₅ are independently hydrogen, aliphatic, heteroaliphatic, aryl, or heteroaryl. By way of further example, in one such embodiment R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic. By way of further example, in one such embodiment R₄ and R₅ are independently hydrogen, alkyl, alkenyl, aminoalkyl, alkanol, aryl, haloalkyl, hydroxyalkyl, ethereal, heteroaryl or heterocyclic. By way of further example, in one such embodiment R₄ and R₅ (in combination with the nitrogen atom to which they are attached) together constitute part of a ring structure, so that the monomer as described by Formula 1a is a nitrogen-containing heterocycle (e.g., piperidine). By way of further example, in one embodiment R₄ and R₅ are independently hydrogen, aliphatic or heteroaliphatic. By way of further example, in one embodiment R₄ and R₅ are independently hydrogen, allyl, or aminoalkyl. By way of further example, in each of the embodiments recited in this paragraph, R₆ may be methylene, ethylene or propylene, and R₆₁ and R₆₂ may independently be hydrogen, allyl or aminoalkyl.

In some embodiments, the crosslinked polymer comprises the residue of an amine corresponding to Formula 1c:

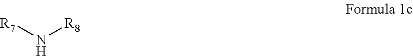

wherein R₇ is hydrogen, aliphatic or heteroaliphatic and R₈ is aliphatic or heteroaliphatic. For example, in one such embodiment, for example, R₇ is hydrogen and R₈ is aliphatic or heteroaliphatic. By way of further example, in one such embodiment R₇ and R₈ are independently aliphatic or heteroaliphatic. By way of further example, in one such embodiment at least one of R₇ and R₈ comprises an allyl moiety. By way of further example, in one such embodiment at least one of R₇ and R₈ comprises an aminoalkyl moiety. By way of further example, in one such embodiment R₇ and R₈ each comprise an allyl moiety. By way of further example, in one such embodiment R₇ and R₈ each comprise an aminoalkyl moiety. By way of further example, in one such embodiment R₇ comprises an allyl moiety and R₈ comprises an aminoalkyl moiety.

In some embodiments, the crosslinked polymer comprises the residue of an amine corresponding to Formula 2:

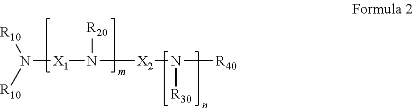

wherein

m and n are independently non-negative integers;

R₁₀, R₂₀, R₃₀, and R₄₀ are independently hydrogen, hydrocarbyl, or substituted hydrocarbyl;

X₁ is

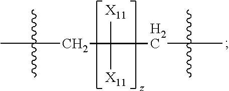

X₂ is hydrocarbyl or substituted hydrocarbyl;

each X₁₁ is independently hydrogen, hydrocarbyl, substituted hydrocarbyl, hydroxyl, amino, boronic acid, or halo; and

z is a non-negative number.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and m and n are independently 0, 1, 2 or 3 and n is 0 or 1.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and R₁₀, R₂₀, R₃₀, and Rₒₒ are independently hydrogen, aliphatic, aryl, heteroaliphatic, or heteroaryl. By way of further example, in one such embodiment R₁₀, R₂₀, R₃₀, and R₄₀ are independently hydrogen, aliphatic, or heteroaliphatic. By way of further example, in one such embodiment R₁₀, R₂₀, R₃₀, and R₄₀ are independently hydrogen, alkyl, allyl, vinyl, or aminoalkyl. By way of further example, in one such embodiment R₁₀, R₂₀, R₃₀, and R₄₀ are independently hydrogen, alkyl, allyl, vinyl, —(CH₂)_{d}NH₂, —(CH₂)_{d}N[(CH₂)ₑNH₂)]₂ where d and e are independently 2-4. In each of the foregoing exemplary embodiments of this paragraph, m and z may independently be 0, 1, 2 or 3 and n is 0 or 1.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and X₂ is aliphatic or heteroaliphatic. For example, in one such embodiment X₂ is aliphatic or heteroaliphatic and R₁₀, R₂₀, R₃₀, and R₄₀ are independently hydrogen, aliphatic, heteroaliphatic. By way of further example, in one such embodiment X₂ is alkyl or aminoalkyl and R₁₀, R₂₀, R₃₀, and R₄₀ are independently hydrogen, aliphatic, or heteroaliphatic. By way of further example, in one such embodiment X₂ is alkyl or aminoalkyl and R₁₀, R₂₀, R₃₀, and R₄₀ are independently hydrogen, alkyl, allyl, vinyl, or aminoalkyl. In each of the foregoing exemplary embodiments of this paragraph, m and z may independently be 0, 1, 2 or 3 and n is 0 or 1.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and m is a positive integer. For example, in one such embodiment m is a positive integer, z is zero and R₂₀ is hydrogen, aliphatic or heteroaliphatic. By way of further example, in one such embodiment m is a positive integer (e.g., 1 to 3), z is a positive integer (e.g., 1 to 2), X₁₁ is hydrogen, aliphatic or heteroaliphatic, and R₂₀ is hydrogen, aliphatic or heteroaliphatic. By way of further example, in one such embodiment m is a positive integer, z is zero, one or two, X₁₁ is hydrogen alkyl, alkenyl, or aminoalkyl, and R₂₀ is hydrogen, alkyl, alkenyl, or aminoalkyl.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and n is a positive integer and R₃₀ is hydrogen, aliphatic or heteroaliphatic. By way of further example, in one such embodiment n is 0 or 1, and R₃₀ is hydrogen, alkyl, alkenyl, or aminoalkyl.

In one embodiment, the crosslinked polymer comprises the residue of an amine corresponding to Formula 2, the crosslinked polymer is prepared by (i) substitution polymerization of the amine corresponding to Formula 2 with a polyfunctional crosslinker (optionally also comprising amine moieties) or (2) radical polymerization of an amine corresponding to Formula 2, and m and n are independently non-negative integers and X₂ is aliphatic or heteroaliphatic. For example, in one such embodiment m is 0 to 2, n is 0 or 1, X₂ is aliphatic or heteroaliphatic, and R₁₀, R₂₀, R₃₀, and R₄₀ are independently hydrogen, aliphatic, or heteroaliphatic. By way of further example, in one such embodiment m is 0 to 2, n is 0 or 1, X₂ is alkyl or aminoalkyl, and R₁₀, R₂₀, R₃₀, and R₄₀ are independently hydrogen, aliphatic, or heteroaliphatic. By way of further example, in one such embodiment m is 0 to 2, n is 0 or 1, X₂ is alkyl or aminoalkyl, and R₁₀, R₂₀, R₃₀, and R₄₀ are independently hydrogen, alkyl, alkenyl, or aminoalkyl.

In some embodiments, the crosslinked polymer comprises the residue of an amine corresponding to Formula 2a and the crosslinked polymer is prepared by substitution polymerization of the amine corresponding to Formula 2a with a polyfunctional crosslinker (optionally also comprising amine moieties):

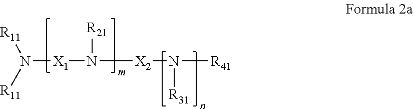

wherein

m and n are independently non-negative integers;

each R₁₁ is independently hydrogen, hydrocarbyl, heteroaliphatic, or heteroaryl;

R₂₁ and R₃₁, are independently hydrogen or heteroaliphatic;

R₄₁ is hydrogen, substituted hydrocarbyl, or hydrocarbyl;

X₁ is

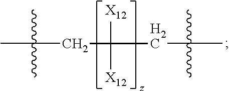

X₂ is alkyl or substituted hydrocarbyl;

each X₁₂ is independently hydrogen, hydroxy, amino, aminoalkyl, boronic acid or halo; and

z is a non-negative number.

In one embodiment, the crosslinked polymer comprises the residue of an amine corresponding to Formula 2a, the crosslinked polymer is prepared by substitution polymerization of the amine corresponding to Formula 1 with a polyfunctional crosslinker (optionally also comprising amine moieties). For example, in one such embodiment, m and z are independently 0, 1, 2 or 3, and n is 0 or 1.

In one embodiment, the crosslinked polymer comprises the residue of an amine corresponding to Formula 2a, the crosslinked polymer is prepared by substitution polymerization of the amine corresponding to Formula 2a with a polyfunctional crosslinker (optionally also comprising amine moieties), and each R₁₁ is independently hydrogen, aliphatic, aminoalkyl, haloalkyl, or heteroaryl, R₂₁ and R₃₁ are independently hydrogen or heteroaliphatic and R₄₁ is hydrogen, aliphatic, aryl, heteroaliphatic, or heteroaryl. For example, in one such embodiment each R₁₁ is hydrogen, aliphatic, aminoalkyl, or haloalkyl, R₂₁ and R₃₁ are independently hydrogen or heteroaliphatic and R₄₁ is hydrogen, alkylamino, aminoalkyl, aliphatic, or heteroaliphatic. By way of further example, in one such embodiment each R₁₁ is hydrogen, aliphatic, aminoalkyl, or haloalkyl, R₂₁ and R₃₁ are hydrogen or aminoalkyl, and R₄₁ is hydrogen, aliphatic, or heteroaliphatic. By way of further example, in one such embodiment each R₁₁ and R₄₁ is independently hydrogen, alkyl, or aminoalkyl, and R₂₁ and R₃₁ are independently hydrogen or heteroaliphatic. By way of further example, in one such embodiment each R₁₁ and R₄₁ is independently hydrogen, alkyl, —(CH₂)_{d}NH₂, —(CH₂)_{d}N[(CH₂)ₑNH₂)]₂ where d and e are independently 2-4, and R₂₁ and R₃₁ are independently hydrogen or heteroaliphatic. In each of the foregoing exemplary embodiments of this paragraph, m and z may independently be 0, 1, 2 or 3, and n is 0 or 1.

Exemplary amines for the synthesis of polymers comprising repeat units corresponding to Formula 2a include, but are not limited to, amines appearing in Table A.

TABLE A

| | | | | MW |
|---|---|---|---|---|
| Abbreviation | IUPAC name | Other names | | (g/mol) |
| C2A3BTA | 1,3-Bis[bis(2- aminoethyl)amino]propane | | 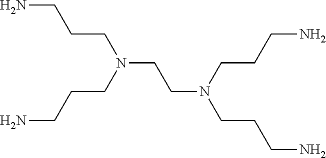 [SMILES: NCCCN(CCCN)CCN(CCCN)CCCN] | 288.48 |
| C2A3G2 | 3-Amino-1-{[2-(bis{2-[bis(3- aminopropyl)amino]eth- yl}amino)ethyl](3- aminopropyl)amino}propane | | 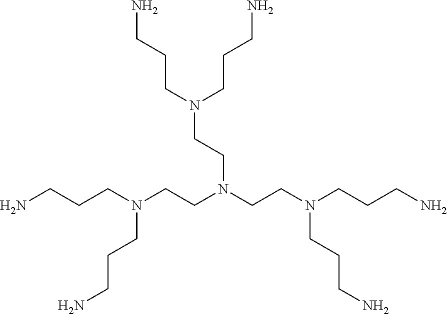 [SMILES: NCCCN(CCCN)CCN(CCN(CCCN)CCCN)CCN(CCCN)CCCN] | 488.81 |
| C2PW | 2-[Bis(2- aminoethyl)amino]ethanamine | 2,2′,2″- Triaminotrieth- ylamine or 2,2′,2″- Nitrilotriethyla- mine | 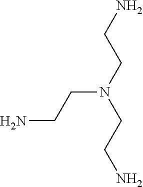 | 146.24 |
| C3PW | Tris(3-aminopropyl)amine | | 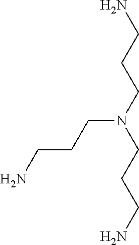 [SMILES: NCCCN(CCCN)CCCN] | 188.32 |
| C4A3BTA | 1,4-Bis[bis(3- aminopropyl)amino]butane | | 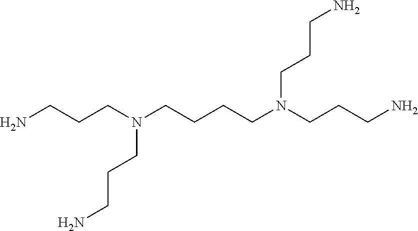 [SMILES: NCCCN(CCCN)CCCCN(CCCN)CCCN] | 316.54 |
| EDA1 | 1,2-Ethanediamine | | 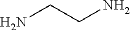 | 60.1 |
| EDA2 | 2-Amino-1-(2- aminoethylamino)ethane | Bis(2- aminoethyl)a- mine or 2,2′- | 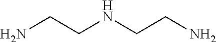 | 103.17 |
| | | Diaminodiethyl- | | |
| | | amine | | |
| EDA3 | 1,2-Bis(2- aminoethylamino)ethane | N,N′-Bis(2- aminoethyl)eth- ane-1,2- | 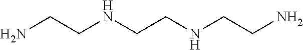 | 146.24 |
| | | diamine | | |
| PDA1 | 1,3-Propanediamine | | 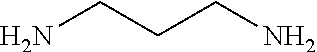 | 74.3 |
| PDA2 | 3,3′-Diaminodipropylamine | | 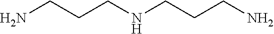 | 131.22 |

Exemplary crosslinkers for the synthesis of polymers comprising the residue of amines corresponding to Formula 2a include but are not limited to crosslinkers appearing in Table B.

TABLE B

| | | | | MW |
|---|---|---|---|---|
| Abbreviation | Common name | IUPAC name | | (g/mol) |
| BCPA | Bis(3- chloropropyl)amino | Bis(3- chloropropyl)amine | 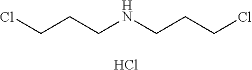 | 206.54 |
| DC2OH | 1,3- dichloroisopropanol | 1,3-Dichloro-2- propanol | 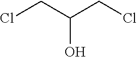 | 128.98 |
| DCE | dichloroethane | 1,2-dichloroethane | 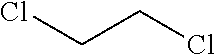 | 98.96 |
| DCP | Dichloropropane | 1,3-Dichloropropane | 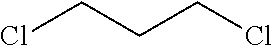 | 112.98 |
| ECH | Epichlorohydrin | 1-chloro-2,3- epoxypropane | 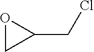 | 92.52 |
| TGA | Triglycidyl amine | Tris[(2- oxiranyl)methyl]amine | 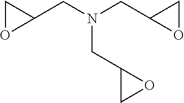 [SMILES: C1OC1CN(CC1CO1)CC1CO1] | 185.22 |
| BCPOH | Bis(3-chloropropyl) amine-OH | 3-Chloro-1-(3- chloropropylamino)-2- propanol | 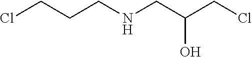 | 186.08 |
| BCPEDA | Bis(chloropropyl) ethylenediamine | 1,2-Bis(3- chloropropylamino)eth- ane | 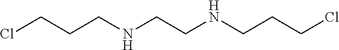 [SMILES: ClCCCNCCNCCCCl] | 213.15 |

In some embodiments, the crosslinked polymer comprises the residue of an amine corresponding to Formula 2b and the crosslinked polymer is prepared by radical polymerization of an amine corresponding to Formula 2b:

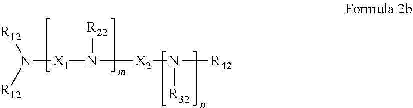

wherein

m and n are independently non-negative integers;

each R₁₂ is independently hydrogen, substituted hydrocarbyl, or hydrocarbyl; R₂₂ and R₃₂ are independently hydrogen substituted hydrocarbyl, or hydrocarbyl;

R₄₂ is hydrogen, hydrocarbyl or substituted hydrocarbyl;

X₁ is

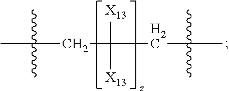

X₂ is alkyl, aminoalkyl, or alkanol;

each X₁₃ is independently hydrogen, hydroxy, alicyclic, amino, aminoalkyl, halogen, alkyl, heteroaryl, boronic acid or aryl;

z is a non-negative number, and

the amine corresponding to Formula 2b comprises at least one allyl group.

In one embodiment, the crosslinked polymer comprises the residue of an amine corresponding to Formula 2b, the crosslinked polymer is prepared by radical polymerization of an amine corresponding to Formula 2b, and m and z are independently 0, 1, 2 or 3, and n is 0 or 1.

In one embodiment, the crosslinked polymer comprises the residue of an amine corresponding to Formula 2b, the crosslinked polymer is prepared by radical polymerization of an amine corresponding to Formula 1, and (i) R₁₂ or R₄₂ independently comprise at least one allyl or vinyl moiety, (ii) m is a positive integer and R₂₂ comprises at least one allyl or vinyl moiety, and/or (iii) n is a positive integer and R₃₂ comprises at least one allyl moiety. For example, in one such embodiment, m and z are independently 0, 1, 2 or 3 and n is 0 or 1. For example, in one such embodiment R₁₂ or R₄₂, in combination comprise at least two allyl or vinyl moieties. By way of further example, in one such embodiment, m is a positive integer and R₁₂, R₂₂ and R₄₂, in combination comprise at least two allyl or vinyl moieties. By way of further example, in one such embodiment, n is a positive integer and R₁₂, R₃₂ and R₄₂, in combination comprise at least two allyl or vinyl moieties. By way of further example, in one such embodiment, m is a positive integer, n is a positive integer and R₁₂, R₂₂, R₃₂ and R₄₂, in combination, comprise at least two allyl or vinyl moieties.

In one embodiment, the crosslinked polymer comprises the residue of an amine corresponding to Formula 2b, the crosslinked polymer is prepared by radical polymerization of an amine corresponding to Formula 2b, and each R₁₂ is independently hydrogen, aminoalkyl, allyl, or vinyl, R₂₂ and R₃₂ are independently hydrogen, alkyl, aminoalkyl, haloalkyl, alkenyl, alkanol, heteroaryl, alicyclic heterocyclic, or aryl, and R₄₂ is hydrogen or substituted hydrocarbyl. For example, in one such embodiment each R₁₂ is aminoalkyl, allyl or vinyl, R₂₂ and R₃₂ are independently hydrogen, alkyl, aminoalkyl, haloalkyl, alkenyl, or alkanol, and R₄₂ is hydrogen or substituted hydrocarbyl. By way of further example, in one such embodiment each R₁₂ and R₄₂ is independently hydrogen, alkyl, allyl, vinyl, —(CH₂)_{d}NH₂ or —(CH₂)_{d}N[(CH₂)ₑNH₂]₂ where d and e are independently 2-4, and R₂₂ and R₃₂ are independently hydrogen or heteroaliphatic.

Exemplary amines and crosslinkers (or the salts thereof, for example the hydrochloric acid, phosphoric acid, sulfuric acid, or hydrobromic acid salts thereof) for the synthesis of polymers described by Formula 2b include but are not limited to the ones in Table C.

TABLE C

| | | | | MW |
|---|---|---|---|---|
| Abbreviation | Common name | IUPAC name | | (g/mol) |
| DABDA1 | Diallylbutyldiamine | 1,4- Bis(allylamino) butane | 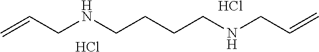 | 241.2 |
| DAEDA1 | Diallylethyldiamine | 1,2- Bis(allylamino) ethane | 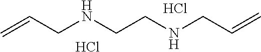 | 213.15 |
| DAEDA2 | Diallyldiethylenetria- mine | 2-(Allylamino)- 1-[2- (allylamino)eth- ylamino]ethane | 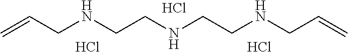 | 292.67 |
| DAPDA | Diallylpropyldiamine | 1,3- Bis(allylamino) propane | 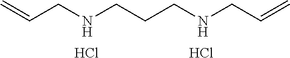 | 227.17 |
| POHDA | Diallylamineiso- propanol | 1,3- Bis(allylamino)- 2-propanol | 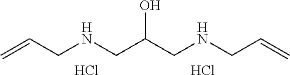 | 243.17 |
| AAH | Allylamine | 2-Propen-1- ylamine | 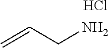 | 93.5 |
| AEAAH | Aminoethylallylamine | 1-(Allylamino)- 2-aminoethane | 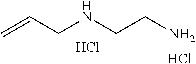 | 173.08 |
| BAEAAH | Bis(2- aminoethyl)allylamine | 1-[N-Allyl(2- aminoeth- yl)amino]-2- aminoethane | 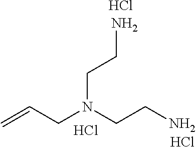 | 252.61 |
| TAA | Triallylamine | N,N,N- triallylamine | 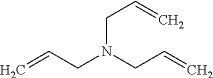 | 137.22 |

In some embodiments, the crosslinked polymer is derived from a reaction of the resulting polymers that utilize monomers described in any of Formulae 1, 1a, 1b, 1c, 2, 2a and 2b or a linear polymer comprised of a repeat unit described by Formula 3 with external crosslinkers or pre-existing polymer functionality that can serve as crosslinking sites. Formula 3 can be a repeat unit of a copolymer or terpolymer where X₁₅ is either a random, alternating, or block copolymer. The repeating unit in Formula 3 can also represent the repeating unit of a polymer that is branched, or hyperbranched, wherein the primary branch point can be from any atom in the main chain of the polymer:

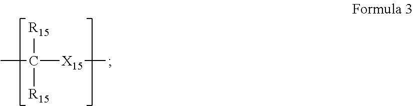

wherein

R₁₅, R₁₆ and R₁₇ are independently hydrogen, hydrocarbyl, substituted hydrocarbyl, hydroxyl, amino, boronic acid or halo;

X₁₅ is

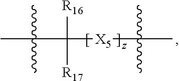

X₅ is hydrocarbyl, substituted hydrocarbyl, oxo (—O—), or amino and

z is a non-negative number.

In one embodiment, R₁₅, R₁₆ and R₁₇ are independently hydrogen, aryl, or heteroaryl, X₅ is hydrocarbyl, substituted hydrocarbyl, oxo or amino, and m and z are non-negative integers. In another embodiment, R₁₅, R₁₆ and R₁₇ are independently aliphatic or heteroaliphatic, X₅ is hydrocarbyl, substituted hydrocarbyl, oxo (—O—) or amino, and m and z are non-negative integers. In another embodiment, R₁₅, R₁₆ and R₁₇ are independently unsaturated aliphatic or unsaturated heteroaliphatic, X₅ is hydrocarbyl, substituted hydrocarbyl, oxo, or amino, and z is a non-negative integer. In another embodiment, R₁₅, R₁₆ and R₁₇ are independently alkyl or heteroalkyl, X₅ is hydrocarbyl, substituted hydrocarbyl, oxo, or amino, and z is a non-negative integer. In another embodiment, R₁₅, R₁₆ and R₁₇ are independently alkylamino, aminoalkyl, hydroxyl, amino, boronic acid, halo, haloalkyl, alkanol, or ethereal, X₅ is hydrocarbyl, substituted hydrocarbyl, oxo, or amino, and z is a non-negative integer. In another embodiment, R₁₅, R₁₆ and R₁₇ are independently hydrogen, hydrocarbyl, substituted hydrocarbyl, hydroxyl, amino, boronic acid or halo, X₅ is oxo, amino, alkylamino, ethereal, alkanol, or haloalkyl, and z is a non-negative integer.

Exemplary crosslinking agents that may be used in radical polymerization reactions include, but are not limited to, one or more multifunctional crosslinking agents such as: 1,4-bis(allylamino)butane, 1,2-bis(allylamino)ethane, 2-(allylamino)-1-[2-(allylamino)ethylamino]ethane, 1,3-bis(allylamino)propane, 1,3-bis(allylamino)-2-propanol, triallylamine, diallylamine, divinylbenzene, 1,7-octadiene, 1,6-heptadiene, 1,8-nonadiene, 1,9-decadiene, 1,4-divinyloxybutane, 1,6-hexamethylenebisacrylamide, ethylene bisacrylamide, N,N′-bis(vinylsulfonylacetyl)ethylene diamine, 1,3-bis(vinylsulfonyl) 2-propanol, vinylsulfone, N,N′-methylenebisacrylamide polyvinyl ether, polyallylether, divinylbenzene, 1,4-divinyloxybutane, and combinations thereof.

Crosslinked polymers derived from the monomers and polymers in formulas 1 through 3 may be synthesized either in solution or bulk or in dispersed media. Examples of solvents that are suitable for the synthesis of polymers of the present disclosure include, but are not limited to water, low boiling alcohols (methanol, ethanol, propanol, butanol), dimethylformamide, dimethylsulfoxide, heptane, chlorobenzene, toluene.

Alternative polymer processes may include, a lone polymerization reaction, stepwise addition of individual starting material monomers via a series of reactions, the stepwise addition of blocks of monomers, combinations or any other method of polymerization such as living polymerization, direct polymerization, indirect polymerization, condensation, radical, emulsion, precipitation approaches, spray dry polymerization or using some bulk crosslinking reaction methods and size reduction processes such as grinding, compressing, extrusion. Processes can be carried out as a batch, semi-continuous and continuous processes. For processes in dispersed media, the continuous phase can be non-polar solvents, such as toluene, benzene, hydrocarbon, halogenated solvents, super critical carbon dioxide. With a direct suspension reaction, water can be used and salt can be used to tune the properties of the suspension.

The starting molecules described in formulas 1 through 3 may be copolymerized with one or more other monomers of the invention, oligomers or other polymerizable groups. Such copolymer architectures can include, but are not limited to, block or block-like polymers, graft copolymers, and random copolymers. Incorporation of monomers described by formulas 1 through 3 can range from 1% to 99%. In some embodiments, the incorporation of comonomer is between 20% and 80%.

Non-limiting examples of comonomers which may be used alone or in combination include: styrene, allylamine hydrochloride, substituted allylamine hydrochloride, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, N-vinyl amide, maleic acid derivatives, vinyl ether, allyl, methallyl monomers and combinations thereof. Functionalized versions of these monomers may also be used. Additional specific monomers or comonomers that may be used in this invention include, but are not limited to, 2-propen-1-ylamine, 1-(allylamino)-2-aminoethane, 1[N-allyl(2-aminoethyl)amino]-2-aminoethane, methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, amethylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N,N-butylmethacrylamide, N-methylolmethacrylamide, N-ethylolmethacrylamide, N-tert-butylacryl amide, N,N-butylacrylamide, N-methylolacrylamide, N-ethylolacrylamide, 4-acryloylmorpholine, vinyl benzoic acid (all isomers), diethylaminostyrene (all isomers), a-methylvinyl benzoic acid (all isomers), diethylamino a-methylstyrene (all isomers), p-vinylbenzene sulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropyl methacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysilylpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, maleic anhydride, N-phenylmaleimide, N-butylmaleimide, N-vinylformamide, N-vinyl acetamide, allylamine, methallylamine, allylalcohol, methyl-vinylether, ethylvinylether, butylvinyltether, butadiene, isoprene, chloroprene, ethylene, vinyl acetate, and combinations thereof.

Additional modification to the preformed crosslinked polymer can be achieved through the addition of modifiers, including but not limited to amine monomers, additional crosslinkers, and polymers. Modification can be accomplished through covalent or non-covalent methods. These modifications can be evenly or unevenly dispersed throughout the preformed polymer material, including modifications biased to the surface of the preformed crosslinked polymer. Furthermore, modifications can be made to change the physical properties of the preformed crosslinked polymer, including but not limited to reactions that occur with remaining reactive groups such as haloalkyl groups and allyl groups in the preformed polymer. Reactions and modifications to the preformed crosslinked polymer can include but are not limited to acid-base reactions, nucleophilic substitution reactions, Michael reactions, non-covalent electrostatic interactions, hydrophobic interactions, physical interactions (crosslinking) and radical reactions.

In one embodiment, the post-polymerization crosslinked amine polymer is a crosslinked amine polymer comprising a structure corresponding to Formula 4:

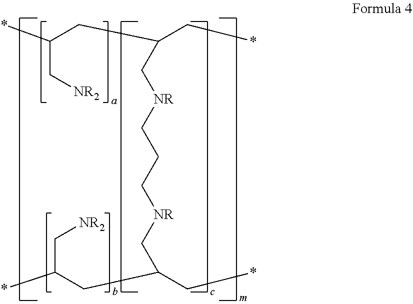

wherein each R is independently hydrogen or an ethylene crosslink between two nitrogen atoms of the crosslinked amine polymer

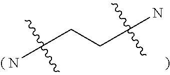

and a, b, c, and m are integers. Typically, m is a large integer indicating an extended polymer network. In one such embodiment, a ratio of the sum of a and b to c (i.e., a+b:c) is in the range of about 1:1 to 5:1. For example, in one such embodiment a ratio of the sum of a and b to c (i.e., a+b:c) is in the range of about 1.5:1 to 4:1. By way of further example, in one such embodiment a ratio of the sum of a and b to c (i.e., a+b:c) is in the range of about 1.75:1 to 3:1. For example, in one such embodiment a ratio of the sum of a and b is 57, c is 24 and m is large integer indicating an extended polymer network. In each of the foregoing embodiments a ratio of the sum of a and b to c (i.e., a+b:c) may be in the range of about 2:1 to 2.5:1. For example, in such embodiments the ratio of the sum of a and b to c (i.e., a+b:c) may be in the range of about 2.1:1 to 2.2:1. By way of further example, in such embodiments the ratio of the sum of a and b to c (i.e., a+b:c) may be in the range of about 2.2:1 to 2.3:1. By way of further example, in such embodiments the ratio of the sum of a and b to c (i.e., a+b:c) may be in the range of about 2.3:1 to 2.4:1. By way of further example, in such embodiments the ratio of the sum of a and b to c (i.e., a+b:c) may be in the range of about 2.4:1 to 2.5:1. In each of the foregoing embodiments, each R may independently be hydrogen or an ethylene crosslink between two nitrogen atoms. Typically, however, 35-95% of the R substituents will be hydrogen and 5-65% will be an ethylene crosslink

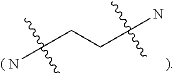

For example, in one such embodiment, 50-95% of the R substituents will be hydrogen and 5-50% will be an ethylene crosslink

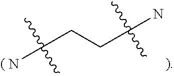

For example, in one such embodiment, 55-90% of the R substituents are hydrogen and 10-45% are an ethylene crosslink

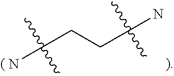

By way of further example, in one such embodiment, 60-90% of the R substituents are hydrogen and 10-40% are an ethylene crosslink. By way of further example, in one such embodiment, 65-90% of the R substituents are hydrogen and 10-35% are an ethylene crosslink.

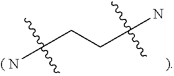

By way of further example, in one such embodiment, 70-90% of the R substituents are hydrogen and 10-30% are an ethylene crosslink. By way of further example, in one such embodiment, 75-85% of the R substituents are hydrogen and 15-25% are an ethylene crosslink. By way of further example, in one such embodiment, 65-75% of the R substituents are hydrogen and 25-35% are an ethylene crosslink. By way of further example, in one such embodiment, 55-65% of the R substituents are hydrogen and 35-45% are an ethylene crosslink. In some embodiments, a, b, c and R are such that the carbon to nitrogen ratio of the polymer of Formula 4 may range from about 2:1 to about 6:1, respectively. For example, in one such embodiment, the carbon to nitrogen ratio of the polymer of Formula 4 may range from about 2.5:1 to about 5:1, respectively. By way of further example, in one such embodiment, the carbon to nitrogen ratio of the polymer of Formula 4 may range from about 3:1 to about 4.5:1, respectively. By way of further example, in one such embodiment, the carbon to nitrogen ratio of the polymer of Formula 4 may range from about 3.25:1 to about 4.25:1, respectively. By way of further example, in one such embodiment, the carbon to nitrogen ratio of the polymer of Formula 4 may range from about 3.4:1 to about 4:1, respectively. By way of further example, in one such embodiment, the carbon to nitrogen ratio of the polymer of Formula 4 may range from about 3.5:1 to about 3.9:1, respectively. By way of further example, in one such embodiment, the carbon to nitrogen ratio of the polymer of Formula 4 may range from about 3.55:1 to about 3.85:1, respectively. In each of the foregoing embodiments recited in this paragraph, the polymer of Formula 4 is derived from monomers and crosslinkers, each of which comprise less than 5 wt % oxygen.

In certain embodiments, polymers in which crosslinking and/or entanglement were increased were found to have lower swelling than those with lower crosslinking and/or entanglement, yet also had a binding capacity for target ion (e.g., chloride) that was as great as or greater than the lower crosslinking and/or entanglement polymers while binding of interfering ions such as phosphate were significantly reduced. The selectivity effect may be introduced in two different manners: 1) Overall capacity was sacrificed for chloride specificity. Crosslinkers that don't include chloride binding sites (e.g., epichlorohydrin) allow for increased crosslinking while overall capacity is decreased proportional to the amount of crosslinker incorporated into the polymer. 2) Overall capacity is preserved for chloride specificity: Crosslinkers that include chloride binding sites (e.g., diallylamines) allow for increased crosslinking while overall capacity is staying the same or is reduced by only a small amount.

As previously noted, crosslinked polymers having a high capacity for chloride binding and high selectivity for chloride over other competing anions such as phosphate may be prepared in a two-step process in accordance with one embodiment of the present disclosure. In general, the selectivity of the polymer is a function of its crosslinking density and the capacity of the polymer is a function of the free amine density of the crosslinked polymer. Advantageously, the two-step process disclosed herein provides both, high capacity for chloride binding, and high selectivity for chloride over other competing ions by relying primarily upon carbon-carbon crosslinking in the first step, and nitrogen-nitrogen crosslinking in the second step.

In the first step, the crosslinking is preferably capacity-sparing, i.e., free amine sparing, crosslinking from carbon to carbon. In the second step, the crosslinking is amine-consuming and is directed towards tuning for selectivity. Based on the desired high capacity, the C—N ratio is preferably optimized to maximize amine functionalities for HCl binding, while still maintaining a spherical polymer particle of controlled particle size to ensure nonabsorption and acceptable mouth feel that is stable under GI conditions. The preferred extent of carbon-carbon crosslinking achieved after the first step is sufficient to permit the resulting bead to swell between 4× and 6× in water (i.e., a Swelling Ratio of 4 to 6).

In one embodiment, crosslinked polymers having a high capacity for chloride binding and high selectivity for chloride over other competing anions such as phosphate may be prepared in a two-step process, and the product of the first polymerization step is preferably in the form of beads whose diameter is controlled in the 5 to 1000 micrometer range, preferably 10 to 500 micrometers and most preferred 40-180 micrometers.

The product of the first polymerization step is preferably in the form of beads whose Swelling Ratio in water is between 2 and 10, more preferably about 3 to about 8, and most preferably about 4 to about 6.

Additionally, if the crosslinked polymer beads resulting from the first polymerization step are protonated, this may reduce the amount of nitrogen-nitrogen crosslinking in the second crosslinking step. Accordingly, in certain embodiments the preformed amine polymer is at least partially deprotonated by treatment with a base, preferably a strong base such as a hydroxide base. For example, in one embodiment the base may be NaOH, KOH, NH₄OH, NaHCO₃, Na₂CO₃, K₂CO₃, LiOH, Li₂CO₃, CsOH or other metal hydroxides. If the charges are removed from the preformed crosslinked amine polymer bead by deprotonation, the bead will tend to collapse and the crosslinking agent used in the second step may not be able to access binding sites on the polymer unless the bead is prevented from collapsing. One means of preventing the crosslinked polymer bead from collapsing is the use of a swelling agent such as water to swell the bead, thereby allowing the second-step crosslinker to access binding sites.

The preformed polymer may be crosslinked to form the post-polymerization crosslinked polymer using any of a range of crosslinking compounds containing at least two amine-reactive functional groups. In one such embodiment, the crosslinker is a compound containing at least two amine-reactive groups selected from the group consisting of halides, epoxides, phosgene, anhydrides, carbamates, carbonates, isocyanates, thioisocyanates, esters, activated esters, carboxylic acids and derivatives thereof, sulfonates and derivatives thereof, acyl halides, aziridines, α,β-unsaturated carbonyls, ketones, aldehydes, and pentafluoroaryl groups. The crosslinker may be, for example, any of the crosslinkers disclosed herein, including a crosslinker selected from Table B. By way of further example, in one such embodiment the crosslinker is a dihalide such as a dichloroalkane.

As noted above, in certain embodiments a swelling agent for the preformed amine polymer may be included in the reaction mixture for the second polymerization step along with the crosslinking agent. In general, the swelling agent and the crosslinking agent may be miscible or immiscible and the swelling agent may be any composition or combination of compositions that have the capacity to swell the preformed amine polymer. Exemplary swelling agents include polar solvents such as water, methanol, ethanol, n-propanol, isopropanol, n-butanol, formic acid, acetic acid, acetonitrile, dimethylformamide, dimethylsulfoxide, nitromethane, propylene carbonate, or a combination thereof. Additionally, the amount of swelling agent included in the reaction mixture will typically be less than absorption capacity of the preformed amine polymer for the swelling agent. For example, it is generally preferred that the weight ratio of swelling agent to preformed polymer in the reaction mixture be less than 4:1. By way of further example, in some embodiments the weight ratio of swelling agent to preformed polymer in the reaction mixture will be less than 3:1. By way of further example, in some embodiments the weight ratio of swelling agent to preformed polymer in the reaction mixture will be less than 2:1. By way of further example, in some embodiments the weight ratio of swelling agent to preformed polymer in the reaction mixture will be less than 1:1. By way of further example, in some embodiments the weight ratio of swelling agent to preformed polymer in the reaction mixture will be less than 0.5:1. By way of further example, in some embodiments the weight ratio of swelling agent to preformed polymer in the reaction mixture will be less than 0.4:1. By way of further example, in some embodiments the weight ratio of swelling agent to preformed polymer in the reaction mixture will be less than 0.3:1. In general, however, the weight ratio of swelling agent to preformed polymer in the reaction mixture will typically be at least 0.05:1, respectively.

In general, the crosslinked polymers may be crosslinked homopolymers or crosslinked copolymers comprising free amine moieties. The free amine moieties may be separated, for example, by the same or varying lengths of repeating linker (or intervening) units. In some embodiments, the polymers comprise repeat units containing an amine moiety and an intervening linker unit. In other embodiments, multiple amine-containing repeat units are separated by one or more linker units. Additionally, the polyfunctional crosslinkers may comprise HCl binding functional groups, e.g., amines, (“active crosslinkers”) or may lack HCl binding functional groups such as amines (“passive crosslinkers”).

In a preferred embodiment, the first polymerization (crosslinking) step yields preformed amine polymer beads having a target size and chloride binding capacity. For example, in one such embodiment the beads have a chloride binding capacity of at least 10 mmol/g in Simulated Gastric Fluid (“SGF”) and a Swelling Ratio in the range of 1 to 6. The resulting preformed amine polymer is then preferably (at least partially) deprotonated with a base and combined with a non-protonating swelling agent to swell the free amine polymer without protonating the amine functions. Furthermore, the amount of the non-protonating swelling agent is selected to tune the subsequent degree of crosslinking effectively forming a template that is then locked into place via the amine consuming crosslinking step. In the second crosslinking step, the swollen, deprotonated preformed amine polymer is crosslinked with a crosslinker containing amine reactive moieties to form a post-polymerization crosslinked polymer.

In general, selectivity for chloride over other competing ions is achieved with highly crosslinked polymers. For example, relatively high chloride binding capacity maybe be attained by reacting a preformed amine polymer bead with neat crosslinker in the presence of a swelling agent (water). While this “non-dispersed” reaction provides access to high selectivity for chloride over competing ions in the SIB assay, it also results in macroscopically (and microscopically) aggregated polymer beads. Accordingly, it is advantageous to include a solvent (e.g., heptane) in the second crosslinking step to disperse the preformed crosslinked polymer beads so as to avoid inter-bead reactions and resulting aggregation. The use of too much solvent (dispersant), however, can dilute the reaction solution to the point where the resulting bead is not sufficiently crosslinked to have the desired selectivity for chloride over other competing anions. By using a crosslinking agent that also functions as a solvent (dispersant), however, sufficient solvent (dispersant) may be included in the reaction mixture to avoid inter-bead reactions and aggregation without diluting the mixture to the point where the degree of amine-consuming crosslinking is insufficient. For example, in an effort to utilize the dispersing properties of a solvent (to avoid aggregation during the reaction) while maintaining reactivity, DCE and DCP were used neat, thus performing a dual purpose role, as both solvent (dispersant) and crosslinker. Interestingly, DCE was discovered to have excellent dispersal properties as a solvent, when compared to similar reactions with DCP and/or heptane. Additionally, less aggregation was observed when the beads were first dispersed in DCE and then in a second operation, the water is added to swell the beads. If water is added to the preformed amine polymer before the bead is dispersed in the DCE, aggregation may occur.

The use of 1,2-dichloroethane (“DCE”) as the crosslinking solvent also generates HCl molecules during the second step. These HCl molecules protonate some of the free amine sites which block the reaction sites for the crosslinking reaction and thereby limit the number of binding sites available for crosslinking. Consequently, the use of DCE creates a self-limiting effect on the secondary crosslinking.

In each of the foregoing embodiments, the reaction mixture may contain a wide range of amounts of crosslinking agents. For example, in one embodiment the crosslinker may be used in large excess relative to the amount of preformed amine polymer in the reaction mixtures. Stated differently, in such embodiments the crosslinking agent is a crosslinking solvent, i.e., it is both a solvent for the reaction mixture and a crosslinking agent for the preformed amine polymer. In such embodiments, other solvents may optionally be included in the reaction mixture but are not required. Alternatively, the preformed amine polymer, swelling agent and crosslinker may be dispersed in a solvent that is miscible with the crosslinker and immiscible with the swelling agent. For example, in some embodiments the swelling agent may be a polar solvent; in some such embodiments, for example, the swelling agent may comprise water, methanol, ethanol, n-propanol, isopropanol, formic acid, acetic acid, acetonitrile, N,N-dimethylformamide, dimethylsulfoxide, nitromethane, or a combination thereof. By way of further example, when the swelling agent comprises a polar solvent, the solvent system for the reaction mixture will typically comprise a non-polar solvent such as pentane, cyclopentane, hexane, cyclohexane, benzene, toluene, 1,4-dioxane, chloroform, diethyl ether, dichloromethane, dichloroethane, dichloropropane, dichlorobutane, or a combination thereof. In certain embodiments, the crosslinker and the solvent may be the same; i.e., the solvent is a crosslinking solvent such as 1,2-dichloroethane, 1,3-dichloropropane, 1,4-dichlorobutane or a combination thereof.

It is notable that in a crosslinking solvent (e.g., a DCE-dispersed reaction), there is a large excess of crosslinker regardless of the amount of crosslinking solvent (e.g., DCE) used to disperse the bead (e.g., both 1 g:3 mL::bead:DCE and 1 g:10 mL::bead:DCE are a large excess of crosslinker, most of which is not consumed during the reaction). Despite this, the relative degree of crosslinking, and the performance in SIB assay, are unaffected by changes in the ratio of reactive crosslinker to polymer bead. This is possible because the reaction is limited by the acid-neutralizing capacity of the polymer bead, rather than the amount of crosslinker (e.g., DCE).

To more efficiently react with DCE or other crosslinker, the amines of the preformed polymer bead preferably have a free electron pair (neutral, deprotonated). As the free amines of the preformed polymer bead react with the crosslinker (e.g., DCE), HCl is produced and the amines become protonated, thus limiting the reaction. For this reason, the preformed amine polymer beads preferably start as the free amine in the second crosslinking step. If the preformed amine polymer bead is protonated after the first step of carbon-carbon crosslinking, amine-consuming crosslinking in the second step will be limited, thus reducing the desired selectivity for chloride over other competing ions. This has been demonstrated by adding known quantities of HCl to preformed amine polymer beads immediately before second step crosslinking with DCE. When less than 3 mol % HCl (to amine in preformed polymer amine bead) is added prior to second step crosslinking, total chloride capacity (SGF) and chloride selectivity in SIB are similar to beads not treated with HCl in the second step. When greater than 5 mol % HCl (to amine in preformed polymer amine bead) is added prior to second step crosslinking, total chloride capacity (SGF) increases and chloride selectivity in SIB decreases, indicating lower incorporation of crosslinker.

The benefits of deprotonated preformed polymer beads in the second step crosslinking highlights the advantages of using two steps to achieve the final product. In the first step, to form the amine polymer bead, all monomers (e.g., allylamine and DAPDA) are protonated to remain in the aqueous phase and to avoid the radical transfer reactions that severely limit the polymerization of non-protonated allylamine (and derivatives). Once the bead is formed through carbon-carbon crosslinks, the bead can then be deprotonated and further crosslinked with an amine reactive crosslinker in a second step.

Given the large excess of dual crosslinker/solvent, mono-incorporation of this reagent can occur leading to alkyl chloride functional groups on the crosslinked polymer bead that are hydrophobic in nature and can increase non-specific interactions with undesirable solutes other than HCl that are more hydrophobic in nature. Washing with ammonium hydroxide solution converts the alkyl-chloride to alkyl-amine functions that are hydrophilic and minimize non-specific interactions with undesirable solutes. Other modifications that yield more hydrophilic groups than alkyl chloride such as —OH are suitable to quench mono-incorporated crosslinker/solvent.

Any of a range of polymerization chemistries may be employed in the first reaction step, provided that the crosslinking mechanism is primarily carbon-carbon crosslinking. Thus, in one exemplary embodiment, the first reaction step comprises radical polymerization. In such reactions, the amine monomer will typically be a mono-functional vinyl, allyl, or acrylamide (e.g., allylamine) and crosslinkers will have two or more vinyl, allyl or acrylamide functionalities (e.g., diallylamine). Concurrent polymerization and crosslinking occurs through radically initiated polymerization of a mixture of the mono- and multifunctional allylamines. The resulting polymer network is thusly crosslinked through the carbon backbone. Each crosslinking reaction forms a carbon-carbon bond (as opposed to substitution reactions in which a carbon-heteroatom bond is formed during crosslinking). During the concurrent polymerization and crosslinking, the amine functionalities of the monomers do not undergo crosslinking reactions and are preserved in the final polymer (i.e., primary amines remain primary, secondary amines remain secondary, and tertiary amines remain tertiary).

In those embodiments in which the first reaction step comprises radical polymerization, a wide range of initiators may be used including cationic and radical initiators. Some examples of suitable initiators that may be used include: the free radical peroxy and azo type compounds, such as azodiisobutyronitrile, azodiisovaleronitrile, dimethylazodiisobutyrate, 2,2′azo bis(isobutyronitrile), 2,2′-azobis(N,N′-dimethyl-eneisobutyramidine)dihydrochloride, 2,2′-azobis(2-amidinopropane)dihydrochloride, 2,2′-azobis(N,N′-dimethyleneisobutyramidine), 1,1′-azo bis(I-cyclohexanecarbo-nitrile), 4,4′-azobis(4-cyanopentanoic acid), 2,2′-azobis(isobutyramide)dihydrate, 2,2′-azobis(2-methylpropane), 2,2′-azobis(2-methylbutyronitrile), VAZO 67, cyanopentanoic acid, the peroxypivalates, dodecylbenzene peroxide, benzoyl peroxide, di-t-butyl hydroperoxide, t-butyl peracetate, acetyl peroxide, dicumyl peroxide, cumylhydroperoxide, dimethyl bis(butylperoxy)hexane.

Exemplary amine-containing polymers as described above are more fully disclosed and exemplified in WO2016/094685 A1 and WO2014/197725 A1, the entire contents of which are incorporated herein by reference.

The composition, nonabsorbable composition, pharmaceutical composition or proton-binding, crosslinked amine polymer of the present invention can comprise or consist essentially of, or be a polymer as defined anywhere herein. For example, the composition, nonabsorbable composition, pharmaceutical composition or crosslinked amine polymer can comprise, consists essentially of, or be the drug substance TRC101, which has the USAN veverimer. veverimer (TRC101) is a non-absorbed free-flowing powder composed of low-swelling, spherical beads, approximately 100 micrometers in diameter; each bead is a single crosslinked, high molecular weight molecule.

The veverimer polymer (TRC101) can be defined chemically as follows:
1) 1,3-Propanediamine, N¹,N³-di-2-propen-1-yl-, polymer with 1,2-dichloroethane and 2-propen-1-amine;
2) N¹,N³-bis(prop-2-en-1-yl)propan-1,3-diamine copolymer with 1,2-dichloroethane and prop-2-en-1-amine.

Veverimer (TRC101) has the following structural formula:

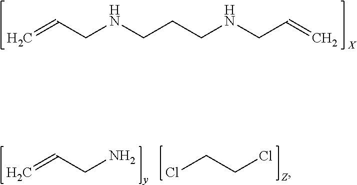

wherein x, y and z are positive integers.

Veverimer (TRC101) has the following molecular formula:

[C₉H₁₈N₂]ₓ.[C₃H₇N]_{y}.[C₂H₄Cl₂]_{z}, wherein x, y and z are positive integers.

Veverimer (TRC101) is obtainable by first copolymerizing allylamine hydrochloride and N,N′-diallyl-1,3-diaminopropane dihydrochloride, or the salts thereof to form a preformed amine polymer, followed by crosslinking the preformed amine polymer with 1,2-dichloroethane. The synthesis of veverimer (TRC101) is described in Exemplary Synthesis A and in WO2016/094685 A1. Veverimer (TRC101) is the polymer with unique ID 019070-A3 FA in Table S-1 of Exemplary Synthesis A. In the present application, veverimer and TRC101 are used interchangeably.

In one embodiment, the pharmaceutical composition comprises a mixture of any of the previously-identified nonabsorbable materials. For example, in one embodiment the pharmaceutical composition comprises a mixture of a cation exchange composition with at least one anion exchange composition, amphoteric ion exchange composition, or neutral composition having the capacity to bind both protons and anions. In another embodiment, the pharmaceutical composition comprises a mixture of an anion exchange composition with at least one cation exchange composition, amphoteric ion exchange composition, or neutral composition having the capacity to bind both protons and anions. In yet another embodiment, the pharmaceutical composition comprises a mixture of a neutral composition having the capacity to bind both protons and anions with at least one cation exchange composition, amphoteric ion exchange composition, or anion exchange composition.

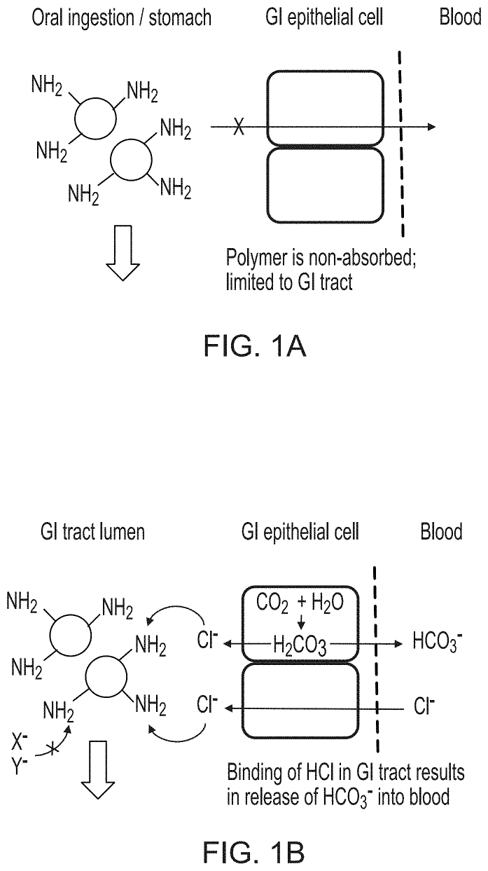

As schematically depicted in FIGS. 1A-1C and in accordance with one embodiment, a nonabsorbable free-amine polymer of the present disclosure is orally ingested and used to treat metabolic acidosis (including by increasing serum bicarbonate and normalizing blood pH) in a mammal by binding HCl in the gastrointestinal (“GI”) tract and removing HCl through the feces. Free-amine polymer is taken orally (FIG. 1A) at compliance enhancing dose targeted to chronically bind sufficient amounts of HCl to enable clinically meaningful increase in serum bicarbonate of 3 mEq/L. In the stomach (FIG. 1B), free amine becomes protonated by binding H. Positive charge on polymer is then available to bind Cl⁻; by controlling access of binding sites through crosslinking and hydrophilicity/hydrophobicity properties, other larger organic anions (e.g., acetate, propionate, butyrate, etc., depicted as X⁻ and Y⁻) are bound to a lesser degree, if at all. The net effect is therefore binding of HCl. In the lower GI tract/colon (FIG. 1C), Cl⁻ is not fully released and HCl is removed from the body through regular bowel movement and fecal excretion, resulting in net alkalinization in the serum. Cl bound in this fashion is not available for exchange via the Cl⁻/HCO₃⁻ antiporter system.

In one embodiment, the polymer is designed to simultaneously maximize efficacy (net HCl binding and excretion) and minimize GI side effects (through low swelling particle design and particle size distribution). Optimized HCl binding may be accomplished through a careful balance of capacity (number of amine binding sites), selectivity (preferred binding of chloride versus other anions, in particular organic anions in the colon) and retention (not releasing significant amounts of chloride in the lower GI tract to avoid the activity of the Cl⁻/HCO₃⁻ exchanger [antiporter] in the colon and intestine; if chloride is not tightly bound to the polymer the Cl/HCO₃⁻ exchanger can mediate uptake of chloride ion from the intestinal lumen and reciprocal exchange for bicarbonate from the serum, thus effectively decreasing serum bicarbonate.

Competing anions that displace chloride lead to a decrease in net bicarbonate through the following mechanisms. First, displacement of chloride from the polymer in the GI lumen, particularly the colon lumen, provides for a facile exchange with bicarbonate in the serum. The colon has an anion exchanger (chloride/bicarbonate antiporter) that moves chloride from the luminal side in exchange for secreted bicarbonate. When free chloride is released from the polymer in the GI tract it will exchange for bicarbonate, which will then be lost in the stool and cause a reduction in total extracellular bicarbonate (Davis, 1983; D'Agostino, 1953). The binding of short chain fatty acids (SCFA) in exchange for bound chloride on the polymer, will result in the depletion of extracellular HCO₃⁻ stores. Short chain fatty acids are the product of bacterial metabolism of complex carbohydrates that are not catabolized by normal digestive processes (Chemlarova, 2007). Short chain fatty acids that reach the colon are absorbed and distributed to various tissues, with the common metabolic fate being the generation of H₂O and CO₂, which is converted to bicarbonate equivalents. Thus, binding of SCFA to the polymer to neutralize the proton charge would be detrimental to overall bicarbonate stores and buffering capacity, necessitating the design of chemical and physical features in the polymer that limit SCFA exchange. Finally, phosphate binding to the polymer should be limited as well, since phosphate represents an additional source of buffering capacity in the situation where ammoniagenesis and/or hydrogen ion secretion is compromised in chronic renal disease.

For each binding of proton, an anion is preferably bound as the positive charge seeks to leave the human body as a neutral polymer. “Binding” of an ion, is more than minimal binding, i.e., at least about 0.2 mmol of ion/g of polymer, at least about 1 mmol of ion/g of polymer in some embodiments, at least about 1.5 mmol of ion/g of polymer in some embodiments, at least about 3 mmol of ion/g of polymer in some embodiments, at least about 5 mmol of ion/g of polymer in some embodiments, at least about 10 mmol of ion/g of polymer in some embodiments, at least about 12 mmol of ion/g of polymer in some embodiments, at least about 13 mmol of ion/g of polymer in some embodiments, or even at least about 14 mmol of ion/g of polymer in some embodiments. In one embodiment, the polymers are characterized by their high capacity of proton binding while at the same time providing selectivity for anions; selectivity for chloride is accomplished by reducing the binding of interfering anions that include but are not limited to phosphate, citrate, acetate, bile acids and fatty acids. For example, in some embodiments, polymers of the present disclosure bind phosphate with a binding capacity of less than about 5 mmol/g, less than about 4 mmol/g, less than about 3 mmol/g, less than about 2 mmol/g or even less than about 1 mmol/g. In some embodiments, polymers of the invention bind bile and fatty acids with a binding capacity of less than about less than about 5 mmol/g, less than about 4 mmol/g, less than about 3 mmol/g, less than about 2 mmol/g, less than about 1 mmol/g in some embodiments, less than about 0.5 mmol/g in some embodiments, less than about 0.3 mmol/g in some embodiments, and less than about 0.1 mmol/g in some embodiments.

### Pharmaceutical Compositions & Administration

In general, the dosage levels of the nonabsorbable compositions for therapeutic and/or prophylactic uses may range from about 0.5 g/day to about 100 g/day. To facilitate patient compliance, it is generally preferred that the dose be in the range of about 1 g/day to about 50 g/day. For example, in one such embodiment, the dose will be about 2 g/day to about 25 g/day. By way of further example, in one such embodiment, the dose will be about 3 g/day to about 25 g/day. By way of further example, in one such embodiment, the dose will be about 4 g/day to about 25 g/day. By way of further example, in one such embodiment, the dose will be about 5 g/day to about 25 g/day. By way of further example, in one such embodiment, the dose will be about 2.5 g/day to about 20 g/day. By way of further example, in one such embodiment, the dose will be about 2.5 g/day to about 15 g/day. By way of further example, in one such embodiment, the dose will be about 1 g/day to about 10 g/day. Optionally, the daily dose may be administered as a single dose (i.e., one time a day), or divided into multiple doses (e.g., two, three or more doses) over the course of a day. In general, the nonabsorbable compositions may be administered as a fixed daily dose or titrated based on the serum bicarbonate values of the patient in need of treatment or other indicators of acidosis. The titration may occur at the onset of treatment or throughout, as required, and starting and maintenance dosage levels may differ from patient to patient based on severity of the underlying disease.

The effectiveness of the nonabsorbable composition may be established in animal models, or in human volunteers and patients. In addition, in vitro, ex vivo and in vivo approaches are useful to establish HCl binding. In vitro binding solutions can be used to measure the binding capacity for proton, chloride and other ions at different pHs. Ex vivo extracts, such as the gastrointestinal lumen contents from human volunteers or from model animals can be used for similar purposes. The selectivity of binding and/or retaining certain ions preferentially over others can also be demonstrated in such in vitro and ex vivo solutions. In vivo models of metabolic acidosis can be used to test the effectiveness of the nonabsorbable composition in normalizing acid/base balance—for example 5/6 nephrectomized rats fed casein-containing chow (as described in Phisitkul S, Hacker C, Simoni J, Tran R M, Wesson D E. Dietary protein causes a decline in the glomerular filtration rate of the remnant kidney mediated by metabolic acidosis and endothelin receptors. Kidney international. 2008; 73(2):192-9), or adenine-fed rats (Terai K, K Mizukami and M Okada. 2008. Comparison of chronic renal failure rats and modification of the preparation protocol as a hyperphosphatemia model. Nephrol. 13: 139-146).

In one embodiment, the nonabsorbable compositions are provided (by oral administration) to an animal, including a human, in a dosing regimen of one, two or even multiple (i.e., at least three) doses per day to treat an acid-base disorder (e.g., metabolic acidosis) and achieve a clinically significant and sustained increase of serum bicarbonate as previously described. For example, in one embodiment a daily dose of the nonabsorbable composition (whether orally administered in a single dose or multiple doses over the course of the day) has sufficient capacity to remove at least 5 mmol of protons, chloride ions or each per day. By way of further example, in one such embodiment a daily dose of the nonabsorbable composition has sufficient capacity to remove at least 10 mmol of protons, chloride ions or each per day. By way of further example, in one such embodiment a daily dose of the nonabsorbable composition has sufficient capacity to remove at least 20 mmol of protons, the conjugate base of a strong acid (e.g., Cl⁻, HSO₄⁻ and SO₄²⁻) and/or a strong acid (e.g., HCl or H₂SO₄) each per day. By way of further example, in one such embodiment a daily dose of the nonabsorbable composition has sufficient capacity to remove at least 30 mmol of protons, the conjugate base of a strong acid, and/or a strong acid each per day. By way of further example, in one such embodiment a daily dose of the nonabsorbable composition has sufficient capacity to remove at least 40 mmol of protons, the conjugate base of a strong acid, and/or a strong acid each per day. By way of further example, in one such embodiment a daily dose of the nonabsorbable composition has sufficient capacity to remove at least 50 mmol of protons, the conjugate base of a strong acid, and/or a strong acid each per day.

The dosage unit form of the pharmaceutical comprising the nonabsorbable composition may be any form appropriate for oral administration. Such dosage unit forms include powders, tablets, pills, lozenges, sachets, cachets, elixirs, suspensions, syrups, soft or hard gelatin capsules, and the like. In one embodiment, the pharmaceutical composition comprises only the nonabsorbable composition. Alternatively, the pharmaceutical composition may comprise a carrier, a diluent, or excipient in addition to the nonabsorbable composition. Examples of carriers, excipients, and diluents that may be used in these formulations as well as others, include foods, drinks, lactose, dextrose, sucrose, sorbitol, mannitol, starches, gum acacia, alginates, tragacanth, gelatin, calcium silicate, microcrystalline cellulose, polyvinylpyrrolidone, cellulose, methyl cellulose, methylhydroxybenzoates, propylhydroxybenzoates, propylhydroxybenzoates, and talc. Pharmaceutical excipients useful in the pharmaceutical compositions further include a binder, such as microcrystalline cellulose, colloidal silica and combinations thereof (Prosolv 90), carbopol, providone and xanthan gum; a flavoring agent, such as sucrose, mannitol, xylitol, maltodextrin, fructose, or sorbitol; a lubricant, such as magnesium stearate, stearic acid, sodium stearyl fumurate and vegetable based fatty acids; and, optionally, a disintegrant, such as croscarmellose sodium, gellan gum, low-substituted hydroxypropyl ether of cellulose, sodium starch glycolate. Other additives may include plasticizers, pigments, talc, and the like. Such additives and other suitable ingredients are well-known in the art; see, e.g., Gennaro A R (ed), Remington's Pharmaceutical Sciences, 20th Edition.

In one embodiment, the nonabsorbable composition may be co-administered with other active pharmaceutical agents depending on the condition being treated. This co-administration may include simultaneous administration of the two agents in the same dosage form, simultaneous administration in separate dosage forms, and separate administration. For example, for the treatment of metabolic acidosis, the nonabsorbable composition may be co-administered with common treatments that are required to treat underlying co-morbidities including but not limited to edema, hypertension, diabetes, obesity, heart failure and complications of Chronic Kidney Disease. These medications and the nonabsorbable composition can be formulated together in the same dosage form and administered simultaneously as long as they do not display any clinically significant drug-drug-interactions. Alternatively, these treatments and the nonabsorbable composition may be separately and sequentially administered with the administration of one being followed by the administration of the other.

In one embodiment, the daily dose of the chronic metabolic acidosis treatment is compliance enhancing (approximately 15 g or less per day) and achieves a clinically significant and sustained increase of serum bicarbonate of approximately 3 mEq/L at these daily doses. The non-absorbed nature of the polymer and the lack of sodium load and/or introduction of other deleterious ions for such an oral drug enable for the first time a safe, chronic treatment of metabolic acidosis without worsening blood pressure/hypertension and/or without causing increased fluid retention and fluid overload. Another benefit is further slowing of the progression of kidney disease and time to onset of lifelong renal replacement therapy (End Stage Renal Disease “ESRD” including 3 times a week dialysis) or need for kidney transplants. Both are associated with significant mortality, low quality of life and significant burden to healthcare systems around the world. In the United States alone, approximately 20% of the 400,000 ESRD patients die and 100,000 new patients start dialysis every year.

A further aspect of the present disclosure is a pharmaceutical product comprising a sealed package and the nonabsorbable composition of the present disclosure within the sealed package. The sealed package is preferably substantially impermeable to moisture and oxygen to increase the stability of the pharmaceutical composition. For example, the dosage unit form may comprise a sealed container (e.g., a sealed sachet) that prevents or reduces ingress of moisture and oxygen upon packaging the nonabsorbable composition in the container. The container size can be optimized to reduce head space in the container after packaging and any head space may be filled with an inert gas such as nitrogen. Furthermore, container material of construction can be chosen to minimize the moisture and oxygen ingress inside the container after packaging. For example, the nonabsorbable composition may be packaged in a multilayer sachet containing at least one or more layer that serves as a barrier layer to moisture and oxygen ingress. In another example, the nonabsorbable composition may be packaged in a single layer or multilayer plastic, metal or glass container that has at least one or more barrier layers incorporated in the structure that limits oxygen and/or moisture ingress after packaging. For example, in one such embodiment the sachet (or other container or package) may comprise a multi-layer laminate of an inner contact layer, an outer layer; and a barrier layer disposed between the contact layer and outer layer. In one exemplary embodiment, the container includes one or more oxygen-scavenging layers.

In further embodiments, enumerated as embodiments 1-849 below, the present disclosure includes:

### Embodiment 1

A method of treating an individual afflicted with an acid-base disorder characterized by a baseline serum bicarbonate value of less than 22 mEq/l, the method comprising oral administration of a daily dose of a pharmaceutical composition having the capacity to bind at least 5 mEq of a target species as it transits the digestive system to increase the serum bicarbonate value to a value within the range of 24 to 29 mEq/l within a treatment period not greater than 1 month, the target species being selected from the group consisting of protons, strong acids, and conjugate bases of strong acids.

### Embodiment 2

A method of treating an individual afflicted with an acid-base disorder characterized by a baseline serum bicarbonate value of less than 22 mEq/l, the method comprising oral administration of a pharmaceutical composition, wherein the pharmaceutical composition given orally binds at least 5 mEq per day on average of a target species in the digestive system to maintain the serum bicarbonate value at a value within the range of 24 to 29 mEq/l, the target species being selected from the group consisting of protons, strong acids, and conjugate bases of strong acids.

### Embodiment 3

The method of embodiment 2 wherein the oral administration is as frequent as at least weekly within the treatment period.

### Embodiment 4

The method of embodiment 2 pharmaceutical composition wherein the oral administration is as frequent as at least semi-weekly within the treatment period.

### Embodiment 5

The method of embodiment 2 pharmaceutical composition wherein the oral administration is as frequent as at least daily within the treatment period.

### Embodiment 6

The method of embodiment 1, 2, 3 or 5 wherein the acid-base disorder is characterized by a baseline serum bicarbonate value of less than 21 mEq/l.

### Embodiment 7

The method of embodiment 1, 2, 3 or 5 wherein the acid-base disorder is characterized by a baseline serum bicarbonate value of less than 20 mEq/l.

### Embodiment 8

The method of embodiment 1, 2, 3 or 5 wherein the acid-base disorder is characterized by a baseline serum bicarbonate value of less than 19 mEq/l.

### Embodiment 9

The method of embodiment 1, 2, 3 or 5 wherein the acid-base disorder is characterized by a baseline serum bicarbonate value of less than 18 mEq/l.

### Embodiment 10

The method of embodiment 1, 2, 3 or 5 wherein the acid-base disorder is characterized by a baseline serum bicarbonate value of less than 17 mEq/l.

### Embodiment 11

The method of embodiment 1, 2, 3 or 5 wherein the acid-base disorder is characterized by a baseline serum bicarbonate value of less than 16 mEq/l.

### Embodiment 12

The method of embodiment 1, 2, 3 or 5 wherein the acid-base disorder is characterized by a baseline serum bicarbonate value of less than 15 mEq/l.

### Embodiment 13

The method of embodiment 1, 2, 3 or 5 wherein the acid-base disorder is characterized by a baseline serum bicarbonate value of less than 14 mEq/l.

### Embodiment 14

The method of embodiment 1, 2, 3 or 5 wherein the acid-base disorder is characterized by a baseline serum bicarbonate value of less than 13 mEq/l.

### Embodiment 15

The method of embodiment 1, 2, 3 or 5 wherein the acid-base disorder is characterized by a baseline serum bicarbonate value of less than 12 mEq/l.

### Embodiment 16

The method of embodiment 1, 2, 3 or 5 wherein the acid-base disorder is characterized by a baseline serum bicarbonate value of less than 11 mEq/l.

### Embodiment 17

The method of embodiment 1, 2, 3 or 5 wherein the acid-base disorder is characterized by a baseline serum bicarbonate value of less than 10 mEq/l.

### Embodiment 18

The method of any preceding enumerated embodiment wherein the acid-base disorder is characterized by a baseline serum bicarbonate value of at least 9 mEq/l.

### Embodiment 19

The method of any of embodiments 1-16 wherein the acid-base disorder is characterized by a baseline serum bicarbonate value of at least 10 mEq/l.

### Embodiment 20

The method of any of embodiments 1-15 wherein the acid-base disorder is characterized by a baseline serum bicarbonate value of at least 11 mEq/l.

### Embodiment 21

The method of any of embodiments 1-14 wherein the acid-base disorder is characterized by a baseline serum bicarbonate value of at least 12 mEq/l.

### Embodiment 22

The method of any of embodiments 1-13 wherein the acid-base disorder is characterized by a baseline serum bicarbonate value of at least 13 mEq/l.

### Embodiment 23

The method of any of embodiments 1-12 wherein the acid-base disorder is characterized by a baseline serum bicarbonate value of at least 14 mEq/l.

### Embodiment 24

The method of any of embodiments 1-11 wherein the acid-base disorder is characterized by a baseline serum bicarbonate value of at least 15 mEq/l.

### Embodiment 25

The method of any of embodiments 1-10 wherein the acid-base disorder is characterized by a baseline serum bicarbonate value of at least 16 mEq/l.

### Embodiment 26

The method of any of embodiments 1-9 wherein the acid-base disorder is characterized by a baseline serum bicarbonate value of at least 17 mEq/l.

### Embodiment 27

The method of any of embodiments 1-8 wherein the acid-base disorder is characterized by a baseline serum bicarbonate value of at least 18 mEq/l.

### Embodiment 28

The method of any of embodiments 1-7 wherein the acid-base disorder is characterized by a baseline serum bicarbonate value of at least 19 mEq/l.

### Embodiment 29

The method of any of embodiments 1-6 wherein the acid-base disorder is characterized by a baseline serum bicarbonate value of at least 20 mEq/l.

### Embodiment 30

The method of embodiment 1, 2, 3 or 5 wherein the acid-base disorder is characterized by a baseline serum bicarbonate value of at least 21 mEq/l.

### Embodiment 34

The method of any preceding enumerated embodiment wherein the method increases the serum bicarbonate value from the baseline serum bicarbonate value to an increased serum bicarbonate value of at least 25 mEq/l.

### Embodiment 35

The method of any preceding enumerated embodiment wherein the method increases the serum bicarbonate value from the baseline serum bicarbonate value to an increased serum bicarbonate value of at least 26 mEq/l.

### Embodiment 36

The method of any preceding enumerated embodiment wherein the method increases the serum bicarbonate value from the baseline serum bicarbonate value to an increased serum bicarbonate value of at least 27 mEq/l.

### Embodiment 37

The method of any preceding enumerated embodiment wherein the method increases the serum bicarbonate value from the baseline serum bicarbonate value to an increased serum bicarbonate value of at least 28 mEq/l.

### Embodiment 38

The method of any preceding enumerated embodiment wherein the method increases the baseline serum bicarbonate value to an increased serum bicarbonate value not in excess of 29 mEq/l.

### Embodiment 39

The method of any of embodiments 1 to 36 wherein the method increases the baseline serum bicarbonate value to an increased serum bicarbonate value not in excess of 28 mEq/l.

### Embodiment 40

The method of any of embodiments 1 to 35 wherein the method increases the baseline serum bicarbonate value to an increased serum bicarbonate value not in excess of 27 mEq/l.

### Embodiment 41

The method of any of embodiments 1 to 34 wherein the method increases the baseline serum bicarbonate value to an increased serum bicarbonate value not in excess of 26 mEq/l.

### Embodiment 42

The method of any of embodiments 1 to 33 wherein the method increases the baseline serum bicarbonate value to an increased serum bicarbonate value not in excess of 25 mEq/l.

### Embodiment 45

The method of any preceding enumerated embodiment wherein the method increases the baseline serum bicarbonate value at least 1 mEq/l.

### Embodiment 46

The method of any preceding enumerated embodiment wherein the method increases the baseline serum bicarbonate value at least 1.5 mEq/l.

### Embodiment 47

The method of any preceding enumerated embodiment wherein the method increases the baseline serum bicarbonate value at least 2 mEq/l.

### Embodiment 48

The method of any preceding enumerated embodiment wherein the method increases the baseline serum bicarbonate value at least 2.5 mEq/l.

### Embodiment 49

The method of any preceding enumerated embodiment wherein the method increases the baseline serum bicarbonate value at least 3 mEq/l.

### Embodiment 50

The method of any preceding enumerated embodiment wherein the method increases the baseline serum bicarbonate value at least 3.5 mEq/l.

### Embodiment 51

The method of any preceding enumerated embodiment wherein the method increases the baseline serum bicarbonate value at least 4 mEq/l.

### Embodiment 52

The method of any preceding enumerated embodiment wherein the method increases the baseline serum bicarbonate value at least 4.5 mEq/l.

### Embodiment 53

The method of any preceding enumerated embodiment wherein the method increases the baseline serum bicarbonate value at least 5 mEq/l.

### Embodiment 54

The method of any preceding enumerated embodiment wherein the method increases the baseline serum bicarbonate value at least 5.5 mEq/l.

### Embodiment 55

The method of any preceding enumerated embodiment wherein the method increases the baseline serum bicarbonate value at least 6 mEq/l.

### Embodiment 56

The method of any preceding enumerated embodiment wherein the method increases the baseline serum bicarbonate value at least 6.5 mEq/l.

### Embodiment 57

The method of any preceding enumerated embodiment wherein the method increases the baseline serum bicarbonate value at least 7 mEq/l.

### Embodiment 58

The method of any preceding enumerated embodiment wherein the method increases the baseline serum bicarbonate value at least 7.5 mEq/l.

### Embodiment 59

The method of any preceding enumerated embodiment wherein the method increases the baseline serum bicarbonate value at least 8 mEq/l.

### Embodiment 60

The method of any preceding enumerated embodiment wherein the method increases the baseline serum bicarbonate value at least 8.5 mEq/l.

### Embodiment 61

The method of any preceding enumerated embodiment wherein the method increases the baseline serum bicarbonate value at least 9 mEq/l.

### Embodiment 62

The method of any preceding enumerated embodiment wherein the method increases the baseline serum bicarbonate value to a value within the range of 24 to 29 mEq/l within a treatment period of less than one month.

### Embodiment 63

The method of any preceding enumerated embodiment wherein the method increases the baseline serum bicarbonate value to a value within the range of 24 to 29 mEq/l within a treatment period of 25 days.

### Embodiment 64

The method of any preceding enumerated embodiment wherein the method increases the baseline serum bicarbonate value to a value within the range of 24 to 29 mEq/l within a treatment period of 3 weeks.

### Embodiment 65

The method of any preceding enumerated embodiment wherein the method increases the baseline serum bicarbonate value to a value within the range of 24 to 29 mEq/l within a treatment period of 15 days.

### Embodiment 66

The method of any preceding enumerated embodiment wherein the method increases the baseline serum bicarbonate value to a value within the range of 24 to 29 mEq/l within a treatment period of 2 weeks.

### Embodiment 67

The method of any preceding enumerated embodiment wherein the method increases the baseline serum bicarbonate value to a value within the range of 24 to 29 mEq/l within a treatment period of 10 days.

### Embodiment 68

The method of any preceding enumerated embodiment wherein the method increases the baseline serum bicarbonate value to a value within the range of 24 to 29 mEq/l within a treatment period of 1 week.

### Embodiment 69

The method of any preceding enumerated embodiment wherein the method increases the baseline serum bicarbonate value to a value within the range of 24 to 29 mEq/l within 6 days of the initiation of the treatment.

### Embodiment 70

The method of any preceding enumerated embodiment wherein the method increases the baseline serum bicarbonate value to a value within the range of 24 to 29 mEq/l within a treatment period of 5 days.

### Embodiment 71

The method of any preceding enumerated embodiment wherein the method increases the baseline serum bicarbonate value to a value within the range of 24 to 29 mEq/l within a treatment period of 4 days.

### Embodiment 72

The method of any preceding enumerated embodiment wherein the method increases the baseline serum bicarbonate value to a value within the range of 24 to 29 mEq/l within a treatment period of 3 days.

### Embodiment 73

The method of any preceding enumerated embodiment wherein the method increases the baseline serum bicarbonate value to a value within the range of 24 to 29 mEq/l within a treatment period of 2 days.

### Embodiment 74

The method of any preceding enumerated embodiment wherein the method increases the baseline serum bicarbonate value to a value within the range of 24 to 29 mEq/l within a treatment period of 1 day.

### Embodiment 75

The method of any preceding enumerated embodiment wherein the method increases the baseline serum bicarbonate value to a value within the range of 24 to 29 mEq/l within a treatment period of 12 hours.

### Embodiment 76

The method of any preceding enumerated embodiment wherein the method increases the baseline serum bicarbonate value to a value within the range of 24 to 29 mEq/l without any change in the individual's diet or dietary habits relative to the period immediately preceding the initiation of treatment.

### Embodiment 77

The method of any preceding enumerated embodiment wherein the method increases the baseline serum bicarbonate value to a value within the range of 24 to 29 mEq/l independent of the individual's diet or dietary habits.

### Embodiment 78

The method of any preceding enumerated embodiment wherein the individual's serum bicarbonate value returns to the baseline value±2.5 mEq/l within 1 month of the cessation of treatment.

### Embodiment 79

The method of any preceding enumerated embodiment wherein the individual's serum bicarbonate value returns to the baseline value±2.5 mEq/l within 3 weeks of the cessation of treatment.

### Embodiment 80

The method of any preceding enumerated embodiment wherein the individual's serum bicarbonate value returns to the baseline value±2.5 mEq/l within 2 weeks of the cessation of treatment.

### Embodiment 81

The method of any preceding enumerated embodiment wherein the individual's serum bicarbonate value returns to the baseline value±2.5 mEq/l within 10 days of the cessation of treatment.

### Embodiment 82

The method of any preceding enumerated embodiment wherein the individual's serum bicarbonate value returns to the baseline value±2.5 mEq/l within 9 days of the cessation of treatment.

### Embodiment 83

The method of any preceding enumerated embodiment wherein the individual's serum bicarbonate value returns to the baseline value±2.5 mEq/l within 8 days of the cessation of treatment.

### Embodiment 84

The method of any preceding enumerated embodiment wherein the individual's serum bicarbonate value returns to the baseline value±2.5 mEq/l within 7 days of the cessation of treatment.

### Embodiment 85

The method of any preceding enumerated embodiment wherein the individual's serum bicarbonate value returns to the baseline value±2.5 mEq/l within 6 days of the cessation of treatment.

### Embodiment 86

The method of any preceding enumerated embodiment wherein the individual's serum bicarbonate value returns to the baseline value±2.5 mEq/l within 5 days of the cessation of treatment.

### Embodiment 87

The method of any preceding enumerated embodiment wherein the individual's serum bicarbonate value returns to the baseline value±2.5 mEq/l within 4 days of the cessation of treatment.

### Embodiment 88

The method of any preceding enumerated embodiment wherein the individual's serum bicarbonate value returns to the baseline value±2.5 mEq/l within 3 days of the cessation of treatment.

### Embodiment 89

The method of any preceding enumerated embodiment wherein the individual's serum bicarbonate value returns to the baseline value±2.5 mEq/l within 2 days of the cessation of treatment.

### Embodiment 90

The method of any preceding enumerated embodiment wherein the individual's serum bicarbonate value returns to the baseline value±2.5 mEq/l within 1 day of the cessation of treatment.

### Embodiment 91

The method of any preceding enumerated embodiment wherein the individual's serum bicarbonate value returns to the baseline value±2 mEq/l within 1 month of the cessation of treatment.

### Embodiment 92

The method of any preceding enumerated embodiment wherein the individual's serum bicarbonate value returns to the baseline value±2 mEq/l within 3 weeks of the cessation of treatment.

### Embodiment 93

The method of any preceding enumerated embodiment wherein the individual's serum bicarbonate value returns to the baseline value±2 mEq/l within 2 weeks of the cessation of treatment.

### Embodiment 94

The method of any preceding enumerated embodiment wherein the individual's serum bicarbonate value returns to the baseline value±2 mEq/l within 10 days of the cessation of treatment.

### Embodiment 95

The method of any preceding enumerated embodiment wherein the individual's serum bicarbonate value returns to the baseline value±2 mEq/l within 9 days of the cessation of treatment.

### Embodiment 96

The method of any preceding enumerated embodiment wherein the individual's serum bicarbonate value returns to the baseline value±2 mEq/l within 8 days of the cessation of treatment.

### Embodiment 97

The method of any preceding enumerated embodiment wherein the individual's serum bicarbonate value returns to the baseline value±2 mEq/l within 7 days of the cessation of treatment.

### Embodiment 98

The method of any preceding enumerated embodiment wherein the individual's serum bicarbonate value returns to the baseline value±2 mEq/l within 6 days of the cessation of treatment.

### Embodiment 99

The method of any preceding enumerated embodiment wherein the individual's serum bicarbonate value returns to the baseline value±2 mEq/l within 5 days of the cessation of treatment.

### Embodiment 100

The method of any preceding enumerated embodiment wherein the individual's serum bicarbonate value returns to the baseline value±2 mEq/l within 4 days of the cessation of treatment.

### Embodiment 101

The method of any preceding enumerated embodiment wherein the individual's serum bicarbonate value returns to the baseline value±2 mEq/l within 3 days of the cessation of treatment.

### Embodiment 102

The method of any preceding enumerated embodiment wherein the individual's serum bicarbonate value returns to the baseline value±2 mEq/l within 2 days of the cessation of treatment.

### Embodiment 103

The method of any preceding enumerated embodiment wherein the individual's serum bicarbonate value returns to the baseline value±2 mEq/l within 1 day of the cessation of treatment.

### Embodiment 104

The method of any preceding enumerated embodiment wherein the individual's serum bicarbonate value returns to the baseline value±1.5 mEq/l within 1 month of the cessation of treatment.

### Embodiment 105

The method of any preceding enumerated embodiment wherein the individual's serum bicarbonate value returns to the baseline value±1.5 mEq/l within 3 weeks of the cessation of treatment.

### Embodiment 106

The method of any preceding enumerated embodiment wherein the individual's serum bicarbonate value returns to the baseline value±1.5 mEq/l within 2 weeks of the cessation of treatment.

### Embodiment 107

The method of any preceding enumerated embodiment wherein the individual's serum bicarbonate value returns to the baseline value±1.5 mEq/l within 10 days of the cessation of treatment.

### Embodiment 108

The method of any preceding enumerated embodiment wherein the individual's serum bicarbonate value returns to the baseline value±1.5 mEq/l within 9 days of the cessation of treatment.

### Embodiment 109

The method of any preceding enumerated embodiment wherein the individual's serum bicarbonate value returns to the baseline value±1.5 mEq/l within 8 days of the cessation of treatment.

### Embodiment 110

The method of any preceding enumerated embodiment wherein the individual's serum bicarbonate value returns to the baseline value±1.5 mEq/l within 7 days of the cessation of treatment.

### Embodiment 111

The method of any preceding enumerated embodiment wherein the individual's serum bicarbonate value returns to the baseline value±1.5 mEq/l within 6 days of the cessation of treatment.

### Embodiment 112

The method of any preceding enumerated embodiment wherein the individual's serum bicarbonate value returns to the baseline value±1.5 mEq/l within 5 days of the cessation of treatment.

### Embodiment 113

The method of any preceding enumerated embodiment wherein the individual's serum bicarbonate value returns to the baseline value±1.5 mEq/l within 4 days of the cessation of treatment.

### Embodiment 114

The method of any preceding enumerated embodiment wherein the individual's serum bicarbonate value returns to the baseline value±1.5 mEq/l within 3 days of the cessation of treatment.

### Embodiment 115

The method of any preceding enumerated embodiment wherein the individual's serum bicarbonate value returns to the baseline value±1.5 mEq/l within 2 days of the cessation of treatment.

### Embodiment 116

The method of any preceding enumerated embodiment wherein the individual's serum bicarbonate value returns to the baseline value±1.5 mEq/l within 1 day of the cessation of treatment.

### Embodiment 117

The method of any preceding enumerated embodiment wherein the individual's serum bicarbonate value returns to the baseline value±1 mEq/l within 1 month of the cessation of treatment.

### Embodiment 118

The method of any preceding enumerated embodiment wherein the individual's serum bicarbonate value returns to the baseline value±1 mEq/l within 3 weeks of the cessation of treatment.

### Embodiment 119

The method of any preceding enumerated embodiment wherein the individual's serum bicarbonate value returns to the baseline value±1 mEq/l within 2 weeks of the cessation of treatment.

### Embodiment 120

The method of any preceding enumerated embodiment wherein the individual's serum bicarbonate value returns to the baseline value±1 mEq/l within 10 days of the cessation of treatment.

### Embodiment 121

The method of any preceding enumerated embodiment wherein the individual's serum bicarbonate value returns to the baseline value±1 mEq/l within 9 days of the cessation of treatment.

### Embodiment 122

The method of any preceding enumerated embodiment wherein the individual's serum bicarbonate value returns to the baseline value±1 mEq/l within 8 days of the cessation of treatment.

### Embodiment 123

The method of any preceding enumerated embodiment wherein the individual's serum bicarbonate value returns to the baseline value±1 mEq/l within 7 days of the cessation of treatment.

### Embodiment 124

The method of any preceding enumerated embodiment wherein the individual's serum bicarbonate value returns to the baseline value±1 mEq/l within 6 days of the cessation of treatment.

### Embodiment 125

The method of any preceding enumerated embodiment wherein the individual's serum bicarbonate value returns to the baseline value±1 mEq/l within 5 days of the cessation of treatment.

### Embodiment 126

The method of any preceding enumerated embodiment wherein the individual's serum bicarbonate value returns to the baseline value±1 mEq/l within 4 days of the cessation of treatment.

### Embodiment 127

The method of any preceding enumerated embodiment wherein the individual's serum bicarbonate value returns to the baseline value±1 mEq/l within 3 days of the cessation of treatment.

### Embodiment 128

The method of any preceding enumerated embodiment wherein the individual's serum bicarbonate value returns to the baseline value±1 mEq/l within 2 days of the cessation of treatment.

### Embodiment 129

The method of any preceding enumerated embodiment wherein the individual's serum bicarbonate value returns to the baseline value±1 mEq/l within 1 day of the cessation of treatment.

### Embodiment 130

The method of any preceding enumerated embodiment wherein, upon cessation of the treatment, the individual's serum bicarbonate value decreases by at least 1 mEq/l within 1 month of the cessation of treatment.

### Embodiment 131

The method of any preceding enumerated embodiment wherein, upon cessation of the treatment, the individual's serum bicarbonate value decreases by at least 1 mEq/l within 3 weeks of the cessation of treatment.

### Embodiment 132

The method of any preceding enumerated embodiment wherein, upon cessation of the treatment, the individual's serum bicarbonate value decreases by at least 1 mEq/l within 2 weeks of the cessation of treatment.

### Embodiment 133

The method of any preceding enumerated embodiment wherein, upon cessation of the treatment, the individual's serum bicarbonate value decreases by at least 1 mEq/l within 10 days of the cessation of treatment.

### Embodiment 134

The method of any preceding enumerated embodiment wherein, upon cessation of the treatment, the individual's serum bicarbonate value decreases by at least 1 mEq/l within 9 days of the cessation of treatment.

### Embodiment 135

The method of any preceding enumerated embodiment wherein, upon cessation of the treatment, the individual's serum bicarbonate value decreases by at least 1 mEq/l within 8 days of the cessation of treatment.

### Embodiment 136

The method of any preceding enumerated embodiment wherein, upon cessation of the treatment, the individual's serum bicarbonate value decreases by at least 1 mEq/l within 7 days of the cessation of treatment.

### Embodiment 137

The method of any preceding enumerated embodiment wherein, upon cessation of the treatment, the individual's serum bicarbonate value decreases by at least 1 mEq/l within 6 days of the cessation of treatment.

### Embodiment 138

The method of any preceding enumerated embodiment wherein, upon cessation of the treatment, the individual's serum bicarbonate value decreases by at least 1 mEq/l within 5 days of the cessation of treatment.

### Embodiment 139

The method of any preceding enumerated embodiment wherein, upon cessation of the treatment, the individual's serum bicarbonate value decreases by at least 1 mEq/l within 4 days of the cessation of treatment.

### Embodiment 140

The method of any preceding enumerated embodiment wherein, upon cessation of the treatment, the individual's serum bicarbonate value decreases by at least 1 mEq/l within 3 days of the cessation of treatment.

### Embodiment 141

The method of any preceding enumerated embodiment wherein, upon cessation of the treatment, the individual's serum bicarbonate value decreases by at least 1 mEq/l within 2 days of the cessation of treatment.

### Embodiment 142

The method of any preceding enumerated embodiment wherein, upon cessation of the treatment, the individual's serum bicarbonate value decreases by at least 1 mEq/l within 1 day of the cessation of treatment.

### Embodiment 143

The method of any preceding enumerated embodiment wherein, upon cessation of the treatment, the individual's serum bicarbonate value decreases by at least 1.5 mEq/l within 1 month of the cessation of treatment.

### Embodiment 144

The method of any preceding enumerated embodiment wherein, upon cessation of the treatment, the individual's serum bicarbonate value decreases by at least 1.5 mEq/l within 3 weeks of the cessation of treatment.

### Embodiment 145

The method of any preceding enumerated embodiment wherein, upon cessation of the treatment, the individual's serum bicarbonate value decreases by at least 1.5 mEq/l within 2 weeks of the cessation of treatment.

### Embodiment 146

The method of any preceding enumerated embodiment wherein, upon cessation of the treatment, the individual's serum bicarbonate value decreases by at least 1.5 mEq/l within 10 days of the cessation of treatment.

### Embodiment 147

The method of any preceding enumerated embodiment wherein, upon cessation of the treatment, the individual's serum bicarbonate value decreases by at least 1.5 mEq/l within 9 days of the cessation of treatment.

### Embodiment 148

The method of any preceding enumerated embodiment wherein, upon cessation of the treatment, the individual's serum bicarbonate value decreases by at least 1.5 mEq/l within 8 days of the cessation of treatment.

### Embodiment 149

The method of any preceding enumerated embodiment wherein, upon cessation of the treatment, the individual's serum bicarbonate value decreases by at least 1.5 mEq/l within 7 days of the cessation of treatment.

### Embodiment 150

The method of any preceding enumerated embodiment wherein, upon cessation of the treatment, the individual's serum bicarbonate value decreases by at least 1.5 mEq/l within 6 days of the cessation of treatment.

### Embodiment 151

The method of any preceding enumerated embodiment wherein, upon cessation of the treatment, the individual's serum bicarbonate value decreases by at least 1.5 mEq/l within 5 days of the cessation of treatment.

### Embodiment 152

The method of any preceding enumerated embodiment wherein, upon cessation of the treatment, the individual's serum bicarbonate value decreases by at least 1.5 mEq/l within 4 days of the cessation of treatment.

### Embodiment 153

The method of any preceding enumerated embodiment wherein, upon cessation of the treatment, the individual's serum bicarbonate value decreases by at least 1.5 mEq/l within 3 days of the cessation of treatment.

### Embodiment 154

The method of any preceding enumerated embodiment wherein, upon cessation of the treatment, the individual's serum bicarbonate value decreases by at least 1.5 mEq/l within 2 days of the cessation of treatment.

### Embodiment 155

The method of any preceding enumerated embodiment wherein, upon cessation of the treatment, the individual's serum bicarbonate value decreases by at least 1.5 mEq/l within 1 day of the cessation of treatment.

### Embodiment 156

The method of any preceding enumerated embodiment wherein, upon cessation of the treatment, the individual's serum bicarbonate value decreases by at least 2 mEq/l within 1 month of the cessation of treatment.

### Embodiment 157

The method of any preceding enumerated embodiment wherein, upon cessation of the treatment, the individual's serum bicarbonate value decreases by at least 2 mEq/l within 3 weeks of the cessation of treatment.

### Embodiment 158

The method of any preceding enumerated embodiment wherein, upon cessation of the treatment, the individual's serum bicarbonate value decreases by at least 2 mEq/l within 2 weeks of the cessation of treatment.

### Embodiment 159

The method of any preceding enumerated embodiment wherein, upon cessation of the treatment, the individual's serum bicarbonate value decreases by at least 2 mEq/l within 10 days of the cessation of treatment.

### Embodiment 160

The method of any preceding enumerated embodiment wherein, upon cessation of the treatment, the individual's serum bicarbonate value decreases by at least 2 mEq/l within 9 days of the cessation of treatment.

### Embodiment 161

The method of any preceding enumerated embodiment wherein, upon cessation of the treatment, the individual's serum bicarbonate value decreases by at least 2 mEq/l within 8 days of the cessation of treatment.

### Embodiment 162

The method of any preceding enumerated embodiment wherein, upon cessation of the treatment, the individual's serum bicarbonate value decreases by at least 2 mEq/l within 7 days of the cessation of treatment.

### Embodiment 163

The method of any preceding enumerated embodiment wherein, upon cessation of the treatment, the individual's serum bicarbonate value decreases by at least 2 mEq/l within 6 days of the cessation of treatment.

### Embodiment 164

The method of any preceding enumerated embodiment wherein, upon cessation of the treatment, the individual's serum bicarbonate value decreases by at least 2 mEq/l within 5 days of the cessation of treatment.

### Embodiment 165

The method of any preceding enumerated embodiment wherein, upon cessation of the treatment, the individual's serum bicarbonate value decreases by at least 2 mEq/l within 4 days of the cessation of treatment.

### Embodiment 166

The method of any preceding enumerated embodiment wherein, upon cessation of the treatment, the individual's serum bicarbonate value decreases by at least 2 mEq/l within 3 days of the cessation of treatment.

### Embodiment 167

The method of any preceding enumerated embodiment wherein, upon cessation of the treatment, the individual's serum bicarbonate value decreases by at least 2 mEq/l within 2 days of the cessation of treatment.

### Embodiment 168

The method of any preceding enumerated embodiment wherein, upon cessation of the treatment, the individual's serum bicarbonate value decreases by at least 2 mEq/l within 1 day of the cessation of treatment.

### Embodiment 169

The method of any preceding enumerated embodiment wherein, upon cessation of the treatment, the individual's serum bicarbonate value decreases by at least 2.5 mEq/l within 1 month of the cessation of treatment.

### Embodiment 170

The method of any preceding enumerated embodiment wherein, upon cessation of the treatment, the individual's serum bicarbonate value decreases by at least 2.5 mEq/l within 3 weeks of the cessation of treatment.

### Embodiment 171

The method of any preceding enumerated embodiment wherein, upon cessation of the treatment, the individual's serum bicarbonate value decreases by at least 2.5 mEq/l within 2 weeks of the cessation of treatment.

### Embodiment 172

The method of any preceding enumerated embodiment wherein, upon cessation of the treatment, the individual's serum bicarbonate value decreases by at least 2.5 mEq/l within 10 days of the cessation of treatment.

### Embodiment 173

The method of any preceding enumerated embodiment wherein, upon cessation of the treatment, the individual's serum bicarbonate value decreases by at least 2.5 mEq/l within 9 days of the cessation of treatment.

### Embodiment 174

The method of any preceding enumerated embodiment wherein, upon cessation of the treatment, the individual's serum bicarbonate value decreases by at least 2.5 mEq/l within 8 days of the cessation of treatment.

### Embodiment 175

The method of any preceding enumerated embodiment wherein, upon cessation of the treatment, the individual's serum bicarbonate value decreases by at least 2.5 mEq/l within 7 days of the cessation of treatment.

### Embodiment 176

The method of any preceding enumerated embodiment wherein, upon cessation of the treatment, the individual's serum bicarbonate value decreases by at least 2.5 mEq/l within 6 days of the cessation of treatment.

### Embodiment 177

The method of any preceding enumerated embodiment wherein, upon cessation of the treatment, the individual's serum bicarbonate value decreases by at least 2.5 mEq/l within 5 days of the cessation of treatment.

### Embodiment 178

The method of any preceding enumerated embodiment wherein, upon cessation of the treatment, the individual's serum bicarbonate value decreases by at least 2.5 mEq/l within 4 days of the cessation of treatment.

### Embodiment 179

The method of any preceding enumerated embodiment wherein, upon cessation of the treatment, the individual's serum bicarbonate value decreases by at least 2.5 mEq/l within 3 days of the cessation of treatment.

### Embodiment 180

The method of any preceding enumerated embodiment wherein, upon cessation of the treatment, the individual's serum bicarbonate value decreases by at least 2.5 mEq/l within 2 days of the cessation of treatment.

### Embodiment 181

The method of any preceding enumerated embodiment wherein, upon cessation of the treatment, the individual's serum bicarbonate value decreases by at least 2.5 mEq/l within 1 day of the cessation of treatment.

### Embodiment 182

The method of any preceding enumerated embodiment wherein, upon cessation of the treatment, the individual's serum bicarbonate value decreases by at least 3 mEq/l within 1 month of the cessation of treatment.

### Embodiment 183

The method of any preceding enumerated embodiment wherein, upon cessation of the treatment, the individual's serum bicarbonate value decreases by at least 3 mEq/l within 3 weeks of the cessation of treatment.

### Embodiment 184

The method of any preceding enumerated embodiment wherein, upon cessation of the treatment, the individual's serum bicarbonate value decreases by at least 3 mEq/l within 2 weeks of the cessation of treatment.

### Embodiment 185

The method of any preceding enumerated embodiment wherein, upon cessation of the treatment, the individual's serum bicarbonate value decreases by at least 3 mEq/l within 10 days of the cessation of treatment.

### Embodiment 186

The method of any preceding enumerated embodiment wherein, upon cessation of the treatment, the individual's serum bicarbonate value decreases by at least 3 mEq/l within 9 days of the cessation of treatment.

### Embodiment 187

The method of any preceding enumerated embodiment wherein, upon cessation of the treatment, the individual's serum bicarbonate value decreases by at least 3 mEq/l within 8 days of the cessation of treatment.

### Embodiment 188

The method of any preceding enumerated embodiment wherein, upon cessation of the treatment, the individual's serum bicarbonate value decreases by at least 3 mEq/l within 7 days of the cessation of treatment.

### Embodiment 189

The method of any preceding enumerated embodiment wherein, upon cessation of the treatment, the individual's serum bicarbonate value decreases by at least 3 mEq/l within 6 days of the cessation of treatment.

### Embodiment 190

The method of any preceding enumerated embodiment wherein, upon cessation of the treatment, the individual's serum bicarbonate value decreases by at least 3 mEq/l within 5 days of the cessation of treatment.

### Embodiment 191

The method of any preceding enumerated embodiment wherein, upon cessation of the treatment, the individual's serum bicarbonate value decreases by at least 3 mEq/l within 4 days of the cessation of treatment.

### Embodiment 192

The method of any preceding enumerated embodiment wherein, upon cessation of the treatment, the individual's serum bicarbonate value decreases by at least 3 mEq/l within 3 days of the cessation of treatment.

### Embodiment 193

The method of any preceding enumerated embodiment wherein, upon cessation of the treatment, the individual's serum bicarbonate value decreases by at least 3 mEq/l within 2 days of the cessation of treatment.

### Embodiment 194

The method of any preceding enumerated embodiment wherein, upon cessation of the treatment, the individual's serum bicarbonate value decreases by at least 3 mEq/l within 1 day of the cessation of treatment.

### Embodiment 195

The method of any preceding enumerated embodiment wherein, upon cessation of the treatment, the individual's serum bicarbonate value decreases by at least 3.5 mEq/l within 1 month of the cessation of treatment.

### Embodiment 196

The method of any preceding enumerated embodiment wherein, upon cessation of the treatment, the individual's serum bicarbonate value decreases by at least 3.5 mEq/l within 3 weeks of the cessation of treatment.

### Embodiment 197

The method of any preceding enumerated embodiment wherein, upon cessation of the treatment, the individual's serum bicarbonate value decreases by at least 3.5 mEq/l within 2 weeks of the cessation of treatment.

### Embodiment 198

The method of any preceding enumerated embodiment wherein, upon cessation of the treatment, the individual's serum bicarbonate value decreases by at least 3.5 mEq/l within 10 days of the cessation of treatment.

### Embodiment 199

The method of any preceding enumerated embodiment wherein, upon cessation of the treatment, the individual's serum bicarbonate value decreases by at least 3.5 mEq/l within 9 days of the cessation of treatment.

### Embodiment 200

The method of any preceding enumerated embodiment wherein, upon cessation of the treatment, the individual's serum bicarbonate value decreases by at least 3.5 mEq/l within 8 days of the cessation of treatment.

### Embodiment 201

The method of any preceding enumerated embodiment wherein, upon cessation of the treatment, the individual's serum bicarbonate value decreases by at least 3.5 mEq/l within 7 days of the cessation of treatment.

### Embodiment 202

The method of any preceding enumerated embodiment wherein, upon cessation of the treatment, the individual's serum bicarbonate value decreases by at least 3.5 mEq/l within 6 days of the cessation of treatment.

### Embodiment 203

The method of any preceding enumerated embodiment wherein, upon cessation of the treatment, the individual's serum bicarbonate value decreases by at least 3.5 mEq/l within 5 days of the cessation of treatment.

### Embodiment 204

The method of any preceding enumerated embodiment wherein, upon cessation of the treatment, the individual's serum bicarbonate value decreases by at least 3.5 mEq/l within 4 days of the cessation of treatment.

### Embodiment 205

The method of any preceding enumerated embodiment wherein, upon cessation of the treatment, the individual's serum bicarbonate value decreases by at least 3.5 mEq/l within 3 days of the cessation of treatment.

### Embodiment 206

The method of any preceding enumerated embodiment wherein, upon cessation of the treatment, the individual's serum bicarbonate value decreases by at least 3.5 mEq/l within 2 days of the cessation of treatment.

### Embodiment 207

The method of any preceding enumerated embodiment wherein, upon cessation of the treatment, the individual's serum bicarbonate value decreases by at least 3.5 mEq/l within 1 day of the cessation of treatment.

### Embodiment 208

The method of any preceding enumerated embodiment wherein, upon cessation of the treatment, the individual's serum bicarbonate value decreases by at least 4 mEq/l within 1 month of the cessation of treatment.

### Embodiment 209

The method of any preceding enumerated embodiment wherein, upon cessation of the treatment, the individual's serum bicarbonate value decreases by at least 4 mEq/l within 3 weeks of the cessation of treatment.

### Embodiment 210

The method of any preceding enumerated embodiment wherein, upon cessation of the treatment, the individual's serum bicarbonate value decreases by at least 4 mEq/l within 2 weeks of the cessation of treatment.

### Embodiment 211

The method of any preceding enumerated embodiment wherein, upon cessation of the treatment, the individual's serum bicarbonate value decreases by at least 4 mEq/l within 10 days of the cessation of treatment.

### Embodiment 212

The method of any preceding enumerated embodiment wherein, upon cessation of the treatment, the individual's serum bicarbonate value decreases by at least 4 mEq/l within 9 days of the cessation of treatment.

### Embodiment 213

The method of any preceding enumerated embodiment wherein, upon cessation of the treatment, the individual's serum bicarbonate value decreases by at least 4 mEq/l within 8 days of the cessation of treatment.

### Embodiment 214

The method of any preceding enumerated embodiment wherein, upon cessation of the treatment, the individual's serum bicarbonate value decreases by at least 4 mEq/l within 7 days of the cessation of treatment.

### Embodiment 215

The method of any preceding enumerated embodiment wherein, upon cessation of the treatment, the individual's serum bicarbonate value decreases by at least 4 mEq/l within 6 days of the cessation of treatment.

### Embodiment 216

The method of any preceding enumerated embodiment wherein, upon cessation of the treatment, the individual's serum bicarbonate value decreases by at least 4 mEq/l within 5 days of the cessation of treatment.

### Embodiment 217

The method of any preceding enumerated embodiment wherein, upon cessation of the treatment, the individual's serum bicarbonate value decreases by at least 4 mEq/l within 4 days of the cessation of treatment.

### Embodiment 218

The method of any preceding enumerated embodiment wherein, upon cessation of the treatment, the individual's serum bicarbonate value decreases by at least 4 mEq/l within 3 days of the cessation of treatment.

### Embodiment 219

The method of any preceding enumerated embodiment wherein, upon cessation of the treatment, the individual's serum bicarbonate value decreases by at least 4 mEq/l within 2 days of the cessation of treatment.

### Embodiment 220

The method of any preceding enumerated embodiment wherein, upon cessation of the treatment, the individual's serum bicarbonate value decreases by at least 4 mEq/l within 1 day of the cessation of treatment.

### Embodiment 221

The method of any preceding enumerated embodiment wherein, upon cessation of the treatment, the individual's serum bicarbonate value decreases by at least 4.5 mEq/l within 1 month of the cessation of treatment.

### Embodiment 222

The method of any preceding enumerated embodiment wherein, upon cessation of the treatment, the individual's serum bicarbonate value decreases by at least 4.5 mEq/l within 3 weeks of the cessation of treatment.

### Embodiment 223

The method of any preceding enumerated embodiment wherein, upon cessation of the treatment, the individual's serum bicarbonate value decreases by at least 4.5 mEq/l within 2 weeks of the cessation of treatment.

### Embodiment 224

The method of any preceding enumerated embodiment wherein, upon cessation of the treatment, the individual's serum bicarbonate value decreases by at least 4.5 mEq/l within 10 days of the cessation of treatment.

### Embodiment 225

The method of any preceding enumerated embodiment wherein, upon cessation of the treatment, the individual's serum bicarbonate value decreases by at least 4.5 mEq/l within 9 days of the cessation of treatment.

### Embodiment 226

The method of any preceding enumerated embodiment wherein, upon cessation of the treatment, the individual's serum bicarbonate value decreases by at least 4.5 mEq/l within 8 days of the cessation of treatment.

### Embodiment 227

The method of any preceding enumerated embodiment wherein, upon cessation of the treatment, the individual's serum bicarbonate value decreases by at least 4.5 mEq/l within 7 days of the cessation of treatment.

### Embodiment 228

The method of any preceding enumerated embodiment wherein, upon cessation of the treatment, the individual's serum bicarbonate value decreases by at least 4.5 mEq/l within 6 days of the cessation of treatment.

### Embodiment 229

The method of any preceding enumerated embodiment wherein, upon cessation of the treatment, the individual's serum bicarbonate value decreases by at least 4.5 mEq/l within 5 days of the cessation of treatment.

### Embodiment 230

The method of any preceding enumerated embodiment wherein, upon cessation of the treatment, the individual's serum bicarbonate value decreases by at least 4.5 mEq/l within 4 days of the cessation of treatment.

### Embodiment 231

The method of any preceding enumerated embodiment wherein, upon cessation of the treatment, the individual's serum bicarbonate value decreases by at least 4.5 mEq/l within 3 days of the cessation of treatment.

### Embodiment 232

The method of any preceding enumerated embodiment wherein, upon cessation of the treatment, the individual's serum bicarbonate value decreases by at least 4.5 mEq/l within 2 days of the cessation of treatment.

### Embodiment 233

The method of any preceding enumerated embodiment wherein, upon cessation of the treatment, the individual's serum bicarbonate value decreases by at least 4.5 mEq/l within 1 day of the cessation of treatment.

### Embodiment 234

The method of any preceding enumerated embodiment wherein, upon cessation of the treatment, the individual's serum bicarbonate value decreases by at least 5 mEq/l within 1 month of the cessation of treatment.

### Embodiment 235

The method of any preceding enumerated embodiment wherein, upon cessation of the treatment, the individual's serum bicarbonate value decreases by at least 5 mEq/l within 3 weeks of the cessation of treatment.

### Embodiment 236

The method of any preceding enumerated embodiment wherein, upon cessation of the treatment, the individual's serum bicarbonate value decreases by at least 5 mEq/l within 2 weeks of the cessation of treatment.

### Embodiment 237

The method of any preceding enumerated embodiment wherein, upon cessation of the treatment, the individual's serum bicarbonate value decreases by at least 5 mEq/l within 10 days of the cessation of treatment.

### Embodiment 238

The method of any preceding enumerated embodiment wherein, upon cessation of the treatment, the individual's serum bicarbonate value decreases by at least 5 mEq/l within 9 days of the cessation of treatment.

### Embodiment 239

The method of any preceding enumerated embodiment wherein, upon cessation of the treatment, the individual's serum bicarbonate value decreases by at least 5 mEq/l within 8 days of the cessation of treatment.

### Embodiment 240

The method of any preceding enumerated embodiment wherein, upon cessation of the treatment, the individual's serum bicarbonate value decreases by at least 5 mEq/l within 7 days of the cessation of treatment.

### Embodiment 241

The method of any preceding enumerated embodiment wherein, upon cessation of the treatment, the individual's serum bicarbonate value decreases by at least 5 mEq/l within 6 days of the cessation of treatment.

### Embodiment 242

The method of any preceding enumerated embodiment wherein, upon cessation of the treatment, the individual's serum bicarbonate value decreases by at least 5 mEq/l within 5 days of the cessation of treatment.

### Embodiment 243

The method of any preceding enumerated embodiment wherein, upon cessation of the treatment, the individual's serum bicarbonate value decreases by at least 5 mEq/l within 4 days of the cessation of treatment.

### Embodiment 244

The method of any preceding enumerated embodiment wherein, upon cessation of the treatment, the individual's serum bicarbonate value decreases by at least 5 mEq/l within 3 days of the cessation of treatment.

### Embodiment 245

The method of any preceding enumerated embodiment wherein, upon cessation of the treatment, the individual's serum bicarbonate value decreases by at least 5 mEq/l within 2 days of the cessation of treatment.

### Embodiment 246

The method of any preceding enumerated embodiment wherein, upon cessation of the treatment, the individual's serum bicarbonate value decreases by at least 5 mEq/l within 1 day of the cessation of treatment.

### Embodiment 247

The method of any preceding enumerated embodiment wherein the baseline serum bicarbonate value is the value of the serum bicarbonate concentration determined at a single time point.

### Embodiment 248

The method of any of embodiments 1 to 246 wherein the baseline serum bicarbonate value is the mean value of at least two serum bicarbonate concentrations determined at different time-points.

### Embodiment 249

The method of any of embodiments 1 to 246 wherein the baseline serum bicarbonate value is the mean value of at least two serum bicarbonate concentrations for serum samples drawn on different days.

### Embodiment 250

The method of embodiment 249 wherein the baseline serum bicarbonate value is the mean value of at least two serum bicarbonate concentrations for serum samples drawn on consecutive days.

### Embodiment 251

The method of embodiment 249 wherein the baseline serum bicarbonate value is the mean value of at least two serum bicarbonate concentrations for serum samples drawn on two consecutive days and prior to the initiation of the treatment.

### Embodiment 252

The method of embodiment 249 wherein the baseline serum bicarbonate value is the mean or median value of at least two serum bicarbonate concentrations for serum samples drawn on non-consecutive days.

### Embodiment 253

The method of embodiment 252 wherein the non-consecutive days are separated by at least two days.

### Embodiment 254

The method of embodiment 252 wherein the non-consecutive days are separated by at least one week.

### Embodiment 255

The method of embodiment 252 wherein the non-consecutive days are separated by at least two weeks.

### Embodiment 256

The method of embodiment 252 wherein the non-consecutive days are separated by at least three weeks.

### Embodiment 257

The method of any preceding enumerated embodiment wherein the individual is being treated for acute metabolic acidosis.

### Embodiment 258

The method of any preceding enumerated embodiment wherein the individual is being treated for chronic metabolic acidosis.

### Embodiment 259

The method of any preceding enumerated embodiment wherein the daily dose has the capacity to remove at least 7.5 mEq of a target species as it transits the digestive system.

### Embodiment 260

The method of any preceding enumerated embodiment wherein the daily dose has the capacity to remove at least 10 mEq of a target species as it transits the digestive system.

### Embodiment 261

The method of any preceding enumerated embodiment wherein the daily dose has the capacity to remove at least 15 mEq of a target species as it transits the digestive system.

### Embodiment 262

The method of any preceding enumerated embodiment wherein the daily dose has the capacity to remove at least 20 mEq of a target species as it transits the digestive system.

### Embodiment 263

The method of any preceding enumerated embodiment wherein the daily dose has the capacity to remove at least 25 mEq of a target species as it transits the digestive system.

### Embodiment 264

The method of any preceding enumerated embodiment wherein the daily dose has the capacity to remove at least 30 mEq of a target species as it transits the digestive system.

### Embodiment 265

The method of any preceding enumerated embodiment wherein the daily dose has the capacity to remove at least 35 mEq of a target species as it transits the digestive system.

### Embodiment 266

The method of any preceding enumerated embodiment wherein the daily dose has the capacity to remove at least 40 mEq of a target species as it transits the digestive system.

### Embodiment 267

The method of any preceding enumerated embodiment wherein the daily dose has the capacity to remove at least 45 mEq of a target species as it transits the digestive system.

### Embodiment 268

The method of any preceding enumerated embodiment wherein the daily dose has the capacity to remove at least 50 mEq of a target species as it transits the digestive system.

### Embodiment 269

The method of any preceding enumerated embodiment wherein the daily dose has the capacity to remove at least 55 mEq of a target species as it transits the digestive system.

### Embodiment 270

The method of any preceding enumerated embodiment wherein the daily dose has the capacity to remove at least 60 mEq of a target species as it transits the digestive system.

### Embodiment 271

The method of any preceding enumerated embodiment wherein the daily dose has the capacity to remove at least 65 mEq of a target species as it transits the digestive system.

### Embodiment 272

The method of any preceding enumerated embodiment wherein the daily dose has the capacity to remove at least 70 mEq of a target species as it transits the digestive system.

### Embodiment 273

The method of any preceding enumerated embodiment wherein the daily dose has the capacity to remove at least 75 mEq of a target species as it transits the digestive system.

### Embodiment 274

The method of any preceding enumerated embodiment wherein the daily dose has the capacity to remove at least 80 mEq of a target species as it transits the digestive system.

### Embodiment 275

The method of any preceding enumerated embodiment wherein the daily dose has the capacity to remove at least 85 mEq of a target species as it transits the digestive system.

### Embodiment 276

The method of any preceding enumerated embodiment wherein the daily dose has the capacity to remove at least 90 mEq of a target species as it transits the digestive system.

### Embodiment 277

The method of any preceding enumerated embodiment wherein the daily dose has the capacity to remove at least 95 mEq of a target species as it transits the digestive system.

### Embodiment 278

The method of any preceding enumerated embodiment wherein the daily dose has the capacity to remove at least 100 mEq of a target species as it transits the digestive system.

### Embodiment 279

The method of any preceding enumerated embodiment wherein the daily dose has the capacity to remove at least 105 mEq of a target species as it transits the digestive system.

### Embodiment 280

The method of any preceding enumerated embodiment wherein the daily dose has the capacity to remove at least 110 mEq of a target species as it transits the digestive system.

### Embodiment 281

The method of any preceding enumerated embodiment wherein the daily dose is no more than 100 g/day.

### Embodiment 282

The method of any preceding enumerated embodiment wherein the daily dose is no more than 90 g/day.

### Embodiment 283

The method of any preceding enumerated embodiment wherein the daily dose is less than 75 g/day.

### Embodiment 284

The method of any preceding enumerated embodiment wherein the daily dose is less than 65 g/day.

### Embodiment 285

The method of any preceding enumerated embodiment wherein the daily dose is less than 50 g/day.

### Embodiment 286

The method of any preceding enumerated embodiment wherein the daily dose is less than 40 g/day.

### Embodiment 287

The method of any preceding enumerated embodiment wherein the daily dose is less than 30 g/day.

### Embodiment 288

The method of any preceding enumerated embodiment wherein the daily dose is less than 25 g/day.

### Embodiment 289

The method of any preceding enumerated embodiment wherein the daily dose is less than 20 g/day.

### Embodiment 290

The method of any preceding enumerated embodiment wherein the daily dose is less than 15 g/day.

### Embodiment 291

The method of any preceding enumerated embodiment wherein the daily dose is less than 10 g/day.

### Embodiment 292

The method of any preceding enumerated embodiment wherein the daily dose is less than 5 g/day.

### Embodiment 293

The method of any preceding enumerated embodiment wherein the individual is treated for at least one day.

### Embodiment 294

The method of any preceding enumerated embodiment wherein the individual is treated for at least one week.

### Embodiment 295

The method of any preceding enumerated embodiment wherein the individual is treated for at least one month.

### Embodiment 296

The method of any preceding enumerated embodiment wherein the individual is treated for at least several months.

### Embodiment 297

The method of any preceding enumerated embodiment wherein the individual is treated for at least six months.

### Embodiment 298

The method of any preceding enumerated embodiment wherein the individual is treated for at least one year.

### Embodiment 299

The method of any preceding enumerated embodiment wherein the pharmaceutical composition is a nonabsorbable composition comprising a population of particles having a median particle diameter size (volume distribution) of at least 3 microns.

### Embodiment 300

The method of any preceding enumerated embodiment wherein the pharmaceutical composition is a nonabsorbable composition comprising a population of particles having a median particle diameter size (volume distribution) in the range of 5 to 1,000 microns.

### Embodiment 301

The method of any preceding enumerated embodiment wherein the pharmaceutical composition is a nonabsorbable composition comprising a population of particles having a median particle diameter size (volume distribution) in the range of 5 to 500 microns.

### Embodiment 302

The method of any preceding enumerated embodiment wherein the pharmaceutical composition is a nonabsorbable composition comprising a population of particles having a median particle diameter size (volume distribution) in the range of 10 to 400 microns.

### Embodiment 303

The method of any preceding enumerated embodiment wherein the pharmaceutical composition is a nonabsorbable composition comprising a population of particles having a median particle diameter size (volume distribution) in the range of 10 to 300 microns.

### Embodiment 304

The method of any preceding enumerated embodiment wherein the pharmaceutical composition is a nonabsorbable composition comprising a population of particles having a median particle diameter size (volume distribution) in the range of 20 to 250 microns.

### Embodiment 305

The method of any preceding enumerated embodiment wherein the pharmaceutical composition is a nonabsorbable composition comprising a population of particles having a median particle diameter size (volume distribution) in the range of 30 to 250 microns.

### Embodiment 306

The method of any preceding enumerated embodiment wherein the pharmaceutical composition is a nonabsorbable composition comprising a population of particles having a median particle diameter size (volume distribution) in the range of 40 to 180 microns.

### Embodiment 307

The method of any preceding enumerated embodiment wherein the pharmaceutical composition is a nonabsorbable composition comprising a population of particles in which less than 7% of the particles in the population (volume distribution) have a diameter less than 10 microns.

### Embodiment 308

The method of any preceding enumerated embodiment wherein the pharmaceutical composition is a nonabsorbable composition comprising a population of particles in which less than 5% of the particles in the particles in the population (volume distribution) have a diameter less than 10 microns.

### Embodiment 309

The method of any preceding enumerated embodiment wherein the pharmaceutical composition is a nonabsorbable composition comprising a population of particles in which less than 2.5% of the particles in the particles in the population (volume distribution) have a diameter less than 10 microns.

### Embodiment 310

The method of any preceding enumerated embodiment wherein the pharmaceutical composition is a nonabsorbable composition comprising a population of particles in which less than 1% of the particles in the particles in the population (volume distribution) have a diameter less than 10 microns.

### Embodiment 311

The method of any preceding enumerated embodiment wherein the pharmaceutical composition is a nonabsorbable composition comprising a population of particles having a particle size range that is (i) large enough to avoid passive or active absorption through the GI tract and (ii) small enough to not cause grittiness or unpleasant mouth feel when ingested as a powder, suspension, gel, and/or tablet.

### Embodiment 312

The method of any preceding enumerated embodiment wherein the pharmaceutical composition is a nonabsorbable composition comprising a population of particles have a Swelling Ratio of less than 9.

### Embodiment 313

The method of any preceding enumerated embodiment wherein the pharmaceutical composition is a nonabsorbable composition comprising a population of particles have a Swelling Ratio of less than 8.

### Embodiment 314

The method of any preceding enumerated embodiment wherein the pharmaceutical composition is a nonabsorbable composition comprising a population of particles have a Swelling Ratio of less than 7.

### Embodiment 315

The method of any preceding enumerated embodiment wherein the pharmaceutical composition is a nonabsorbable composition comprising a population of particles have a Swelling Ratio of less than 6.

### Embodiment 316

The method of any preceding enumerated embodiment wherein the pharmaceutical composition is a nonabsorbable composition comprising a population of particles have a Swelling Ratio of less than 5.

### Embodiment 317

The method of any preceding enumerated embodiment wherein the pharmaceutical composition is a nonabsorbable composition comprising a population of particles have a Swelling Ratio of less than 4.

### Embodiment 318

The method of any preceding enumerated embodiment wherein the pharmaceutical composition is a nonabsorbable composition comprising a population of particles have a Swelling Ratio of less than 3.

### Embodiment 319

The method of any preceding enumerated embodiment wherein the pharmaceutical composition is a nonabsorbable composition comprising a population of particles have a Swelling Ratio of less than 2.

### Embodiment 320

The method of any preceding enumerated embodiment wherein the nonabsorbable composition has a theoretical binding capacity for the target species of at least about 0.5 mEq/g.

### Embodiment 321

The method of any preceding enumerated embodiment wherein the nonabsorbable composition has a theoretical binding capacity for the target species of at least about 1 mEq/g.

### Embodiment 322

The method of any preceding enumerated embodiment wherein the nonabsorbable composition has a theoretical binding capacity for the target species of at least about 2 mEq/g.

### Embodiment 323

The method of any preceding enumerated embodiment wherein the nonabsorbable composition has a theoretical binding capacity for the target species of at least about 3 mEq/g.

### Embodiment 324

The method of any preceding enumerated embodiment wherein the nonabsorbable composition has a theoretical binding capacity for the target species of at least about 4 mEq/g.

### Embodiment 325

The method of any preceding enumerated embodiment wherein the nonabsorbable composition has a theoretical binding capacity for the target species of at least about 5 mEq/g.

### Embodiment 326

The method of any preceding enumerated embodiment wherein the nonabsorbable composition has a theoretical binding capacity for the target species of at least about 7.5 mEq/g.

### Embodiment 327

The method of any preceding enumerated embodiment wherein the nonabsorbable composition has a theoretical binding capacity for the target species of at least about 10 mEq/g.

### Embodiment 328

The method of any preceding enumerated embodiment wherein the nonabsorbable composition has a theoretical binding capacity for the target species of at least about 12.5 mEq/g.

### Embodiment 329

The method of any preceding enumerated embodiment wherein the nonabsorbable composition has a theoretical binding capacity for the target species of at least about 15 mEq/g.

### Embodiment 330

The method of any preceding enumerated embodiment wherein the nonabsorbable composition has a theoretical binding capacity for the target species of at least about 20 mEq/g.

### Embodiment 331

The method of any preceding enumerated embodiment wherein the nonabsorbable composition has a theoretical binding capacity for the target species of at least about 25 mEq/g.

### Embodiment 332

The method of any preceding enumerated embodiment wherein the nonabsorbable composition has a theoretical binding capacity for the target species of at least about 30 mEq/g.

### Embodiment 333

The method of any preceding enumerated embodiment wherein the nonabsorbable composition has a theoretical binding capacity for the target species of at least about 35 mEq/g.

### Embodiment 334

The method of any preceding enumerated embodiment wherein the nonabsorbable composition has a theoretical binding capacity for the target species in the range of 2 to 25 mEq/g.

### Embodiment 335

The method of any preceding enumerated embodiment wherein the nonabsorbable composition has a theoretical binding capacity for the target species in the range of 3 to 25 mEq/g.

### Embodiment 336

The method of any preceding enumerated embodiment wherein the nonabsorbable composition has a theoretical binding capacity for the target species in the range of 5 to 25 mEq/g.

### Embodiment 337

The method of any preceding enumerated embodiment wherein the nonabsorbable composition has a theoretical binding capacity for the target species in the range of 10 to 25 mEq/g.

### Embodiment 338

The method of any preceding enumerated embodiment wherein the nonabsorbable composition has a theoretical binding capacity for the target species in the range of 5 to 20 mEq/g.

### Embodiment 339

The method of any preceding enumerated embodiment wherein the nonabsorbable composition has a theoretical binding capacity for the target species in the range of 6 to 20 mEq/g.

### Embodiment 340

The method of any preceding enumerated embodiment wherein the nonabsorbable composition has a theoretical binding capacity for the target species in the range of 7.5 to 20 mEq/g.

### Embodiment 341

The method of any preceding enumerated embodiment wherein the nonabsorbable composition has a theoretical binding capacity for the target species in the range of 10 to 20 mEq/g.

### Embodiment 342

The method of any preceding enumerated embodiment wherein the theoretical binding capacity for the target species is the theoretical binding capacity as determined in a SGF assay.

### Embodiment 343

The method of any preceding enumerated embodiment wherein the target species comprises protons.

### Embodiment 344

The method of any preceding enumerated embodiment wherein the target species comprises the conjugate base of a strong acid.

### Embodiment 345

The method of any preceding enumerated embodiment wherein the target species comprises the conjugate base of a strong acid selected from the group consisting of chloride, bisulfate and sulfate ions.

### Embodiment 346

The method of any preceding enumerated embodiment wherein the target species comprises chloride ions.

### Embodiment 347

The method of any preceding enumerated embodiment wherein the target species comprises a strong acid.

### Embodiment 348

The method of any preceding enumerated embodiment wherein the target species comprises hydrochloric acid.

### Embodiment 349

The method of any preceding enumerated embodiment wherein the nonabsorbable composition is characterized by a chloride ion binding capacity of at least 1 mEq/g in a SIB assay.

### Embodiment 350

The method of any preceding enumerated embodiment wherein the nonabsorbable composition is characterized by a chloride ion binding capacity of at least 1.5 mEq/g in a SIB assay.

### Embodiment 351

The method of any preceding enumerated embodiment wherein the nonabsorbable composition is characterized by a chloride ion binding capacity of at least 2 mEq/g in a SIB assay.

### Embodiment 352

The method of any preceding enumerated embodiment wherein the nonabsorbable composition is characterized by a chloride ion binding capacity of at least 2.5 mEq/g in a SIB assay.

### Embodiment 353

The method of any preceding enumerated embodiment wherein the nonabsorbable composition is characterized by a chloride ion binding capacity of at least 3 mEq/g in a SIB assay.

### Embodiment 354

The method of any preceding enumerated embodiment wherein the nonabsorbable composition is characterized by a chloride ion binding capacity of at least 3.5 mEq/g in a SIB assay.

### Embodiment 355

The method of any preceding enumerated embodiment wherein the nonabsorbable composition is characterized by a chloride ion binding capacity of at least 4 mEq/g in a SIB assay.

### Embodiment 356

The method of any preceding enumerated embodiment wherein the nonabsorbable composition is characterized by a chloride ion binding capacity of at least 4.5 mEq/g in a SIB assay.

### Embodiment 357

The method of any preceding enumerated embodiment wherein the nonabsorbable composition is characterized by a chloride ion binding capacity of at least 5 mEq/g in a SIB assay.

### Embodiment 358

The method of any preceding enumerated embodiment wherein the nonabsorbable composition is characterized by a chloride ion binding capacity of at least 5.5 mEq/g in a SIB assay.

### Embodiment 359

The method of any preceding enumerated embodiment wherein the nonabsorbable composition is characterized by a chloride ion binding capacity of at least 6 mEq/g in a SIB assay.

### Embodiment 360

The method of any preceding enumerated embodiment wherein the ratio of the amount of bound chloride to bound phosphate in a SIB assay is at least 0.1:1, respectively.

### Embodiment 361

The method of any preceding enumerated embodiment wherein the ratio of the amount of bound chloride to bound phosphate in a SIB assay is at least 0.2:1, respectively.

### Embodiment 362

The method of any preceding enumerated embodiment wherein the ratio of the amount of bound chloride to bound phosphate in a SIB assay is at least 0.25:1, respectively.

### Embodiment 363

The method of any preceding enumerated embodiment wherein the ratio of the amount of bound chloride to bound phosphate in a SIB assay is at least 0.3:1, respectively.

### Embodiment 364

The method of any preceding enumerated embodiment wherein the ratio of the amount of bound chloride to bound phosphate in a SIB assay is at least 0.35:1, respectively.

### Embodiment 365

The method of any preceding enumerated embodiment wherein the ratio of the amount of bound chloride to bound phosphate in a SIB assay is at least 0.4:1, respectively.

### Embodiment 366

The method of any preceding enumerated embodiment wherein the ratio of the amount of bound chloride to bound phosphate in a SIB assay is at least 0.45:1, respectively.

### Embodiment 367

The method of any preceding enumerated embodiment wherein the ratio of the amount of bound chloride to bound phosphate in a SIB assay is at least 0.5:1, respectively.

### Embodiment 368

The method of any preceding enumerated embodiment wherein the ratio of the amount of bound chloride to bound phosphate in a SIB assay is at least 2:3, respectively.

### Embodiment 369

The method of any preceding enumerated embodiment wherein the ratio of the amount of bound chloride to bound phosphate in a SIB assay is at least 0.75:1, respectively.

### Embodiment 370

The method of any preceding enumerated embodiment wherein the ratio of the amount of bound chloride to bound phosphate in a SIB assay is at least 0.9:1, respectively.

### Embodiment 371

The method of any preceding enumerated embodiment wherein the ratio of the amount of bound chloride to bound phosphate in a SIB assay is at least 1:1, respectively.

### Embodiment 372

The method of any preceding enumerated embodiment wherein the ratio of the amount of bound chloride to bound phosphate in a SIB assay is at least 1.25:1, respectively.

### Embodiment 373

The method of any preceding enumerated embodiment wherein the ratio of the amount of bound chloride to bound phosphate in a SIB assay is at least 1.5:1, respectively.

### Embodiment 374

The method of any preceding enumerated embodiment wherein the ratio of the amount of bound chloride to bound phosphate in a SIB assay is at least 1.75:1, respectively.

### Embodiment 375

The method of any preceding enumerated embodiment wherein the ratio of the amount of bound chloride to bound phosphate in a SIB assay is at least 2:1, respectively.

### Embodiment 376

The method of any preceding enumerated embodiment wherein the ratio of the amount of bound chloride to bound phosphate in a SIB assay is at least 2.25:1, respectively.

### Embodiment 377

The method of any preceding enumerated embodiment wherein the ratio of the amount of bound chloride to bound phosphate in a SIB assay is at least 2.5:1, respectively.

### Embodiment 378

The method of any preceding enumerated embodiment wherein the ratio of the amount of bound chloride to bound phosphate in a SIB assay is at least 2.75:1, respectively.

### Embodiment 379

The method of any preceding enumerated embodiment wherein the ratio of the amount of bound chloride to bound phosphate in a SIB assay is at least 3:1, respectively.

### Embodiment 380

The method of any preceding enumerated embodiment wherein the ratio of the amount of bound chloride to bound phosphate in a SIB assay is at least 4:1, respectively.

### Embodiment 381

The method of any preceding enumerated embodiment wherein the ratio of the amount of bound chloride to bound phosphate in a SIB assay is at least 5:1, respectively.

### Embodiment 382

The method of any preceding enumerated embodiment wherein the ratio of the amount of bound chloride to bound phosphate in a SIB assay is at least 6:1, respectively.

### Embodiment 383

The method of any preceding enumerated embodiment wherein the ratio of the amount of bound chloride toc bound phosphate in a SIB assay is at least 7:1, respectively.

### Embodiment 384

The method of any preceding enumerated embodiment wherein the ratio of the amount of bound chloride to bound phosphate in a SIB assay is at least 8:1, respectively.

### Embodiment 385

The method of any preceding enumerated embodiment wherein the ratio of the amount of bound chloride to bound phosphate in a SIB assay is at least 9:1, respectively.

### Embodiment 386

The method of any preceding enumerated embodiment wherein the ratio of the amount of bound chloride to bound phosphate in a SIB assay is at least 10:1, respectively.

### Embodiment 387

The method of any preceding enumerated embodiment wherein the ratio of the amount of bound chloride to bound phosphate in a SIB assay is at least 12.5:1, respectively.

### Embodiment 388

The method of any preceding enumerated embodiment wherein the ratio of the amount of bound chloride to bound phosphate in a SIB assay is at least 15:1, respectively.

### Embodiment 389

The method of any preceding enumerated embodiment wherein the daily dose has the capacity to remove at least about 5 mEq/day of the target species.

### Embodiment 390

The method of any preceding enumerated embodiment wherein the daily dose has the capacity to remove at least about 6 mEq/day of the target species.

### Embodiment 391

The method of any preceding enumerated embodiment wherein the daily dose has the capacity to remove at least about 7 mEq/day of the target species.

### Embodiment 392

The method of any preceding enumerated embodiment wherein the daily dose has the capacity to remove at least about 8 mEq/day of the target species.

### Embodiment 393

The method of any preceding enumerated embodiment wherein the daily dose has the capacity to remove at least about 9 mEq/day of the target species.

### Embodiment 394

The method of any preceding enumerated embodiment wherein the daily dose has the capacity to remove at least about 10 mEq/day of the target species.

### Embodiment 395

The method of any preceding enumerated embodiment wherein the daily dose has the capacity to remove at least about 11 mEq/day of the target species.

### Embodiment 396

The method of any preceding enumerated embodiment wherein the daily dose has the capacity to remove at least about 12 mEq/day of the target species.

### Embodiment 397

The method of any preceding enumerated embodiment wherein the daily dose has the capacity to remove at least about 13 mEq/day of the target species.

### Embodiment 398

The method of any preceding enumerated embodiment wherein the daily dose has the capacity to remove at least about 14 mEq/day of the target species.

### Embodiment 399

The method of any preceding enumerated embodiment wherein the daily dose has the capacity to remove at least about 15 mEq/day of the target species.

### Embodiment 400

The method of any preceding enumerated embodiment wherein the daily dose has the capacity to remove at least about 16 mEq/day of the target species.

### Embodiment 401

The method of any preceding enumerated embodiment wherein the daily dose has the capacity to remove at least about 17 mEq/day of the target species.

### Embodiment 402

The method of any preceding enumerated embodiment wherein the daily dose has the capacity to remove at least about 18 mEq/day of the target species.

### Embodiment 403

The method of any preceding enumerated embodiment wherein the daily dose has the capacity to remove at least about 19 mEq/day of the target species.

### Embodiment 404

The method of any preceding enumerated embodiment wherein the daily dose has the capacity to remove at least about 20 mEq/day of the target species.

### Embodiment 405

The method of any preceding enumerated embodiment wherein the daily dose has the capacity to remove at least about 21 mEq/day of the target species.

### Embodiment 406

The method of any preceding enumerated embodiment wherein the daily dose has the capacity to remove at least about 22 mEq/day of the target species.

### Embodiment 407

The method of any preceding enumerated embodiment wherein the daily dose has the capacity to remove at least about 23 mEq/day of the target species.

### Embodiment 408

The method of any preceding enumerated embodiment wherein the daily dose has the capacity to remove at least about 24 mEq/day of the target species.

### Embodiment 409

The method of any preceding enumerated embodiment wherein the daily dose has the capacity to remove at least about 25 mEq/day of the target species.

### Embodiment 410

The method of any preceding enumerated embodiment wherein the daily dose has the capacity to remove at least about 26 mEq/day of the target species.

### Embodiment 411

The method of any preceding enumerated embodiment wherein the daily dose has the capacity to remove at least about 27 mEq/day of the target species.

### Embodiment 412

The method of any preceding enumerated embodiment wherein the daily dose has the capacity to remove at least about 28 mEq/day of the target species.

### Embodiment 413

The method of any preceding enumerated embodiment wherein the daily dose has the capacity to remove at least about 29 mEq/day of the target species.

### Embodiment 414

The method of any preceding enumerated embodiment wherein the daily dose has the capacity to remove at least about 30 mEq/day of the target species.

### Embodiment 415

The method of any preceding enumerated embodiment wherein the daily dose has the capacity to remove at least about 31 mEq/day of the target species.

### Embodiment 416

The method of any preceding enumerated embodiment wherein the daily dose has the capacity to remove at least about 32 mEq/day of the target species.

### Embodiment 417

The method of any preceding enumerated embodiment wherein the daily dose has the capacity to remove at least about 33 mEq/day of the target species.

### Embodiment 418

The method of any preceding enumerated embodiment wherein the daily dose has the capacity to remove at least about 34 mEq/day of the target species.

### Embodiment 419

The method of any preceding enumerated embodiment wherein the daily dose has the capacity to remove at least about 35 mEq/day of the target species.

### Embodiment 420

The method of any preceding enumerated embodiment wherein the daily dose has the capacity to remove at least about 36 mEq/day of the target species.

### Embodiment 421

The method of any preceding enumerated embodiment wherein the daily dose has the capacity to remove at least about 37 mEq/day of the target species.

### Embodiment 422

The method of any preceding enumerated embodiment wherein the daily dose has the capacity to remove at least about 38 mEq/day of the target species.

### Embodiment 423

The method of any preceding enumerated embodiment wherein the daily dose has the capacity to remove at least about 39 mEq/day of the target species.

### Embodiment 424

The method of any preceding enumerated embodiment wherein the daily dose has the capacity to remove at least about 40 mEq/day of the target species.

### Embodiment 425

The method of any preceding enumerated embodiment wherein the daily dose has the capacity to remove at least about 41 mEq/day of the target species.

### Embodiment 426

The method of any preceding enumerated embodiment wherein the daily dose has the capacity to remove at least about 42 mEq/day of the target species.

### Embodiment 427

The method of any preceding enumerated embodiment wherein the daily dose has the capacity to remove at least about 43 mEq/day of the target species.

### Embodiment 428

The method of any preceding enumerated embodiment wherein the daily dose has the capacity to remove at least about 44 mEq/day of the target species.

### Embodiment 429

The method of any preceding enumerated embodiment wherein the daily dose has the capacity to remove at least about 45 mEq/day of the target species.

### Embodiment 430

The method of any preceding enumerated embodiment wherein the daily dose has the capacity to remove at least about 46 mEq/day of the target species.

### Embodiment 431

The method of any preceding enumerated embodiment wherein the daily dose has the capacity to remove at least about 47 mEq/day of the target species.

### Embodiment 432

The method of any preceding enumerated embodiment wherein the daily dose has the capacity to remove at least about 48 mEq/day of the target species.

### Embodiment 433

The method of any preceding enumerated embodiment wherein the daily dose has the capacity to remove at least about 49 mEq/day of the target species.

### Embodiment 434

The method of any preceding enumerated embodiment wherein the daily dose has the capacity to remove at least about 50 mEq/day of the target species.

### Embodiment 435

The method of any preceding enumerated embodiment wherein the daily dose removes less than 60 mEq/day of the target species.

### Embodiment 436

The method of any preceding enumerated embodiment wherein the daily dose removes less than 55 mEq/day of the target species.

### Embodiment 437

The method of any of embodiments 1 to 433 wherein the daily dose removes less than 50 mEq/day of the target species.

### Embodiment 438

The method of any of embodiments 1 to 428 wherein the daily dose removes less than 45 mEq/day of the target species.

### Embodiment 439

The method of any of embodiments 1 to 423 wherein the daily dose removes less than 40 mEq/day of the target species.

### Embodiment 440

The method of any of embodiments 1 to 418 wherein the daily dose removes less than 35 mEq/day of the target species.

### Embodiment 441

The method of any of embodiments 1 to 417 wherein the daily dose removes less than 34 mEq/day of the target species.

### Embodiment 442

The method of any of embodiments 1 to 416 wherein the daily dose removes less than 33 mEq/day of the target species.

### Embodiment 443

The method of any of embodiments 1 to 415 wherein the daily dose removes less than 32 mEq/day of the target species.

### Embodiment 444

The method of any of embodiments 1 to 414 wherein the daily dose removes less than 31 mEq/day of the target species.

### Embodiment 445

The method of any of embodiments 1 to 413 wherein the daily dose removes less than 30 mEq/day of the target species.

### Embodiment 446

The method of any of embodiments 1 to 412 wherein the daily dose removes less than 29 mEq/day of the target species.

### Embodiment 447

The method of any of embodiments 1 to 411 wherein the daily dose removes less than 28 mEq/day of the target species.

### Embodiment 448

The method of any of embodiments 1 to 410 wherein the daily dose removes less than 27 mEq/day of the target species.

### Embodiment 449

The method of any of embodiments 1 to 409 wherein the daily dose removes less than 26 mEq/day of the target species.

### Embodiment 450

The method of any of embodiments 1 to 408 wherein the daily dose removes less than 25 mEq/day of the target species.

### Embodiment 451

The method of any of embodiments 1 to 407 wherein the daily dose removes less than 24 mEq/day of the target species.

### Embodiment 452

The method of any of embodiments 1 to 406 wherein the daily dose removes less than 23 mEq/day of the target species.

### Embodiment 453

The method of any of embodiments 1 to 405 wherein the daily dose removes less than 22 mEq/day of the target species.

### Embodiment 454

The method of any of embodiments 1 to 404 wherein the daily dose removes less than 21 mEq/day of the target species.

### Embodiment 455

The method of any of embodiments 1 to 403 wherein the daily dose removes less than 20 mEq/day of the target species.

### Embodiment 456

The method of any of embodiments 1 to 402 wherein the daily dose removes less than 19 mEq/day of the target species.

### Embodiment 457

The method of any of embodiments 1 to 401 wherein the daily dose removes less than 18 mEq/day of the target species.

### Embodiment 458

The method of any of embodiments 1 to 400 wherein the daily dose removes less than 17 mEq/day of the target species.

### Embodiment 459

The method of any of embodiments 1 to 399 wherein the daily dose removes less than 16 mEq/day of the target species.

### Embodiment 460

The method of any of embodiments 1 to 398 wherein the daily dose removes less than 15 mEq/day of the target species.

### Embodiment 461

The method of any of embodiments 1 to 397 wherein the daily dose removes less than 14 mEq/day of the target species.

### Embodiment 462

The method of any of embodiments 1 to 396 wherein the daily dose removes less than 13 mEq/day of the target species.

### Embodiment 463

The method of any of embodiments 1 to 395 wherein the daily dose removes less than 12 mEq/day of the target species.

### Embodiment 464

The method of any of embodiments 1 to 394 wherein the daily dose removes less than 11 mEq/day of the target species.

### Embodiment 465

The method of any of embodiments 1 to 393 wherein the daily dose removes less than 10 mEq/day of the target species.

### Embodiment 466

The method of any of embodiments 1 to 392 wherein the daily dose removes less than 9 mEq/day of the target species.

### Embodiment 467

The method of any of embodiments 1 to 391 wherein the daily dose removes less than 8 mEq/day of the target species.

### Embodiment 468

The method of any of embodiments 1 to 390 wherein the daily dose removes less than 7 mEq/day of the target species.

### Embodiment 469

The method of any of embodiments 1 to 389 wherein the daily dose removes less than 6 mEq/day of the target species.

### Embodiment 470

The method of any preceding enumerated embodiment wherein the nonabsorbable composition is a cation exchange material comprising an insoluble (in the gastric environment) support structure and exchangeable cations.

### Embodiment 471

The method of any preceding enumerated embodiment wherein the nonabsorbable composition is a cation exchange material comprising an insoluble (in the gastric environment) support structure and exchangeable cations wherein the cation exchange material is organic, inorganic or a composite thereof.

### Embodiment 472

The method of any preceding enumerated embodiment wherein the nonabsorbable composition is a cation exchange material comprising exchangeable cations selected from the group consisting of lithium, sodium, potassium, calcium, magnesium, iron and combinations thereof.

### Embodiment 473

The method of any preceding enumerated embodiment wherein the nonabsorbable composition is a cation exchange material comprising exchangeable cations selected from the group consisting of sodium, potassium, calcium, magnesium, and combinations thereof.

### Embodiment 474

The method of any preceding enumerated embodiment wherein the nonabsorbable composition is a cation exchange material comprising exchangeable cations selected from the group consisting of sodium, potassium, and combinations thereof.

### Embodiment 475

The method of any preceding enumerated embodiment wherein the nonabsorbable composition is a cation exchange material comprising a combination of exchangeable cations that establish or maintain electrolyte homeostasis.

### Embodiment 476

The method of any preceding enumerated embodiment wherein the nonabsorbable composition is a cation exchange material optionally containing exchangeable sodium ions provided, however, that the amount of the sodium ions in a daily dose is insufficient to increase the patient's serum sodium ion concentration to a value outside the range of 135 to 145 mEq/l.

### Embodiment 477

The method of any preceding enumerated embodiment wherein the nonabsorbable composition is a cation exchange material optionally containing exchangeable potassium ions provided, however, that the amount of the sodium ions in a daily dose is insufficient to increase the patient's serum potassium ion concentration to a value outside the range of 3.7 to 5.2 mEq/L.

### Embodiment 478

The method of any preceding enumerated embodiment wherein the nonabsorbable composition is a cation exchange material optionally containing exchangeable magnesium ions provided, however, that the amount of the magnesium ions in a daily dose is insufficient to increase the patient's serum magnesium ion concentration to a value outside the range of 1.7 to 2.2 mg/dL.

### Embodiment 479

The method of any preceding enumerated embodiment wherein the nonabsorbable composition is a cation exchange material optionally containing exchangeable calcium ions provided, however, that the amount of the calcium ions in a daily dose is insufficient to increase the patient's serum calcium ion concentration to a value outside the range of 8.5 to 10.2 mg/dL.

### Embodiment 480

The method of any preceding enumerated embodiment wherein the nonabsorbable composition is a cation exchange material optionally containing a combination of exchangeable cations selected from the group consisting of sodium, potassium, calcium, magnesium, and combinations thereof, designed to maintain serum Na⁺ levels within the range of 135 to 145 mEq/l, serum K⁺ levels within the range of 3.7 to 5.2 mEq/L, serum Mg²⁺ levels within the range of 1.7 to 2.2 mg/dL and serum Ca²⁺ levels within the range of 8.5 to 10.2 mg/dL.

### Embodiment 481

The method of any preceding enumerated embodiment wherein the nonabsorbable composition is a cation exchange material containing exchangeable sodium ions and the composition contains less than 12% by weight sodium.

### Embodiment 482

The method of any preceding enumerated embodiment wherein the nonabsorbable composition is a cation exchange material containing exchangeable sodium ions and the composition contains less than 9% by weight sodium.

### Embodiment 483

The method of any preceding enumerated embodiment wherein the nonabsorbable composition is a cation exchange material containing exchangeable sodium ions and the composition contains less than 6% by weight sodium.

### Embodiment 484

The method of any preceding enumerated embodiment wherein the nonabsorbable composition is a cation exchange material containing exchangeable sodium ions and the composition contains less than 3% by weight sodium.

### Embodiment 485

The method of any preceding enumerated embodiment wherein the nonabsorbable composition is a cation exchange material containing exchangeable sodium ions and the composition contains less than 1% by weight sodium.

### Embodiment 486

The method of any preceding enumerated embodiment wherein the nonabsorbable composition is a cation exchange material containing exchangeable sodium ions and the composition contains less than 0.1% by weight sodium.

### Embodiment 487

The method of any preceding enumerated embodiment wherein the nonabsorbable composition is a cation exchange material containing exchangeable sodium ions and the composition contains less than 0.01% by weight sodium.

### Embodiment 488

The method of any preceding enumerated embodiment wherein the nonabsorbable composition is a cation exchange material containing exchangeable sodium ions and the composition contains between 0.05 and 3% by weight sodium.

### Embodiment 489

The method of any preceding enumerated embodiment wherein the nonabsorbable composition is a polymeric material having the capacity to bind protons in aqueous solutions.

### Embodiment 490

The method of any preceding enumerated embodiment wherein the nonabsorbable composition is a polymeric material having the capacity to bind protons in aqueous solutions and the nonabsorbable composition is selected from the group consisting of crosslinked polymeric materials containing a polyanion backbone.

### Embodiment 491

The method of any preceding enumerated embodiment wherein the nonabsorbable composition is a polymeric material having the capacity to bind protons in aqueous solutions and the nonabsorbable composition is selected from the group consisting of crosslinked polymeric materials containing a polyanion backbone wherein the polyanion backbone is selected from the group consisting of poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof.

### Embodiment 492

The method of any preceding enumerated embodiment wherein the nonabsorbable composition is a polymeric material having the capacity to bind protons in aqueous solutions, the nonabsorbable composition is selected from the group consisting of crosslinked polymeric materials containing a polyanion backbone, and the polyanion backbone is coordinated to exchangeable monovalent cations, divalent cations, or a combination thereof.

### Embodiment 493

The method of any preceding enumerated embodiment wherein the nonabsorbable composition is a cation exchange resin comprising a polyanion backbone that exchanges cations for protons and has an average pKa of at least 4.

### Embodiment 494

The method of any preceding enumerated embodiment wherein the nonabsorbable composition is a cation exchange resin comprising a polyanion backbone that exchanges cations for protons and has an average pKa of 4-5.

### Embodiment 495

The method of any preceding enumerated embodiment wherein the nonabsorbable composition is a cation exchange resin comprising a polyanion backbone that exchanges cations for protons and has an average pKa of 5-6.

### Embodiment 496

The method of any preceding enumerated embodiment wherein the nonabsorbable composition is a cation exchange resin comprising a polyanion backbone that exchanges cations for protons and has an average pKa of 6-7.

### Embodiment 497

The method of any preceding enumerated embodiment wherein the nonabsorbable composition is a cation exchange resin comprising a polyanion backbone that exchanges cations for protons and has an average pKa of at least 7.

### Embodiment 498

The method of any preceding enumerated embodiment wherein the nonabsorbable composition is a cation exchange resin selected from the group consisting of poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof.

### Embodiment 499

The method of any preceding enumerated embodiment wherein the nonabsorbable composition is a cation exchange resin selected from the group consisting of poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof wherein the polyanion backbone is further functionalized with functional groups to affect the pKa.

### Embodiment 500

The method of any preceding enumerated embodiment wherein the nonabsorbable composition is a cation exchange resin selected from the group consisting of poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof wherein the polyanion backbone is further functionalized with functional groups to affect the pKa, the functional groups being electron withdrawing or electron donating functional groups.

### Embodiment 501

The method of any preceding enumerated embodiment wherein the nonabsorbable composition is a cation exchange resin selected from the group consisting of poly(carboxylic acids), poly(acrylic acids), poly(sulfonic acids), poly(maleic acids), poly(phenols), functionalized polyols and poly(alcohols), poly(hydroxamic acids), poly(imides) and copolymers thereof wherein the polyanion backbone is further functionalized with functional groups to affect the pKa, the functional groups being electron withdrawing or electron donating functional groups selected from the group consisting of flouro, chloro, amino, hydroxyl, alkoxy, phenyl, subphyla, nitroxyl, and cyano.

### Embodiment 502

The method of any preceding enumerated embodiment wherein the pharmaceutical composition is a nonabsorbable composition comprising a ceramic material.

### Embodiment 503

The method of any preceding enumerated embodiment wherein the pharmaceutical composition is a nonabsorbable composition comprising a ceramic material and the ceramic material is microporous or mesoporous.

### Embodiment 504

The method of any preceding enumerated embodiment wherein the pharmaceutical composition is a nonabsorbable composition comprising a ceramic material and the ceramic material is microporous.

### Embodiment 505

The method of any preceding enumerated embodiment wherein the pharmaceutical composition is a nonabsorbable composition comprising a ceramic material and the ceramic material is mesoporous.

### Embodiment 506

The method of any preceding enumerated embodiment wherein the pharmaceutical composition is a nonabsorbable composition comprising a ceramic material and the ceramic material is a cation exchange ceramic composition.

### Embodiment 507

The method of any preceding enumerated embodiment wherein the pharmaceutical composition is a nonabsorbable composition comprising a ceramic material and the ceramic material comprises a molecular sieve.

### Embodiment 508

The method of any preceding enumerated embodiment wherein the pharmaceutical composition is a nonabsorbable composition comprising a ceramic material and the ceramic material comprises a molecular sieve selected from the group consisting of silicas, metalloaluminates, aluminophosphates and gallogerminates.

### Embodiment 509

The method of any preceding enumerated embodiment wherein the pharmaceutical composition is a nonabsorbable composition comprising a ceramic material and the ceramic material comprises a silica molecular sieve.

### Embodiment 510

The method of any preceding enumerated embodiment wherein the pharmaceutical composition is a nonabsorbable composition comprising a ceramic material and the ceramic material comprises a titanoslicate molecular sieve.

### Embodiment 511

The method of any preceding enumerated embodiment wherein the pharmaceutical composition is a nonabsorbable composition comprising a ceramic material and the ceramic material comprises a metallosilicate molecular sieve.

### Embodiment 512

The method of any preceding enumerated embodiment wherein the pharmaceutical composition is a nonabsorbable composition comprising a ceramic material and the ceramic material comprises a zeolite, a borosilicate, a gallosilicate, a ferrisilicate or a chromosilicate molecular sieve.

### Embodiment 513

The method of any preceding enumerated embodiment wherein the pharmaceutical composition is a nonabsorbable composition comprising a ceramic material and the ceramic material comprises a molecular sieve.

### Embodiment 514

The method of any preceding enumerated embodiment wherein the nonabsorbable composition is an anion exchange material comprising an insoluble (in the gastric environment) support structure and exchangeable anions.

### Embodiment 515

The method of any preceding enumerated embodiment wherein the nonabsorbable composition is an anion exchange material comprising an insoluble (in the gastric environment) support structure and exchangeable anions and the anion exchange material is organic, inorganic, or a composite thereof.

### Embodiment 516

The method of any preceding enumerated embodiment wherein the nonabsorbable composition is a strongly basic anion exchange material.

### Embodiment 517

The method of any preceding enumerated embodiment wherein the nonabsorbable composition is a weakly basic anion exchange material.

### Embodiment 518

The method of any preceding enumerated embodiment wherein the nonabsorbable composition is an anion exchange material comprising quaternary amine moieties, phosphonium salts, N-heteroaromatic salts, or combinations thereof.

### Embodiment 519

The method of any preceding enumerated embodiment wherein the nonabsorbable composition is an anion exchange material comprising a poly(ionic liquid), wherein the side chain is selected from the group consisting of salts of tetraalkyl ammonium, imidazolium, pyridinium, pyrrolidonium, guanidinium, piperidinium, and tetraalkyl phosphonium cations and combinations thereof.

### Embodiment 520

The method of any preceding enumerated embodiment wherein the nonabsorbable composition is an anion exchange material having the capacity to induce an increase in the individual's serum bicarbonate value, at least in part, by delivering a physiologically significant amount of hydroxide, carbonate, citrate or other bicarbonate equivalent, or a combination thereof.

### Embodiment 521

The method of any preceding enumerated embodiment wherein the nonabsorbable composition is an anion exchange material comprising at least 1 mEq/g of an anion selected from the group consisting of hydroxide, carbonate, citrate or other bicarbonate equivalent anion, or a combination thereof.

### Embodiment 522

The method of any preceding enumerated embodiment wherein the nonabsorbable composition is an anion exchange material comprising at least 2 mEq/g of an anion selected from the group consisting of hydroxide, carbonate, citrate or other bicarbonate equivalent anion.

### Embodiment 523

The method of any preceding enumerated embodiment wherein the nonabsorbable composition is an anion exchange material comprising at least 5 mEq/g of an anion selected from the group consisting of hydroxide, carbonate, citrate or other bicarbonate equivalent anion.

### Embodiment 524

The method of any preceding enumerated embodiment wherein the nonabsorbable composition is an anion exchange material comprising at least 10 mEq/g of an anion selected from the group consisting of hydroxide, carbonate, citrate or other bicarbonate equivalent anion.

### Embodiment 525

The method of any of embodiments 1 to 523 wherein the nonabsorbable composition is an anion exchange material comprising less than 10 mEq/g of an anion selected from the group consisting of hydroxide, carbonate, citrate or other bicarbonate equivalent anion, or a combination thereof.

### Embodiment 526

The method of any of embodiments 1 to 522 wherein the nonabsorbable composition is an anion exchange material comprising less than 5 mEq/g of an anion selected from the group consisting of hydroxide, carbonate, citrate or other bicarbonate equivalent anion.

### Embodiment 527

The method of any of embodiments 1 to 522 wherein the nonabsorbable composition is an anion exchange material comprising less than 2.5 mEq/g of an anion selected from the group consisting of hydroxide, carbonate, citrate or other bicarbonate equivalent anion.

### Embodiment 528

The method of any of embodiments 1 to 520 wherein the nonabsorbable composition is an anion exchange material comprising less than 1 mEq/g of an anion selected from the group consisting of hydroxide, carbonate, citrate or other bicarbonate equivalent anion.

### Embodiment 529

The method of any of embodiments 1 to 519 wherein the nonabsorbable composition is an anion exchange material comprising less than 0.1 mEq/g of an anion selected from the group consisting of hydroxide, carbonate, citrate or other bicarbonate equivalent anion.

### Embodiment 530

The method of any of embodiments 521 to 529 wherein the bicarbonate equivalent anion is selected from the group consisting of acetate, lactate and the conjugate bases of other short chain carboxylic acids.

### Embodiment 531

The method of any preceding enumerated embodiment wherein the nonabsorbable composition is an amphoteric ion exchange resin.

### Embodiment 532

The method of any preceding enumerated embodiment wherein the nonabsorbable composition is a neutral composition having the capacity to bind both protons and anions.

### Embodiment 533

The method of any preceding enumerated embodiment wherein the nonabsorbable composition is a neutral composition having the capacity to bind both protons and anions selected from the group consisting of polymers functionalized with propylene oxide, polymers functionalized with Michael acceptors, expanded porphyrins, covalent organic frameworks, and polymers containing amine and/or phosphine functional groups.

### Embodiment 534

The method of any preceding enumerated embodiment wherein the nonabsorbable composition (i) removes more chloride ions than bicarbonate equivalent anions (ii) removes more chloride ions than phosphate anions, and (iii) removes more chloride ions than the conjugate bases of bile and fatty acids.

### Embodiment 535

The method of any preceding enumerated embodiment wherein the treatment with the nonabsorbable composition does not have a clinically significant impact upon the serum or colon levels of a metabolically relevant species.

### Embodiment 536

The method of any preceding enumerated embodiment wherein the treatment with the nonabsorbable composition does not have a clinically significant impact upon the serum or colon levels of a metabolically relevant cationic species.

### Embodiment 537

The method of any preceding enumerated embodiment wherein the treatment with the nonabsorbable composition does not have a clinically significant impact upon the serum or colon levels of a metabolically relevant anionic species.

### Embodiment 538

The method of any preceding enumerated embodiment wherein the treatment with the nonabsorbable composition does not have a clinically significant impact upon the serum potassium levels of a statistically significant number of individuals.

### Embodiment 539

The method of any preceding enumerated embodiment wherein the treatment with the nonabsorbable composition does not have a clinically significant impact upon the serum phosphate levels of a statistically significant number of individuals.

### Embodiment 540

The method of any preceding enumerated embodiment wherein the treatment with the nonabsorbable composition does not have a clinically significant impact upon the serum low density lipoprotein (LDL) levels of a statistically significant number of individuals.

### Embodiment 541

The method of any preceding enumerated embodiment wherein the pharmaceutical composition is a nonabsorbable composition comprising a proton-binding, crosslinked amine polymer comprising the residue of an amine corresponding to Formula 1:

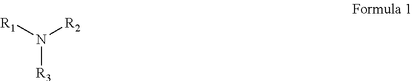

wherein R₁, R₂ and R₃ are independently hydrogen, hydrocarbyl, or substituted hydrocarbyl provided, however, at least one of R₁, R₂ and R₃ is other than hydrogen.

### Embodiment 542

The method of any preceding enumerated embodiment wherein the pharmaceutical composition is a nonabsorbable composition comprising a proton-binding, crosslinked amine polymer comprising the residue of an amine corresponding to Formula 1:

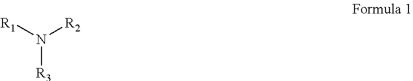

wherein R₁, R₂ and R₃ are independently hydrogen, hydrocarbyl, or substituted hydrocarbyl provided, however, at least one of R₁, R₂ and R₃ is other than hydrogen, and the crosslinked amine polymer has (i) an equilibrium proton binding capacity of at least 5 mmol/g and a chloride ion binding capacity of at least 5 mmol/g in an aqueous simulated gastric fluid buffer (“SGF”) containing 35 mM NaCl and 63 mM HCl at pH 1.2 and 37° C., and (ii) an equilibrium swelling ratio in deionized water of about 2 or less.

### Embodiment 543

The method of any preceding enumerated embodiment wherein the pharmaceutical composition is a nonabsorbable composition comprising the residue of an amine corresponding to Formula 1:

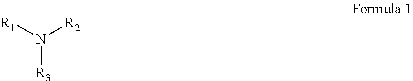

wherein R₁, R₂ and R₃ are independently hydrogen, hydrocarbyl, substituted hydrocarbyl provided, however, at least one of R₁, R₂ and R₃ is other than hydrogen, the crosslinked amine polymer has an equilibrium swelling ratio in deionized water of about 5 or less, and the crosslinked amine polymer binds a molar ratio of chloride ions to interfering ions of at least 0.35:1, respectively, in an interfering ion buffer at 37° C. wherein the interfering ions are phosphate ions and the interfering ion buffer is a buffered solution at pH 5.5 of 36 mM chloride and 20 mM phosphate.

### Embodiment 544

The method of any preceding enumerated embodiment wherein the nonabsorbable composition has an equilibrium chloride binding capacity of at least 7.5 mmol/g in an aqueous simulated gastric fluid buffer (“SGF”) containing 35 mM NaCl and 63 mM HCl at pH 1.2 and 37° C.

### Embodiment 545

The method of any preceding enumerated embodiment wherein the nonabsorbable composition has an equilibrium chloride binding capacity of at least 10 mmol/g in an aqueous simulated gastric fluid buffer (“SGF”) containing 35 mM NaCl and 63 mM HCl at pH 1.2 and 37° C.

### Embodiment 546

The method of any of embodiments 541 to 545 wherein R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ is not hydrogen.

### Embodiment 547

The method of any of embodiments 541 to 545 wherein R₁, R₂ and R₃ are independently hydrogen, aliphatic or heteroaliphatic provided, however, at least one of R₁, R₂ and R₃ is other than hydrogen.

### Embodiment 548

The method of any of embodiments 541 to 547 wherein the crosslinked amine polymer is prepared by substitution polymerization of the amine with a polyfunctional crosslinker, optionally also comprising amine moieties.

### Embodiment 549

The method of any of embodiments 541 to 548 wherein the crosslinked amine polymer comprises the residue of an amine corresponding to Formula 1a and the crosslinked amine polymer is prepared by radical polymerization of an amine corresponding to Formula 1a:

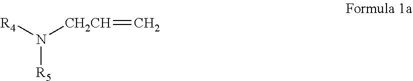

wherein R₄ and R₅ are independently hydrogen, hydrocarbyl, or substituted hydrocarbyl.

### Embodiment 550

The method of embodiment 549 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 551

The method of embodiment 549 wherein R₄ and R₅ are independently hydrogen, aliphatic or heteroaliphatic.

### Embodiment 552

The method of any preceding enumerated embodiment wherein the pharmaceutical composition is a nonabsorbable composition comprising a crosslinked amine polymer containing the residue of an amine corresponding to Formula 1b and the crosslinked amine polymer is prepared by substitution polymerization of the amine corresponding to Formula 1b with a polyfunctional crosslinker:

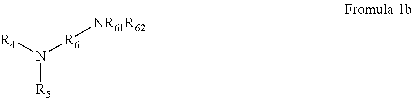

wherein R₄ and R₅ are independently hydrogen, hydrocarbyl, or substituted hydrocarbyl, R₆ is aliphatic and R₆₁ and R₆₂ are independently hydrogen, aliphatic, or heteroaliphatic.

### Embodiment 553

The method of embodiment 552 wherein R₄ and R₅ are independently hydrogen, saturated hydrocarbon, unsaturated aliphatic, aryl, heteroaryl, heteroalkyl, or unsaturated heteroaliphatic.

### Embodiment 554

The method of embodiment 552 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 555

The method of embodiment 552 wherein R₄ and R₅ are independently hydrogen, allyl, or aminoalkyl.

### Embodiment 556

The method of any preceding enumerated embodiment wherein the pharmaceutical composition is in a dosage unit form.

### Embodiment 557

The method of embodiment 556 wherein the dosage unit form is a capsule, tablet or sachet dosage form.

### Embodiment 558

The method of any preceding enumerated embodiment wherein the pharmaceutical composition comprises a pharmaceutically acceptable carrier, excipient, or diluent.

### Embodiment 559

The method of any preceding enumerated embodiment wherein the daily dose is administered once-a-day (QD).

### Embodiment 560

The method of any preceding enumerated embodiment wherein the daily dose is administered twice-a-day (BID).

### Embodiment 561

The method of any preceding enumerated embodiment wherein the daily dose is administered three times a day.

### Embodiment 562

The method of any preceding enumerated embodiments wherein the daily dose is obtained from a pharmaceutical product comprising a sealed container and the nonabsorbable composition within the sealed container.

### Embodiment 563

The method of embodiment 562 wherein the sealed container comprises a moisture barrier.

### Embodiment 564

The method of embodiment 562 or 563 wherein the sealed container comprises an oxygen barrier.

### Embodiment 565

The method of any of embodiments 562 to 564 wherein the sealed container is a sealed sachet.

### Embodiment 566

The method of any of embodiments 562 to 564 wherein the sealed container comprises a multi-layer laminate of an inner contact layer, an outer layer; and a barrier layer disposed between the contact layer and outer layer.

### Embodiment 567

The method of any of embodiments 562 to 564 wherein the sealed container comprises a multi-layer laminate of an inner contact layer, an outer layer; and an oxygen-barrier layer disposed between the contact layer and outer layer.

### Embodiment 568

The method of any of embodiments 562 to 564 wherein the sealed container comprises a multi-layer laminate of an inner contact layer, an outer layer; and a moisture-barrier layer disposed between the contact layer and outer layer.

### Embodiment 569

The method of any of embodiments 562 to 564 wherein the sealed container comprises a multi-layer laminate of an inner contact layer, an outer layer; and an oxygen-barrier layer and a moisture-barrier layer disposed between the contact layer and outer layer.

### Embodiment 570

The method of any of embodiments 562 to 564 wherein the sealed container comprises a multi-layer laminate of an inner contact layer, an outer layer; and an oxygen-scavenging layer disposed between the contact layer and the outer layer.

### Embodiment 571

A composition for use in a method of treating metabolic acidosis in an adult human patient wherein in said treatment 0.1-12 g of said composition is administered to the patient per day, said composition being a nonabsorbable composition having the capacity to remove protons from the patient, wherein the nonabsorbable composition is characterized by a chloride ion binding capacity of at least 2.5 mEq/g in a Simulated Small Intestine Inorganic Buffer (“SIB”) assay.

### Embodiment 572

A composition for use in a method of treating metabolic acidosis in an adult human patient, said patient having a serum bicarbonate level of less than 20 mEq/L prior to treatment, said composition being a nonabsorbable composition having the capacity to remove protons from the patient.

### Embodiment 573

The composition for use according to embodiment 572, wherein the patient's serum bicarbonate level is less than 19 mEq/L prior to treatment.

### Embodiment 574

The composition for use according to embodiment 572, wherein the patient's serum bicarbonate level is less than 18 mEq/L prior to treatment.

### Embodiment 575

The composition for use according to embodiment 572, wherein the patient's serum bicarbonate level is less than 17 mEq/L prior to treatment.

### Embodiment 576

The composition for use according to embodiment 572, wherein the patient's serum bicarbonate level is less than 16 mEq/L prior to treatment.

### Embodiment 577

The composition for use according to embodiment 572, wherein the patient's serum bicarbonate level is less than 15 mEq/L prior to treatment.

### Embodiment 578

The composition for use according to embodiment 572, wherein the patient's serum bicarbonate level is less than 14 mEq/L prior to treatment.

### Embodiment 579

The composition for use according to embodiment 572, wherein the patient's serum bicarbonate level is less than 13 mEq/L prior to treatment.

### Embodiment 580

The composition for use according to embodiment 572, wherein the patient's serum bicarbonate level is less than 12 mEq/L prior to treatment.

### Embodiment 581

The composition for use according to embodiment 572, wherein the patient's serum bicarbonate level is less than 11 mEq/L prior to treatment.

### Embodiment 582

The composition for use according to embodiment 572, wherein the patient's serum bicarbonate level is less than 10 mEq/L prior to treatment.

### Embodiment 583

The composition for use according to embodiment 572 to 582 wherein said patient's serum bicarbonate value is increased by at least 1 mEq/L over 15 days of treatment.

### Embodiment 584

The composition of embodiment 572 to 583 wherein in said treatment 0.1-12 g of said polymer is administered to the patient per day.

### Embodiment 585

The composition of any one of embodiments 572 to 584 wherein the nonabsorbable composition is characterized by a chloride ion binding capacity of at least 2.5 mEq/g in a Simulated Small Intestine Inorganic Buffer (“SIB”) assay.

### Embodiment 586

A composition for use in a method of treating metabolic acidosis in an adult human patient by increasing that patient's serum bicarbonate value by at least 1 mEq/L over 15 days of treatment, said composition being a nonabsorbable composition having the capacity to remove protons from the patient.

### Embodiment 587

The composition of embodiment 571 to 586 wherein in said treatment 0.1-12 g of said polymer is administered to the patient per day.

### Embodiment 588

The composition of any one of embodiments 572 to 587 wherein the nonabsorbable composition is characterized by a chloride ion binding capacity of at least 2.5 mEq/g in a Simulated Small Intestine Inorganic Buffer (“SIB”) assay.

### Embodiment 589

The composition according to any one of embodiments 586 to 588 wherein the patient's serum bicarbonate level value is increased by at least 1 mEq/L over 15 days of treatment.

### Embodiment 590

The composition for use according to any one of embodiments 586 to 589, wherein the increase in serum bicarbonate level is at least 1.5 mEq/L.

### Embodiment 591

The composition for use according to any one of embodiments 586 to 590, wherein the increase in serum bicarbonate level is at least 2 mEq/L.

### Embodiment 592

The composition for use according to any one of embodiments 586 to 591, wherein the increase in serum bicarbonate level is at least 2.5 mEq/L.

### Embodiment 593

The composition for use according to any one of embodiments 586 to 592, wherein the increase in serum bicarbonate level is at least 3 mEq/L.

### Embodiment 594

The composition for use according to any one of embodiments 586 to 593, wherein the increase in serum bicarbonate level is at least 3.5 mEq/L.

### Embodiment 595

The composition for use according to any one of embodiments 586 to 594, wherein the increase in serum bicarbonate level is at least 4 mEq/L.

### Embodiment 596

The composition for use according to any one of embodiments 586 to 595, wherein the increase in serum bicarbonate level is at least 4.5 mEq/L.

### Embodiment 597

The composition for use according to any one of embodiments 586 to 596, wherein the increase in serum bicarbonate level is at least 5 mEq/L.

### Embodiment 598

The composition for use according to embodiment any one of embodiments 586 to 597, wherein the increase is observed during 14 days of treatment.

### Embodiment 599

The composition for use according to embodiment any one of embodiments 586 to 598, wherein the increase is observed during 13 days of treatment.

### Embodiment 600

The composition for use according to embodiment any one of embodiments 586 to 599, wherein the increase is observed during 12 days of treatment.

### Embodiment 601

The composition for use according to embodiment any one of embodiments 586 to 600, wherein the increase is observed during 11 days of treatment.

### Embodiment 602

The composition for use according to embodiment any one of embodiments 586 to 601, wherein the increase is observed during 10 days of treatment.

### Embodiment 603

The composition for use according to embodiment any one of embodiments 586 to 602, wherein the increase is observed during 9 days of treatment.

### Embodiment 604

The composition for use according to embodiment any one of embodiments 586 to 603, wherein the increase is observed during 8 days of treatment.

### Embodiment 605

The composition for use according to embodiment any one of embodiments 586 to 604, wherein the increase is observed during 7 days of treatment.

### Embodiment 606

The composition for use according to embodiment any one of embodiments 586 to 605, wherein the increase is observed during 6 days of treatment.

### Embodiment 607

The composition for use according to embodiment any one of embodiments 586 to 606, wherein the increase is observed during 5 days of treatment.

### Embodiment 608

The composition for use according to embodiment any one of embodiments 586 to 607, wherein the increase is observed during 4 days of treatment.

### Embodiment 609

The composition for use according to embodiment any one of embodiments 586 to 608, wherein the increase is observed during 3 days of treatment.

### Embodiment 610

The composition for use according to embodiment any one of embodiments 586 to 609, wherein the increase is observed during 2 days of treatment.

### Embodiment 611

The composition for use according to embodiment any one of embodiments 586 to 610, wherein the increase is observed during 1 day of treatment.

### Embodiment 612

The composition for use according to any one of embodiments 571 to 611 wherein the specified number of days of treatment are the first days of treatment with the composition.

### Embodiment 613

The composition for use according to embodiment 572-601, wherein in said treatment 0.1-12 g of said polymer is administered to the patient per day.

### Embodiment 614

The composition for use according to embodiment 613, wherein in said treatment 1-11 g of said polymer is administered to the patient per day.

### Embodiment 615

The composition for use according to embodiment 613, wherein in said treatment 2-10 g of said polymer is administered to the patient per day.

### Embodiment 616

The composition for use according to embodiment 613, wherein in said treatment 3-9 g of said polymer is administered to the patient per day.

### Embodiment 617

The composition for use according to embodiment 613, wherein in said treatment 3-8 g of said polymer is administered to the patient per day.

### Embodiment 618

The composition for use according to embodiment 613, wherein in said treatment 3-7 g of said polymer is administered to the patient per day.

### Embodiment 619

The composition for use according to embodiment 613, wherein in said treatment 3-6 g of said polymer is administered to the patient per day.

### Embodiment 620

The composition for use according to embodiment 613, wherein in said treatment 3.5-5.5 g of said polymer is administered to the patient per day.

### Embodiment 621

The composition for use according to embodiment 613, wherein in said treatment 4-5 g of said polymer is administered to the patient per day.

### Embodiment 622

The composition for use according to embodiment 613, wherein in said treatment 1-3 g of said polymer is administered to the patient per day.

### Embodiment 623

The composition for use according to embodiment 571 or 572, wherein about 0.5 g of the composition is administered to the patient per day.

### Embodiment 624

The composition for use according to embodiment 571 or 572, wherein about 1 g of the composition is administered to the patient per day.

### Embodiment 625

The composition for use according to embodiment 571 or 572, wherein about 1.5 g of the composition is administered to the patient per day.

### Embodiment 626

The composition for use according to embodiment 571 or 572, wherein about 2 g of the composition is administered to the patient per day.

### Embodiment 627

The composition for use according to embodiment 571 or 572, wherein about 2.5 g of the composition is administered to the patient per day.

### Embodiment 628

The composition for use according to embodiment 571 or 572, wherein about 3 g of the composition is administered to the patient per day.

### Embodiment 629

The composition for use according to embodiment 571 or 572, wherein about 3.5 g of the composition is administered to the patient per day.

### Embodiment 630

The composition for use according to embodiment 571 or 572, wherein about 4.0 g of the composition is administered to the patient per day.

### Embodiment 631

The composition for use according to embodiment 571 or 572, wherein about 4.5 g of the composition is administered to the patient per day.

### Embodiment 632

The composition for use according to embodiment 571 or 572, wherein about 5.0 g of the composition is administered to the patient per day.

### Embodiment 633

The composition for use according to any one of embodiments 571 to 632, wherein the chloride ion binding capacity in a Simulated Small Intestine Inorganic Buffer (“SIB”) assay is at least 3 mEq/g.

### Embodiment 634

The composition for use according to any one of embodiments 571 to 633, wherein the chloride ion binding capacity in a Simulated Small Intestine Inorganic Buffer (“SIB”) assay is at least 3.5 mEq/g.

### Embodiment 635

The composition for use according to any one of embodiments 571 to 634, wherein the chloride ion binding capacity in a Simulated Small Intestine Inorganic Buffer (“SIB”) assay is at least 4 mEq/g.

### Embodiment 636

The composition for use according to any one of embodiments 571 to 635, wherein the chloride ion binding capacity in a Simulated Small Intestine Inorganic Buffer (“SIB”) assay is at least 4.5 mEq/g.

### Embodiment 637

The composition for use according to any one of embodiments 571 to 636, wherein the chloride ion binding capacity in a Simulated Small Intestine Inorganic Buffer (“SIB”) assay is at least 5 mEq/g.

### Embodiment 638

The composition for use according any one of embodiments 571 to 637, wherein the chloride ion binding capacity in a SIB assay is less than 10 mEq/g.

### Embodiment 639

The composition for use according any one of embodiments 571 to 638, wherein the chloride ion binding capacity in a SIB assay is less than 9 mEq/g.

### Embodiment 640

The composition for use according any one of embodiments 571 to 639, wherein the chloride ion binding capacity in a SIB assay is less than 8 mEq/g.

### Embodiment 641

The composition for use according any one of embodiments 571 to 640, wherein the chloride ion binding capacity in a SIB assay is less than 7 mEq/g.

### Embodiment 642

The composition for use according any one of embodiments 571 to 641, wherein the chloride ion binding capacity in a SIB assay is less than 6 mEq/g.

### Embodiment 643

The composition for use according any one of embodiments 571 to 642, wherein the chloride ion binding capacity in a SIB assay is less than 5 mEq/g.

### Embodiment 644

A composition for use in a method of treating metabolic acidosis in an adult human patient wherein in said treatment>12-100 g of said composition is administered to the patient per day, said composition being a nonabsorbable composition having the capacity to remove protons from the patient, wherein the nonabsorbable composition is characterized by a chloride ion binding capacity of less than 2.5 mEq/g in a Simulated Small Intestine Inorganic Buffer (“SIB”) assay.

### Embodiment 645

The composition according to embodiments 644 wherein the patient's serum bicarbonate value is increased by at least 1 mEq/L over 15 days of treatment.

### Embodiment 646

A composition for use in a method of treating metabolic acidosis in an adult human patient by increasing that patient's serum bicarbonate value by at least 1 mEq/L over 15 days of treatment, wherein in said treatment>12-100 g of said polymer is administered to the patient per day, said composition being a nonabsorbable composition having the capacity to remove protons from the patient, wherein the nonabsorbable composition is characterized by a chloride ion binding capacity of at least 2.5 mEq/g in a Simulated Small Intestine Inorganic Buffer (“SIB”) assay.

### Embodiment 647

The composition for use according to embodiment 645 or 646, wherein the increase in serum bicarbonate level is at least 1 mEq/L.

### Embodiment 648

The composition for use according to embodiment 645 or 646, wherein the increase in serum bicarbonate level is at least 1.5 mEq/L.

### Embodiment 649

The composition for use according to embodiment 645 or 646, wherein the increase in serum bicarbonate level is at least 2 mEq/L.

### Embodiment 650

The composition for use according to embodiment 645 or 646, wherein the increase in serum bicarbonate level is at least 2.5 mEq/L.

### Embodiment 651

The composition for use according to embodiment 645 or 646, wherein the increase in serum bicarbonate level is at least 3 mEq/L.

### Embodiment 652

The composition for use according to embodiment 645 or 646, wherein the increase in serum bicarbonate level is at least 3.5 mEq/L.

### Embodiment 653

The composition for use according to embodiment 645 or 646, wherein the increase in serum bicarbonate level is at least 4 mEq/L.

### Embodiment 654

The composition for use according to embodiment 645 or 646, wherein the increase in serum bicarbonate level is at least 4.5 mEq/L.

### Embodiment 655

The composition for use according to embodiment 645 or 646, wherein the increase in serum bicarbonate level is at least 5 mEq/L.

### Embodiment 656

The composition for use according to embodiment 645 or 646, wherein the increase is observed during 14 days of treatment.

### Embodiment 657

The composition for use according to embodiment 645 or 646, wherein the increase is observed during 13 days of treatment.

### Embodiment 658

The composition for use according to embodiment 645 or 646, wherein the increase is observed during 12 days of treatment.

### Embodiment 659

The composition for use according to embodiment 645 or 646, wherein the increase is observed during 11 days of treatment.

### Embodiment 660

The composition for use according to embodiment 645 or 646, wherein the increase is observed during 10 days of treatment.

### Embodiment 661

The composition for use according to embodiment 645 or 646, wherein the increase is observed during 9 days of treatment.

### Embodiment 662

The composition for use according to embodiment 645 or 646, wherein the increase is observed during 8 days of treatment.

### Embodiment 663

The composition for use according to embodiment 645 or 646, wherein the increase is observed during 7 days of treatment.

### Embodiment 664

The composition for use according to embodiment 645 or 646, wherein the increase is observed during 6 days of treatment.

### Embodiment 665

The composition for use according to embodiment 645 or 646, wherein the increase is observed during 5 days of treatment.

### Embodiment 666

The composition for use according to embodiment 645 or 646, wherein the increase is observed during 4 days of treatment.

### Embodiment 667

The composition for use according to embodiment 645 or 646, wherein the increase is observed during 3 days of treatment.

### Embodiment 668

The composition for use according to embodiment 645 or 646, wherein the increase is observed during 2 days of treatment.

### Embodiment 669

The composition for use according to embodiment 645 or 646, wherein the increase is observed during 1 day of treatment.

### Embodiment 670

The composition for use according to any one of embodiments 644 to 654 wherein the specified number of days of treatment are the first days of treatment with the composition.

### Embodiment 671

A composition for use according to embodiment 644 to 670 wherein 12-100 g is administered to the patient per day.

### Embodiment 672

A composition for use according to embodiment 644 to 671 wherein 20-90 g is administered to the patient per day.

### Embodiment 673

A composition for use according to embodiment 644 to 672 wherein 20-80 g is administered to the patient per day.

### Embodiment 674

A composition for use according to embodiment 644 to 673 wherein 20-70 g is administered to the patient per day.

### Embodiment 675

A composition for use according to embodiment 644 to 674 wherein 20-60 g is administered to the patient per day.

### Embodiment 676

A composition for use according to embodiment 644 to 675 wherein 20-50 g is administered to the patient per day.

### Embodiment 677

A composition for use according to embodiment 644 to 676 wherein 20-40 g is administered to the patient per day.

### Embodiment 678

A composition for use according to embodiment 644 to 677 wherein 20-35 g is administered to the patient per day.

### Embodiment 679

A composition for use according to embodiment 644 to 678 wherein 20-30 g is administered to the patient per day.

### Embodiment 680

A composition for use according to embodiment 644 to 679 wherein 20-25 g is administered to the patient per day.

### Embodiment 681

The composition for use according to any one of embodiments 644 to 680, wherein the chloride ion binding capacity in a Simulated Small Intestine Inorganic Buffer (“SIB”) assay is less than 2 mEq/g.

### Embodiment 682

The composition for use according to any one of embodiments 644 to 681, wherein the chloride ion binding capacity in a Simulated Small Intestine Inorganic Buffer (“SIB”) assay is less than 1.5 mEq/g.

### Embodiment 683

The composition for use according to any one of embodiments 644 to 682, wherein the chloride ion binding capacity in a Simulated Small Intestine Inorganic Buffer (“SIB”) assay is less than 1 mEq/g.

### Embodiment 684

The composition for use according to any one of embodiments 644 to 683, wherein the chloride ion binding capacity in a Simulated Small Intestine Inorganic Buffer (“SIB”) assay is less than 0.75 mEq/g.

### Embodiment 685

The composition for use according to any one of embodiments 644 to 684, wherein the chloride ion binding capacity in a Simulated Small Intestine Inorganic Buffer (“SIB”) assay is greater than 0.5 mEq/g.

### Embodiment 686

The composition for use according to any one of embodiments 644 to 685, wherein the chloride ion binding capacity in a Simulated Small Intestine Inorganic Buffer (“SIB”) assay is greater than 1 mEq/g.

### Embodiment 687

The composition for use according to any one of embodiments 644 to 686, wherein the chloride ion binding capacity in a Simulated Small Intestine Inorganic Buffer (“SIB”) assay is greater than 1.5 mEq/g.

### Embodiment 688

The composition for use according to any one of embodiments 644 to 687, wherein the chloride ion binding capacity in a Simulated Small Intestine Inorganic Buffer (“SIB”) assay is greater than 2 mEq/g.

### Embodiment 689

The composition for use according to any preceding embodiment wherein the composition is administered once per day in order to provide the total specified daily dose.

### Embodiment 690

The composition for use according to any preceding embodiment wherein the composition is administered twice per day in order to provide the total specified daily dose.

### Embodiment 691

The composition for use according to any preceding embodiment wherein the composition is administered three times per day in order to provide the total specified daily dose.

### Embodiment 692

The composition for use according to any preceding enumerated embodiment wherein said composition is administered orally.

### Embodiment 693

The composition for use according to any one of embodiments 571 to 692 wherein the composition is a pharmaceutical composition comprising a proton-binding, crosslinked amine polymer comprising the residue of an amine corresponding to Formula 1:

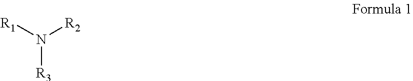

wherein R₁, R₂ and R₃ are independently hydrogen, hydrocarbyl, or substituted hydrocarbyl provided, however, at least one of R₁, R₂ and R₃ is other than hydrogen, and the crosslinked amine polymer has (i) an equilibrium proton binding capacity of at least 5 mmol/g and a chloride ion binding capacity of at least 5 mmol/g in an aqueous simulated gastric fluid buffer (“SGF”) containing 35 mM NaCl and 63 mM HCl at pH 1.2 and 37° C., and (ii) an equilibrium swelling ratio in deionized water of about 2 or less.

### Embodiment 694

The composition for use according to any one of embodiments 571 to 692 wherein the composition is a pharmaceutical composition comprising a proton-binding, crosslinked amine polymer comprising the residue of an amine corresponding to Formula 1:

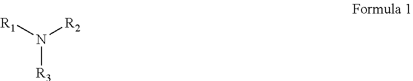

wherein R₁, R₂ and R₃ are independently hydrogen, hydrocarbyl, substituted hydrocarbyl provided, however, at least one of R₁, R₂ and R₃ is other than hydrogen, the crosslinked amine polymer has an equilibrium swelling ratio in deionized water of about 5 or less, and the crosslinked amine polymer binds a molar ratio of chloride ions to interfering ions of at least 0.35:1, respectively, in an interfering ion buffer at 37° C. wherein the interfering ions are phosphate ions and the interfering ion buffer is a buffered solution at pH 5.5 of 36 mM chloride and 20 mM phosphate.

### Embodiment 695

The composition for use according to any one of embodiments 571 to 692 wherein the composition is the pharmaceutical composition of embodiment 682 wherein the crosslinked amine polymer has an equilibrium chloride binding capacity of at least 7.5 mmol/g in an aqueous simulated gastric fluid buffer (“SGF”) containing 35 mM NaCl and 63 mM HCl at pH 1.2 and 37° C.

### Embodiment 696

The composition for use according to any one of embodiments 571 to 692 wherein the composition is the pharmaceutical composition of embodiment 682 wherein the crosslinked amine polymer has an equilibrium chloride binding capacity of at least 10 mmol/g in an aqueous simulated gastric fluid buffer (“SGF”) containing 35 mM NaCl and 63 mM HCl at pH 1.2 and 37° C.

### Embodiment 697

The composition for use according to any one of embodiments 571 to 692 wherein the composition is the pharmaceutical composition of embodiment 683 wherein the crosslinked amine polymer has an equilibrium swelling ratio in deionized water of about 4 or less.

### Embodiment 698

The composition for use according to any one of embodiments 571 to 692 wherein the composition is the pharmaceutical composition of embodiment 683 wherein the crosslinked amine polymer has an equilibrium swelling ratio in deionized water of about 3 or less.

### Embodiment 699

The composition for use according to any one of embodiments 571 to 692 wherein the composition is the pharmaceutical composition of embodiment 683 wherein the crosslinked amine polymer has an equilibrium swelling ratio in deionized water of about 2 or less.

### Embodiment 700

The composition for use according to any one of embodiments 571 to 692 wherein the composition is the pharmaceutical composition of any preceding enumerated embodiment wherein R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ is not hydrogen.

### Embodiment 701

The composition for use according to any one of embodiments 571 to 692 wherein the composition is the pharmaceutical composition of any preceding enumerated embodiment wherein R₁, R₂ and R₃ are independently hydrogen, aliphatic or heteroaliphatic provided, however, at least one of R₁, R₂ and R₃ is other than hydrogen.

### Embodiment 702

The composition for use according to any one of embodiments 571 to 692 wherein the composition is the pharmaceutical composition of any preceding enumerated embodiment wherein the crosslinked amine polymer is prepared by substitution polymerization of the amine with a polyfunctional crosslinker, optionally also comprising amine moieties.

### Embodiment 703

The composition for use according to any one of embodiments 571 to 692 wherein the composition is the pharmaceutical composition of any of embodiments 693 to 701 wherein the crosslinked amine polymer comprises the residue of an amine corresponding to Formula 1a and the crosslinked amine polymer is prepared by radical polymerization of an amine corresponding to Formula 1a:

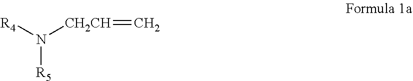

wherein R₄ and R₅ are independently hydrogen, hydrocarbyl, or substituted hydrocarbyl.

### Embodiment 704

The composition for use according to any one of embodiments 571 to 692 wherein the composition is the pharmaceutical composition of embodiment 703 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 705

The composition for use according to any one of embodiments 571 to 692 wherein the composition is the pharmaceutical composition of embodiment 703 wherein R₄ and R₅ are independently hydrogen, aliphatic or heteroaliphatic.

### Embodiment 706

The composition for use according to any one of embodiments 571 to 692 wherein the composition is the pharmaceutical composition of any of embodiments 693 to 701 wherein the crosslinked amine polymer comprises the residue of an amine corresponding to Formula 1 b and the crosslinked amine polymer is prepared by substitution polymerization of the amine corresponding to Formula 1b with a polyfunctional crosslinker:

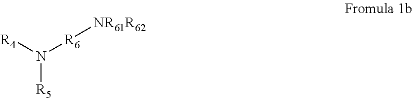

wherein R₄ and R₅ are independently hydrogen, hydrocarbyl, or substituted hydrocarbyl, R₆ is aliphatic and R₆₁ and R₆₂ are independently hydrogen, aliphatic, or heteroaliphatic.

### Embodiment 707

The composition for use according to any one of embodiments 571 to 692 wherein the composition is the pharmaceutical composition of embodiment 706 wherein R₄ and R₅ are independently hydrogen, saturated hydrocarbon, unsaturated aliphatic, aryl, heteroaryl, heteroalkyl, or unsaturated heteroaliphatic.

### Embodiment 708

The composition for use according to any one of embodiments 571 to 692 wherein the composition is the pharmaceutical composition of embodiment 706 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 709

The composition for use according to any one of embodiments 571 to 692 wherein the composition is the pharmaceutical composition of embodiment 706 wherein R₄ and R₅ are independently hydrogen, allyl, or aminoalkyl.

### Embodiment 710

The composition for use according to any one of embodiments 571 to 692 wherein the composition is the pharmaceutical composition of any preceding enumerated embodiment wherein the crosslinked amine polymer comprises the residue of an amine corresponding to Formula 1c:

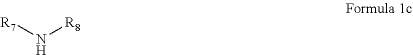

wherein R₇ is hydrogen, aliphatic or heteroaliphatic and R₈ is aliphatic or heteroaliphatic.

### Embodiment 711

The composition for use according to any one of embodiments 571 to 692 wherein the composition is the pharmaceutical composition of any of embodiments 693 to 701 wherein the crosslinked amine polymer comprises the residue of an amine corresponding to Formula 2:

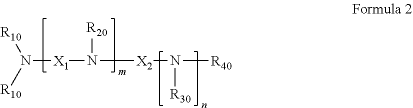

wherein

m and n are independently non-negative integers;

R₁₀, R₂₀, R₃₀, and R₄₀ are independently hydrogen, hydrocarbyl, or substituted hydrocarbyl;

X₁ is

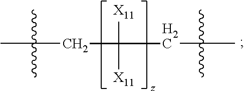

X₂ is hydrocarbyl or substituted hydrocarbyl;

each X₁₁ is independently hydrogen, hydrocarbyl, substituted hydrocarbyl, hydroxy, or amino; and

z is a non-negative number.

### Embodiment 712

The composition for use according to any one of embodiments 571 to 692 wherein the composition is the pharmaceutical composition of embodiment 711 wherein R₁₀, R₂₀, R₃₀, and R₄₀ are independently hydrogen, aliphatic, aryl, heteroaliphatic, or heteroaryl, m and z are independently 0-3 and n is 0 or 1.

### Embodiment 713

The composition for use according to any one of embodiments 571 to 692 wherein the composition is the pharmaceutical composition of embodiment 711 or 712 wherein X₂ is aliphatic or heteroaliphatic.

### Embodiment 714

The composition for use according to any one of embodiments 571 to 692 wherein the composition is the pharmaceutical composition of embodiment 711, 712 or 713 wherein m is 1-3 and X₁₁ is hydrogen, aliphatic or heteroaliphatic.

### Embodiment 715

The composition for use according to any one of embodiments 571 to 692 wherein the composition is the pharmaceutical composition of any of embodiments 693 to 701 wherein the crosslinked amine polymer comprises the residue of an amine corresponding to Formula 2a:

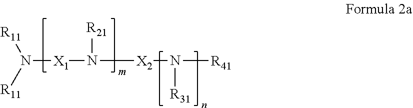

wherein

m and n are independently non-negative integers;

each R₁₁ is independently hydrogen, hydrocarbyl, heteroaliphatic, or heteroaryl;

R₂₁ and R₃₁, are independently hydrogen or heteroaliphatic;

R₄₁ is hydrogen, substituted hydrocarbyl, or hydrocarbyl;

X₁ is

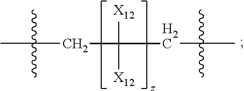

X₂ is alkyl or substituted hydrocarbyl;

each X₁₂ is independently hydrogen, hydroxy, amino, aminoalkyl, boronic acid or halo; and

z is a non-negative number.

### Embodiment 716

The composition for use according to any one of embodiments 571 to 692 wherein the composition is the pharmaceutical composition of embodiment 715 wherein m and z are independently 0-3 and n is 0 or 1.

### Embodiment 717

The composition for use according to any one of embodiments 571 to 692 wherein the composition is the pharmaceutical composition of embodiment 715 or 716 wherein R₁₁ is independently hydrogen, aliphatic, aminoalkyl, haloalkyl, or heteroaryl, R₂₁ and R₃₁ are independently hydrogen or heteroaliphatic and R₄₁ is hydrogen, aliphatic, aryl, heteroaliphatic, or heteroaryl.

### Embodiment 718

The composition for use according to any one of embodiments 571 to 692 wherein the composition is the pharmaceutical composition of embodiment 715 or 716 wherein each R₁₁ is hydrogen, aliphatic, aminoalkyl, or haloalkyl, R₂₁ and R₃₁ are hydrogen or aminoalkyl, and R₄₁ is hydrogen, aliphatic, or heteroaliphatic.

### Embodiment 719

The composition for use according to any one of embodiments 571 to 692 wherein the composition is the pharmaceutical composition of any of embodiments 693 to 701 wherein the crosslinked amine polymer comprises the residue of an amine corresponding to Formula 2b:

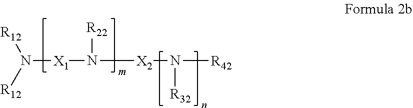

wherein

m and n are independently non-negative integers;

each R₁₂ is independently hydrogen, substituted hydrocarbyl, or hydrocarbyl;

R₂₂ and R₃₂ are independently hydrogen substituted hydrocarbyl, or hydrocarbyl;

R₄₂ is hydrogen, hydrocarbyl or substituted hydrocarbyl;

X₁ is

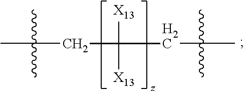

X₂ is alkyl, aminoalkyl, or alkanol;

each X₁₃ is independently hydrogen, hydroxy, alicyclic, amino, aminoalkyl, halogen, alkyl, heteroaryl, boronic acid or aryl;

z is a non-negative number; and

the amine corresponding to Formula 2b comprises at least one allyl group.

### Embodiment 720

The composition for use according to any one of embodiments 571 to 692 wherein the composition is the pharmaceutical composition of embodiment 719 wherein m and z are independently 0-3 and n is 0 or 1.

### Embodiment 721

The composition for use according to any one of embodiments 571 to 692 wherein the composition is the pharmaceutical composition of embodiment 719 or 720 wherein R₁₂ or R₄₂ independently comprise at least one allyl or vinyl moiety.

### Embodiment 722

The composition for use according to any one of embodiments 571 to 692 wherein the composition is the pharmaceutical composition of embodiment 719 or 720 wherein (i) m is a positive integer and R₁₂, R₂₂ and R₄₂, in combination comprise at least two allyl or vinyl moieties or (ii) n is a positive integer and R₁₂, R₃₂ and R₄₂, in combination, comprise at least two allyl or vinyl moieties.

### Embodiment 723

The composition for use according to any one of embodiments 571 to 692 wherein the composition is the pharmaceutical composition of embodiment 719 or 720 wherein the crosslinked amine polymer comprises the residue of an amine appearing in Table A.

### Embodiment 724

The composition for use according to any one of embodiments 571 to 692 wherein the composition is the pharmaceutical composition of embodiment 719, 720 or 723 wherein the crosslinked amine polymer is crosslinked with a crosslinking agent appearing in Table B.

### Embodiment 725

The composition for use according to any one of embodiments 571 to 692 wherein the composition is the pharmaceutical composition of any preceding enumerated embodiment wherein the crosslinked amine polymer comprises a repeat unit corresponding to Formula 3:

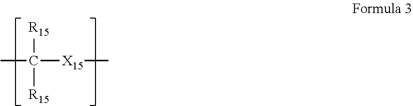

wherein

R₁₅, R₁₆ and R₁₇ are independently hydrogen, hydrocarbyl, substituted hydrocarbyl, hydroxyl, amino, boronic acid or halo;

X₁₅ is

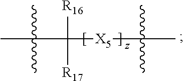

X₅ is hydrocarbyl, substituted hydrocarbyl, oxo (—O—), or amino; and z is a non-negative number.

### Embodiment 726

The composition for use according to any one of embodiments 571 to 692 wherein the composition is the pharmaceutical composition of embodiment 725 wherein R₁₅, R₁₆ and R₁₇ are independently aliphatic or heteroaliphatic.

### Embodiment 727

The composition for use according to any one of embodiments 571 to 692 wherein the composition is the pharmaceutical composition of embodiment 725 or 726 wherein X₅ is oxo, amino, alkylamino, ethereal, alkanol, or haloalkyl.

### Embodiment 728

The composition for use according to any one of embodiments 571 to 692 wherein the composition is the pharmaceutical composition of any of embodiments 693 to 701 wherein the crosslinked amine polymer is prepared by (i) substitution polymerization of polyfunctional reagents at least one of which comprises amine moieties, (2) radical polymerization of a monomer comprising at least one amine moiety or nitrogen containing moiety, or (3) crosslinking of an amine-containing intermediate with a crosslinking agent, optionally containing amine moieties.

### Embodiment 729

The composition for use according to any one of embodiments 571 to 692 wherein the composition is the pharmaceutical composition of embodiment 728 wherein the crosslinked amine polymer is a crosslinked homopolymer or a crosslinked copolymer.

### Embodiment 730

The composition for use according to any one of embodiments 571 to 692 wherein the composition is the pharmaceutical composition of embodiment 728 wherein the crosslinked amine polymer comprises free amine moieties, separated by the same or varying lengths of repeating linker units.

### Embodiment 731

The composition for use according to any one of embodiments 571 to 692 wherein the composition is the pharmaceutical composition of embodiment 728 wherein the crosslinked amine polymer is prepared by polymerizing an amine-containing monomer with a crosslinking agent in a substitution polymerization reaction.

### Embodiment 732

The composition for use according to any one of embodiments 571 to 692 wherein the composition is the pharmaceutical composition of embodiment 731 wherein the amine-containing monomer is a linear amine possessing at least two reactive amine moieties to participate in the substitution polymerization reaction.

### Embodiment 733

The composition for use according to any one of embodiments 571 to 692 wherein the composition is the pharmaceutical composition of embodiment 731 or 732 wherein the amine-containing monomer is 1,3-Bis[bis(2-aminoethyl)amino]propane, 3-Amino-1-{[2-(bis{2-[bis(3-aminopropyl)amino]ethyl}amino)ethyl](3-aminopropyl)amino}propane, 2-[Bis(2-aminoethyl)amino]ethanamine, Tris(3-aminopropyl)amine, 1,4-Bis[bis(3-aminopropyl)amino]butane, 1,2-Ethanediamine, 2-Amino-1-(2-aminoethylamino)ethane, 1,2-Bis(2-aminoethylamino)ethane, 1,3-Propanediamine, 3,3′-Diaminodipropylamine, 2,2-dimethyl-1,3-propanediamine, 2-methyl-1,3-propanediamine, N,N′-dimethyl-1,3-propanediamine, N-methyl-1,3-diaminopropane, 3,3′-diamino-N-methyldipropylamine, 1,3-diaminopentane, 1,2-diamino-2-methylpropane, 2-methyl-1,5-diaminopentane, 1,2-diaminopropane, 1,10-diaminodecane, 1,8-diaminooctane, 1,9-diaminooctane, 1,7-diaminoheptane, 1,6-diaminohexane, 1,5-diaminopentane, 3-bromopropylamine hydrobromide, N,2-dimethyl-1,3-propanediamine, N-isopropyl-1,3-diaminopropane, N,N′-bis(2-aminoethyl)-1,3-propanediamine, N,N′-bis(3-aminopropyl)ethylenediamine, N,N′-bis(3-aminopropyl)-1,4-butanediamine tetrahydrochloride, 1,3-diamino-2-propanol, N-ethylethylenediamine, 2,2′-diamino-N-methyldiethylamine, N,N′-diethylethylenediamine, N-isopropylethylenediamine, N-methylethylenediamine, N,N′-di-tert-butylethylenediamine, N,N′-diisopropylethylenediamine, N,N′-dimethylethylenediamine, N-butylethylenediamine, 2-(2-aminoethylamino)ethanol, 1,4,7,10,13,16-hexaazacyclooctadecane, 1,4,7,10-tetraazacyclododecane, 1,4,7-triazacyclononane, N,N′-bis(2-hydroxyethyl)ethylenediamine, piperazine, bis(hexamethylene)triamine, N-(3-hydroxypropyl)ethylenediamine, N-(2-Aminoethyl)piperazine, 2-Methylpiperazine, Homopiperazine, 1,4,8,11-Tetraazacyclotetradecane, 1,4,8,12-Tetraazacyclopentadecane, 2-(Aminomethyl)piperidine, or 3-(Methylamino)pyrrolidino.

### Embodiment 734

The composition for use according to any one of embodiments 571 to 692 wherein the composition is the pharmaceutical composition of any of embodiments 728, 730, 732, and 733 wherein the crosslinking agent is selected from the group consisting of dihaloalkanes, haloalkyloxiranes, alkyloxirane sulfonates, di(haloalkyl)amines, tri(haloalkyl) amines, diepoxides, triepoxides, tetraepoxides, bis (halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, 1-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy) diphenylmethane, bisphenol F diglycidyl ether, 1,4-bis(2′,3′epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4-h-chromene-2-carboxylate, bis[4-(2,3-epoxy-propylthio)phenyl]-sulfide, 1,3-bis(3-glycidoxypropyl) tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy) propyl]dimethylsilyloxy]-1,3,5,7,9,11,14-heptacyclopentyltricyclo [7,3,3,15, 11]heptasiloxane, 4,4′methylenebis(N, N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate, 3-chloro-1-(3-chloropropylamino-2-propanol, 1,2-bis(3-chloropropylamino)ethane, Bis(3-chloropropyl)amine, 1,3-Dichloro-2-propanol, 1,3-Dichloropropane, 1-chloro-2,3-epoxypropane, tris[(2-oxiranyl)methyl]amine, and combinations thereof.

### Embodiment 735

The composition for use according to any one of embodiments 571 to 692 wherein the composition is the pharmaceutical composition of embodiment 728 wherein the preparation of the crosslinked amine polymer comprises radical polymerization of an amine monomer comprising at least one amine moiety or nitrogen containing moiety.

### Embodiment 736

The composition for use according to any one of embodiments 571 to 692 wherein the composition is the pharmaceutical composition of any preceding enumerated embodiment wherein the crosslinked amine polymer has an equilibrium swelling ratio in deionized water of about 1.5 or less.

### Embodiment 737

The composition for use according to any one of embodiments 571 to 692 wherein the composition is the pharmaceutical composition of any preceding enumerated embodiment wherein the crosslinked amine polymer has an equilibrium swelling ratio in deionized water of about 1 or less.

### Embodiment 738

The composition for use according to any one of embodiments 571 to 692 wherein the composition is the pharmaceutical composition of any preceding enumerated embodiment wherein the crosslinked amine polymer has a chloride ion to phosphate ion binding molar ratio of at least 0.5:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 739

The composition for use according to any one of embodiments 571 to 692 wherein the composition is the pharmaceutical composition of any preceding enumerated embodiment wherein the crosslinked amine polymer has a chloride ion to phosphate ion binding molar ratio of at least 1:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 740

The composition for use according to any one of embodiments 571 to 692 wherein the composition is the pharmaceutical composition of any preceding enumerated embodiment wherein the crosslinked amine polymer has a chloride ion to phosphate ion binding molar ratio of at least 2:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 741

The composition for use according to any one of embodiments 571 to 692 wherein the composition is the pharmaceutical composition of any preceding enumerated embodiment wherein the crosslinked amine polymer has a proton binding capacity of at least 10 mmol/g and a chloride ion binding capacity of at least 10 mmol/g in an aqueous simulated gastric fluid buffer (“SGF”) containing 35 mM NaCl and 63 mM HCl at pH 1.2 and 37° C.

### Embodiment 742

The composition for use according to any one of embodiments 571 to 692 wherein the composition is the pharmaceutical composition of any preceding enumerated embodiment wherein the crosslinked amine polymer has an equilibrium proton binding capacity of at least 12 mmol/g and a chloride ion binding capacity of at least 12 mmol/g in an aqueous simulated gastric fluid buffer (“SGF”) containing 35 mM NaCl and 63 mM HCl at pH 1.2 and 37° C.

### Embodiment 743

The composition for use according to any one of embodiments 571 to 692 wherein the composition is the pharmaceutical composition of any preceding enumerated embodiment wherein the crosslinked amine polymer has an equilibrium proton binding capacity of at least 14 mmol/g and a chloride ion binding capacity of at least 14 mmol/g in an aqueous simulated gastric fluid buffer (“SGF”) containing 35 mM NaCl and 63 mM HCl at pH 1.2 and 37° C.

### Embodiment 744

The composition for use according to any one of embodiments 571 to 692 wherein the composition is the pharmaceutical composition of any preceding enumerated embodiment wherein the percentage of quaternized amines is less than 40%.

### Embodiment 745

The composition for use according to any one of embodiments 571 to 692 wherein the composition is the pharmaceutical composition of any preceding enumerated embodiment wherein the percentage of quaternized amines is less than 30%.

### Embodiment 746

The composition for use according to any one of embodiments 571 to 692 wherein the composition is the pharmaceutical composition of any preceding enumerated embodiment wherein the percentage of quaternized amines is less than 20%.

### Embodiment 747

The composition for use according to any one of embodiments 571 to 692 wherein the composition is the pharmaceutical composition of any preceding enumerated embodiment wherein the percentage of quaternized amines is less than 10%.

### Embodiment 748

The composition for use according to any one of embodiments 571 to 692 wherein the composition is the pharmaceutical composition of any preceding enumerated embodiment wherein the percentage of quaternized amines is less than 5%.

### Embodiment 749

The composition for use according to any one of embodiments 571 to 692 wherein the composition is the pharmaceutical composition of any preceding enumerated embodiment wherein the crosslinked amine polymer is a gel or a bead having a mean particle size of 40 to 180 micrometers.

### Embodiment 750

The composition for use according to any one of embodiments 571 to 692 wherein the composition is the pharmaceutical composition of any preceding enumerated embodiment wherein the crosslinked amine polymer is a gel or a bead having a mean particle size of 60 to 160 micrometers.

### Embodiment 751

The composition for use according to any one of embodiments 571 to 692 wherein the composition is the pharmaceutical composition of any preceding enumerated embodiment wherein the crosslinked amine polymer is a gel or a bead having a mean particle size of 80 to 140 micrometers.

### Embodiment 752

The composition for use according to any one of embodiments 571 to 692 wherein the composition is the pharmaceutical composition of any one of embodiments 749 to 751 wherein less than about 0.5 volume percent of the particles have a diameter of less than about 10 micrometers.

### Embodiment 753

The composition for use according to any one of embodiments 571 to 692 wherein the composition is the pharmaceutical composition of any one of embodiments 749 to 751 wherein less than about 5 volume percent of the particles have a diameter of less than about 20 micrometers.

### Embodiment 754

The composition for use according to any one of embodiments 571 to 692 wherein the composition is the pharmaceutical composition of any one of embodiments 749 to 751 wherein less than about 0.5 volume percent of the particles have a diameter of less than about 20 micrometers.

### Embodiment 755

The composition for use according to any one of embodiments 571 to 692 wherein the composition is the pharmaceutical composition of any one of embodiments 749 to 751 wherein less than about 5 volume percent of the particles have a diameter of less than about 30 micrometers.

### Embodiment 756

The composition for use according to any one of embodiments 571 to 692 wherein the composition is the pharmaceutical composition of any preceding enumerated embodiment in a dosage unit form.

### Embodiment 757

The composition for use according to any one of embodiments 571 to 692 wherein the composition is the pharmaceutical composition of embodiment 756 wherein the dosage unit form is a capsule, tablet or sachet dosage form.

### Embodiment 758

The composition for use according to any one of embodiments 571 to 692 wherein the composition is the pharmaceutical composition of any preceding enumerated embodiment wherein the pharmaceutical composition comprises a pharmaceutically acceptable carrier, excipient, or diluent.

### Embodiment 759

The composition for use according to any one of embodiments 571 to 692 wherein the composition is a method of treating and acid/base disorder in an animal including a human by removing HCl through oral administration of a pharmaceutical composition of any of the preceding enumerated embodiments.

### Embodiment 760

The composition for use according to any one of embodiments 571 to 692 wherein the composition is the method of treatment of embodiment 759 wherein the acid/base disorder is metabolic acidosis.

### Embodiment 761

The composition for use according to any one of embodiments 571 to 692 wherein the composition is the method of treatment of embodiment 759 wherein the pH is controlled or normalized.

### Embodiment 762

The composition for use according to any one of embodiments 571 to 692 wherein the composition is the method of treatment of embodiment 759 wherein the serum bicarbonate is controlled or normalized.

### Embodiment 763

The composition for use according to any one of embodiments 571 to 692 wherein the composition is the method of treatment of embodiment 759 wherein less than 1 g of sodium or potassium is administered per day.

### Embodiment 764

The composition for use according to any one of embodiments 571 to 692 wherein the composition is the method of treatment of embodiment 759 wherein less than 0.5 g of sodium or potassium is administered per day.

### Embodiment 765

The composition for use according to any one of embodiments 571 to 692 wherein the composition is the method of treatment of embodiment 759 wherein less than 0.1 g of sodium or potassium is administered per day.

### Embodiment 766

The composition for use according to any one of embodiments 571 to 692 wherein the composition is the method of treatment of embodiment 759 wherein no sodium or potassium is administered.

### Embodiment 767

The composition for use according to any one of embodiments 571 to 692 wherein the composition is the pharmaceutical composition of any of embodiments 682-755 wherein a dose of the pharmaceutical composition is titrated based on the serum bicarbonate values of a patient in need of treatment or other indicators of acidosis.

### Embodiment 768

The composition for use according to any one of embodiments 571 to 692 wherein the composition is a polymer comprising a structure corresponding to Formula 4:

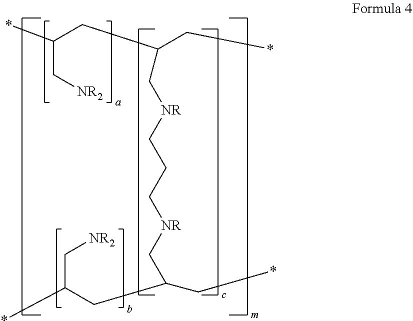

wherein each R is independently hydrogen or an ethylene crosslink between two nitrogen atoms of the crosslinked amine polymer

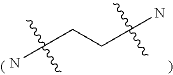

and a, b, c, and m are integers.

### Embodiment 769

The composition for use according to any one of embodiments 571 to 692 wherein the composition is the polymer of embodiment 768 wherein m is a large integer indicating an extended polymer network.

### Embodiment 770

The composition for use according to any one of embodiments 571 to 692 wherein the composition is the polymer of embodiment 768 or 769 wherein a ratio of the sum of a and b to c (i.e., a+b:c) is in the range of about 1:1 to 5:1.

### Embodiment 771

The composition for use according to any one of embodiments 571 to 692 wherein the composition is the polymer of embodiment 768 or 769 wherein a ratio of the sum of a and b to c (i.e., a+b:c) is in the range of about 1.5:1 to 4:1.

### Embodiment 772

The composition for use according to any one of embodiments 571 to 692 wherein the composition is the polymer of embodiment 768 or 769 wherein a ratio of the sum of a and b to c (i.e., a+b:c) is in the range of about 1.75:1 to 3:1.

### Embodiment 773

The composition for use according to any one of embodiments 571 to 692 wherein the composition is the polymer of embodiment 768 or 769 wherein a ratio of the sum of a and b to c (i.e., a+b:c) is in the range of about 2:1 to 2.5:1.

### Embodiment 774

The composition for use according to any one of embodiments 571 to 692 wherein the composition is the polymer of embodiment 768 or 769 wherein the sum of a and b is 57 and c is 24.

### Embodiment 775

The composition for use according to any one of embodiments 571 to 692 wherein the composition is the polymer of any of embodiments 768 or 774 wherein 50-95% of the R substituents are hydrogen and 5-50% are an ethylene crosslink between two nitrogens of the crosslinked amine polymer.

### Embodiment 776

The composition for use according to any one of embodiments 571 to 692 wherein the composition is the polymer of any of embodiments 768 or 774 wherein 55-90% of the R substituents are hydrogen and 10-45% are an ethylene crosslink between two nitrogens of the crosslinked amine polymer.

### Embodiment 777

The composition for use according to any one of embodiments 571 to 692 wherein the composition is the polymer of any of embodiments 768 or 774 wherein 60-90% of the R substituents are hydrogen and 10-40% are an ethylene crosslink between two nitrogens of the crosslinked amine polymer.

### Embodiment 778

The composition for use according to any one of embodiments 571 to 692 wherein the composition is the polymer of any of embodiments 768 to 774 wherein 65-90% of the R substituents are hydrogen and 10-35% are an ethylene crosslink between two nitrogens of the crosslinked amine polymer.

### Embodiment 779

The composition for use according to any one of embodiments 571 to 692 wherein the composition is the polymer of any of embodiments 768 to 774 wherein 70-90% of the R substituents are hydrogen and 10-30% are an ethylene crosslink between two nitrogens of the crosslinked amine polymer.

### Embodiment 780

The composition for use according to any one of embodiments 571 to 692 wherein the composition is the polymer of any of embodiments 768 to 774 wherein 75-85% of the R substituents are hydrogen and 15-25% are an ethylene crosslink between two nitrogens of the crosslinked amine polymer.

### Embodiment 781

The composition for use according to any one of embodiments 571 to 692 wherein the composition is the polymer of any of embodiments 768 to 774 wherein 80-85% of the R substituents are hydrogen and 15-20% are an ethylene crosslink between two nitrogens of the crosslinked amine polymer.

### Embodiment 782

The composition for use according to any one of embodiments 571 to 692 wherein the composition is the polymer of any of embodiments 768 to 774 wherein about 81% of the R substituents are hydrogen and about 19% are an ethylene crosslink.

### Embodiment 783

The composition for use according to any one of embodiments 571 to 592 wherein the method of treatment further includes the feature or features set out in any one of embodiments 1 to 570, or part thereof.

### Embodiment 784

A composition for use in a method of treating metabolic acidosis in an adult human patient wherein said treatment is administered to the patient less frequently than once per day, said composition being a nonabsorbable composition having the capacity to remove protons from the patient.

### Embodiment 785

The composition of embodiment 784, wherein the composition is administered on a regular schedule.

### Embodiment 786

The composition of embodiment 784, wherein the regular schedule is once every two days.

### Embodiment 787

The composition of embodiment 785, wherein the regular schedule is once every three days.

### Embodiment 788

The composition of embodiment 785, wherein the regular schedule is twice a week.

### Embodiment 789

The composition of embodiment 785, wherein the regular schedule is three times a week.

### Embodiment 790

The composition of embodiment 785, wherein the regular schedule is four times a week.

### Embodiment 791

The composition of any one of embodiments 784 to 790 wherein the composition is as defined in any preceding enumerated embodiment.

### Embodiment 792

The composition of any one of embodiments 784 to 791 wherein the method of treatment is as defined in any preceding enumerated embodiment.

### Embodiment 793

A method of increasing serum bicarbonate levels in an individual afflicted with an acid-base disorder, the method comprising oral administration of a pharmaceutical composition to increase the individual's serum bicarbonate levels wherein:

(i) the pharmaceutical composition binds a target species in the individual's digestive system when given orally, the target species being selected from the group consisting of protons, strong acids, and conjugate bases of strong acids and

(ii) the pharmaceutical composition increases the serum bicarbonate level by at least 1 mEq/l in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise at least 25 subjects, each cohort is prescribed the same diet during the study and the study lasts at least two weeks.

### Embodiment 794

The method of embodiment 793 wherein the first cohort receives a daily dose of the pharmaceutical composition that does not exceed 100 g/day.

### Embodiment 795

The method of embodiment 793 wherein the first cohort receives a daily dose of the pharmaceutical composition that does not exceed 50 g/day.

### Embodiment 796

The method of embodiment 793 wherein the first cohort receives a daily dose of the pharmaceutical composition that does not exceed 30 g/day.

### Embodiment 797

The method of embodiment 793 wherein the first cohort receives a daily dose of the pharmaceutical composition that does not exceed 25 g/day.

### Embodiment 798

The method of embodiment 793 wherein the first cohort receives a daily dose of the pharmaceutical composition that does not exceed 20 g/day.

### Embodiment 799

The method of embodiment 793 wherein the first cohort receives a daily dose of the pharmaceutical composition that does not exceed 15 g/day.

### Embodiment 800

The method of embodiment 793 wherein the first cohort receives a daily dose of the pharmaceutical composition that does not exceed 10 g/day.

### Embodiment 801

The method of embodiment 793 wherein the first cohort receives a daily dose of the pharmaceutical composition that does not exceed 5 g/day.

### Embodiment 802

The method of any of embodiments 793 to 801 wherein the target species is protons.

### Embodiment 803

The method of any of embodiments 793 to 801 wherein the target species is chloride ions.

### Embodiment 804

The method of any of embodiments 793 to 801 wherein the target species is a strong acid.

### Embodiment 805

The method of any of embodiments 793 to 801 wherein the target species is HCl.

### Embodiment 806

The method of any of embodiments 793 to 805 wherein the pharmaceutical composition is not absorbed when ingested.

### Embodiment 807

The method of any of embodiments 793 to 806 wherein the composition is a pharmaceutical composition comprising a proton-binding, crosslinked amine polymer comprising the residue of an amine corresponding to Formula 1:

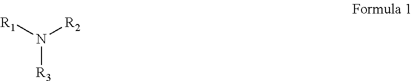

wherein R₁, R₂ and R₃ are independently hydrogen, hydrocarbyl, or substituted hydrocarbyl provided, however, at least one of R₁, R₂ and R₃ is other than hydrogen, and the crosslinked amine polymer has (i) an equilibrium proton binding capacity of at least 5 mmol/g and a chloride ion binding capacity of at least 5 mmol/g in an aqueous simulated gastric fluid buffer (“SGF”) containing 35 mM NaCl and 63 mM HCl at pH 1.2 and 37° C., and (ii) an equilibrium swelling ratio in deionized water of about 2 or less.

### Embodiment 808

The method of any of embodiments 793 to 806 wherein the composition is a pharmaceutical composition comprising a proton-binding, crosslinked amine polymer comprising the residue of an amine corresponding to Formula 1:

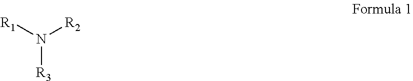

wherein R₁, R₂ and R₃ are independently hydrogen, hydrocarbyl, substituted hydrocarbyl provided, however, at least one of R₁, R₂ and R₃ is other than hydrogen, the crosslinked amine polymer has an equilibrium swelling ratio in deionized water of about 5 or less, and the crosslinked amine polymer binds a molar ratio of chloride ions to interfering ions of at least 0.35:1, respectively, in an interfering ion buffer at 37° C. wherein the interfering ions are phosphate ions and the interfering ion buffer is a buffered solution at pH 5.5 of 36 mM chloride and 20 mM phosphate.

### Embodiment 809

The method of embodiments 807 or 808 wherein the crosslinked amine polymer has an equilibrium chloride binding capacity of at least 7.5 mmol/g in an aqueous simulated gastric fluid buffer (“SGF”) containing 35 mM NaCl and 63 mM HCl at pH 1.2 and 37° C.

### Embodiment 810

The method of embodiments 807 or 808 wherein the crosslinked amine polymer has an equilibrium chloride binding capacity of at least 10 mmol/g in an aqueous simulated gastric fluid buffer (“SGF”) containing 35 mM NaCl and 63 mM HCl at pH 1.2 and 37° C.

### Embodiment 811

The method of embodiments 807 or 808 wherein the crosslinked amine polymer has an equilibrium swelling ratio in deionized water of about 4 or less.

### Embodiment 812

The method of embodiments 807 or 808 wherein the crosslinked amine polymer has an equilibrium swelling ratio in deionized water of about 3 or less.

### Embodiment 813

The method of embodiments 807 or 808 wherein the crosslinked amine polymer has an equilibrium swelling ratio in deionized water of about 2 or less.

### Embodiment 814

The method of any of embodiments 807 to 813 wherein R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ is not hydrogen.

### Embodiment 815

The method of any of embodiments 807 to 813 wherein R₁, R₂ and R₃ are independently hydrogen, aliphatic or heteroaliphatic provided, however, at least one of R₁, R₂ and R₃ is other than hydrogen.

### Embodiment 816

The method of any of embodiments 807 to 813 wherein the crosslinked amine polymer is prepared by substitution polymerization of the amine with a polyfunctional crosslinker, optionally also comprising amine moieties.

### Embodiment 817

The method of any of embodiments 807 to 816 wherein the potential renal acid load (PRAL value) of the diet is, on average, 0.82 mEq/d).

### Embodiment 818

The method of any of embodiments 807 to 817 wherein eligible subjects for the study have chronic kidney disease (CKD Stage 3-4; eGFR 20-<60 mL/min/1.73 m²) and a baseline serum bicarbonate value at the start of the study between 12 and 20 mEq/L.

### Embodiment 819

The method of any of embodiments 807 to 818 wherein the pharmaceutical composition increases the serum bicarbonate level by at least 2 mEq/l in the placebo controlled study.

### Embodiment 820

The method of any of embodiments 807 to 818 wherein the pharmaceutical composition increases the serum bicarbonate level by at least 3 mEq/l in the placebo controlled study.

### Embodiment 821

The method or composition of any preceding enumerated embodiment wherein the individual or adult human patient has chronic kidney disease.

### Embodiment 822

The method or composition of any preceding enumerated embodiment wherein the individual or adult human patient is not yet in need for kidney replacement therapy (dialysis or transplant).

### Embodiment 823

The method or composition of any preceding enumerated embodiment wherein the individual or adult human patient has not yet reached end stage renal disease (“ESRD”).

### Embodiment 824

The method or composition of any preceding enumerated embodiment wherein the individual or adult human patient has a mGFR of at least 15 mL/min/1.73 m².

### Embodiment 825

The method or composition of any preceding enumerated embodiment wherein the individual or adult human patient has an eGFR of at least 15 mL/min/1.73 m².

### Embodiment 826

The method or composition of any preceding enumerated embodiment wherein the individual or adult human patient has a mGFR of at least 30 mL/min/1.73 m².

### Embodiment 827

The method or composition of any preceding enumerated embodiment wherein the individual or adult human patient has an eGFR of at least 30 mL/min/1.73 m².

### Embodiment 828

The method or composition of any preceding enumerated embodiment wherein the individual or adult human patient has a mGFR of less than 45 mL/min/1.73 m² for at least three months.

### Embodiment 829

The method or composition of any preceding enumerated embodiment wherein the individual or adult human patient has an eGFR of less than 45 mL/min/1.73 m² for at least three months.

### Embodiment 830

The method or composition of any preceding enumerated embodiment wherein the individual or adult human patient has a mGFR of less than 60 mL/min/1.73 m² for at least three months.

### Embodiment 831

The method or composition of any preceding enumerated embodiment wherein the individual or adult human patient has an eGFR of less than 60 mL/min/1.73 m² for at least three months.

### Embodiment 832

The method or composition of any preceding enumerated embodiment wherein the individual or adult human patient has Stage 3A CKD, Stage 3B CKD, or Stage 4 CKD.

### Embodiment 833

A method of treating an individual afflicted with an acid-base disorder characterized by a baseline serum bicarbonate value of less than 22 mEq/l, the method comprising oral administration of a daily dose of a pharmaceutical composition containing a nonabsorbable composition;

wherein said oral administration increases the individual's serum bicarbonate value from baseline to an increased serum bicarbonate value that exceeds the baseline serum bicarbonate value by at least 1 mEq/l; and wherein the treatment enables the increased serum bicarbonate value to be sustained over a prolonged period of at least one week, at least one month, at least two months, at least three months, at least six months, or at least one year.

### Embodiment 834

The method or pharmaceutical composition of embodiment 833, wherein the method or pharmaceutical composition is one of any preceding enumerated embodiments.

### Embodiment 835

The method of any preceding enumerated embodiment wherein the treatment decreases the individual's anion gap by at least 1 mEq/L.

### Embodiment 836

The method of any preceding enumerated embodiment wherein the treatment decreases the individual's anion gap by at least 2 mEq/L.

### Embodiment 837

The method of any preceding enumerated embodiment wherein the treatment decreases the individual's anion gap by at least 3 mEq/L.

### Embodiment 838

The method of any preceding enumerated embodiment wherein the treatment decreases the individual's anion gap by at least 4 mEq/L.

### Embodiment 839

The method of any preceding enumerated embodiment wherein the treatment decreases the individual's anion gap by at least 5 mEq/L.

### Embodiment 840

The method of any preceding enumerated embodiment wherein the treatment decreases the individual's anion gap by 1-2 mEq/L.

### Embodiment 841

The method of any preceding enumerated embodiment wherein the treatment decreases the individual's anion gap by 1-3 mEq/L.

### Embodiment 842

The method of any preceding enumerated embodiment wherein the treatment decreases the individual's anion gap by 1-4 mEq/L.

### Embodiment 843

The method of any preceding enumerated embodiment wherein the treatment decreases the individual's anion gap by 1-5 mEq/L.

### Embodiment 844

The method of any preceding enumerated embodiment wherein the treatment decreases the individual's anion gap by 2-3 mEq/L.

### Embodiment 845

The method of any preceding enumerated embodiment wherein the treatment decreases the individual's anion gap by 2-4 mEq/L.

### Embodiment 846

The method of any preceding enumerated embodiment wherein the treatment decreases the individual's anion gap by 2-5 mEq/L.

### Embodiment 846

The method of any preceding enumerated embodiment wherein the treatment decreases the individual's anion gap by 3-4 mEq/L.

### Embodiment 847

The method of any preceding enumerated embodiment wherein the treatment decreases the individual's anion gap by 3-5 mEq/L.

### Embodiment 848

The method of any preceding enumerated embodiment wherein the treatment decreases the individual's anion gap by 4-5 mEq/L.

### Embodiment 849

The method of any preceding enumerated embodiment wherein the treatment decreases the individual's anion gap by less than 1 mEq/L (e.g. 0.5 mEq/L, or 0.75 mEq/L).

### Embodiment 850

A method of improving the quality of life of a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

### Embodiment 851

A method of improving the quality of life of a patient afflicted with chronic kidney disease and an acid-base disorder, the method comprising oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in quality of life is statistically significant compared to a placebo control group for a period of at least twelve weeks as assessed by a Quality of Life (QoL) questionnaire.

### Embodiment 852

A method of improving quality of life of a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to statistically significantly increase the patient's quality of life compared to a placebo control.

### Embodiment 853

A method of improving quality of life of a patient afflicted with metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the patient's quality of life compared to a placebo control group over the period, wherein the improvement in quality of life is statistically significant.

### Embodiment 854

A pharmaceutical composition for improving the quality of life of a human patient afflicted with chronic kidney disease and an acid-base disorder, the patient having a baseline serum bicarbonate level of ≤22 mEq/L prior to treatment, the composition being a nonabsorbable composition having the capacity to: (a) remove a target species from the patient selected from the group consisting of protons, strong acids, and conjugate bases of strong acids; and (b) improve the patient's quality of life compared to a placebo control in a statistically significant manner over at least a twelve-week period.

### Embodiment 855

A pharmaceutical composition for improving the quality of life of a human patient suffering from a disease or disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to improve the patient's quality of life compared to a placebo control in a statistically significant manner over at least the twelve-week period.

### Embodiment 856

A pharmaceutical composition for improving the quality of life of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in quality of life compared to a placebo control is statistically significant over the twelve-week period.

### Embodiment 857

A method of improving the physical function of a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

### Embodiment 858

A method of improving the physical function of a patient afflicted with chronic kidney disease and an acid-base disorder, the method comprising oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in physical function is statistically significant compared to a placebo control group at least twelve weeks after initiation of treatment as assessed by the patient's answers to question 3 of the Kidney Disease Quality of Life Short Form (KDQOL-SF).

### Embodiment 859

A method of improving the physical function of a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to statistically significantly increase the patient's physical function score based on answers to question 3 of the Kidney Disease Quality of Life Short Form (KDQOL-SF) compared to the patient's baseline physical function score.

### Embodiment 860

A method of improving the physical function of a patient afflicted with metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the physical function score of the patient compared to a placebo control group at the end of the period, wherein the improvement in the physical function score is statistically significant.

### Embodiment 861

A pharmaceutical composition for improving the physical function score of a human patient afflicted with chronic kidney disease and an acid-base disorder, the patient having a baseline serum bicarbonate level of ≤22 mEq/L prior to treatment, the composition being a nonabsorbable composition having the capacity to: (a) remove a target species from the patient selected from the group consisting of protons, strong acids, and conjugate bases of strong acids; and (b) improve the patient's physical function score based on answers to question 3 of the Kidney Disease Quality of Life Short Form (KDQOL-SF) compared to a placebo control in a statistically significant manner at the end of at least a twelve-week period.

### Embodiment 862

A pharmaceutical composition for improving the physical function score of a human patient suffering from a disease or disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to improve the patient's physical function score based on answers to question 3 of the Kidney Disease Quality of Life Short Form (KDQOL-SF) compared to a placebo control in a statistically significant manner at the end of an at least the twelve-week period.

### Embodiment 863

A pharmaceutical composition for improving the physical function score of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in physical function score is a statistically significant improvement over a baseline physical function score based on answers to question 3 of the Kidney Disease Quality of Life Short Form (KDQOL-SF) compared to a placebo control at the end of the at least twelve-week period.

### Embodiment 864

A method of slowing the progression of kidney disease in a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

### Embodiment 865

A method of slowing the progression of kidney disease in a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to increase the patient's serum bicarbonate by at least 1 mEq/L.

### Embodiment 866

A method of slowing the progression of kidney disease in a patient afflicted with chronic kidney disease and metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to slow the progression of kidney disease.

### Embodiment 867

A pharmaceutical composition for slowing the progression of kidney disease in a human patient afflicted with chronic kidney disease and an acid-base disorder, the patient having a baseline serum bicarbonate level of 22 mEq/L prior to treatment, the composition being a nonabsorbable composition having the capacity to: (a) remove a target species from the patient selected from the group consisting of protons, strong acids, and conjugate bases of strong acids; and (b) slow the progression of kidney disease in a human patient over at least a twelve-week period.

### Embodiment 868

A pharmaceutical composition for slowing the progression of kidney disease in a human patient afflicted with chronic kidney disease and an acid-base disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to slow the progression of kidney disease over at least the twelve-week period.

### Embodiment 869

A pharmaceutical composition for slowing the progression of kidney disease in a human patient also suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the progression of kidney disease in the patient is slowed over the twelve-week period compared to a placebo control group not receiving the pharmaceutical composition.

### Embodiment 870

A pharmaceutical composition for use in a method of treating an acid-base disorder in a patient, wherein the method of treatment improves the quality of life of the patient.

### Embodiment 871

A pharmaceutical composition for use in a method of treating an acid-base disorder in a patient, wherein the method of treatment improves the physical function of the patient.

### Embodiment 872

The method/composition of any preceding enumerated embodiment wherein the pharmaceutical composition is a nonabsorbable composition comprising a proton-binding, crosslinked amine polymer comprising the residue of an amine corresponding to Formula 1:

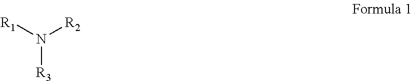

wherein R₁, R₂ and R₃ are independently hydrogen, hydrocarbyl, or substituted hydrocarbyl provided, however, at least one of R₁, R₂ and R₃ is other than hydrogen.

### Embodiment 872

The method/composition of embodiment 871 wherein R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ is not hydrogen.

### Embodiment 873

The method/composition of any of embodiments 871 to 872 wherein R₁, R₂ and R₃ are independently hydrogen, aliphatic or heteroaliphatic provided, however, at least one of R₁, R₂ and R₃ is other than hydrogen.

### Embodiment 874

The method/composition of any of embodiments 871 to 873 wherein the crosslinked amine polymer is prepared by substitution polymerization of the amine with a polyfunctional crosslinker, optionally also comprising amine moieties.

### Embodiment 875

The method/composition of any of embodiments 871 to 874 wherein the crosslinked amine polymer comprises the residue of an amine corresponding to Formula 1a and the crosslinked amine polymer is prepared by radical polymerization of an amine corresponding to Formula 1a:

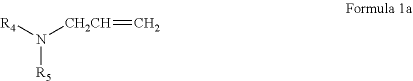

wherein R₄ and R₅ are independently hydrogen, hydrocarbyl, or substituted hydrocarbyl.

### Embodiment 876

The method/composition of embodiment 875 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 877

The method/composition of embodiment 875 wherein R₄ and R₅ are independently hydrogen, aliphatic or heteroaliphatic.

### Embodiment 878

The method/composition of any preceding enumerated embodiment wherein the pharmaceutical composition is a nonabsorbable composition comprising a crosslinked amine polymer containing the residue of an amine corresponding to Formula 1b and the crosslinked amine polymer is prepared by substitution polymerization of the amine corresponding to Formula 1 b with a polyfunctional crosslinker:

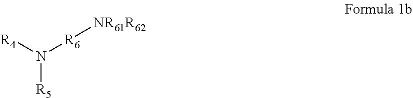

wherein R₄ and R₅ are independently hydrogen, hydrocarbyl, or substituted hydrocarbyl, R₆ is aliphatic and R₆₁ and R₆₂ are independently hydrogen, aliphatic, or heteroaliphatic.

### Embodiment 879

The method/composition of embodiment 878 wherein R₄ and R₅ are independently hydrogen, saturated hydrocarbon, unsaturated aliphatic, aryl, heteroaryl, heteroalkyl, or unsaturated heteroaliphatic.

### Embodiment 880

The method/composition of embodiment 878 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 881

The method/composition of embodiment 878 wherein R₄ and R₅ are independently hydrogen, allyl, or aminoalkyl.

### Embodiment 882

The method/composition according to any one of embodiments 871 to 881 wherein the crosslinked amine polymer comprises the residue of an amine corresponding to Formula 1c:

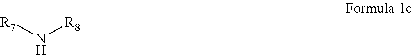

wherein R₇ is hydrogen, aliphatic or heteroaliphatic and R₈ is aliphatic or heteroaliphatic.

### Embodiment 883

The method/composition according to any one of the preceding enumerated embodiments wherein the crosslinked amine polymer comprises the residue of an amine corresponding to Formula 2:

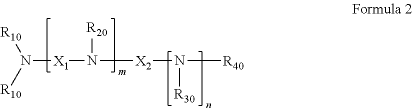

wherein
m and n are independently non-negative integers;
R₁₀, R₂₀, R₃₀, and R₄₀ are independently hydrogen, hydrocarbyl, or substituted hydrocarbyl;
X₁ is

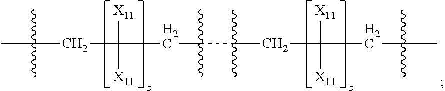

X₂ is hydrocarbyl or substituted hydrocarbyl;
each X₁₁ is independently hydrogen, hydrocarbyl, substituted hydrocarbyl, hydroxy, or amino; and
z is a non-negative number.

### Embodiment 884

The method/composition according to embodiment 883 wherein R₁₀, R₂₀, R₃₀, and R₄₀ are independently hydrogen, aliphatic, aryl, heteroaliphatic, or heteroaryl, m and z are independently 0-3 and n is 0 or 1.

### Embodiment 885

The method/composition according to embodiment 883 wherein X₂ is aliphatic or heteroaliphatic.

### Embodiment 886

The method/composition according to embodiment 883, wherein m is 1-3 and X₁₁ is hydrogen, aliphatic or heteroaliphatic.

### Embodiment 887

The method/composition according to embodiment 883, wherein the crosslinked amine polymer comprises the residue of an amine corresponding to Formula 2a:

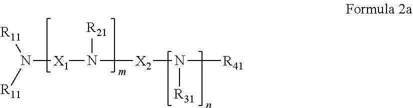

wherein
m and n are independently non-negative integers;
each R₁₁ is independently hydrogen, hydrocarbyl, heteroaliphatic, or heteroaryl;
R₂₁ and R₃₁, are independently hydrogen or heteroaliphatic;
R₄₁ is hydrogen, substituted hydrocarbyl, or hydrocarbyl;
X₁ is

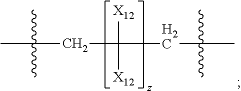

X₂ is alkyl or substituted hydrocarbyl;
each X₁₂ is independently hydrogen, hydroxy, amino, aminoalkyl, boronic acid or halo; and
z is a non-negative number.

### Embodiment 888

The method/composition according to embodiment 887, wherein m and z are independently 0-3 and n is 0 or 1.

### Embodiment 889

The method/composition according to any one of embodiments 887 to 888 wherein R₁₁ is independently hydrogen, aliphatic, aminoalkyl, haloalkyl, or heteroaryl, R₂₁ and R₃₁ are independently hydrogen or heteroaliphatic and R₄₁ is hydrogen, aliphatic, aryl, heteroaliphatic, or heteroaryl.

### Embodiment 890

The method/composition according to any one of embodiments 887 to 889 each R₁₁ is hydrogen, aliphatic, aminoalkyl, or haloalkyl, R₂₁ and R₃₁ are hydrogen or aminoalkyl, and R₄₁ is hydrogen, aliphatic, or heteroaliphatic.

### Embodiment 891

The method/composition according to any one of embodiments 887 to 890 wherein the crosslinked amine polymer comprises the residue of an amine corresponding to Formula 2b:

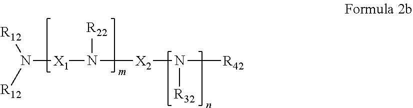

wherein
m and n are independently non-negative integers;
each R₁₂ is independently hydrogen, substituted hydrocarbyl, or hydrocarbyl;
R₂₂ and R₃₂ are independently hydrogen substituted hydrocarbyl, or hydrocarbyl;
R₄₂ is hydrogen, hydrocarbyl or substituted hydrocarbyl;
X₁ is

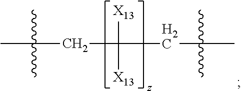

X₂ is alkyl, aminoalkyl, or alkanol;
each X₁₃ is independently hydrogen, hydroxy, alicyclic, amino, aminoalkyl, halogen, alkyl, heteroaryl, boronic acid or aryl;
z is a non-negative number; and
the amine corresponding to Formula 2b comprises at least one allyl group.

### Embodiment 892

The method/composition according to embodiment 891, wherein m and z are independently 0-3 and n is 0 or 1.

### Embodiment 893

The method/composition according to any one of embodiments 891 to 892 wherein R₁₂ or R₄₂ independently comprise at least one allyl or vinyl moiety.

### Embodiment 894

The method/composition according to any one of embodiments 891 to 893 wherein (i) m is a positive integer and R₁₂, R₂₂ and R₄₂, in combination comprise at least two allyl or vinyl moieties or (ii) n is a positive integer and R₁₂, R₃₂ and R₄₂, in combination, comprise at least two allyl or vinyl moieties.

### Embodiment 895

The method/composition according to any preceding enumerated embodiment wherein the crosslinked amine polymer comprises the residue of an amine appearing in Table A.

### Embodiment 896

The method/composition according to any preceding enumerated embodiment wherein the crosslinked amine polymer is crosslinked with a crosslinking agent appearing in Table B.

### Embodiment 897

The method/composition according to any preceding enumerated embodiment wherein the crosslinked amine polymer comprises a repeat unit corresponding to Formula 3:

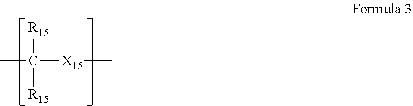

wherein
R₁₅, R₁₆ and R₁₇ are independently hydrogen, hydrocarbyl, substituted hydrocarbyl, hydroxyl, amino, boronic acid or halo;
X₁₅ is

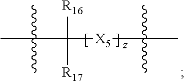

X₅ is hydrocarbyl, substituted hydrocarbyl, oxo (O), or amino; and
z is a non-negative number.

### Embodiment 898

The method/composition according to embodiment 897 wherein R₁₅, R₁₆ and R₁₇ are independently aliphatic or heteroaliphatic.

### Embodiment 899

The method/composition according to any one of embodiments 897 to 898 wherein X₅ is oxo, amino, alkylamino, ethereal, alkanol, or haloalkyl.

### Embodiment 900

The method/composition according to any one of embodiments 897 to 899 wherein the crosslinked amine polymer is prepared by (i) substitution polymerization of polyfunctional reagents at least one of which comprises amine moieties, (2) radical polymerization of a monomer comprising at least one amine moiety or nitrogen containing moiety, or (3) crosslinking of an amine-containing intermediate with a crosslinking agent, optionally containing amine moieties.

### Embodiment 901

The method/composition according to any preceding enumerated embodiment wherein the crosslinked amine polymer is a crosslinked homopolymer or a crosslinked copolymer.

### Embodiment 902

The method/composition according to according to any preceding enumerated embodiment wherein the crosslinked amine polymer comprises free amine moieties, separated by the same or varying lengths of repeating linker units.

### Embodiment 903

The method/composition according to any preceding enumerated embodiment wherein the crosslinked amine polymer is prepared by polymerizing an amine-containing monomer with a crosslinking agent in a substitution polymerization reaction.

### Embodiment 904

The method/composition according to any preceding enumerated embodiment wherein the amine-containing monomer is a linear amine possessing at least two reactive amine moieties to participate in the substitution polymerization reaction.

### Embodiment 905

The method/composition according to any preceding enumerated embodiment wherein the amine-containing monomer is 1,3-Bis[bis(2-aminoethyl)amino]propane, 3-Amino-1-{[2-(bis{2-[bis(3-aminopropyl)amino]ethyl}amino)ethyl](3-aminopropyl)amino}propane, 2-[Bis(2-aminoethyl)amino]ethanamine, Tris(3-aminopropyl)amine, 1,4-Bis[bis(3-aminopropyl)amino]butane, 1,2-Ethanediamine, 2-Amino-1-(2-aminoethylamino)ethane, 1,2-Bis(2-aminoethylamino)ethane, 1,3-Propanediamine, 3,3′-Diaminodipropylamine, 2,2-dimethyl-1,3-propanediamine, 2-methyl-1,3-propanediamine, N,N′-dimethyl-1,3-propanediamine, N-methyl-1,3-diaminopropane, 3,3′-diamino-N-methyldipropylamine, 1,3-diaminopentane, 1,2-diamino-2-methylpropane, 2-methyl-1,5-diaminopentane, 1,2-diaminopropane, 1,10-diaminodecane, 1,8-diaminooctane, 1,9-diaminooctane, 1,7-diaminoheptane, 1,6-diaminohexane, 1,5-diaminopentane, 3-bromopropylamine hydrobromide, N,2-dimethyl-1,3-propanediamine, N-isopropyl-1,3-diaminopropane, N,N′-bis(2-aminoethyl)-1,3-propanediamine, N,N′-bis(3-aminopropyl)ethylenediamine, N,N′-bis(3-aminopropyl)-1,4-butanediamine tetrahydrochloride, 1,3-diamino-2-propanol, N-ethylethylenediamine, 2,2′-diamino-N-methyldiethylamine, N,N′-diethylethylenediamine, N-isopropylethylenediamine, N-methylethylenediamine, N,N′-di-tert-butylethylenediamine, N,N′-diisopropylethylenediamine, N,N′-dimethylethylenediamine, N-butylethylenediamine, 2-(2-aminoethylamino)ethanol, 1,4,7,10,13,16-hexaazacyclooctadecane, 1,4,7,10-tetraazacyclododecane, 1,4,7-triazacyclononane, N,N′-bis(2-hydroxyethyl)ethylenediamine, piperazine, bis(hexamethylene)triamine, N-(3-hydroxypropyl)ethylenediamine, N-(2-Aminoethyl)piperazine, 2-Methylpiperazine, Homopiperazine, 1,4,8,11-Tetraazacyclotetradecane, 1,4,8,12-Tetraazacyclopentadecane, 2-(Aminomethyl)piperidine, or 3-(Methylamino)pyrrolidino.

### Embodiment 906

The method/composition according to any preceding enumerated embodiment wherein the crosslinking agent is selected from the group consisting of dihaloalkanes, haloalkyloxiranes, alkyloxirane sulfonates, di(haloalkyl)amines, tri(haloalkyl) amines, diepoxides, triepoxides, tetraepoxides, bis (halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, I-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy) diphenylmethane, bisphenol F diglycidyl ether, 1,4-bis(2′,3′epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4-h-chromene-2-carboxylate, bis[4-(2,3-epoxy-propylthio)phenyl]-sulfide, 1,3-bis(3-glycidoxypropyl) tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo [7,3,3,15, 11]heptasiloxane, 4,4′methylenebis(N, N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate, 3-chloro-1-(3-chloropropylamino-2-propanol, 1,2-bis(3-chloropropylamino)ethane, Bis(3-chloropropyl)amine, 1,3-Dichloro-2-propanol, 1,3-Dichloropropane, 1-chloro-2,3-epoxypropane, tris[(2-oxiranyl)methyl]amine, and combinations thereof.

### Embodiment 907

The method/composition according to any preceding enumerated embodiment wherein the preparation of the crosslinked amine polymer comprises radical polymerization of an amine monomer comprising at least one amine moiety or nitrogen containing moiety.

### Embodiment 908

The method/composition according to any preceding enumerated embodiment wherein the crosslinked amine polymer has an equilibrium swelling ratio in deionized water of about 1.5 or less.

### Embodiment 909

The method/composition according to any preceding enumerated embodiment wherein the crosslinked amine polymer has an equilibrium swelling ratio in deionized water of about 1 or less.

### Embodiment 910

The method/composition according to any preceding enumerated embodiment wherein the crosslinked amine polymer has a chloride ion to phosphate ion binding molar ratio of at least 0.5:1, respectively, in an aqueous simulated small intestine inorganic buffer (“SIB”) containing 36 mM NaCl, 20 mM NaH2PO4, and 50 mM 2-(N-morpholino)ethanesulfonic acid (MES) buffered to pH 5.5 and at 37° C.

### Embodiment 911

The method/composition according to any preceding enumerated embodiment wherein the pharmaceutical composition comprises a polymer comprising a structure corresponding to Formula 4:

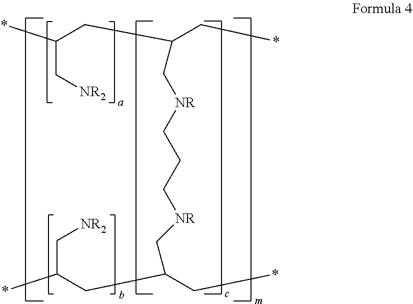

wherein each R is independently hydrogen or an ethylene crosslink between two

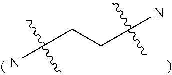

nitrogen atoms of the crosslinked amine polymer and a, b, c, and m are integers.

### Embodiment 912

The method/composition according to embodiment 911 wherein m is a large integer indicating an extended polymer network.

### Embodiment 913

The method/composition according to embodiment 911 or 912 wherein a ratio of the sum of a and b to c (i.e., a+b:c) is in the range of about 1:1 to 5:1.

### Embodiment 914

The method/composition according to any one of embodiments 9111 to 913 wherein a ratio of the sum of a and b to c (i.e., a+b:c) is in the range of about 1.5:1 to 4:1.

### Embodiment 915

The method/composition according to any one of embodiments 911 to 914 wherein a ratio of the sum of a and b to c (i.e., a+b:c) is in the range of about 1.75:1 to 3:1.

### Embodiment 916

The method/composition according to any one of embodiments 911 to 915 wherein a ratio of the sum of a and b to c (i.e., a+b:c) is in the range of about 2:1 to 2.5:1.

### Embodiment 917

The method/composition according to any one of embodiments 911 to 916 wherein the sum of a and b is 57 and c is 24.

### Embodiment 918

The method/composition according to any preceding enumerated embodiment wherein 50-95% of the R substituents are hydrogen and 5-50% are an ethylene crosslink between two nitrogens of the crosslinked amine polymer.

### Embodiment 919

The method/composition according to any preceding enumerated embodiment wherein 55-90% of the R substituents are hydrogen and 10-45% are an ethylene crosslink between two nitrogens of the crosslinked amine polymer.

### Embodiment 920

The method/composition according to any preceding enumerated embodiment wherein 60-90% of the R substituents are hydrogen and 10-40% are an ethylene crosslink between two nitrogens of the crosslinked amine polymer.

### Embodiment 921

The method/composition according to any preceding enumerated embodiment wherein 65-90% of the R substituents are hydrogen and 10-35% are an ethylene crosslink between two nitrogens of the crosslinked amine polymer.

### Embodiment 922

The method/composition according to any one of embodiments 911 to 921 wherein the R substituents are hydrogen and 10-30% are an ethylene crosslink between two nitrogens of the crosslinked amine polymer.

### Embodiment 923

The method/composition according to any preceding enumerated embodiment wherein the composition is the polymer of any of embodiments 768 to 774 wherein 75-85% of the R substituents are hydrogen and 15-25% are an ethylene crosslink between two nitrogens of the crosslinked amine polymer.

### Embodiment 924

The method/composition according to any preceding enumerated embodiment wherein the R substituents are hydrogen and 15-20% are an ethylene crosslink between two nitrogens of the crosslinked amine polymer.

### Embodiment 925

The method/composition according to any preceding enumerated embodiment wherein the R substituents are hydrogen and about 19% are an ethylene crosslink.

### Embodiment 926

The method/composition of any preceding enumerated embodiment wherein the pharmaceutical composition is characterized by a chloride ion binding capacity of at least 1 mEq/g in a SIB assay.

### Embodiment 927

The method/composition of any preceding enumerated embodiment wherein the pharmaceutical composition is characterized by a chloride ion binding capacity of at least 1.5 mEq/g in a SIB assay.

### Embodiment 928

The method/composition of any preceding enumerated embodiment wherein the pharmaceutical composition is characterized by a chloride ion binding capacity of at least 2 mEq/g in a SIB assay.

### Embodiment 929

The method/composition of any preceding enumerated embodiment wherein the pharmaceutical composition is characterized by a chloride ion binding capacity of at least 2.5 mEq/g in a SIB assay.

### Embodiment 930

The method/composition of any preceding enumerated embodiment wherein the pharmaceutical composition is characterized by a chloride ion binding capacity of at least 3 mEq/g in a SIB assay.

### Embodiment 931

The method/composition of any preceding enumerated embodiment wherein the pharmaceutical composition is characterized by a chloride ion binding capacity of at least 3.5 mEq/g in a SIB assay.

### Embodiment 932

The method/composition of any preceding enumerated embodiment wherein the pharmaceutical composition is characterized by a chloride ion binding capacity of at least 4 mEq/g in a SIB assay.

### Embodiment 933

The method/composition of any preceding enumerated embodiment wherein the pharmaceutical composition is characterized by a chloride ion binding capacity of at least 4.5 mEq/g in a SIB assay.

### Embodiment 934

The method/composition of any preceding enumerated embodiment wherein the pharmaceutical composition is characterized by a chloride ion binding capacity of at least 5 mEq/g in a SIB assay.

### Embodiment 935

The method/composition of any preceding enumerated embodiment wherein the pharmaceutical composition is characterized by a chloride ion binding capacity of at least 5.5 mEq/g in a SIB assay.

### Embodiment 936

The method/composition of any preceding enumerated embodiment wherein the pharmaceutical composition is characterized by a chloride ion binding capacity of at least 6 mEq/g in a SIB assay.

### Embodiment 937

The method/composition of any preceding enumerated embodiment wherein the binding capacity of the pharmaceutical composition is characterized by a ratio of the amount of bound chloride to bound phosphate in a SIB assay of at least 0.1:1, respectively.

### Embodiment 938

The method/composition of any preceding enumerated embodiment wherein the binding capacity of the pharmaceutical composition is characterized by a ratio of the amount of bound chloride to bound phosphate in a SIB assay of at least 0.2:1, respectively.

### Embodiment 939

The method/composition of any preceding enumerated embodiment wherein the binding capacity of the pharmaceutical composition is characterized by a ratio of the amount of bound chloride to bound phosphate in a SIB assay of at least 0.25:1, respectively.

### Embodiment 940

The method/composition of any preceding enumerated embodiment wherein the binding capacity of the pharmaceutical composition is characterized by a ratio of the amount of bound chloride to bound phosphate in a SIB assay of at least 0.3:1, respectively.

### Embodiment 941

The method/composition of any preceding enumerated embodiment wherein the binding capacity of the pharmaceutical composition is characterized by a ratio of the amount of bound chloride to bound phosphate in a SIB assay of at least 0.35:1, respectively.

### Embodiment 942

The method/composition of any preceding enumerated embodiment wherein the binding capacity of the pharmaceutical composition is characterized by a ratio of the amount of bound chloride to bound phosphate in a SIB assay of at least 0.4:1, respectively.

### Embodiment 943

The method/composition of any preceding enumerated embodiment wherein the binding capacity of the pharmaceutical composition is characterized by a ratio of the amount of bound chloride to bound phosphate in a SIB assay of at least 0.45:1, respectively.

### Embodiment 944

The method/composition of any preceding enumerated embodiment wherein the binding capacity of the pharmaceutical composition is characterized by a ratio of the amount of bound chloride to bound phosphate in a SIB assay of at least 0.5:1, respectively.

### Embodiment 945

The method/composition of any preceding enumerated embodiment wherein the binding capacity of the pharmaceutical composition is characterized by a ratio of the amount of bound chloride to bound phosphate in a SIB assay of at least 2:3, respectively.

### Embodiment 946

The method/composition of any preceding enumerated embodiment wherein the binding capacity of the pharmaceutical composition is characterized by a ratio of the amount of bound chloride to bound phosphate in a SIB assay of at least 0.75:1, respectively.

### Embodiment 947

The method/composition of any preceding enumerated embodiment wherein the binding capacity of the pharmaceutical composition is characterized by a ratio of the amount of bound chloride to bound phosphate in a SIB assay of at least 0.9:1, respectively.

### Embodiment 948

The method/composition of any preceding enumerated embodiment wherein the binding capacity of the pharmaceutical composition is characterized by a ratio of the amount of bound chloride to bound phosphate in a SIB assay of at least 1:1, respectively.

### Embodiment 949

The method/composition of any preceding enumerated embodiment wherein the binding capacity of the pharmaceutical composition is characterized by a ratio of the amount of bound chloride to bound phosphate in a SIB assay of at least 1.25:1, respectively.

### Embodiment 950

The method/composition of any preceding enumerated embodiment wherein the binding capacity of the pharmaceutical composition is characterized by a ratio of the amount of bound chloride to bound phosphate in a SIB assay of at least 1.5:1, respectively.

### Embodiment 951

The method/composition of any preceding enumerated embodiment wherein the binding capacity of the pharmaceutical composition is characterized by a ratio of the amount of bound chloride to bound phosphate in a SIB assay of at least 1.75:1, respectively.

### Embodiment 952

The method/composition of any preceding enumerated embodiment wherein the binding capacity of the pharmaceutical composition is characterized by a ratio of the amount of bound chloride to bound phosphate in a SIB assay of at least 2:1, respectively.

### Embodiment 953

The method/composition of any preceding enumerated embodiment wherein the binding capacity of the pharmaceutical composition is characterized by a ratio of the amount of bound chloride to bound phosphate in a SIB assay of at least 2.25:1, respectively.

### Embodiment 954

The method/composition of any preceding enumerated embodiment wherein the binding capacity of the pharmaceutical composition is characterized by a ratio of the amount of bound chloride to bound phosphate in a SIB assay of at least 2.5:1, respectively.

### Embodiment 955

The method/composition of any preceding enumerated embodiment wherein the binding capacity of the pharmaceutical composition is characterized by a ratio of the amount of bound chloride to bound phosphate in a SIB assay of at least 2.75:1, respectively.

### Embodiment 956

The method/composition of any preceding enumerated embodiment wherein the binding capacity of the pharmaceutical composition is characterized by a ratio of the amount of bound chloride to bound phosphate in a SIB assay of at least 3:1, respectively.

### Embodiment 957

The method/composition of any preceding enumerated embodiment wherein the binding capacity of the pharmaceutical composition is characterized by a ratio of the amount of bound chloride to bound phosphate in a SIB assay of at least 4:1, respectively.

### Embodiment 958

The method/composition of any preceding enumerated embodiment wherein the binding capacity of the pharmaceutical composition is characterized by a ratio of the amount of bound chloride to bound phosphate in a SIB assay of at least 5:1, respectively.

### Embodiment 959

The method/composition of any preceding enumerated embodiment wherein the binding capacity of the pharmaceutical composition is characterized by a ratio of the amount of bound chloride to bound phosphate in a SIB assay of at least 6:1, respectively.

### Embodiment 960

The method/composition of any preceding enumerated embodiment wherein the binding capacity of the pharmaceutical composition is characterized by a ratio of the amount of bound chloride to bound phosphate in a SIB assay of at least 7:1, respectively.

### Embodiment 961

The method/composition of any preceding enumerated embodiment wherein the binding capacity of the pharmaceutical composition is characterized by a ratio of the amount of bound chloride to bound phosphate in a SIB assay of at least 8:1, respectively.

### Embodiment 962

The method/composition of any preceding enumerated embodiment wherein the binding capacity of the pharmaceutical composition is characterized by a ratio of the amount of bound chloride to bound phosphate in a SIB assay of at least 9:1, respectively.

### Embodiment 963

The method/composition of any preceding enumerated embodiment wherein the binding capacity of the pharmaceutical composition is characterized by a ratio of the amount of bound chloride to bound phosphate in a SIB assay of at least 10:1, respectively.

### Embodiment 964

The method/composition of any preceding enumerated embodiment wherein the binding capacity of the pharmaceutical composition is characterized by a ratio of the amount of bound chloride to bound phosphate in a SIB assay of at least 12.5:1, respectively.

### Embodiment 965

The method/composition of any preceding enumerated embodiment wherein the binding capacity of the pharmaceutical composition is characterized by a ratio of the amount of bound chloride to bound phosphate in a SIB assay of at least 15:1, respectively.

### Embodiment 966

The method/composition of any preceding enumerated embodiment wherein the pharmaceutical composition has an equilibrium chloride binding capacity of at least 5 mmol/g in an aqueous simulated gastric fluid buffer (“SGF”) containing 35 mM NaCl and 63 mM HCl at pH 1.2 and 37° C.

### Embodiment 967

The method/composition of any preceding enumerated embodiment wherein the pharmaceutical composition has an equilibrium chloride binding capacity of at least 7.5 mmol/g in an aqueous simulated gastric fluid buffer (“SGF”) containing 35 mM NaCl and 63 mM HCl at pH 1.2 and 37° C.

### Embodiment 968

The method/composition of any preceding enumerated embodiment wherein the pharmaceutical composition has an equilibrium chloride binding capacity of at least 10 mmol/g in an aqueous simulated gastric fluid buffer (“SGF”) containing 35 mM NaCl and 63 mM HCl at pH 1.2 and 37° C.

### Embodiment 969

The method/composition of any preceding enumerated embodiment, wherein the pharmaceutical composition increases the serum bicarbonate level by at least 1 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

### Embodiment 970

The method/composition of any preceding enumerated embodiment, wherein the pharmaceutical composition increases the serum bicarbonate level by at least 2 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

### Embodiment 971

The method/composition of any preceding enumerated embodiment, wherein the pharmaceutical composition increases the serum bicarbonate level by at least 2.5 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

### Embodiment 972

The method/composition of any preceding enumerated embodiment, wherein the pharmaceutical composition has the capacity to change the patient's baseline serum bicarbonate level by at least 2 mEq/L in at the end of an at least twelve week placebo controlled study.

### Embodiment 973

The method/composition of any preceding enumerated embodiment, wherein the pharmaceutical composition has the capacity to change the patient's baseline serum bicarbonate level by at least 3 mEq/L in at the end of an at least twelve week placebo controlled study.

### Embodiment 974

The method/composition of any preceding enumerated embodiment, wherein the pharmaceutical composition has the capacity to change the patient's baseline serum bicarbonate level by at least 4 mEq/L in at the end of an at least twelve week placebo controlled study.

### Embodiment 975

The method/composition of any preceding enumerated embodiment, wherein the patient population for each cohort is at least 25 patients.

### Embodiment 976

The method/composition of any preceding enumerated embodiment, wherein the patient population for each cohort is at least 50 patients.

### Embodiment 977

The method/composition of any preceding enumerated embodiment, wherein the patient population for each cohort is at least 100 patients.

### Embodiment 978

The method/composition of any preceding enumerated embodiment, wherein the patient population for each cohort is at least 150 patients.

### Embodiment 979

The method/composition of any preceding enumerated embodiment, wherein the patient population for each cohort is at least 200 patients.

### Embodiment 980

The method/composition of any preceding enumerated embodiment, wherein improvement in quality of life or physical function is assessed by a questionnaire answered by a first cohort at the end of the period, relative to a second cohort who answered the same questionnaire at the end of the period, wherein the first cohort's subjects receive the pharmaceutical composition and the second cohort's subjects receive a placebo.

### Embodiment 981

The method/composition of any preceding enumerated embodiment, wherein improvement in quality of life or physical function is assessed by a questionnaire, which is a clinically validated assessment for evaluating a patient's physical and mental health.

### Embodiment 982

The method/composition of any preceding enumerated embodiment, wherein the questionnaire comprises questions concerning parameters selected from the group consisting of symptoms/problems related to the disease/condition, effects of the disease/condition, burden of the disease/condition, work status, cognitive function, quality of social interaction, sleep, social support, physical functioning, pain, general health, emotional well-being, social function, energy/fatigue, and combinations thereof.

### Embodiment 983

The method/composition of any preceding enumerated embodiment, wherein the questionnaire comprises questions concerning how the patient's health limits the patient's ability to engage in physical activities selected from the group: vigorous activities; moderate activities; lifting or carrying groceries; climbing several flights of stairs; climbing one flight of stairs; bending, kneeling or stooping; walking more than one mile; walking several blocks; walking one block; and bathing or dressing.

### Embodiment 984

The method/composition of any preceding enumerated embodiment, wherein the patient achieves at least about a 10% improvement on the quality of life scale relative to the placebo control.

### Embodiment 985

The method/composition of any preceding enumerated embodiment, wherein the patient achieves at least about a 25% improvement on the quality of life scale relative to the placebo control.

### Embodiment 986

The method/composition of any preceding enumerated embodiment, wherein the patient achieves at least about a 50% improvement on the quality of life scale relative to the placebo control.

### Embodiment 987

The method/composition of any preceding enumerated embodiment, wherein the patient achieves at least about a 75% improvement on the quality of life scale relative to the placebo control.

### Embodiment 988

The method/composition of any preceding enumerated embodiment, wherein the improvement of the physical function comprises: (a) an improvement in the patient's baseline physical function score of at least 1.5 points based on the patient's answers to question 3 of the Kidney Disease Quality of Life Short Form (KDQOL-SF); (b) an improvement in the patient's baseline repeated chair stand times of at least −1.5 seconds; or (c) an improvement in the patient's baseline physical function score of at least 1.5 points based on the patient's answers to question 3 of the KDQOL-SF and an improvement in the patient's baseline repeated chair stand times of at least −1.5 seconds. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in patient's baseline repeated chair stand times is seen after treatment for a period of at least about 12 weeks and a period of at least about 40 weeks. In another aspect of this and other embodiments, the improvement in patient's baseline repeated chair stand times is seen after treatment for a period of at least about 12 weeks and a period of at least about 52 weeks. In another aspect of this and other embodiments, the improvement in patient's baseline repeated chair stand times is seen after treatment for a period of at least about 12 weeks, a period of at least about 40 weeks and a period of at least about 52 weeks. In another aspect of this and other embodiments, in the Repeated Chair Stand Test, after about twelve weeks of treatment, there is a trend toward significance for the difference between treated and placebo-treated patients. In a further aspect of this and other embodiments, improvement in the patient's baseline repeated chair stand times is at least about 0.5, 0.75, 1.0, 1.1, 1.2, 1.3, or 1.4 seconds. In another aspect of this and other embodiments, the improvement in the patient's baseline physical function score is based on the patient's performance in the Single Chair Stand and/or Repeated Chair Stand protocols as depicted in FIG. 22. In another aspect of this and other embodiments, the patient's KDQOL-SF score is calculated as follows: 1 (limited a lot)=0; 2 (limited a little)=50; 3 (not limited)=100. Total score=sum of all 10, divided by 10.

### Embodiment 989

The method/composition of any preceding enumerated embodiment, wherein the improvement in the quality of life of the patient comprises a decrease or prevention of further bone loss and/or a decrease or prevention of further muscle loss in the patient.

### Embodiment 990

The method of any preceding enumerated embodiment, wherein the improvement in physical function score further includes an improvement in the patient's baseline repeated chair stand times compared to a placebo control of at least −1.5 seconds over the period. In one aspect of this and other embodiments, the improvement in the patient's baseline repeated chair stand is seen after treatment for a period of at least about 52 weeks, at least about 40 weeks, at least about 26 weeks, or at least about 12 weeks. In another aspect of this and other embodiments, the improvement in the patient's baseline repeated chair stand is seen after treatment for a period of at least about 12 weeks and at least about 40 weeks. In another aspect of this and other embodiments, the improvement in the patient's baseline repeated chair stand is seen after treatment for a period of at least about 12 weeks and at least about 52 weeks. In another aspect of this and other embodiments, the improvement in the patient's baseline repeated chair stand is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients. In another aspect of this and other embodiments, in the Repeated Chair Stand Test, after about twelve weeks of treatment, there is a trend toward significance for the difference between treated and placebo-treated patients. In a further aspect of this and other embodiments, improvement in the patient's baseline repeated chair stand times is at least about 0.5, 0.75, 1.0, 1.1, 1.2, 1.3, or 1.4 seconds. In another aspect of this and other embodiments, the improvement in the patient's baseline physical function score is based on the patient's performance in the Single Chair Stand and/or Repeated Chair Stand protocols as depicted in FIG. 22. In another aspect of this and other embodiments, the patient's KDQOL-SF score is calculated as follows: 1 (limited a lot)=0; 2 (limited a little)=50; 3 (not limited)=100. Total score=sum of all 10, divided by 10.

### Embodiment 991

The method/composition of any preceding enumerated embodiment, wherein the patient achieves at least about a 1.5 point improvement on the KDQOL-SF scale relative to the placebo control.

### Embodiment 992

The method/composition of any preceding enumerated embodiment, wherein the patient achieves at least about a 3.0 point improvement on the KDQOL-SF scale relative to the placebo control.

### Embodiment 993

The method/composition of any preceding enumerated embodiment, wherein the patient achieves at least about a 4.5 point improvement on the KDQOL-SF scale relative to the placebo control.

### Embodiment 994

The method/composition of any preceding enumerated embodiment, wherein the patient achieves at least about a 6.0 point improvement on the KDQOL-SF scale relative to the placebo control.

### Embodiment 995

The method/composition of any preceding enumerated embodiment, wherein the disease or disorder is characterized by a baseline serum bicarbonate value of less than 18 mEq/L.

### Embodiment 996

The method/composition of any preceding enumerated embodiment, wherein the disease or disorder is characterized by a baseline serum bicarbonate value of at least 12 mEq/L.

### Embodiment 997

The method/composition of any preceding enumerated embodiment, wherein the disease or disorder is characterized by a baseline serum bicarbonate value of at least 15 mEq/L.

### Embodiment 998

The method/composition of any preceding enumerated embodiment, wherein the patient's baseline serum bicarbonate value increases by at least 1 mEq/L during the period.

### Embodiment 999

The method/composition of any preceding enumerated embodiment, wherein the patient's baseline serum bicarbonate value increases by at least 2 mEq/L during the period.

### Embodiment 1000

The method/composition of any preceding enumerated embodiment, wherein the patient's baseline serum bicarbonate value increases by at least 3 mEq/L during the period.

### Embodiment 1001

The method/composition of any preceding enumerated embodiment, wherein a daily dose of the pharmaceutical composition is administered to the patient and the daily dose has the capacity to remove at least about 10 mEq/day, at least about 15 mEq/day, at least about 20 mEq/day, at least about 25 mEq/day, or at least about 30 mEq/day of the target species.

### Embodiment 1002

The method/composition of any preceding enumerated embodiment, wherein a daily dose of the pharmaceutical composition is administered to the patient and the daily dose has the capacity to remove less than 50 mEq/day or less than 35 mEq/day of the target species.

### Embodiment 1003

The method/composition of any preceding enumerated embodiment, wherein the period is at least three weeks, at least one month, at least two months, at least six months, at least 12 months, at least 18 months, or at least 24 months.

### Embodiment 1004

The method/composition of any preceding enumerated embodiment, wherein the pharmaceutical composition has the capacity to bind a target species selected from the group consisting of protons, strong acids, and conjugate bases of strong acids.

### Embodiment 1005

The method/composition of any preceding enumerated embodiment, wherein the conjugate base of a strong acid is selected from the group consisting of chloride, bisulfate and sulfate ions.

### Embodiment 1006

The method/composition of any preceding enumerated embodiment, wherein the target species comprises chloride ions.

### Embodiment 1007

The method/composition of any preceding enumerated embodiment, wherein the target species comprises hydrochloric acid.

### Embodiment 62A

The method/composition of any preceding enumerated embodiment, wherein the pharmaceutical composition is characterized by a chloride ion binding capacity of at least 1 mEq/g in a SIB assay.

### Embodiment 1008

The method/composition of any preceding enumerated embodiment, wherein the pharmaceutical composition is characterized by a chloride ion binding capacity of at least 1.5 mEq/g in a SIB assay.

### Embodiment 1009

The method/composition of any preceding enumerated embodiment, wherein the pharmaceutical composition is characterized by a chloride ion binding capacity of at least 2 mEq/g in a SIB assay.

### Embodiment 1010

The method/composition of any preceding enumerated embodiment, wherein the pharmaceutical composition has the capacity to bind chloride and phosphate ions in a SIB assay at a ratio that is at least 0.25:1, respectively.

### Embodiment 1011

The method/composition of any preceding enumerated embodiment, wherein the pharmaceutical composition has the capacity to bind chloride and phosphate ions in a SIB assay at a ratio that is at least 0.5:1, respectively.

### Embodiment 1012

The method/composition of any preceding enumerated embodiment, wherein the pharmaceutical composition has the capacity to bind chloride and phosphate ions in a SIB assay at a ratio that is at least 1:1, respectively.

### Embodiment 1013

The method/composition of any preceding enumerated embodiment, wherein the effective amount of the pharmaceutical composition or TRC101 comprises at least about 1 gm/day.

### Embodiment 1014

The method/composition of any preceding enumerated embodiment, wherein the effective amount of the pharmaceutical composition or TRC101 comprises from about 1-9 gm/day.

### Embodiment 1015

The method/composition of any preceding enumerated embodiment, wherein the effective amount of the pharmaceutical composition or TRC101 comprises about 4-6 gm/day.

### Embodiment 1016

The method/composition of any preceding enumerated embodiment, wherein the effective amount of the pharmaceutical composition or TRC101 comprises about 6 gm/day.

### Embodiment 1017

The method/composition of any preceding enumerated embodiment, wherein the effective amount of the pharmaceutical composition or TRC101 is administered to the patient in an oral dosage form once-a-day.

### Embodiment 1018

The method/composition of any preceding enumerated embodiment, wherein the effective amount of the pharmaceutical composition or TRC101 is adjusted to maintain the patient's serum bicarbonate level in a range between 22-29 mEq/L.

### Embodiment 1019

The method/composition of any preceding enumerated embodiment, wherein the improvement in the patient's baseline repeated chair stand times represents an improvement for at least one repetition.

### Embodiment 1020

The method/composition of any preceding enumerated embodiment, wherein the improvement in the patient's baseline repeated chair stand times represents an improvement for at least two repetitions.

### Embodiment 1021

The method/composition of any preceding enumerated embodiment, wherein the improvement in the patient's baseline repeated chair stand times represents an improvement for at least three repetitions.

### Embodiment 1022

The method/composition of any preceding enumerated embodiment, wherein the improvement in the patient's baseline repeated chair stand times represents an improvement for at least four repetitions.

### Embodiment 1023

The method/composition of any preceding enumerated embodiment, wherein the improvement in the patient's baseline repeated chair stand times represents an improvement for at least five repetitions.

### Embodiment 1024

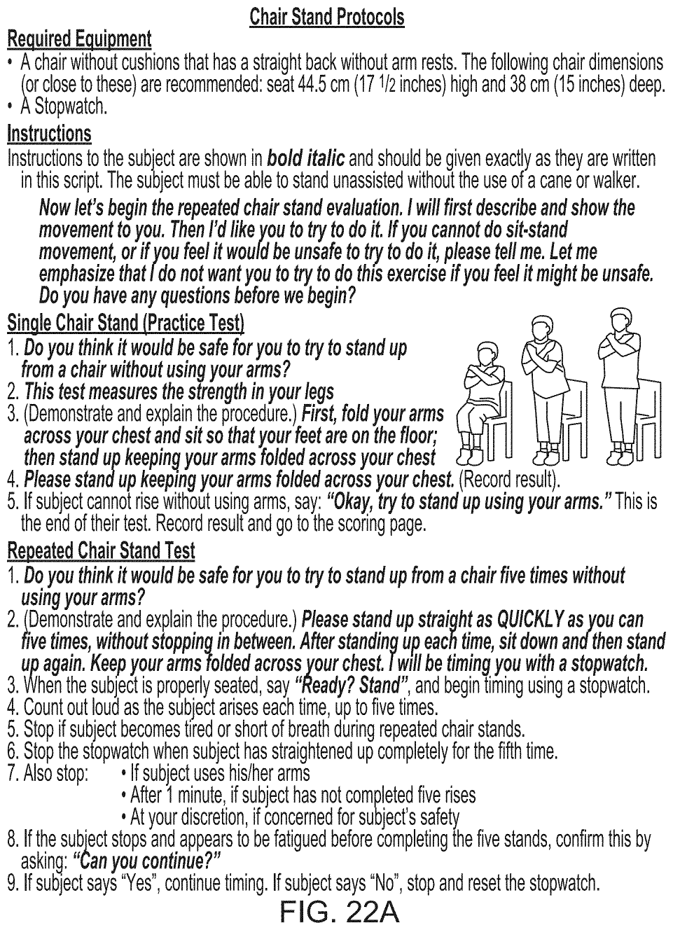

The method/composition of any preceding enumerated embodiment, wherein the improvement in the patient's baseline physical function score is based on the patient's answers to at least one question to Question 3 the of KDQOL-SF as depicted in FIG. 21.

### Embodiment 1025

The method/composition of any preceding enumerated embodiment, wherein the improvement in the patient's baseline physical function score is based on the patient's answers to at least five questions to Question 3 the of KDQOL-SF as depicted in FIG. 21.

### Embodiment 1026

The method/composition of any preceding enumerated embodiment, wherein the improvement in the patient's baseline physical function score is based on the patient's answers to at least seven questions to Question 3 of the KDQOL-SF as depicted in FIG. 21.

### Embodiment 1027

The method/composition of any preceding enumerated embodiment, wherein the improvement in the patient's baseline physical function score is based on the patient's answers to all questions to Question 3 of the KDQOL-SF as depicted in FIG. 21.

### Embodiment 1028

A pharmaceutical composition for use in a method of treating an acid-base disorder in a patient, wherein the method of treatment improves the quality of life of the patient.

### Embodiment 1029

A pharmaceutical composition for use in a method of treating an acid-base disorder in a patient, wherein the method of treatment improves the physical function of the patient.

### Embodiment 1030

The pharmaceutical composition for use in a method of treating an acid-base disorder of any preceding enumerated embodiment wherein the pharmaceutical composition is a nonabsorbable composition comprising a proton-binding, crosslinked amine polymer comprising the residue of an amine corresponding to Formula 1:

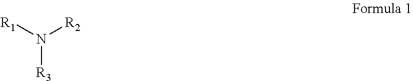

wherein R₁, R₂ and R₃ are independently hydrogen, hydrocarbyl, or substituted hydrocarbyl provided, however, at least one of R₁, R₂ and R₃ is other than hydrogen.

### Embodiment 1031

The pharmaceutical composition for use in a method of treating an acid-base disorder of embodiment 1030 wherein R₁, R₂ and R₃ are independently hydrogen, alkyl, alkenyl, allyl, vinyl, aryl, aminoalkyl, alkanol, haloalkyl, hydroxyalkyl, ethereal, heteroaryl or heterocyclic provided, however, each of R₁, R₂ and R₃ is not hydrogen.

### Embodiment 1032

The pharmaceutical composition for use in a method of treating an acid-base disorder of any of embodiments 1030 to 1031 wherein R₁, R₂ and R₃ are independently hydrogen, aliphatic or heteroaliphatic provided, however, at least one of R₁, R₂ and R₃ is other than hydrogen.

### Embodiment 1033

The pharmaceutical composition for use in a method of treating an acid-base disorder of any of embodiments 1030 to 1032 wherein the crosslinked amine polymer is prepared by substitution polymerization of the amine with a polyfunctional crosslinker, optionally also comprising amine moieties.

### Embodiment 1034

The pharmaceutical composition for use in a method of treating an acid-base disorder of any of embodiments 1030 to 1033 wherein the crosslinked amine polymer comprises the residue of an amine corresponding to Formula 1a and the crosslinked amine polymer is prepared by radical polymerization of an amine corresponding to Formula 1a:

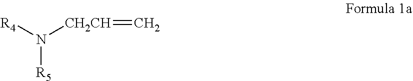

wherein R₄ and R₅ are independently hydrogen, hydrocarbyl, or substituted hydrocarbyl.

### Embodiment 1035

The pharmaceutical composition for use in a method of treating an acid-base disorder of embodiment 1034 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 1036

The pharmaceutical composition for use in a method of treating an acid-base disorder of embodiment 1034 wherein R₄ and R₅ are independently hydrogen, aliphatic or heteroaliphatic.

### Embodiment 1037

The pharmaceutical composition for use in a method of treating an acid-base disorder of any preceding enumerated embodiment wherein the pharmaceutical composition is a nonabsorbable composition comprising a crosslinked amine polymer containing the residue of an amine corresponding to Formula 1b and the crosslinked amine polymer is prepared by substitution polymerization of the amine corresponding to Formula 1b with a polyfunctional crosslinker:

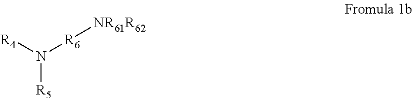

wherein R₄ and R₅ are independently hydrogen, hydrocarbyl, or substituted hydrocarbyl, R₆ is aliphatic and R₆₁ and R₆₂ are independently hydrogen, aliphatic, or heteroaliphatic.

### Embodiment 1038

The pharmaceutical composition for use in a method of treating an acid-base disorder of embodiment 1037 wherein R₄ and R₅ are independently hydrogen, saturated hydrocarbon, unsaturated aliphatic, aryl, heteroaryl, heteroalkyl, or unsaturated heteroaliphatic.

### Embodiment 1039

The pharmaceutical composition for use in a method of treating an acid-base disorder of embodiment 1037 wherein R₄ and R₅ are independently hydrogen, alkyl, alkenyl, allyl, vinyl, aryl, aminoalkyl, alkanol, haloalkyl, hydroxyalkyl, ethereal, heteroaryl or heterocyclic.

### Embodiment 1040

The pharmaceutical composition for use in a method of treating an acid-base disorder of embodiment 1037 wherein R₄ and R₅ are independently hydrogen, allyl, or aminoalkyl.

### Embodiment 1041

The pharmaceutical composition for use in a method of treating an acid-base disorder according to any one of embodiments 2E1 to 1040 wherein the crosslinked amine polymer comprises the residue of an amine corresponding to Formula 1c:

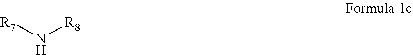

wherein R₇ is hydrogen, aliphatic or heteroaliphatic and R₈ is aliphatic or heteroaliphatic.

### Embodiment 1042

The pharmaceutical composition for use in a method of treating an acid-base disorder according to any one of the preceding enumerated embodiments wherein the crosslinked amine polymer comprises the residue of an amine corresponding to Formula 2:

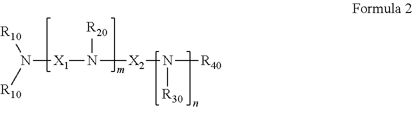

wherein
m and n are independently non-negative integers;
R₁₀, R₂₀, R₃₀, and R₄₀ are independently hydrogen, hydrocarbyl, or substituted hydrocarbyl;
X₁ is

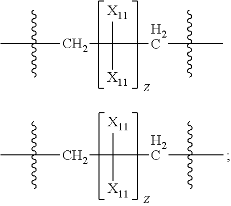

X₂ is hydrocarbyl or substituted hydrocarbyl;
each X₁₁ is independently hydrogen, hydrocarbyl, substituted hydrocarbyl, hydroxy, or amino; and
z is a non-negative number.

### Embodiment 1043

The pharmaceutical composition for use in a method of treating an acid-base disorder according to embodiment 1042 wherein R₁₀, R₂₀, R₃₀, and R₄₀ are independently hydrogen, aliphatic, aryl, heteroaliphatic, or heteroaryl, m and z are independently 0-3 and n is 0 or 1.

### Embodiment 1044

The pharmaceutical composition for use in a method of treating an acid-base disorder according to embodiment wherein X₂ is aliphatic or heteroaliphatic.

### Embodiment 1045

The pharmaceutical composition for use in a method of treating an acid-base disorder according to embodiment 1042, wherein m is 1-3 and X₁₁ is hydrogen, aliphatic or heteroaliphatic.

### Embodiment 1046

The pharmaceutical composition for use in a method of treating an acid-base disorder according to embodiment 1042, wherein the crosslinked amine polymer comprises the residue of an amine corresponding to Formula 2a:

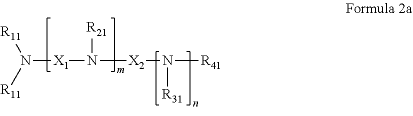

wherein
m and n are independently non-negative integers;
each R₁₁ is independently hydrogen, hydrocarbyl, heteroaliphatic, or heteroaryl;
R₂₁ and R₃₁, are independently hydrogen or heteroaliphatic;
R₄₁ is hydrogen, substituted hydrocarbyl, or hydrocarbyl;
X₁ is

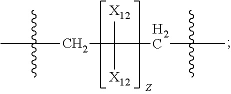

X₂ is alkyl or substituted hydrocarbyl;
each X₁₂ is independently hydrogen, hydroxy, amino, aminoalkyl, boronic acid or halo; and
z is a non-negative number.

### Embodiment 1047

The pharmaceutical composition for use in a method of treating an acid-base disorder according to embodiment 1046, wherein m and z are independently 0-3 and n is 0 or 1.

### Embodiment 1048

The pharmaceutical composition for use in a method of treating an acid-base disorder according to any one of embodiments 1046 to 1047 wherein R₁₁ is independently hydrogen, aliphatic, aminoalkyl, haloalkyl, or heteroaryl, R₂₁ and R₃₁ are independently hydrogen or heteroaliphatic and R₄₁ is hydrogen, aliphatic, aryl, heteroaliphatic, or heteroaryl.

### Embodiment 1049

The pharmaceutical composition for use in a method of treating an acid-base disorder according to any one of embodiments 1046 to 1048 each R₁₁ is hydrogen, aliphatic, aminoalkyl, or haloalkyl, R₂₁ and R₃₁ are hydrogen or aminoalkyl, and R₄₁ is hydrogen, aliphatic, or heteroaliphatic.

### Embodiment 1050

The pharmaceutical composition for use in a method of treating an acid-base disorder according to any one of embodiments 1046 to 1049 wherein the crosslinked amine polymer comprises the residue of an amine corresponding to Formula 2b:

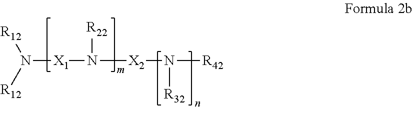

wherein
m and n are independently non-negative integers;
each R₁₂ is independently hydrogen, substituted hydrocarbyl, or hydrocarbyl;
R₂₂ and R₃₂ are independently hydrogen substituted hydrocarbyl, or hydrocarbyl;
R₄₂ is hydrogen, hydrocarbyl or substituted hydrocarbyl;
X₁ is

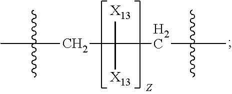

X₂ is alkyl, aminoalkyl, or alkanol;
each X₁₃ is independently hydrogen, hydroxy, alicyclic, amino, aminoalkyl, halogen, alkyl, heteroaryl, boronic acid or aryl;
z is a non-negative number; and
the amine corresponding to Formula 2b comprises at least one allyl group.

### Embodiment 1051

The pharmaceutical composition for use in a method of treating an acid-base disorder according to embodiment 1050, wherein m and z are independently 0-3 and n is 0 or 1.

### Embodiment 1052

The pharmaceutical composition for use in a method of treating an acid-base disorder according to any one of embodiments 1050 to 1051 wherein R₁₂ or R₄₂ independently comprise at least one allyl or vinyl moiety.

### Embodiment 1053

The pharmaceutical composition for use in a method of treating an acid-base disorder according to any one of embodiments 1050 to 1052 wherein (i) m is a positive integer and R₁₂, R₂₂ and R₄₂, in combination comprise at least two allyl or vinyl moieties or (ii) n is a positive integer and R₁₂, R₃₂ and R₄₂, in combination, comprise at least two allyl or vinyl moieties.

### Embodiment 1054

The pharmaceutical composition for use in a method of treating an acid-base disorder according to any preceding enumerated embodiment wherein the crosslinked amine polymer comprises the residue of an amine appearing in Table A.

### Embodiment 1055

The pharmaceutical composition for use in a method of treating an acid-base disorder according to any preceding enumerated embodiment wherein the crosslinked amine polymer is crosslinked with a crosslinking agent appearing in Table B.

### Embodiment 1056

The pharmaceutical composition for use in a method of treating an acid-base disorder according to any preceding enumerated embodiment wherein the crosslinked amine polymer comprises a repeat unit corresponding to Formula 3:

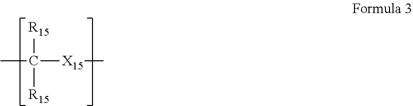

wherein
R₁₅, R₁₆ and R₁₇ are independently hydrogen, hydrocarbyl, substituted hydrocarbyl, hydroxyl, amino, boronic acid or halo;
X₁₅ is

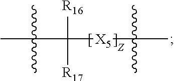

X₅ is hydrocarbyl, substituted hydrocarbyl, oxo (O), or amino; and
z is a non-negative number.

### Embodiment 1057

The pharmaceutical composition for use in a method of treating an acid-base disorder according to embodiment 1056 wherein R₁₅, R₁₆ and R₁₇ are independently aliphatic or heteroaliphatic.

### Embodiment 1058

The pharmaceutical composition for use in a method of treating an acid-base disorder according to any one of embodiments 1056 to 1057 wherein X₅ is oxo, amino, alkylamino, ethereal, alkanol, or haloalkyl.

### Embodiment 1059

The pharmaceutical composition for use in a method of treating an acid-base disorder according to any one of embodiments 1056 to 1058 wherein the crosslinked amine polymer is prepared by (i) substitution polymerization of polyfunctional reagents at least one of which comprises amine moieties, (2) radical polymerization of a monomer comprising at least one amine moiety or nitrogen containing moiety, or (3) crosslinking of an amine-containing intermediate with a crosslinking agent, optionally containing amine moieties.

### Embodiment 1060

The pharmaceutical composition for use in a method of treating an acid-base disorder according to any preceding enumerated embodiment wherein the crosslinked amine polymer is a crosslinked homopolymer or a crosslinked copolymer.

### Embodiment 1061

The pharmaceutical composition for use in a method of treating an acid-base disorder according to according to any preceding enumerated embodiment wherein the crosslinked amine polymer comprises free amine moieties, separated by the same or varying lengths of repeating linker units.

### Embodiment 1062

The pharmaceutical composition for use in a method of treating an acid-base disorder according to any preceding enumerated embodiment wherein the crosslinked amine polymer is prepared by polymerizing an amine-containing monomer with a crosslinking agent in a substitution polymerization reaction.

### Embodiment 1063

The pharmaceutical composition for use in a method of treating an acid-base disorder according to any preceding enumerated embodiment wherein the amine-containing monomer is a linear amine possessing at least two reactive amine moieties to participate in the substitution polymerization reaction.

### Embodiment 1064

The pharmaceutical composition for use in a method of treating an acid-base disorder according to any preceding enumerated embodiment wherein the amine-containing monomer is 1,3-Bis[bis(2-aminoethyl)amino]propane, 3-Amino-1-{[2-(bis{2-[bis(3-aminopropyl)amino]ethyl}amino)ethyl](3-aminopropyl)amino}propane, 2-[Bis(2-aminoethyl)amino]ethanamine, Tris(3-aminopropyl)amine, 1,4-Bis[bis(3-aminopropyl)amino]butane, 1,2-Ethanediamine, 2-Amino-1-(2-aminoethylamino)ethane, 1,2-Bis(2-aminoethylamino)ethane, 1,3-Propanediamine, 3,3′-Diaminodipropylamine, 2,2-dimethyl-1,3-propanediamine, 2-methyl-1,3-propanediamine, N,N′-dimethyl-1,3-propanediamine, N-methyl-1,3-diaminopropane, 3,3′-diamino-N-methyldipropylamine, 1,3-diaminopentane, 1,2-diamino-2-methylpropane, 2-methyl-1,5-diaminopentane, 1,2-diaminopropane, 1,10-diaminodecane, 1,8-diaminooctane, 1,9-diaminooctane, 1,7-diaminoheptane, 1,6-diaminohexane, 1,5-diaminopentane, 3-bromopropylamine hydrobromide, N,2-dimethyl-1,3-propanediamine, N-isopropyl-1,3-diaminopropane, N,N′-bis(2-aminoethyl)-1,3-propanediamine, N,N′-bis(3-aminopropyl)ethylenediamine, N,N′-bis(3-aminopropyl)-1,4-butanediamine tetrahydrochloride, 1,3-diamino-2-propanol, N-ethylethylenediamine, 2,2′-diamino-N-methyldiethylamine, N,N′-diethylethylenediamine, N-isopropylethylenediamine, N-methylethylenediamine, N,N′-di-tert-butylethylenediamine, N,N′-diisopropylethylenediamine, N,N′-dimethylethylenediamine, N-butylethylenediamine, 2-(2-aminoethylamino)ethanol, 1,4,7,10,13,16-hexaazacyclooctadecane, 1,4,7,10-tetraazacyclododecane, 1,4,7-triazacyclononane, N,N′-bis(2-hydroxyethyl)ethylenediamine, piperazine, bis(hexamethylene)triamine, N-(3-hydroxypropyl)ethylenediamine, N-(2-Aminoethyl)piperazine, 2-Methylpiperazine, Homopiperazine, 1,4,8,11-Tetraazacyclotetradecane, 1,4,8,12-Tetraazacyclopentadecane, 2-(Aminomethyl)piperidine, or 3-(Methylamino)pyrrolidino.

### Embodiment 1065

The pharmaceutical composition for use in a method of treating an acid-base disorder according to any preceding enumerated embodiment wherein the crosslinking agent is selected from the group consisting of dihaloalkanes, haloalkyloxiranes, alkyloxirane sulfonates, di(haloalkyl)amines, tri(haloalkyl) amines, diepoxides, triepoxides, tetraepoxides, bis (halomethyl)benzenes, tri(halomethyl)benzenes, tetra(halomethyl)benzenes, epihalohydrins such as epichlorohydrin and epibromohydrin poly(epichlorohydrin), (iodomethyl)oxirane, glycidyl tosylate, glycidyl 3-nitrobenzenesulfonate, 4-tosyloxy-1,2-epoxybutane, bromo-1,2-epoxybutane, 1,2-dibromoethane, 1,3-dichloropropane, 1,2-dichloroethane, l-bromo-2-chloroethane, 1,3-dibromopropane, bis(2-chloroethyl)amine, tris(2-chloroethyl)amine, and bis(2-chloroethyl)methylamine, 1,3-butadiene diepoxide, 1,5-hexadiene diepoxide, diglycidyl ether, 1,2,7,8-diepoxyoctane, 1,2,9,10-diepoxydecane, ethylene glycol diglycidyl ether, propylene glycol diglycidyl ether, 1,4-butanediol diglycidyl ether, 1,2 ethanedioldiglycidyl ether, glycerol diglycidyl ether, 1,3-diglycidyl glyceryl ether, N,N-diglycidylaniline, neopentyl glycol diglycidyl ether, diethylene glycol diglycidyl ether, 1,4-bis(glycidyloxy)benzene, resorcinol digylcidyl ether, 1,6-hexanediol diglycidyl ether, trimethylolpropane diglycidyl ether, 1,4-cyclohexanedimethanol diglycidyl ether, 1,3-bis-(2,3-epoxypropyloxy)-2-(2,3-dihydroxypropyloxy)propane, 1,2-cyclohexanedicarboxylic acid diglycidyl ester, 2,2′-bis(glycidyloxy) diphenylmethane, bisphenol F diglycidyl ether, 1,4-bis(2′,3′epoxypropyl)perfluoro-n-butane, 2,6-di(oxiran-2-ylmethyl)-1,2,3,5,6,7-hexahydropyrrolo[3,4-f]isoindol-1,3,5,7-tetraone, bisphenol A diglycidyl ether, ethyl 5-hydroxy-6,8-di(oxiran-2-ylmethyl)-4-oxo-4-h-chromene-2-carboxylate, bis[4-(2,3-epoxy-propylthio)phenyl]-sulfide, 1,3-bis(3-glycidoxypropyl) tetramethyldisiloxane, 9,9-bis[4-(glycidyloxy)phenyl]fluorine, triepoxyisocyanurate, glycerol triglycidyl ether, N,N-diglycidyl-4-glycidyloxyaniline, isocyanuric acid (S,S,S)-triglycidyl ester, isocyanuric acid (R,R,R)-triglycidyl ester, triglycidyl isocyanurate, trimethylolpropane triglycidyl ether, glycerol propoxylate triglycidyl ether, triphenylolmethane triglycidyl ether, 3,7,14-tris[[3-(epoxypropoxy)propyl]dimethylsilyloxy]-1,3,5,7,9,11,14-heptacyclopentyltricyclo [7,3,3,15, 11]heptasiloxane, 4,4′methylenebis(N, N-diglycidylaniline), bis(halomethyl)benzene, bis(halomethyl)biphenyl and bis(halomethyl)naphthalene, toluene diisocyanate, acrylol chloride, methyl acrylate, ethylene bisacrylamide, pyrometallic dianhydride, succinyl dichloride, dimethylsuccinate, 3-chloro-1-(3-chloropropylamino-2-propanol, 1,2-bis(3-chloropropylamino)ethane, Bis(3-chloropropyl)amine, 1,3-Dichloro-2-propanol, 1,3-Dichloropropane, 1-chloro-2,3-epoxypropane, tris[(2-oxiranyl)methyl]amine, and combinations thereof.

### Embodiment 1066

The pharmaceutical composition for use in a method of treating an acid-base disorder according to any preceding enumerated embodiment wherein the preparation of the crosslinked amine polymer comprises radical polymerization of an amine monomer comprising at least one amine moiety or nitrogen containing moiety.

### Embodiment 1067

The pharmaceutical composition for use in a method of treating an acid-base disorder according to any preceding enumerated embodiment wherein the crosslinked amine polymer has an equilibrium swelling ratio in deionized water of about 1.5 or less.

### Embodiment 1068

The pharmaceutical composition for use in a method of treating an acid-base disorder according to any preceding enumerated embodiment wherein the crosslinked amine polymer has an equilibrium swelling ratio in deionized water of about 1 or less.

### Embodiment 1069

The pharmaceutical composition for use in a method of treating an acid-base disorder according to any preceding enumerated embodiment wherein the crosslinked amine polymer has a chloride ion to phosphate ion binding molar ratio of at least 0.5:1, respectively, in an aqueous simulated small intestine inorganic buffer (“SIB”) containing 36 mM NaCl, 20 mM NaH2PO4, and 50 mM 2-(N-morpholino)ethanesulfonic acid (MES) buffered to pH 5.5 and at 37° C.

### Embodiment 1070

The pharmaceutical composition for use in a method of treating an acid-base disorder according to any preceding enumerated embodiment wherein the pharmaceutical composition comprises a polymer comprising a structure corresponding to Formula 4:

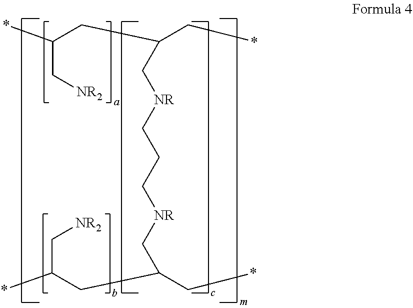

wherein each R is independently hydrogen or an ethylene crosslink between
two nitrogen atoms of the crosslinked amine polymer

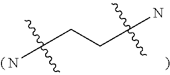

and a, b, c, and m are integers.

### Embodiment 1071

The pharmaceutical composition for use in a method of treating an acid-base disorder according to embodiment 1070 wherein m is a large integer indicating an extended polymer network.

### Embodiment 1072

The pharmaceutical composition for use in a method of treating an acid-base disorder according to embodiment 1070 or 1071 wherein a ratio of the sum of a and b to c (i.e., a+b:c) is in the range of about 1:1 to 5:1.

### Embodiment 1073

The pharmaceutical composition for use in a method of treating an acid-base disorder according to any one of embodiments 1070 to 1072 wherein a ratio of the sum of a and b to c (i.e., a+b:c) is in the range of about 1.5:1 to 4:1.

### Embodiment 1074

The pharmaceutical composition for use in a method of treating an acid-base disorder according to any one of embodiments 1070 to 1073 wherein a ratio of the sum of a and b to c (i.e., a+b:c) is in the range of about 1.75:1 to 3:1.

### Embodiment 1075

The pharmaceutical composition for use in a method of treating an acid-base disorder according to any one of embodiments 1070 to 1074 wherein a ratio of the sum of a and b to c (i.e., a+b:c) is in the range of about 2:1 to 2.5:1.

### Embodiment 1076

The pharmaceutical composition for use in a method of treating an acid-base disorder according to any one of embodiments 1070 to 1075 wherein the sum of a and b is 57 and c is 24.

### Embodiment 1077

The pharmaceutical composition for use in a method of treating an acid-base disorder according to any preceding enumerated embodiment wherein 50-95% of the R substituents are hydrogen and 5-50% are an ethylene crosslink between two nitrogens of the crosslinked amine polymer.

### Embodiment 1078

The pharmaceutical composition for use in a method of treating an acid-base disorder according to any preceding enumerated embodiment wherein 55-90% of the R substituents are hydrogen and 10-45% are an ethylene crosslink between two nitrogens of the crosslinked amine polymer.

### Embodiment 1079

The pharmaceutical composition for use in a method of treating an acid-base disorder according to any preceding enumerated embodiment wherein 60-90% of the R substituents are hydrogen and 10-40% are an ethylene crosslink between two nitrogens of the crosslinked amine polymer.

### Embodiment 1080

The pharmaceutical composition for use in a method of treating an acid-base disorder according to any preceding enumerated embodiment wherein 65-90% of the R substituents are hydrogen and 10-35% are an ethylene crosslink between two nitrogens of the crosslinked amine polymer.

### Embodiment 1081

The pharmaceutical composition for use in a method of treating an acid-base disorder according to any one of embodiments 1070 to 1082 wherein the R substituents are hydrogen and 10-30% are an ethylene crosslink between two nitrogens of the crosslinked amine polymer.

### Embodiment 1082

The pharmaceutical composition for use in a method of treating an acid-base disorder according to any preceding enumerated embodiment wherein the composition is the polymer of any of embodiments 768 to 774 wherein 75-85% of the R substituents are hydrogen and 15-25% are an ethylene crosslink between two nitrogens of the crosslinked amine polymer.

### Embodiment 1083

The pharmaceutical composition for use in a method of treating an acid-base disorder according to any preceding enumerated embodiment wherein the R substituents are hydrogen and 15-20% are an ethylene crosslink between two nitrogens of the crosslinked amine polymer.

### Embodiment 1084

The pharmaceutical composition for use in a method of treating an acid-base disorder according to any preceding enumerated embodiment wherein the R substituents are hydrogen and about 19% are an ethylene crosslink.

### Embodiment 1085

The pharmaceutical composition for use in a method of treating an acid-base disorder of any preceding enumerated embodiment wherein the pharmaceutical composition is characterized by a chloride ion binding capacity of at least 1 mEq/g in a SIB assay.

### Embodiment 1086

The pharmaceutical composition for use in a method of treating an acid-base disorder of any preceding enumerated embodiment wherein the pharmaceutical composition is characterized by a chloride ion binding capacity of at least 1.5 mEq/g in a SIB assay.

### Embodiment 1087

The pharmaceutical composition for use in a method of treating an acid-base disorder of any preceding enumerated embodiment wherein the pharmaceutical composition is characterized by a chloride ion binding capacity of at least 2 mEq/g in a SIB assay.

### Embodiment 1088

The pharmaceutical composition for use in a method of treating an acid-base disorder of any preceding enumerated embodiment wherein the pharmaceutical composition is characterized by a chloride ion binding capacity of at least 2.5 mEq/g in a SIB assay.

### Embodiment 1089

The pharmaceutical composition for use in a method of treating an acid-base disorder of any preceding enumerated embodiment wherein the pharmaceutical composition is characterized by a chloride ion binding capacity of at least 3 mEq/g in a SIB assay.

### Embodiment 1090

The pharmaceutical composition for use in a method of treating an acid-base disorder of any preceding enumerated embodiment wherein the pharmaceutical composition is characterized by a chloride ion binding capacity of at least 3.5 mEq/g in a SIB assay.

### Embodiment 1091

The pharmaceutical composition for use in a method of treating an acid-base disorder of any preceding enumerated embodiment wherein the pharmaceutical composition is characterized by a chloride ion binding capacity of at least 4 mEq/g in a SIB assay.

### Embodiment 1092

The pharmaceutical composition for use in a method of treating an acid-base disorder of any preceding enumerated embodiment wherein the pharmaceutical composition is characterized by a chloride ion binding capacity of at least 4.5 mEq/g in a SIB assay.

### Embodiment 1093

The pharmaceutical composition for use in a method of treating an acid-base disorder of any preceding enumerated embodiment wherein the pharmaceutical composition is characterized by a chloride ion binding capacity of at least 5 mEq/g in a SIB assay.

### Embodiment 1094

The pharmaceutical composition for use in a method of treating an acid-base disorder of any preceding enumerated embodiment wherein the pharmaceutical composition is characterized by a chloride ion binding capacity of at least 5.5 mEq/g in a SIB assay.

### Embodiment 1095

The pharmaceutical composition for use in a method of treating an acid-base disorder of any preceding enumerated embodiment wherein the pharmaceutical composition is characterized by a chloride ion binding capacity of at least 6 mEq/g in a SIB assay.

### Embodiment 1096

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.1:1, respectively.

### Embodiment 1097

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.2:1, respectively.

### Embodiment 1098

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.25:1, respectively.

### Embodiment 1099

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.3:1, respectively.

### Embodiment 1100

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.35:1, respectively.

### Embodiment 1101

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.4:1, respectively.

### Embodiment 1102

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.45:1, respectively.

### Embodiment 1103

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.5:1, respectively.

### Embodiment 1104

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 2:3, respectively.

### Embodiment 1105

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.75:1, respectively.

### Embodiment 1106

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 0.9:1, respectively.

### Embodiment 1107

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 1:1, respectively.

### Embodiment 1108

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 1.25:1, respectively.

### Embodiment 1109

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 1.5:1, respectively.

### Embodiment 1110

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 1.75:1, respectively.

### Embodiment 1111

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 2:1, respectively.

### Embodiment 1112

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 2.25:1, respectively.

### Embodiment 1113

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 2.5:1, respectively.

### Embodiment 1114

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 2.75:1, respectively.

### Embodiment 1115

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 3:1, respectively.

### Embodiment 1116

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 4:1, respectively.

### Embodiment 1117

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 5:1, respectively.

### Embodiment 1118

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 6:1, respectively.

### Embodiment 1119

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 7:1, respectively.

### Embodiment 1120

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 8:1, respectively.

### Embodiment 1121

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 9:1, respectively.

### Embodiment 1122

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 10:1, respectively.

### Embodiment 1123

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 12.5:1, respectively.

### Embodiment 1124

The pharmaceutical composition for use in a method of treating an acid-base disorder of any preceding enumerated embodiment wherein the binding capacity of the pharmaceutical composition is characterized by a ratio of the amount of bound chloride to bound phosphate in a SIB assay of at least 15:1, respectively.

### Embodiment 1125

The pharmaceutical composition for use in a method of treating an acid-base disorder of any preceding enumerated embodiment wherein the pharmaceutical composition has an equilibrium chloride binding capacity of at least 5 mmol/g in an aqueous simulated gastric fluid buffer (“SGF”) containing 35 mM NaCl and 63 mM HCl at pH 1.2 and 37° C.

### Embodiment 1126

The pharmaceutical composition for use in a method of treating an acid-base disorder of any preceding enumerated embodiment wherein the pharmaceutical composition has an equilibrium chloride binding capacity of at least 7.5 mmol/g in an aqueous simulated gastric fluid buffer (“SGF”) containing 35 mM NaCl and 63 mM HCl at pH 1.2 and 37° C.

### Embodiment 1127

The pharmaceutical composition for use in a method of treating an acid-base disorder of any preceding enumerated embodiment wherein the pharmaceutical composition has an equilibrium chloride binding capacity of at least 10 mmol/g in an aqueous simulated gastric fluid buffer (“SGF”) containing 35 mM NaCl and 63 mM HCl at pH 1.2 and 37° C.

### Embodiment 1128

The pharmaceutical composition for use in a method of treating an acid-base disorder of any preceding enumerated embodiment, wherein the acid-base disorder is metabolic acidosis.

### Embodiment 1129

The pharmaceutical composition for use in a method of treating an acid-base disorder of any preceding enumerated embodiment, wherein the patient is afflicted with chronic kidney disease.

### Embodiment 1130

The pharmaceutical composition for use in a method of treating an acid-base disorder of any preceding enumerated embodiment, wherein the pharmaceutical composition comprises a polymer as defined anywhere in the description.

### Embodiment 1131

The pharmaceutical composition for use in a method of treating an acid-base disorder of any preceding enumerated embodiment, wherein the acid-based disorder is characterized by a baseline serum bicarbonate value of less than about 21 mEq/l.

### Embodiment 1132

The pharmaceutical composition for use in a method of treating an acid-base disorder of any preceding enumerated embodiment, wherein the acid-based disorder is characterized by a baseline serum bicarbonate value of less than about 20 mEq/l.

### Embodiment 1133

The pharmaceutical composition for use in a method of treating an acid-base disorder of any preceding enumerated embodiment, wherein the acid-based disorder is characterized by a baseline serum bicarbonate value of less than about 19 mEq/l.

### Embodiment 1134

The pharmaceutical composition for use in a method of treating an acid-base disorder of any preceding enumerated embodiment, wherein the acid-based disorder is characterized by a baseline serum bicarbonate value of less than about 18 mEq/l.

### Embodiment 1135

The pharmaceutical composition for use in a method of treating an acid-base disorder of any preceding enumerated embodiment, wherein the acid-based disorder is characterized by a baseline serum bicarbonate value of less than about 17 mEq/l.

### Embodiment 1136

The pharmaceutical composition for use in a method of treating an acid-base disorder of any preceding enumerated embodiment, wherein the acid-based disorder is characterized by a baseline serum bicarbonate value of less than about 16 mEq/l.

### Embodiment 1137

The pharmaceutical composition for use in a method of treating an acid-base disorder of any preceding enumerated embodiment, wherein the acid-based disorder is characterized by a baseline serum bicarbonate value of less than about 15 mEq/l.

### Embodiment 1138

The pharmaceutical composition for use in a method of treating an acid-base disorder of any preceding enumerated embodiment, wherein the acid-based disorder is characterized by a baseline serum bicarbonate value of less than about 14 mEq/l.

### Embodiment 1139

The pharmaceutical composition for use in a method of treating an acid-base disorder of any preceding enumerated embodiment, wherein the acid-based disorder is characterized by a baseline serum bicarbonate value of less than about 13 mEq/l.

### Embodiment 1140

The pharmaceutical composition for use in a method of treating an acid-base disorder of any preceding enumerated embodiment, wherein the acid-based disorder is characterized by a baseline serum bicarbonate value of less than about 12 mEq/l.

### Embodiment 1141

The pharmaceutical composition for use in a method of treating an acid-base disorder of any preceding enumerated embodiment, wherein the acid-based disorder is characterized by a baseline serum bicarbonate value of less than about 11 mEq/l.

### Embodiment 1142

The pharmaceutical composition for use in a method of treating an acid-base disorder of any preceding enumerated embodiment, wherein the acid-based disorder is characterized by a baseline serum bicarbonate value of less than about 10 mEq/l.

### Embodiment 1143

The pharmaceutical composition for use in a method of treating an acid-base disorder of any preceding enumerated embodiment, wherein the acid-based disorder is characterized by a baseline serum bicarbonate value of at least about 10 mEq/l.

### Embodiment 1144

The pharmaceutical composition for use in a method of treating an acid-base disorder of any preceding enumerated embodiment, wherein the acid-based disorder is characterized by a baseline serum bicarbonate value of at least about 11 mEq/l.

### Embodiment 1145

The pharmaceutical composition for use in a method of treating an acid-base disorder of any preceding enumerated embodiment, wherein the acid-based disorder is characterized by a baseline serum bicarbonate value of at least about 12 mEq/l.

### Embodiment 1146

The pharmaceutical composition for use in a method of treating an acid-base disorder of any preceding enumerated embodiment, wherein the acid-based disorder is characterized by a baseline serum bicarbonate value of at least about 13 mEq/l.

### Embodiment 1147

The pharmaceutical composition for use in a method of treating an acid-base disorder of any preceding enumerated embodiment, wherein the acid-based disorder is characterized by a baseline serum bicarbonate value of at least about 14 mEq/l.

### Embodiment 1148

The pharmaceutical composition for use in a method of treating an acid-base disorder of any preceding enumerated embodiment, wherein the acid-based disorder is characterized by a baseline serum bicarbonate value of at least about 15 mEq/l.

### Embodiment 1149

The pharmaceutical composition for use in a method of treating an acid-base disorder of any preceding enumerated embodiment, wherein the acid-based disorder is characterized by a baseline serum bicarbonate value of at least about 16 mEq/l.

### Embodiment 1150

The pharmaceutical composition for use in a method of treating an acid-base disorder of any preceding enumerated embodiment, wherein the acid-based disorder is characterized by a baseline serum bicarbonate value of at least about 17 mEq/l.

### Embodiment 1151

The pharmaceutical composition for use in a method of treating an acid-base disorder of any preceding enumerated embodiment, wherein the acid-based disorder is characterized by a baseline serum bicarbonate value of at least about 18 mEq/l.

### Embodiment 1152

The pharmaceutical composition for use in a method of treating an acid-base disorder of any preceding enumerated embodiment, wherein the acid-based disorder is characterized by a baseline serum bicarbonate value of at least about 19 mEq/l.

### Embodiment 1153

The pharmaceutical composition for use in a method of treating an acid-base disorder of any preceding enumerated embodiment, wherein the acid-based disorder is characterized by a baseline serum bicarbonate value of at least about 20 mEq/l.

### Embodiment 1154

The pharmaceutical composition for use in a method of treating an acid-base disorder of any preceding enumerated embodiment, wherein the acid-based disorder is characterized by a baseline serum bicarbonate value of at least about 21 mEq/l.

### Embodiment 1155

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 22 mEg/I.

### Embodiment 1156

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 23 mEg/I.

### Embodiment 1157

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 24 mEg/I.

### Embodiment 1158

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 25 mEg/I.

### Embodiment 1159

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 26 mEg/I.

### Embodiment 1160

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 27 mEg/I.

### Embodiment 1161

The pharmaceutical composition for use in a method of treating an acid-base disorder of any preceding enumerated embodiment, wherein the method increases the serum bicarbonate value from the baseline serum bicarbonate value to an increased serum bicarbonate value of at least about 28 mEg/I.

### Embodiment 1162

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 29 mEq/l.

### Embodiment 1163

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 28 mEq/l.

### Embodiment 1164

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 27 mEq/l.

### Embodiment 1165

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 26 mEq/l.

### Embodiment 1166

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 25 mEq/l.

### Embodiment 1167

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 24 mEq/l.

### Embodiment 1168

The pharmaceutical composition for use in a method of treating an acid-base disorder of any preceding enumerated embodiment, wherein the method increases the serum bicarbonate value from the baseline serum bicarbonate value to an increased serum bicarbonate value not in excess of about 23 mEq/l.

### Embodiment 1169

The pharmaceutical composition for use in a method of treating an acid-base disorder of any preceding enumerated embodiment, wherein the method maintains the patient's serum bicarbonate within the range of 22 to 29 mEq/l.

### Embodiment 1170

The pharmaceutical composition for use in a method of treating an acid-base disorder of any preceding enumerated embodiment, wherein the method maintains the patient's serum bicarbonate within the range of 22 to 28 mEq/l.

### Embodiment 1171

The pharmaceutical composition for use in a method of treating an acid-base disorder of any preceding enumerated embodiment, wherein the method maintains the patient's serum bicarbonate within the range of 22 to 27 mEq/l.

### Embodiment 1172

The pharmaceutical composition for use in a method of treating an acid-base disorder of any preceding enumerated embodiment, wherein the method maintains the patient's serum bicarbonate within the range of 22 to 26 mEq/l.

### Embodiment 1173

The pharmaceutical composition for use in a method of treating an acid-base disorder of any preceding enumerated embodiment, wherein the method maintains the patient's serum bicarbonate within the range of 22 to 25 mEq/l.

### Embodiment 1174

The pharmaceutical composition for use in a method of treating an acid-base disorder of any preceding enumerated embodiment, wherein the method maintains the patient's serum bicarbonate within the range of 22 to 24 mEq/l.

### Embodiment 1175

The pharmaceutical composition for use in a method of treating an acid-base disorder of any preceding enumerated embodiment, wherein the method maintains the patient's serum bicarbonate within the range of 22 to 23 mEq/l.

### Embodiment 1176

The pharmaceutical composition for use in a method of treating an acid-base disorder of any preceding enumerated embodiment, wherein the method increases the patient's serum bicarbonate value by at least about 1 mEq/l relative to the patient's baseline serum bicarbonate value.

### Embodiment 1177

The pharmaceutical composition for use in a method of treating an acid-base disorder of any preceding enumerated embodiment, wherein the method increases the patient's serum bicarbonate value by at least about 1.5 mEq/l relative to the patient's baseline serum bicarbonate value.

### Embodiment 1178

The pharmaceutical composition for use in a method of treating an acid-base disorder of any preceding enumerated embodiment, wherein the method increases the patient's serum bicarbonate value by at least about 2 mEq/l relative to the patient's baseline serum bicarbonate value.

### Embodiment 1179

The pharmaceutical composition for use in a method of treating an acid-base disorder of any preceding enumerated embodiment, wherein the method increases the patient's serum bicarbonate value by at least about 2.5 mEq/l relative to the patient's baseline serum bicarbonate value.

### Embodiment 1180

The pharmaceutical composition for use in a method of treating an acid-base disorder of any preceding enumerated embodiment, wherein the method increases the patient's serum bicarbonate value by at least about 3 mEq/l relative to the patient's baseline serum bicarbonate value.

### Embodiment 1181

The pharmaceutical composition for use in a method of treating an acid-base disorder of any preceding enumerated embodiment, wherein the method increases the patient's serum bicarbonate value by at least about 3.5 mEq/l relative to the patient's baseline serum bicarbonate value.

### Embodiment 1182

The pharmaceutical composition for use in a method of treating an acid-base disorder of any preceding enumerated embodiment, wherein the method increases the patient's serum bicarbonate value by at least about 4 mEq/l relative to the patient's baseline serum bicarbonate value.

### Embodiment 1183

The pharmaceutical composition for use in a method of treating an acid-base disorder of any preceding enumerated embodiment, wherein the method increases the patient's serum bicarbonate value by at least about 4.5 mEq/l relative to the patient's baseline serum bicarbonate value.

### Embodiment 1184

The pharmaceutical composition for use in a method of treating an acid-base disorder of any preceding enumerated embodiment, wherein the method increases the patient's serum bicarbonate value by at least about 5 mEq/l relative to the patient's baseline serum bicarbonate value.

### Embodiment 1185

The pharmaceutical composition for use in a method of treating an acid-base disorder of any preceding enumerated embodiment, wherein the method increases the patient's serum bicarbonate value by at least about 5.5 mEq/l relative to the patient's baseline serum bicarbonate value.

### Embodiment 1186

The pharmaceutical composition for use in a method of treating an acid-base disorder of any preceding enumerated embodiment, wherein the method increases the patient's serum bicarbonate value by at least about 6 mEq/l relative to the patient's baseline serum bicarbonate value.

### Embodiment 1187

The pharmaceutical composition for use in a method of treating an acid-base disorder of any preceding enumerated embodiment, wherein the method increases the patient's serum bicarbonate value by at least about 6.5 mEq/l relative to the patient's baseline serum bicarbonate value.

### Embodiment 1188

The pharmaceutical composition for use in a method of treating an acid-base disorder of any preceding enumerated embodiment, wherein the method increases the patient's serum bicarbonate value by at least about 7 mEq/l relative to the patient's baseline serum bicarbonate value.

### Embodiment 1189

The pharmaceutical composition for use in a method of treating an acid-base disorder of any preceding enumerated embodiment, wherein the method increases the patient's serum bicarbonate value by at least about 7.5 mEq/l relative to the patient's baseline serum bicarbonate value.

### Embodiment 1190

The pharmaceutical composition for use in a method of treating an acid-base disorder of any preceding enumerated embodiment, wherein the method increases the patient's serum bicarbonate value by at least about 8 mEq/l relative to the patient's baseline serum bicarbonate value.

### Embodiment 1191

The pharmaceutical composition for use in a method of treating an acid-base disorder of any preceding enumerated embodiment, wherein the method increases the patient's serum bicarbonate value by at least about 8.5 mEq/l relative to the patient's baseline serum bicarbonate value.

### Embodiment 1192

The pharmaceutical composition for use in a method of treating an acid-base disorder of any preceding enumerated embodiment, wherein the method increases the patient's serum bicarbonate value by at least about 9 mEq/l relative to the patient's baseline serum bicarbonate value.

### Embodiment 1193

The pharmaceutical composition for use in a method of treating an acid-base disorder of any preceding enumerated embodiment, wherein the patient's increased serum bicarbonate value is sustained for a period of at least about one week.

### Embodiment 1194

The pharmaceutical composition for use in a method of treating an acid-base disorder of any preceding enumerated embodiment, wherein the patient's increased serum bicarbonate value is sustained for a period of about one week.

### Embodiment 1195

The pharmaceutical composition for use in a method of treating an acid-base disorder of any preceding enumerated embodiment, wherein the patient's increased serum bicarbonate value is sustained for a period of at least about two weeks.

### Embodiment 1196

The pharmaceutical composition for use in a method of treating an acid-base disorder of any preceding enumerated embodiment, wherein the patient's increased serum bicarbonate value is sustained for a period of about two weeks.

### Embodiment 1197

The pharmaceutical composition for use in a method of treating an acid-base disorder of any preceding enumerated embodiment, wherein the patient's increased serum bicarbonate value is sustained for a period of at least about three weeks.

### Embodiment 1198

The pharmaceutical composition for use in a method of treating an acid-base disorder of any preceding enumerated embodiment, wherein the patient's increased serum bicarbonate value is sustained for a period of about three weeks.

### Embodiment 1199

The pharmaceutical composition for use in a method of treating an acid-base disorder of any preceding enumerated embodiment, wherein the patient's increased serum bicarbonate value is sustained for a period of at least about four weeks.

### Embodiment 1200

The pharmaceutical composition for use in a method of treating an acid-base disorder of any preceding enumerated embodiment, wherein the patient's increased serum bicarbonate value is sustained for a period of about four weeks.

### Embodiment 1201

The pharmaceutical composition for use in a method of treating an acid-base disorder of any preceding enumerated embodiment, wherein the patient's increased serum bicarbonate value is sustained for a period of at least about five weeks.

### Embodiment 1202

The pharmaceutical composition for use in a method of treating an acid-base disorder of any preceding enumerated embodiment, wherein the patient's increased serum bicarbonate value is sustained for a period of about five weeks.

### Embodiment 1203

The pharmaceutical composition for use in a method of treating an acid-base disorder of any preceding enumerated embodiment, wherein the patient's increased serum bicarbonate value is sustained for a period of at least about six weeks.

### Embodiment 1204

The pharmaceutical composition for use in a method of treating an acid-base disorder of any preceding enumerated embodiment, wherein the patient's increased serum bicarbonate value is sustained for a period of about six weeks.

### Embodiment 1205

The pharmaceutical composition for use in a method of treating an acid-base disorder of any preceding enumerated embodiment, wherein the patient's increased serum bicarbonate value is sustained for a period of at least about seven weeks.

### Embodiment 1206

The pharmaceutical composition for use in a method of treating an acid-base disorder of any preceding enumerated embodiment, wherein the patient's increased serum bicarbonate value is sustained for a period of about seven weeks.

### Embodiment 1207

The pharmaceutical composition for use in a method of treating an acid-base disorder of any preceding enumerated embodiment, wherein the patient's increased serum bicarbonate value is sustained for a period of at least about eight weeks.

### Embodiment 1208

The pharmaceutical composition for use in a method of treating an acid-base disorder of any preceding enumerated embodiment, wherein the patient's increased serum bicarbonate value is sustained for a period of about eight weeks.

### Embodiment 1209

The pharmaceutical composition for use in a method of treating an acid-base disorder of any preceding enumerated embodiment, wherein the patient's increased serum bicarbonate value is sustained for a period of at least about nine weeks.

### Embodiment 1210

The pharmaceutical composition for use in a method of treating an acid-base disorder of any preceding enumerated embodiment, wherein the patient's increased serum bicarbonate value is sustained for a period of about nine weeks.

### Embodiment 1211

The pharmaceutical composition for use in a method of treating an acid-base disorder of any preceding enumerated embodiment, wherein the patient's increased serum bicarbonate value is sustained for a period of at least about ten weeks.

### Embodiment 1212

The pharmaceutical composition for use in a method of treating an acid-base disorder of any preceding enumerated embodiment, wherein the patient's increased serum bicarbonate value is sustained for a period of about ten weeks.

### Embodiment 1213

The pharmaceutical composition for use in a method of treating an acid-base disorder of any preceding enumerated embodiment, wherein the patient's increased serum bicarbonate value is sustained for a period of at least about 11 weeks.

### Embodiment 1214

The pharmaceutical composition for use in a method of treating an acid-base disorder of any preceding enumerated embodiment, wherein the patient's increased serum bicarbonate value is sustained for a period of about 11 weeks.

### Embodiment 1215

The pharmaceutical composition for use in a method of treating an acid-base disorder of any preceding enumerated embodiment, wherein the patient's increased serum bicarbonate value is sustained for a period of at least about 12 weeks.

### Embodiment 1216

The pharmaceutical composition for use in a method of treating an acid-base disorder of any preceding enumerated embodiment, wherein the patient's increased serum bicarbonate value is sustained for a period of about 12 weeks.

### Embodiment 1217

The pharmaceutical composition for use in a method of treating an acid-base disorder of any preceding enumerated embodiment, wherein the patient's increased serum bicarbonate value is sustained for a period of at least about 13 weeks.

### Embodiment 1218

The pharmaceutical composition for use in a method of treating an acid-base disorder of any preceding enumerated embodiment, wherein the patient's increased serum bicarbonate value is sustained for a period of about 13 weeks.

### Embodiment 1219

The pharmaceutical composition for use in a method of treating an acid-base disorder of any preceding enumerated embodiment, wherein the patient's increased serum bicarbonate value is sustained for a period of at least about 14, 15, 16, 17, 18, 19 or 20 weeks.

### Embodiment 1220

The pharmaceutical composition for use in a method of treating an acid-base disorder of any preceding enumerated embodiment, wherein the patient's increased serum bicarbonate value is sustained for a period of about 14, 15, 16, 17, 18, 19 or 20 weeks.

### Embodiment 1221

The pharmaceutical composition for use in a method of treating an acid-base disorder of any preceding enumerated embodiment, wherein the patient's increased serum bicarbonate value is sustained for a period of at least about six months.

### Embodiment 1222

The pharmaceutical composition for use in a method of treating an acid-base disorder of any preceding enumerated embodiment, wherein the patient's increased serum bicarbonate value is sustained for a period of about six months.

### Embodiment 1223

The pharmaceutical composition for use in a method of treating an acid-base disorder of any preceding enumerated embodiment, wherein the amount of the active ingredient of the pharmaceutical composition is about 1 g/day.

### Embodiment 1224

The pharmaceutical composition for use in a method of treating an acid-base disorder of any preceding enumerated embodiment, wherein the amount of the active ingredient of the pharmaceutical composition is about 1.5 g/day.

### Embodiment 1225

The pharmaceutical composition for use in a method of treating an acid-base disorder of any preceding enumerated embodiment, wherein the amount of the active ingredient of the pharmaceutical composition is about 2 g/day.

### Embodiment 1226

The pharmaceutical composition for use in a method of treating an acid-base disorder of any preceding enumerated embodiment, wherein the amount of the active ingredient of the pharmaceutical composition is about 2.5 g/day.

### Embodiment 1227

The pharmaceutical composition for use in a method of treating an acid-base disorder of any preceding enumerated embodiment, wherein the amount of the active ingredient of the pharmaceutical composition is about 3 g/day.

### Embodiment 1228

The pharmaceutical composition for use in a method of treating an acid-base disorder of any preceding enumerated embodiment, wherein the amount of the active ingredient of the pharmaceutical composition is about 3.5 g/day.

### Embodiment 1229

The pharmaceutical composition for use in a method of treating an acid-base disorder of any preceding enumerated embodiment, wherein the amount of the active ingredient of the pharmaceutical composition is about 4 g/day.

### Embodiment 1230

The pharmaceutical composition for use in a method of treating an acid-base disorder of any preceding enumerated embodiment, wherein the amount of the active ingredient of the pharmaceutical composition is about 4.5 g/day.

### Embodiment 1231

The pharmaceutical composition for use in a method of treating an acid-base disorder of any preceding enumerated embodiment, wherein the amount of the active ingredient of the pharmaceutical composition is about 5 g/day.

### Embodiment 1232

The pharmaceutical composition for use in a method of treating an acid-base disorder of any preceding enumerated embodiment, wherein the amount of the active ingredient of the pharmaceutical composition is about 5.5 g/day.

### Embodiment 1233

The pharmaceutical composition for use in a method of treating an acid-base disorder of any preceding enumerated embodiment, wherein the amount of the active ingredient of the pharmaceutical composition is about 6 g/day.

### Embodiment 1234

The pharmaceutical composition for use in a method of treating an acid-base disorder of any preceding enumerated embodiment, wherein the amount of the active ingredient of the pharmaceutical composition is about 6.5 g/day.

### Embodiment 1235

The pharmaceutical composition for use in a method of treating an acid-base disorder of any preceding enumerated embodiment, wherein the amount of the active ingredient of the pharmaceutical composition is about 7 g/day.

### Embodiment 1236

The pharmaceutical composition for use in a method of treating an acid-base disorder of any preceding enumerated embodiment, wherein the amount of the active ingredient of the pharmaceutical composition is about 7.5 g/day.

### Embodiment 1237

The pharmaceutical composition for use in a method of treating an acid-base disorder of any preceding enumerated embodiment, wherein the amount of the active ingredient of the pharmaceutical composition is about 8 g/day.

### Embodiment 1238

The pharmaceutical composition for use in a method of treating an acid-base disorder of any preceding enumerated embodiment, wherein the amount of the active ingredient of the pharmaceutical composition is about 8.5 g/day.

### Embodiment 1239

The pharmaceutical composition for use in a method of treating an acid-base disorder of any preceding enumerated embodiment, wherein the amount of the active ingredient of the pharmaceutical composition is about 9 g/day.

### Embodiment 1240

The pharmaceutical composition for use in a method of treating an acid-base disorder of any preceding enumerated embodiment, wherein the amount of the active ingredient of the pharmaceutical composition is about 9.5 g/day.

### Embodiment 1241

The pharmaceutical composition for use in a method of treating an acid-base disorder of any preceding enumerated embodiment, wherein the amount of the active ingredient of the pharmaceutical composition is about 10 g/day.

### Embodiment 1242

The pharmaceutical composition for use in a method of treating an acid-base disorder of any preceding enumerated embodiment, wherein the amount of the active ingredient of the pharmaceutical composition is about 15 g/day.

### Embodiment 1243

The pharmaceutical composition for use in a method of treating an acid-base disorder of any preceding enumerated embodiment, wherein the amount of the active ingredient of the pharmaceutical composition is about 20 g/day.

### Embodiment 1244

The pharmaceutical composition for use in a method of treating an acid-base disorder of any preceding enumerated embodiment, wherein the amount of the active ingredient of the pharmaceutical composition is about 25 g/day.

### Embodiment 1245

The pharmaceutical composition for use in a method of treating an acid-base disorder of any preceding enumerated embodiment, wherein the pharmaceutical compositions is administered to the patient in an oral dosage form once-a-day.

### Embodiment 1246

The pharmaceutical composition for use in a method of treating an acid-base disorder of any preceding enumerated embodiment, wherein the active ingredient is a composition as defined in any of the numbered embodiments in the description.

### Embodiment 1247

The pharmaceutical composition for use in a method of treating an acid-base disorder of any preceding enumerated embodiment, wherein the quality of life of the patient improves after treatment for a period of at least about one week.

### Embodiment 1248

The pharmaceutical composition for use in a method of treating an acid-base disorder of any preceding enumerated embodiment, wherein the quality of life of the patient improves after treatment for a period of about one week.

### Embodiment 1249

The pharmaceutical composition for use in a method of treating an acid-base disorder of any preceding enumerated embodiment, wherein the quality of life of the patient improves after treatment for a period of at least about two weeks.

### Embodiment 1250

The pharmaceutical composition for use in a method of treating an acid-base disorder of any preceding enumerated embodiment, wherein the quality of life of the patient improves after treatment for a period of about two weeks.

### Embodiment 1251

The pharmaceutical composition for use in a method of treating an acid-base disorder of any preceding enumerated embodiment, wherein the quality of life of the patient improves after treatment for a period of at least about three weeks.

### Embodiment 1252

The pharmaceutical composition for use in a method of treating an acid-base disorder of any preceding enumerated embodiment, wherein the quality of life of the patient improves after treatment for a period of about three weeks.

### Embodiment 1253

The pharmaceutical composition for use in a method of treating an acid-base disorder of any preceding enumerated embodiment, wherein the quality of life of the patient improves after treatment for a period of at least about four weeks.

### Embodiment 1254

The pharmaceutical composition for use in a method of treating an acid-base disorder of any preceding enumerated embodiment, wherein the quality of life of the patient improves after treatment for a period of about four weeks.

### Embodiment 1255

The pharmaceutical composition for use in a method of treating an acid-base disorder of any preceding enumerated embodiment, wherein the quality of life of the patient improves after treatment for a period of at least about five weeks.

### Embodiment 1256

The pharmaceutical composition for use in a method of treating an acid-base disorder of any preceding enumerated embodiment, wherein the quality of life of the patient improves after treatment for a period of about five weeks.

### Embodiment 1257

The pharmaceutical composition for use in a method of treating an acid-base disorder of any preceding enumerated embodiment, wherein the quality of life of the patient improves after treatment for a period of at least about six weeks.

### Embodiment 1258

The pharmaceutical composition for use in a method of treating an acid-base disorder of any preceding enumerated embodiment, wherein the quality of life of the patient improves after treatment for a period of about six weeks.

### Embodiment 1259

The pharmaceutical composition for use in a method of treating an acid-base disorder of any preceding enumerated embodiment, wherein the quality of life of the patient improves after treatment for a period of at least about seven weeks.

### Embodiment 1260

The pharmaceutical composition for use in a method of treating an acid-base disorder of any preceding enumerated embodiment, wherein the quality of life of the patient improves after treatment for a period of about seven weeks.

### Embodiment 1261

The pharmaceutical composition for use in a method of treating an acid-base disorder of any preceding enumerated embodiment, wherein the quality of life of the patient improves after treatment for a period of at least about eight weeks.

### Embodiment 1262

The pharmaceutical composition for use in a method of treating an acid-base disorder of any preceding enumerated embodiment, wherein the quality of life of the patient improves after treatment for a period of about eight weeks.

### Embodiment 1263

The pharmaceutical composition for use in a method of treating an acid-base disorder of any preceding enumerated embodiment, wherein the quality of life of the patient improves after treatment for a period of at least about nine weeks.

### Embodiment 1264

The pharmaceutical composition for use in a method of treating an acid-base disorder of any preceding enumerated embodiment, wherein the quality of life of the patient improves after treatment for a period of about nine weeks.

### Embodiment 1265

The pharmaceutical composition for use in a method of treating an acid-base disorder of any preceding enumerated embodiment, wherein the quality of life of the patient improves after treatment for a period of at least about ten weeks.

### Embodiment 1266

The pharmaceutical composition for use in a method of treating an acid-base disorder of any preceding enumerated embodiment, wherein the quality of life of the patient improves after treatment for a period of about ten weeks.

### Embodiment 1267

The pharmaceutical composition for use in a method of treating an acid-base disorder of any preceding enumerated embodiment, wherein the quality of life of the patient improves after treatment for a period of at least about 11 weeks.

### Embodiment 1268

The pharmaceutical composition for use in a method of treating an acid-base disorder of any preceding enumerated embodiment, wherein the quality of life of the patient improves after treatment for a period of about 11 weeks.

### Embodiment 1269

The pharmaceutical composition for use in a method of treating an acid-base disorder of any preceding enumerated embodiment, wherein the quality of life of the patient improves after treatment for a period of at least about 52 weeks. In other aspects of this embodiment, the quality of life of the patient improves after treatment for a period of at least about 40 weeks, at least about 26 weeks, or at least about 12 weeks. In other aspects of this embodiment, the quality of life of the patient improves after treatment for a period of at least about 12 weeks and after treatment for a period of at least about 40 weeks. In other aspects of this embodiment, the quality of life of the patient improves after treatment for a period of at least about 12 weeks and after a treatment period of at least about 52 weeks. In other aspects of this embodiment, the quality of life of the patient improves after treatment for a period of at least about 12 weeks, at least about 40 weeks and at least 52 weeks.

### Embodiment 1270

The pharmaceutical composition for use in a method of treating an acid-base disorder of any preceding enumerated embodiment, wherein the quality of life of the patient improves after treatment for a period of about 52 weeks. In other aspects of this embodiment, the quality of life of the patient improves after treatment for a period of at least about 40 weeks, at least about 26 weeks, or at least about 12 weeks. In other aspects of this embodiment, the quality of life of the patient improves after treatment for a period of at least about 12 weeks and at least about 40 weeks. In other aspects of this embodiment, the quality of life of the patient improves after treatment for a period of at least about 12 weeks and at least about 52 weeks. In other aspects of this embodiment, the quality of life of the patient improves after treatment for a period of at least about 12 weeks, at least about 40 weeks and least about 52 weeks.

### Embodiment 1271

The pharmaceutical composition for use in a method of treating an acid-base disorder of any preceding enumerated embodiment, wherein the quality of life of the patient improves after treatment for a period of at least about 13 weeks.

### Embodiment 1272

The pharmaceutical composition for use in a method of treating an acid-base disorder of any preceding enumerated embodiment, wherein the quality of life of the patient improves after treatment for a period of about 13 weeks.

### Embodiment 1273

The pharmaceutical composition for use in a method of treating an acid-base disorder of any preceding enumerated embodiment, wherein the quality of life of the patient improves after treatment for a period of at least about 14, 15, 16, 17, 18, 19 or 20 weeks.

### Embodiment 1274

The pharmaceutical composition for use in a method of treating an acid-base disorder of any preceding enumerated embodiment, wherein the quality of life of the patient improves after treatment for a period of about 14, 15, 16, 17, 18, 19 or 20 weeks.

### Embodiment 1275

The pharmaceutical composition for use in a method of treating an acid-base disorder of any preceding enumerated embodiment, wherein the quality of life of the patient improves after treatment for a period of at least about six months.

### Embodiment 1276

The pharmaceutical composition for use in a method of treating an acid-base disorder of any preceding enumerated embodiment, wherein the quality of life of the patient improves after treatment for a period of about six months.

### Embodiment 1277

The pharmaceutical composition for use in a method of treating an acid-base disorder of any preceding enumerated embodiment, wherein the physical function of the patient improves after treatment for a period of at least about one week.

### Embodiment 1278

The pharmaceutical composition for use in a method of treating an acid-base disorder of any preceding enumerated embodiment, wherein the physical function of the patient improves after treatment for a period of about one week.

### Embodiment 1279

The pharmaceutical composition for use in a method of treating an acid-base disorder of any preceding enumerated embodiment, wherein the physical function of the patient improves after treatment for a period of at least about two weeks.

### Embodiment 1280

The pharmaceutical composition for use in a method of treating an acid-base disorder of any preceding enumerated embodiment, wherein the physical function of the patient improves after treatment for a period of about two weeks.

### Embodiment 1281

The pharmaceutical composition for use in a method of treating an acid-base disorder of any preceding enumerated embodiment, wherein the physical function of the patient improves after treatment for a period of at about least three weeks.

### Embodiment 1282

The pharmaceutical composition for use in a method of treating an acid-base disorder of any preceding enumerated embodiment, wherein the physical function of the patient improves after treatment for a period of about three weeks.

### Embodiment 1283

The pharmaceutical composition for use in a method of treating an acid-base disorder of any preceding enumerated embodiment, wherein the physical function of the patient improves after treatment for a period of at least about four weeks.

### Embodiment 1284

The pharmaceutical composition for use in a method of treating an acid-base disorder of any preceding enumerated embodiment, wherein the physical function of the patient improves after treatment for a period of about four weeks.

### Embodiment 1285

The pharmaceutical composition for use in a method of treating an acid-base disorder of any preceding enumerated embodiment, wherein the physical function of the patient improves after treatment for a period of at least about five weeks.

### Embodiment 1286

The pharmaceutical composition for use in a method of treating an acid-base disorder of any preceding enumerated embodiment, wherein the physical function of the patient improves after treatment for a period of about five weeks.

### Embodiment 1287

The pharmaceutical composition for use in a method of treating an acid-base disorder of any preceding enumerated embodiment, wherein the physical function of the patient improves after treatment for a period of at least about six weeks.

### Embodiment 1289

The pharmaceutical composition for use in a method of treating an acid-base disorder of any preceding enumerated embodiment, wherein the physical function of the patient improves after treatment for a period of about six weeks.

### Embodiment 1290

The pharmaceutical composition for use in a method of treating an acid-base disorder of any preceding enumerated embodiment, wherein the physical function of the patient improves after treatment for a period of at least about seven weeks.

### Embodiment 1291

The pharmaceutical composition for use in a method of treating an acid-base disorder of any preceding enumerated embodiment, wherein the physical function of the patient improves after treatment for a period of about seven weeks.

### Embodiment 1292

The pharmaceutical composition for use in a method of treating an acid-base disorder of any preceding enumerated embodiment, wherein the physical function of the patient improves after treatment for a period of at least about eight weeks.

### Embodiment 1293

The pharmaceutical composition for use in a method of treating an acid-base disorder of any preceding enumerated embodiment, wherein the physical function of the patient improves after treatment for a period of about eight weeks.

### Embodiment 1294

The pharmaceutical composition for use in a method of treating an acid-base disorder of any preceding enumerated embodiment, wherein the physical function of the patient improves after treatment for a period of at least about nine weeks.

### Embodiment 1295

The pharmaceutical composition for use in a method of treating an acid-base disorder of any preceding enumerated embodiment, wherein the physical function of the patient improves after treatment for a period of about nine weeks.

### Embodiment 1296

The pharmaceutical composition for use in a method of treating an acid-base disorder of any preceding enumerated embodiment, wherein the physical function of the patient improves after treatment for a period of at least about ten weeks.

### Embodiment 1297

The pharmaceutical composition for use in a method of treating an acid-base disorder of any preceding enumerated embodiment, wherein the physical function of the patient improves after treatment for a period of about ten weeks.

### Embodiment 1298

The pharmaceutical composition for use in a method of treating an acid-base disorder of any preceding enumerated embodiment, wherein the physical function of the patient improves after treatment for a period of at least about 11 weeks.

### Embodiment 1299

The pharmaceutical composition for use in a method of treating an acid-base disorder of any preceding enumerated embodiment, wherein the physical function of the patient improves after treatment for a period of about 11 weeks.

### Embodiment 1300

The pharmaceutical composition for use in a method of treating an acid-base disorder of any preceding enumerated embodiment, wherein the physical function of the patient improves after treatment for a period of at least about 52 weeks. In other aspects of this embodiment, the quality of life of the patient improves after treatment for a period of at least about 40 weeks, at least about 26 weeks, or at least about 12 weeks. In other aspects of this embodiment, the quality of life of the patient improves after treatment for a period of at least about 12 weeks and after treatment for a period of at least about 40 weeks. In other aspects of this embodiment, the quality of life of the patient improves after treatment for a period of at least about 12 weeks and after treatment for a period of at least about 52 weeks. In other aspects of this embodiment, the quality of life of the patient improves after treatment for a period of at least about 12 weeks and the quality of life of the patient improves after treatment for a period of at least about 40 weeks and the quality of life of the patient improves after treatment for a period of at least about 52 weeks.

### Embodiment 1301

The pharmaceutical composition for use in a method of treating an acid-base disorder of any preceding enumerated embodiment, wherein the physical function of the patient improves after treatment for a period of about 52 weeks. In other aspects of this embodiment, the quality of life of the patient improves after treatment for a period of at least about 40 weeks, at least about 26 weeks, or at least about 12 weeks. In other aspects of this embodiment, the quality of life of the patient improves after treatment for a period of at least about 12 weeks and at least about 40 weeks. In other aspects of this embodiment, the quality of life of the patient improves after treatment for a period of at least about 12 weeks and at least about 52 weeks. In other aspects of this embodiment, the quality of life of the patient improves after treatment for a period of at least about 12 weeks, at least about 40 weeks, and at least about 52 weeks.

### Embodiment 1302

The pharmaceutical composition for use in a method of treating an acid-base disorder of any preceding enumerated embodiment, wherein the physical function of the patient improves after treatment for a period of at least about 13 weeks.

### Embodiment 1303

The pharmaceutical composition for use in a method of treating an acid-base disorder of any preceding enumerated embodiment, wherein the physical function of the patient improves after treatment for a period of about 13 weeks.

### Embodiment 1304

The pharmaceutical composition for use in a method of treating an acid-base disorder of any preceding enumerated embodiment, wherein the physical function of the patient improves after treatment for a period of at least about 14, 15, 16, 17, 18, 19 or 20 weeks.

### Embodiment 1305

The pharmaceutical composition for use in a method of treating an acid-base disorder of any preceding enumerated embodiment, wherein the physical function of the patient improves after treatment for a period of about 14, 15, 16, 17, 18, 19 or 20 weeks.

### Embodiment 1306

The pharmaceutical composition for use in a method of treating an acid-base disorder of any preceding enumerated embodiment, wherein the physical function of the patient improves after treatment for a period of at least about six months.

### Embodiment 1307

The pharmaceutical composition for use in a method of treating an acid-base disorder of any preceding enumerated embodiment, wherein the physical function of the patient improves after treatment for a period of about six months.

### Embodiment 1308

The pharmaceutical composition for use in a method of treating an acid-base disorder of any preceding enumerated embodiment, wherein the improvement in the quality of life of a patient can be determined by Quality of Life questionnaire.

### Embodiment 1309

The pharmaceutical composition for use in a method of treating an acid-base disorder of any preceding enumerated embodiment, wherein the improvement in the quality of life of a patient can be determined by comparing the scores of a Quality of Life questionnaire obtained before and after treatment.

### Embodiment 1310

The pharmaceutical composition for use in a method of treating an acid-base disorder of any preceding enumerated embodiment, wherein the improvement in the quality of life of a patient can be determined by a Kidney Disease Quality of Life (KDQOL) Physical Function Assessment.

### Embodiment 1311

The pharmaceutical composition for use in a method of treating an acid-base disorder of any preceding enumerated embodiment, wherein the improvement in the physical function of the patient can be determined by a Kidney Disease Quality of Life (KDQOL) Physical Function Assessment according to Goldfarb D. S. et al., Kidney International (2009) 75, 15-24.

### Embodiment 1312

The pharmaceutical composition for use in a method of treating an acid-base disorder of any preceding enumerated embodiment, wherein the improvement in the physical function of the patient can be determined by an increase the patient's physical function score based on answers to question 3 of the Kidney Disease Quality of Life Short Form (KDQOL SF) compared to the patient's baseline physical function score.

### Embodiment 1313

The pharmaceutical composition for use in a method of treating an acid-base disorder of any preceding enumerated embodiment, wherein the improvement in the physical function of the patient can be determined by an improvement in the patient's ability to perform vigorous activity, for example, one or more of running, lifting heavy objects, participating in strenuous sports.

### Embodiment 1314

The pharmaceutical composition for use in a method of treating an acid-base disorder of any preceding enumerated embodiment, wherein the improvement in the physical function of the patient can be determined by an improvement in the patient's ability to perform moderate activity, for example, one or more of moving a table, pushing a vacuum cleaner, bowling, or playing golf.

### Embodiment 1315

The pharmaceutical composition for use in a method of treating an acid-base disorder of any preceding enumerated embodiment, wherein the improvement in the physical function of the patient can be determined by an improvement in the patient's ability to lift or carry groceries, for example 1, 2, 3, 4 or 5 kg of groceries.

### Embodiment 1316

The pharmaceutical composition for use in a method of treating an acid-base disorder of any preceding enumerated embodiment, wherein the improvement in the physical function of the patient can be determined by an improvement in the patient's ability to climb several flights of stairs, for example 100 steps.

### Embodiment 1317

The pharmaceutical composition for use in a method of treating an acid-base disorder of any preceding enumerated embodiment, wherein the improvement in the physical function of the patient can be determined by an improvement in the patient's ability to climb one flight of stairs, for example 20 steps.

### Embodiment 1318

The pharmaceutical composition for use in a method of treating an acid-base disorder of any preceding enumerated embodiment, wherein the improvement in the physical function of the patient can be determined by an improvement in the patient's ability to bend, kneel, or stoop.

### Embodiment 1319

The pharmaceutical composition for use in a method of treating an acid-base disorder of any preceding enumerated embodiment, wherein the improvement in the physical function of the patient can be determined by an improvement in the patient's ability to walk more than a mile.

### Embodiment 1320

The pharmaceutical composition for use in a method of treating an acid-base disorder of any preceding enumerated embodiment, wherein the improvement in the physical function of the patient can be determined by an improvement in the patient's ability to walk one block, for example 500 feet.

### Embodiment 1321

The pharmaceutical composition for use in a method of treating an acid-base disorder of any preceding enumerated embodiment, wherein the improvement in the physical function of the patient can be determined by an improvement in the patient's ability to walk several blocks, for example 2000 feet.

### Embodiment 1322

The pharmaceutical composition for use in a method of treating an acid-base disorder of any preceding enumerated embodiment, wherein the improvement in the physical function of the patient can be determined by an improvement in the patient's ability to bath or dress themselves.

### Embodiment 1323

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 1 relative to the patient's baseline physical function score.

### Embodiment 1324

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 1.5 relative to the patient's baseline physical function score.

### Embodiment 1325

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 2 relative to the patient's baseline physical function score.

### Embodiment 1326

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 2.5 relative to the patient's baseline physical function score.

### Embodiment 1327

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 3 relative to the patient's baseline physical function score.

### Embodiment 1328

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 3.5 relative to the patient's baseline physical function score.

### Embodiment 1329

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 4 relative to the patient's baseline physical function score.

### Embodiment 1330

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 4.5 relative to the patient's baseline physical function score.

### Embodiment 1331

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 5 relative to the patient's baseline physical function score.

### Embodiment 1332

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 5.5 relative to the patient's baseline physical function score.

### Embodiment 1333

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 6 relative to the patient's baseline physical function score.

### Embodiment 1334

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 6.5 relative to the patient's baseline physical function score.

### Embodiment 1335

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 7 relative to the patient's baseline physical function score.

### Embodiment 1336

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 7.5 relative to the patient's baseline physical function score.

### Embodiment 1337

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 8 relative to the patient's baseline physical function score.

### Embodiment 1338

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 8.5 relative to the patient's baseline physical function score.

### Embodiment 1339

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 9 relative to the patient's baseline physical function score.

### Embodiment 1340

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 10 relative to the patient's baseline physical function score.

### Embodiment 1341

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 11 relative to the patient's baseline physical function score.

### Embodiment 1342

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 12 relative to the patient's baseline physical function score.

### Embodiment 1343

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 13 relative to the patient's baseline physical function score.

### Embodiment 1344

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 14 relative to the patient's baseline physical function score.

### Embodiment 1345

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 15 relative to the patient's baseline physical function score.

### Embodiment 1346

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 20 relative to the patient's baseline physical function score.

### Embodiment 1347

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 25 relative to the patient's baseline physical function score.

### Embodiment 1348

The pharmaceutical composition for use in a method of treating an acid-base disorder of any preceding enumerated embodiment, wherein the patient's physical function score based on answers to question 3 of the Kidney Disease Quality of Life Short Form (KDQOL SF) is improved by at least about 30 relative to the patient's baseline physical function score.

### Embodiment 1349

The pharmaceutical composition for use in a method of treating an acid-base disorder of any preceding enumerated embodiment, wherein the improvement in patient's physical function score is achieved at the end of the time period stated in any preceding enumerated embodiment.

### Embodiment 1350

The pharmaceutical composition for use in a method of treating an acid-base disorder of any preceding enumerated embodiment, wherein the improvement in the physical function of the patient can be determined by a repeated chair stand test.

### Embodiment 1351

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1 second relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, in the Repeated Chair Stand Test, after about twelve weeks of treatment, there is a trend toward significance for the difference between treated and placebo-treated patients. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks, and at least about 52 weeks. In another aspect of this and other embodiments, the improvement in the patient's baseline repeated chair stand times is seen after treatment for a period of at least about 12 weeks, at least about 40 weeks, and least about 52 weeks.

### Embodiment 1352

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.1 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks, and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks, and at least about 52 weeks. In another aspect of this and other embodiments, in the Repeated Chair Stand Test, after about twelve weeks of treatment, there is a trend toward significance for the difference between treated and placebo-treated patients.

### Embodiment 1353

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.2 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks, and at least about 52 weeks. In another aspect of this and other embodiments, in the Repeated Chair Stand Test, after about twelve weeks of treatment, there is a trend toward significance for the difference between treated and placebo-treated patients.

### Embodiment 1354

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.3 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks, and at least about 52 weeks. In another aspect of this and other embodiments, in the Repeated Chair Stand Test, after about twelve weeks of treatment, there is a trend toward significance for the difference between treated and placebo-treated patients.

### Embodiment 1355

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.4 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1356

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.5 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1357

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.6 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In one aspect of this and other embodiments, the improvement in the patient's baseline repeated chair stand times is seen after treatment for a period of at least about 12 weeks and at least about 40 weeks. In one aspect of this and other embodiments, the improvement in the patient's baseline repeated chair stand times is seen after treatment for a period of at least about 12 weeks and at least about 52 weeks. In one aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1358

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.7 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1359

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.8 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1360

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 1.9 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1361

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 2 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1362

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 3 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1363

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 4 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1364

The pharmaceutical composition for use in a method of treating an acid-base disorder of any preceding enumerated embodiment, wherein the improvement in the physical function of the patient is determined by a repeated chair stand test, wherein the time for the patient to complete 5 chair sit-to-stand repetitions improves by at least about 5 seconds relative to baseline. In one aspect of this and other embodiments, the improvement in the patient's baseline repeated chair stand times is seen after treatment for a period of at least about 52 weeks, at least about 40 weeks, at least about 26 weeks, or at least about 12 weeks. In another aspect of this and other embodiments, the improvement in the patient's baseline repeated chair stand times is seen after treatment for a period of at least about 12 weeks and at least about 40 weeks. In another aspect of this and other embodiments, the improvement in the patient's baseline repeated chair stand times is seen after treatment for a period of at least about 12 weeks and at least about 52 weeks. In another aspect of this and other embodiments, the improvement in the patient's baseline repeated chair stand times is seen after treatment for a period of at least about 12 weeks, at least about 40 weeks and at least about 52 weeks. In another aspect of this and other embodiments, in the Repeated Chair Stand Test, after about twelve weeks of treatment, there is a trend toward significance for the difference between treated and placebo-treated patients.

### Embodiment 1365

The pharmaceutical composition for use in a method of treating an acid-base disorder of any preceding enumerated embodiment, wherein the improvement in the time taken for the patient to complete 5 chair sit-to-stand repetitions is achieved at the end of the time period stated in any preceding enumerated embodiment.

### Embodiment 1366

The pharmaceutical composition for use in a method of treating an acid-base disorder of any preceding enumerated embodiment, wherein in said method of treating the quality of life of the patient is determined by one or more of the approaches stated in the claims.

### Embodiment 1367

The pharmaceutical composition for use in a method of treating an acid-base disorder of any preceding enumerated embodiment, wherein in said method of treating the physical function of the patient is determined by one or more of the approaches stated in the claims.

### Embodiment 1368

The method/composition of any preceding enumerated embodiment, wherein the improvement of the physical function comprises an improvement in the patient's baseline physical function score of at least 1.5 points based on the patient's answers to question 3 of the Kidney Disease Quality of Life Short Form (KDQOL-SF).

### Embodiment 1369

The method/composition of any preceding enumerated embodiment, wherein the improvement of the physical function comprises an improvement in the patient's baseline repeated chair stand times of at least −1.5 seconds.

### Embodiment 1370

The method/composition of any preceding enumerated embodiment, wherein the improvement of the physical function comprises an improvement in the patient's baseline physical function score of at least 1.5 points based on the patient's answers to question 3 of the KDQOL-SF and an improvement in the patient's baseline repeated chair stand times of at least −1.5 seconds.

### Embodiment 1371

The method/composition of any preceding enumerated embodiment, wherein the improvement in patient's baseline repeated chair stand times is seen after treatment for a period of at least about 12 weeks.

### Embodiment 1372

The method/composition of any preceding enumerated embodiment, wherein the improvement in patient's baseline repeated chair stand times is seen after treatment for a period of at least about 26 weeks.

### Embodiment 1373

The method/composition of any preceding enumerated embodiment, wherein the improvement in patient's baseline repeated chair stand times is seen after treatment for a period of at least about 40 weeks.

### Embodiment 1374

The method/composition of any preceding enumerated embodiment, wherein the improvement in patient's baseline repeated chair stand times is seen after treatment for a period of at least about 52 weeks.

### Embodiment 1375

The method/composition of any preceding enumerated embodiment, wherein the improvement in patient's baseline repeated chair stand times is seen after treatment for a period of at least about 12 weeks and at least about 40 weeks.

### Embodiment 1376

The method/composition of any preceding enumerated embodiment, wherein the improvement in patient's baseline repeated chair stand times is seen after treatment for a period of at least about 12 weeks and at least about 52 weeks.

### Embodiment 1377

The method/composition of any preceding enumerated embodiment, wherein the improvement in patient's baseline repeated chair stand times is seen after treatment for a period of at least about 12 weeks, at least about 40 weeks and at least about 52 weeks.

### Embodiment 1378

The method/composition of any preceding enumerated embodiment, wherein the improvement in patient's quality of life is seen after treatment for a period of at least about 12 weeks.

### Embodiment 1379

The method/composition of any preceding enumerated embodiment, wherein the improvement in patient's quality of life is seen after treatment for a period of at least about 26 weeks.

### Embodiment 1380

The method/composition of any preceding enumerated embodiment, wherein the improvement in patient's quality of life is seen after treatment for a period of at least about 40 weeks.

### Embodiment 1381

The method/composition of any preceding enumerated embodiment, wherein the improvement in patient's quality of life is seen after treatment for a period of at least about 52 weeks.

### Embodiment 1382

The method/composition of any preceding enumerated embodiment, wherein the improvement in patient's quality of life is seen after treatment for a period of at least about 12 weeks and at least about 40 weeks.

### Embodiment 1383

The method/composition of any preceding enumerated embodiment, wherein the improvement in patient's quality of life is seen after treatment for a period of at least about 12 weeks and at least about 52 weeks.

### Embodiment 1384

The method/composition of any preceding enumerated embodiment, wherein the improvement in patient's quality of life is seen after treatment for a period of at least about 12 weeks, at least about 40 weeks and at least about 52 weeks.

### Embodiment 1385

The method/composition of any preceding enumerated embodiment, wherein the improvement in patient's physical function is seen after treatment for a period of at least about 12 weeks.

### Embodiment 1386

The method/composition of any preceding enumerated embodiment, wherein the improvement in patient's physical function is seen after treatment for a period of at least about 26 weeks.

### Embodiment 1387

The method/composition of any preceding enumerated embodiment, wherein the improvement in patient's physical function is seen after treatment for a period of at least about 40 weeks.

### Embodiment 1388

The method/composition of any preceding enumerated embodiment, wherein the improvement in patient's physical function is seen after treatment for a period of at least about 52 weeks.

### Embodiment 1389

The method/composition of any preceding enumerated embodiment, wherein the improvement in patient's physical function is seen after treatment for a period of at least about 12 weeks and at least about 40 weeks.

### Embodiment 1390

The method/composition of any preceding enumerated embodiment, wherein the improvement in patient's physical function is seen after treatment for a period of at least about 12 weeks and at least about 52 weeks.

### Embodiment 1391

The method/composition of any preceding enumerated embodiment, wherein the improvement in patient's physical function is seen after treatment for a period of at least about 12 weeks, at least about 40 weeks and at least about 52 weeks.

### Embodiment 1392

The method/composition of any preceding enumerated embodiment, wherein the increase in patient's baseline serum bicarbonate value is seen after treatment for a period of at least about 12 weeks.

### Embodiment 1393

The method/composition of any preceding enumerated embodiment, wherein the improvement in patient's baseline serum bicarbonate value is seen after treatment for a period of at least about 26 weeks.

### Embodiment 1394

The method/composition of any preceding enumerated embodiment, wherein the improvement in patient's baseline serum bicarbonate value is seen after treatment for a period of at least about 40 weeks.

### Embodiment 1395

The method/composition of any preceding enumerated embodiment, wherein the improvement in patient's baseline serum bicarbonate value is seen after treatment for a period of at least about 52 weeks.

### Embodiment 1396

The method/composition of any preceding enumerated embodiment, wherein the improvement in patient's baseline serum bicarbonate value is seen after treatment for a period of at least about 12 weeks and at least about 40 weeks.

### Embodiment 1397

The method/composition of any preceding enumerated embodiment, wherein the improvement in patient's baseline serum bicarbonate value is seen after treatment for a period of at least about 12 weeks and at least about 52 weeks.

### Embodiment 1398

The method/composition of any preceding enumerated embodiment, wherein the improvement in patient's baseline serum bicarbonate value is seen after treatment for a period of at least about 12 weeks, at least about 40 weeks and at least about 52 weeks.

### Embodiment 1399

The method/composition of any preceding enumerated embodiment wherein the composition comprises a proton-binding, crosslinked amine polymer comprising the residues of allylamine (2-propen-1-ylamine), diallylpropyldiamine (1,3-bis(allylamino)propane) and 1,2-dichloroethane. wherein the composition comprises a proton-binding, crosslinked amine polymer comprising the residue of allylamine (2-propen-1-ylamine).

### Embodiment 1401

The method/composition of any preceding enumerated embodiment wherein the composition comprises a proton-binding, crosslinked amine polymer comprising the residue of diallylpropyldiamine (1,3-bis(allylamino)propane).

### Embodiment 1402

The method/composition of any preceding enumerated embodiment wherein the composition comprises a proton-binding, crosslinked amine polymer comprising the residue of 1,2-dichloroethane.

### Embodiment 1403

The method/composition of any preceding enumerated embodiment wherein the composition comprises a proton-binding, crosslinked amine polymer consisting essentially of the residues of allylamine (2-propen-1-ylamine), diallylpropyldiamine (1,3-bis(allylamino)propane) and 1,2-dichloroethane.

### Embodiment 1404

The method/composition of any preceding enumerated embodiment wherein the composition comprises a 1,3-propanediamine, N¹,N³-di-2-propen-1-yl-, polymer with 1,2-dichloroethane and 2-propen-1-amine.

### Embodiment 1405

The method/composition of any preceding enumerated embodiment wherein the composition comprises a N¹,N³-bis(prop-2-en-1-yl)propan-1,3-diamine copolymer with 1,2-dichloroethane and prop-2-en-1-amine.

### Embodiment 1406

The method/composition of any preceding enumerated embodiment wherein the composition comprises a proton-binding, crosslinked amine polymer comprising the residues of

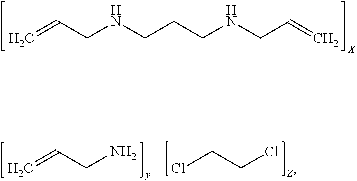

wherein x, y and z are positive integers.

### Embodiment 1407

The method/composition of any preceding enumerated embodiment wherein the composition comprises a proton-binding, crosslinked amine polymer comprising the molecular formula [C₉H₁₈N₂]ₓ.[C₃H₇N]_{y}.[C₂H₄Cl₂]_{z}, wherein x, y and z are positive integers.

### Embodiment 1408

A pharmaceutical composition for use in a method of treating an acid-base disorder in a patient, wherein the method increases the serum bicarbonate value of the patient from the baseline serum bicarbonate value to an increased serum bicarbonate value after treatment with the pharmaceutical composition for a period of at least about 1, 2, 4, 6, 8, 10, 12, 14, 16, 20, 24, 28, 34, 40, 46, 52, 53 and/or 54 weeks.

### Embodiment 1409

The method/composition of enumerated embodiment 1408, wherein the method/composition is the method/composition described in any preceding enumerated embodiment.

### Embodiment 1410

The method/composition of any preceding enumerated embodiment, wherein the increase in serum bicarbonate level is at least about 4 mEq/L after treatment for a period of at least about 4 weeks.

### Embodiment 1411

The method/composition of any preceding enumerated embodiment, wherein the increase in serum bicarbonate level is at least about 4 mEq/L after treatment for a period of at least about 6 weeks.

### Embodiment 1412

The method/composition of any preceding enumerated embodiment, wherein the increase in serum bicarbonate level is at least about 4 mEq/L after treatment for a period of at least about 8 weeks.

### Embodiment 1413

The method/composition of any preceding enumerated embodiment, wherein the increase in serum bicarbonate level is at least about 4 mEq/L after treatment for a period of at least about 10 weeks.

### Embodiment 1414

The method/composition of any preceding enumerated embodiment, wherein the increase in serum bicarbonate level is at least about 4 mEq/L after treatment for a period of at least about 12 weeks.

### Embodiment 1415

The method/composition of any preceding enumerated embodiment, wherein the increase in serum bicarbonate level is at least about 4 mEq/L after treatment for a period of at least about 14 weeks.

### Embodiment 1416

The method/composition of any preceding enumerated embodiment, wherein the increase in serum bicarbonate level is at least about 4 mEq/L after treatment for a period of at least about 16 weeks.

### Embodiment 1417

The method/composition of any preceding enumerated embodiment, wherein the increase in serum bicarbonate level is at least about 4 mEq/L after treatment for a period of at least about 20 weeks.

### Embodiment 1418

The method/composition of any preceding enumerated embodiment, wherein the increase in serum bicarbonate level is at least about 4 mEq/L after treatment for a period of at least about 24 weeks.

### Embodiment 1419

The method/composition of any preceding enumerated embodiment, wherein the increase in serum bicarbonate level is at least about 4 mEq/L after treatment for a period of at least about 28 weeks.

### Embodiment 1420

The method/composition of any preceding enumerated embodiment, wherein the increase in serum bicarbonate level is at least about 4 mEq/L after treatment for a period of at least about 34 weeks.

### Embodiment 1421

The method/composition of any preceding enumerated embodiment, wherein the increase in serum bicarbonate level is at least about 4 mEq/L after treatment for a period of at least about 40 weeks.

### Embodiment 1422

The method/composition of any preceding enumerated embodiment, wherein the increase in serum bicarbonate level is at least about 4 mEq/L after treatment for a period of at least about 46 weeks.

### Embodiment 1423

The method/composition of any preceding enumerated embodiment, wherein the increase in serum bicarbonate level is at least about 4 mEq/L after treatment for a period of at least about 52 weeks.

### Embodiment 1424

The method/composition of any preceding enumerated embodiment, wherein the increase in serum bicarbonate level is at least about 4 mEq/L after treatment for a period of at least about 4, 6, 8, 10, 12, 14, 16, 20, 24, 28, 34, 40, 46 and 52 weeks.

### Embodiment 1425

The method/composition of any preceding enumerated embodiment, wherein the patient achieves at least about a 6 point improvement on the KDQOL-Physical Functioning Domain relative to the placebo control after treatment for a period of at least about 12 weeks.

### Embodiment 1426

The method/composition of any preceding enumerated embodiment, wherein the patient achieves at least about a 7 point improvement on the KDQOL-Physical Functioning Domain relative to the placebo control after treatment for a period of at least about 40 weeks.

### Embodiment 1427

The method/composition of any preceding enumerated embodiment, wherein the patient achieves at least about a 11 point improvement on the KDQOL-Physical Functioning Domain relative to the placebo control after treatment for a period of at least about 52 weeks.

### Embodiment 1428

The method/composition of any preceding enumerated embodiment, wherein the time for the patient to complete 5 chair sit-to-stand repetitions improves by at least about 2 seconds relative to baseline, wherein the improvement in the patient's baseline repeated chair stand times is seen after treatment for a period of at least about 12 weeks.

### Embodiment 1429

The method/composition of any preceding enumerated embodiment, wherein the time for the patient to complete 5 chair sit-to-stand repetitions improves by at least about 3 seconds relative to baseline, wherein the improvement in the patient's baseline repeated chair stand times is seen after treatment for a period of at least about 40 weeks.

### Embodiment 1430

The method/composition of any preceding enumerated embodiment, wherein the time for the patient to complete 5 chair sit-to-stand repetitions improves by at least about 4 seconds relative to baseline, wherein the improvement in the patient's baseline repeated chair stand times is seen after treatment for a period of at least about 52 weeks.

### Embodiment 1431

A method of treating an individual afflicted with chronic kidney disease, the method comprising administering the composition described in any preceding enumerated embodiment.

### Embodiment 1432

The method of enumerated embodiment 1431 wherein the method of treatment is the method of treatment described in any preceding enumerated embodiment.

### Embodiment 1433

The method of enumerated embodiment 1431 or 1432 wherein the rate of progression of the individual's chronic kidney disease is decreased.

### Embodiment 1434

The method of enumerated embodiment 1433, wherein the rate of progression decreases for at least about 1 month.

### Embodiment 1435

The method of enumerated embodiment 1433, wherein the rate of progression decreases for at least about 4 months.

### Embodiment 1436

The method of enumerated embodiment 1433, wherein the rate of progression decreases for at least about 6 months.

### Embodiment 1437

The method of enumerated embodiment 1433, wherein the rate of progression decreases for at least about 12 months.

### Embodiment 1438

A method of decreasing the rate of progression of chronic kidney disease in an individual, the method comprising administering the composition described in any preceding enumerated embodiment.

### Embodiment 1439

The method of enumerated embodiment 1438 wherein the method includes the method of treatment described in any preceding enumerated embodiment.

### Embodiment 1440

The method of any one of enumerated embodiments 1432-1439 wherein the individual is afflicted with metabolic acidosis.

### Embodiment 1441

The method of enumerated embodiment 1440 wherein the metabolic acidosis is eubicarbonatemic metabolic acidosis.

### Embodiment 1442

The method of enumerated embodiment 1440 wherein the metabolic acidosis is characterized by a serum bicarbonate level of about 22-24 mEq/L.

### Embodiment 1443

The method of enumerated embodiment 1440 wherein the metabolic acidosis is characterized by a urine citrate excretion level of about 180-370 mg/day.

### Embodiment 1444

The method of enumerated embodiment 1440 wherein the metabolic acidosis is characterized by a urine ammonium excretion level of about 200 mmol/day.

### Embodiment 1445

The method of enumerated embodiment 1440 wherein the metabolic acidosis is characterized by a net acid excretion (NAE) level of less than about 50 mEq/day.

### Embodiment 1446

The method of enumerated embodiment 1440 wherein the metabolic acidosis is characterized by a plasma endothelin (ET-1) level of about 2-5 pg/mL.

### Embodiment 1447

The method of enumerated embodiment 1440 wherein the metabolic acidosis is characterized by a urine endothelin level (ET-1) to creatine (ET-1/creatine) ratio of greater than about 4.

### Embodiment 1448

The method of enumerated embodiment 1440 wherein the metabolic acidosis is characterized by a plasma aldosterone level of about 9-64 ng/dL in adults lying down.

### Embodiment 1449

The method of enumerated embodiment 1440 wherein the metabolic acidosis is characterized by a plasma aldosterone level of about 9-140 ng/dL in children lying down.

### Embodiment 1450

The method of any one of enumerated embodiments 1440-1449 wherein the metabolic acidosis is characterized by a blood serum or blood plasma bicarbonate value not in excess of about 25 mEq/l.

### Embodiment 1451

The method of any one of enumerated embodiments 1440-1449 wherein the metabolic acidosis is characterized by a blood serum or blood plasma bicarbonate value not in excess of about 24 mEq/l.

### Embodiment 1452

The method of any one of enumerated embodiments 1440-1449 wherein the metabolic acidosis is characterized by a blood serum or blood plasma bicarbonate value not in excess of about 23 mEq/l.

### Embodiment 1453

The method of any one of enumerated embodiments 1432-1439 and 1440-1452 wherein the metabolic acidosis is characterized by a blood serum or blood plasma bicarbonate value of less than about 22 mEq/l.

### Embodiment 1454

The method of any one of enumerated embodiments 1450-1453 wherein the metabolic acidosis is characterized by a blood serum bicarbonate value.

### Embodiment 1455

The method of any one of enumerated embodiments 1450-1453 wherein the metabolic acidosis is characterized by a blood plasma bicarbonate value.

### Embodiment 1456

The method of any one of enumerated embodiments 1433-1455 wherein the rate of decrease in the progression of chronic kidney disease is measurable by a decreased rate of change in eGFR.

### Embodiment 1457

The method of enumerated embodiment 1456 wherein the individual or adult human patient has a baseline eGFR value of at least about 15 mL/min/1.73 m².

### Embodiment 1458

The method of enumerated embodiment 1456 wherein the individual or adult human patient has a baseline eGFR value of at least about 30 mL/min/1.73 m².

### Embodiment 1459

The method of enumerated embodiment 1456 wherein the individual or adult human patient has a baseline eGFR value of less than about 45 mL/min/1.73 m² for at least three months.

### Embodiment 1460

The method of enumerated embodiment 1456 wherein the individual or adult human patient has a baseline eGFR value of less than about 60 mL/min/1.73 m² for at least three months.

### Embodiment 1461

The method of any one of enumerated embodiments 1456-1460 wherein the decreased rate of change in eGFR value is less than about 1 mL/min/1.73 m² over a period of about 1 month.

### Embodiment 1462

The method of any one of enumerated embodiments 1456-1460 wherein the decreased rate of change in eGFR value is less than about 5 mL/min/1.73 m² over a period of about 1 month.

### Embodiment 1463

The method of any one of enumerated embodiments 1456-1460 wherein the decreased rate of change in eGFR value is less than about 10 mL/min/1.73 m² over a period of about 1 month.

### Embodiment 1464

The method of any one of enumerated embodiments 1456-1460 wherein the decreased rate of change in eGFR value is less than about 15 mL/min/1.73 m² over a period of about 1 month.

### Embodiment 1465

The method of any one of enumerated embodiments 1456-1460 wherein the decreased rate of change in eGFR value is less than about 20 mL/min/1.73 m² over a period of about 1 month.

### Embodiment 1466

The method of any one of enumerated embodiments 1456-1460 wherein the decreased rate of change in eGFR value is less than about 25 mL/min/1.73 m² over a period of about 1 month.

### Embodiment 1467

The method of any one of enumerated embodiments 1456-1466 wherein the decreased rate of change in eGFR occurs to the extent that eGFR stops decreasing.

### Embodiment 1468

The method of any one of enumerated embodiments 1456-1466 wherein the decreased rate of change in eGFR occurs to the extent that there is an improvement in eGFR.

### Embodiment 1469

The method of any one of enumerated embodiments 1433-1466 wherein the delay in the progression of chronic kidney disease is measurable by reduced change in mGFR, or a halt in change to mGFR, or improvement in mGFR.

### Embodiment 1470

The method of enumerated embodiment 1469 wherein the individual or adult human patient has a baseline mGFR value of at least about 15 mL/min/1.73 m².

### Embodiment 1471

The method of enumerated embodiment 1469 wherein the individual or adult human patient has a baseline mGFR value of at least about 30 mL/min/1.73 m².

### Embodiment 1472

The method of enumerated embodiment 1469 wherein the individual or adult human patient has a baseline mGFR value of less than about 45 mL/min/1.73 m² for at least three months.

### Embodiment 1473

The method of enumerated embodiment 1469 wherein the individual or adult human patient has a baseline mGFR value of less than about 60 mL/min/1.73 m² for at least three months.

### Embodiment 1474

The method of any one of enumerated embodiments 1469-1473 wherein the decreased rate of change in mGFR value is less than about 1 mL/min/1.73 m² over a period of about 1 month.

### Embodiment 1475

The method of any one of enumerated embodiments 1469-1473 wherein the decreased rate of change in mGFR value is less than about 5 mL/min/1.73 m² over a period of about 1 month.

### Embodiment 1476

The method of any one of enumerated embodiments 1456-1475, wherein the outcome specified as being measurable is actually measured as part of the method of treatment.

### Embodiment 1477

The method of any one of enumerated embodiments 1433-1476 wherein the delay in the progression of chronic kidney disease includes the individual's stage of chronic kidney disease remaining constant.

### Embodiment 1478

The method of enumerated embodiment 1477, wherein the patient remains at stage 1 of chronic kidney disease.

### Embodiment 1479

The method of enumerated embodiment 1477, wherein the patient remains at stage 2 of chronic kidney disease.

### Embodiment 1480

The method of enumerated embodiment 1477, wherein the patient remains at stage 3A of chronic kidney disease.

### Embodiment 1481

The method of enumerated embodiment 1477, wherein the patient remains at stage 3B of chronic kidney disease.

### Embodiment 1482

The method of enumerated embodiment 1477, wherein the patient remains at stage 4 of chronic kidney disease.

### Embodiment 1483

The method of enumerated embodiment 1477, wherein the patient remains at stage 5 of chronic kidney disease.

### Embodiment 1484

The method of any one of enumerated embodiments 1478-1483, wherein the patient remains at the claimed stage of chronic kidney disease for at least about 1 month.

### Embodiment 1485

The method of any one of enumerated embodiments 1478-1483, wherein the patient remains at the claimed stage of chronic kidney disease for at least about 4 months.

### Embodiment 1486

The method of any one of enumerated embodiments 1478-1483, wherein the patient remains at the claimed stage of chronic kidney disease for at least about 6 months.

### Embodiment 1487

The method of any one of enumerated embodiments 1478-1483, wherein the patient remains at the claimed stage of chronic kidney disease for at least about 12 months.

### Embodiment 1488

The method of any one of enumerated embodiments 1432-1487 wherein the individual does not require dialysis to manage their chronic kidney disease during the method.

### Embodiment 1489

The method of enumerated embodiment 1488, where the individual did not require dialysis when starting treatment.

### Embodiment 1490

The method or composition of any preceding enumerated embodiment, wherein the composition binds less than about 5 mmol/g of fatty acids.

### Embodiment 1491

The method or composition of any preceding enumerated embodiment, wherein the composition binds less than about 4 mmol/g of fatty acids.

### Embodiment 1492

The method or composition of any preceding enumerated embodiment, wherein the composition binds less than about 3 mmol/g of fatty acids.

### Embodiment 1493

The method or composition of any preceding enumerated embodiment, wherein the composition binds less than about 2 mmol/g of fatty acids.

### Embodiment 1494

The method or composition of any preceding enumerated embodiment, wherein the composition binds less than about 1 mmol/g of fatty acids.

### Embodiment 1495

The method or composition of any preceding enumerated embodiment, wherein the composition binds less than about 0.3 mmol/g of fatty acids.

### Embodiment 1496

The method or composition of any preceding enumerated embodiment, wherein the composition binds less than about 0.1 mmol/g of fatty acids.

### Embodiment 1497

The method or composition of any one of enumerated embodiments 1490-1496, wherein the fatty acid is a short chain fatty acid, such as formic acid, acetic acid, propionic acid, butyric acid, isobutyric acid, valeric acid, isovaleric acid, or lactic acid.

### Embodiment 1498

The method or composition of any preceding enumerated embodiment, wherein the individual's or adult human patient's baseline blood serum or blood plasma sodium level is unchanged during treatment.

### Embodiment 1499

The method or composition of any preceding enumerated embodiment, wherein the individual's or adult human patient's baseline blood serum or blood plasma magnesium level is unchanged during treatment.

### Embodiment 1500

The method or composition of any preceding enumerated embodiment, wherein the individual's or adult human patient's baseline blood serum or blood plasma calcium level is unchanged during treatment.

### Embodiment 1501

The method or composition of any preceding enumerated embodiment, wherein the individual's or adult human patient's baseline blood serum or blood plasma potassium level is unchanged during treatment.

### Embodiment 1502

The method of any of enumerated embodiments 1498-1501 wherein the specified baseline blood serum level is unchanged.

### Embodiment 1503

The method of any of enumerated embodiments 1498-1501 wherein the specified baseline blood plasma level is unchanged.

### Embodiment 1504

A nonabsorbable composition for use in a method of treating an acid-base disorder in a patient, wherein the nonabsorbable composition has an equilibrium proton binding capacity of at least 5 mmol/g and a chloride ion binding capacity of at least 5 mmol/g in an aqueous simulated gastric fluid buffer (“SGF”) containing 35 mM NaCl and 63 mM HCl at pH 1.2 and 37° C.

### Embodiment 1505

The nonabsorbable composition of enumerated embodiment 1504, wherein the method/composition is the method/composition described in any preceding enumerated embodiment.

### Embodiment 1506

The nonabsorbable composition of enumerated embodiment 1504 or 1505, wherein the nonabsorbable composition has an equilibrium proton binding capacity of at least 10 mmol/g and a chloride ion binding capacity of at least 10 mmol/g in an aqueous simulated gastric fluid buffer (“SGF”) containing 35 mM NaCl and 63 mM HCl at pH 1.2 and 37° C.

### Embodiment 1507

The nonabsorbable composition of enumerated embodiment 1504 or 1505, wherein the nonabsorbable composition has an equilibrium proton binding capacity of at least 12 mmol/g and a chloride ion binding capacity of at least 12 mmol/g in an aqueous simulated gastric fluid buffer (“SGF”) containing 35 mM NaCl and 63 mM HCl at pH 1.2 and 37° C.

### Embodiment 1508

The nonabsorbable composition of enumerated embodiment 1504 or 1505, wherein the nonabsorbable composition has an equilibrium proton binding capacity of at least 14 mmol/g and a chloride ion binding capacity of at least 14 mmol/g in an aqueous simulated gastric fluid buffer (“SGF”) containing 35 mM NaCl and 63 mM HCl at pH 1.2 and 37° C.

### Embodiment 1509

The nonabsorbable composition of any one of enumerated embodiments 1504-1508, wherein the nonabsorbable composition has a chloride ion to phosphate ion binding molar ratio of at least 1:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1510

The nonabsorbable composition of any one of enumerated embodiments 1504-1508, wherein the nonabsorbable composition has a chloride ion to phosphate ion binding molar ratio of at least 2:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1511

The nonabsorbable composition of any one of enumerated embodiments 1504-1508, wherein the nonabsorbable composition has a chloride ion to phosphate ion binding molar ratio of at least 3:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1512

The nonabsorbable composition of any one of enumerated embodiments 1504-1508, wherein the nonabsorbable composition has a chloride ion to phosphate ion binding molar ratio of at least 4:1, respectively, in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1513

The nonabsorbable composition of any one of enumerated embodiments 1504-1512, wherein the nonabsorbable composition has a chloride ion binding capacity of at least 2 mmol/g in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1514

The nonabsorbable composition of any one of enumerated embodiments 1504-1512, wherein the nonabsorbable composition has a chloride ion binding capacity of at least 3 mmol/g in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1515

The nonabsorbable composition of any one of enumerated embodiments 1504-1512, wherein the nonabsorbable composition has a chloride ion binding capacity of at least 4 mmol/g in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1516

The nonabsorbable composition of any one of enumerated embodiments 1504-1512, wherein the nonabsorbable composition has a chloride ion binding capacity of at least 5 mmol/g in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1517

The nonabsorbable composition of any one of enumerated embodiments 1504-1512, wherein the nonabsorbable composition has a chloride ion binding capacity of at least 6 mmol/g in an aqueous simulated small intestine inorganic buffer (“SIB”) containing 36 mM NaCl, 20 mM NaH₂PO₄, and 50 mM 2-(N-morpholino)ethanesulfonic acid (MES) buffered to pH 5.5 and at 37° C.

### Embodiment 1518

The nonabsorbable composition of any one of enumerated embodiments 1504-1517, wherein the nonabsorbable composition comprises any polymer described herein, for example any polymer comprising the residue of an amine corresponding any one of formulae 1-4.

### Embodiment 1519

The method/composition of any preceding enumerated embodiment, wherein the method/composition achieves an annualized Death/Dialysis/≥50% eGFR decline (DD50) rate of less than about 5% in a clinical trial.

### Embodiment 1520

The method/composition of any one of enumerated embodiments 1434-1437, wherein the decrease in the rate of progression may be determined relative to the baseline rate of progression prior to treatment.

### Embodiment 1521

The method/composition of any preceding enumerated embodiment wherein the nonabsorbable composition is veverimer.

### Embodiment 1522

The method/composition of any one of enumerated embodiments 1456-1468, wherein the eGFR is an average value of three readings.

### Embodiment 1523

The method/composition of any preceding enumerated embodiment, wherein the blood pressure of the patient after treatment is unchanged relative to the blood pressure of the patient before treatment.

### Embodiment 1524

The method/composition of any preceding enumerated embodiment, wherein the blood pressure of the patient during treatment is unchanged relative to the blood pressure of the patient before treatment.

### Embodiment 1525

The method/composition of any preceding enumerated embodiment, wherein there is not a significant change in the blood pressure of the patient after treatment relative to the blood pressure of the patient before treatment.

### Embodiment 1526

The method/composition of any preceding enumerated embodiment, wherein there is not a significant change in the blood pressure of the patient during treatment relative to the blood pressure of the patient before treatment.

### Embodiment 1527

The method/composition of any preceding enumerated embodiment, wherein the method/composition does not adversely effect blood pressure of treated patient or individual.

### Embodiment 1528

The method/composition of any preceding enumerated embodiment, wherein the method/composition acts without developing hypotension in the patient after treatment.

### Embodiment 1529

The method/composition of any preceding enumerated embodiment, wherein the method/composition acts without developing hypotension in the patient during treatment.

### Embodiment 1530

The method/composition of any preceding enumerated embodiment, wherein the method/composition acts without worsening hypotension in the patient after treatment relative to a level of hypotension in the patient before treatment.

### Embodiment 1531

The method/composition of any preceding enumerated embodiment, wherein the method/composition acts without worsening hypotension in the patient during treatment relative to a level of hypotension in the patient before treatment.

### Embodiment 1531

The method/composition of any preceding enumerated embodiment, wherein the method/composition acts without developing hypertension in the patient after treatment.

### Embodiment 1532

The method/composition of any preceding enumerated embodiment, wherein the method/composition acts without developing hypertension in the patient during treatment.

### Embodiment 1533

The method/composition of any preceding enumerated embodiment, wherein the method/composition acts without worsening hypertension in the patient after treatment relative to a level of hypertension in the patient before treatment.

### Embodiment 1534

The method/composition of any preceding enumerated embodiment, wherein the method/composition acts without worsening hypertension in the patient during treatment relative to a level of hypertension in the patient before treatment.

Having described the invention in detail, it will be apparent that modifications and variations are possible without departing the scope of the invention defined in the appended claims. Indeed, as part of the present invention, it is contemplated that each enumerated embodiment may be combined with one or more other enumerated embodiment in any chemically and/or clinically relevant manner. Furthermore, it should be appreciated that all examples in the present disclosure are provided as non-limiting examples.

### EXAMPLES

### Exemplary Synthetic Approaches for the Preparation of Nonabsorbed Polymers for the Treatment of Acid-Base Imbalance (Reproduced from WO2016/094685 A1)

### Exemplary Synthesis A

Step 1: Two aqueous stock solutions of monomer (50% w/w) were prepared by independently dissolving 43.83 g allylamine hydrochloride and 45.60 g diallylpropyldiamine (“DAPDA”) in water. A 3-neck, 2 L round bottom flask with four side baffles equipped with an overhead stirrer (stirring at 180 rpm), Dean-Stark apparatus and condenser, and nitrogen inlet, was charged with 12 g surfactant (Stepan Sulfonic 100) dissolved in 1,200 g of a heptane/chlorobenzene solution (26/74 v/v), followed by the aqueous stock solutions, and an additional portion of water (59.14 g). In a separate vessel, a 15 wt % solution of initiator 2,2′-azobis(2-methylpropionamidine)-dihydrochloride (“V-50”) (9.08 g) in water was prepared. The two mixtures were independently sparged with nitrogen while the reaction vessel was brought to 67° C. in an oil bath (approximately 30 min). Under inert atmosphere, the initiator solution was added to the reaction mixture, and subsequently heated at 67° C. for 16 hours. A second aliquot of initiator solution (equal to the first) and the reaction mixture, were sparged with nitrogen for 30 minutes and combined before increasing the temperature to 115° C. for a final dehydration step (Dean-Stark). The reaction was held at 115° C. until water stopped collecting in the Dean-Stark trap (6 h, 235 mL removed, >90% of total water, Tᵢₙₜₑₘₐₗ>99° C.). The reaction was allowed to cool to room temperature, and the stirring stopped to allow the beads to settle. The organic phase was removed from the bead cake by decanting. The beads were purified by washing (MeOH two times, H₂O once, 1N HCl two times, H₂O once, 1N NaOH three times, and then H₂O until the pH of solution after washing was 7) and dried by lyophilization.

Step 2: Dry preformed amine polymer beads (15.00 g) prepared in accordance with Step 1 were added to a 250 mL round bottom flask equipped with a stir paddle and nitrogen gas inlet. To the beads was added 1,2-dichloroethane (DCE) (90 mL, resulting in a 1:6 bead to DCE (g/mL) ratio). The beads were dispersed in the DCE using mechanical agitation (˜150 rpm stirring). Water (3.75 mL, resulting in a 0.25:1 water to bead mass ratio) was added directly to the dispersion, and stirring was continued for 30 minutes. After 30 minutes, the flask was immersed into an oil bath held at 70° C. The reaction was held in the oil bath and agitated using mechanical stirring under a nitrogen atmosphere for 16 hours. Methanol (100 mL) was added to the reaction and, solvent was removed by decanting. The beads were then filtered, and then purified by washing (MeOH two times, H₂O once, 1N HCl two times, H₂O once, 1N NaOH three times, and then H₂O until the pH of solution after washing was 7). The purified beads were then dried by lyophilization for 48 hours. Swelling ratio, particle size, chloride binding capacity in SGF and chloride binding capacity (SIB-Cl) and phosphate binding capacity (SIB-P) in SIB are presented in Table S-1 for the resulting polymers.

TABLE S-1

| | | | Particle Size | | | Binding (mmol/g | | |
|---|---|---|---|---|---|---|---|---|
| | Water: | | (microns) | | | dry weight) | | |
| Unique ID | Bead | Swelling | D10 | D50 | D90 | SGF | SIB-Cl | SIB-P |
| Averaged from | — | 5.0 | 79 | 129 | 209 | 13.9 | 2.0 | 6.0 |
| 019069-A1 FA | | | | | | | | |
| pooled batch* | | | | | | | | |
| 030008-A1 FA | 0.00 | 1.9 | NM | NM | NM | 11.8 | 2.4 | 4.0 |
| 019070-A1 FA | 0.05 | 1.5 | 64 | 99 | 155 | 11.1 | 2.4 | 3.5 |
| 019070-A2 FA | 0.15 | 1.1 | 64 | 97 | 147 | 11.0 | 3.3 | 2.5 |
| 019070-A3 FA | 0.25 | 1.2 | 63 | 102 | 168 | 10.4 | 4.4 | 1.4 |
| 019070-A4 FA | 0.35 | 0.7 | 59 | 91 | 140 | 10.7 | 4.5 | 1.3 |
| 019070-A5 FA | 0.45 | 1.6 | 63 | 105 | 184 | 11.1 | 3.7 | 2.5 |

*Averaged data from 4 batches of preformed polyamine bead

### Exemplary Syntheses B-E

Step 1 Exemplary Synthesis B: To a 500 mL round bottom flask, polyallylamine (14 g, 15 kDa), and water (28 mL) were added. The solution was purged with nitrogen and stirred overhead at 220 rpm for 1 hour to completely dissolve the polymer. Next, 30 wt % aqueous NaOH (7 mL) was added and stirred for 5 minutes. A premade solution of DCE (175 mL), n-heptane (105 mL), and Span 80 (2.8 g) was added to the aqueous solution. The solution was heated to 70° C. and stirred for 16 hours. The Dean-Stark step was initiated by adding cyclohexane (100 mL) and heating the reaction to 95° C. to remove the water (>90%) from the beads. Swelling ratio, chloride binding capacity in SGF and chloride binding capacity (SIB-Cl) and phosphate binding capacity (SIB-P) in SIB are presented in Table S-2 (entries 018013-A1 FA and 015026-A1 FA) for the resulting polymer with SGF, SIB—Cl and SIB-P values expressed in mmol/g dry bead.

Step 1 Exemplary Synthesis C: To a 100 mL round bottom flask, DCP (31 mL), n-heptane (19 mL), and Span 80 (0.5 g) were added. A separate aqueous stock solution of polyallylamine (2.3 g, 900 kDa), Aq NaOH (1 mL, 30 wt %), and water (4 mL) was prepared. The aqueous stock solution was added to the organic solution in the round bottom flask. The solution was purged with nitrogen for 15 minutes, heated to 70° C., and stirred for 16 hours. Methanol (30 mL) was added to the reaction mixture and the organic solvent removed by decanting. The resulting beads were purified and isolated by washing the beads using, MeOH, HCl, aqueous sodium hydroxide, and water. The beads were dried using lyophilization techniques. Swelling ratio, chloride binding capacity in SGF and chloride binding capacity (SIB-Cl) and phosphate binding capacity (SIB-P) in SIB are presented in Table S-2 (018001-A2b FA) for the resulting polymer with SGF, SIB-Cl and SIB-P values expressed in mmol/g dry bead.

Step 1 Exemplary Synthesis D: Polyallylamine 15 kDa (3.0 g) and water (9.05 g) were dissolved in a conical flask. Sodium hydroxide (0.71 g) was added to the solution and the mixture was stirred for 30 minutes. To a 100 mL round bottom flask, equipped side arm and overhead stirrer was added 0.38 g of sorbitan sesquioleate and 37.9 g of toluene. The overhead stirrer was switched on to provide agitation to the reaction solution. Dichloropropanol (0.41 g) was added directly to the polyallylamine solution while stirring. The resulting aqueous polyallylamine solution was added to the toluene solution in the 100 mL flask. The reaction was heated to 50° C. for 16 hours. After this time, the reaction was heated to 80° C. for 1 hour and then cooled to room temperature. The resulting beads were purified and isolated by washing the beads using, MeOH, HCl, aqueous sodium hydroxide, and water. The beads were dried using lyophilization techniques. Swelling ratio, chloride binding capacity in SGF and chloride binding capacity (SIB-Cl) and phosphate binding capacity (SIB-P) in SIB are presented in Table S-2 (entries 002054-A3 FA and 011021-A6 FA) for the resulting polymer with SGF, SIB-Cl and SIB-P values expressed in mmol/g dry bead.

Step 1 Exemplary Synthesis E: Polyallylamine 15 kDa (3.1 g) and water (9.35 g) were dissolved in a conical flask. Sodium hydroxide (0.73 g) was added to the solution and the mixture was stirred for 30 minutes. To a 100 mL round bottom flask, equipped side arm and overhead stirrer was added 0.31 g of sorbitan trioleate and 39.25 g of toluene. The overhead stirrer was switched on to provide agitation to the reaction solution. The aqueous polyallylamine solution was added to the toluene solution in the 100 mL flask. Epichlorohydrin (0.30 g) was added directly to the reaction mixture using a syringe. The reaction was heated to 50° C. for 16 hours. After this time the reaction was heated to 80° C. for 1 hour and then cooled to room temperature. The resulting beads were purified and isolated by washing the beads using, MeOH, HCl, aqueous sodium hydroxide, and water. The beads were dried using lyophilization techniques. Swelling ratio, chloride binding capacity in SGF and chloride binding capacity (SIB-Cl) and phosphate binding capacity (SIB-P) in SIB are presented in Table S-2 (entries 002050-A1 FA and 002050-A2 FA) for the resulting polymer with SGF, SIB-Cl and SIB-P values expressed in mmol/g dry bead.

TABLE S-2

| | | | Binding (mmol/g | | |
|---|---|---|---|---|---|
| | | | dry weight) | | |
| Unique ID | Crosslinker | Swelling | SGF | SIB-Cl | SIB-P |
| 018013-A1 FA | DCE | 6.1 | 16.9 | 2.2 | 7.3 |
| 015026-A1 FA | DCE | 5.9 | 16.6 | 2.0 | 7.2 |
| 018001-A2b FA | DCP | 4.6 | 15.9 | 1.9 | 7.1 |
| 002054-A3 FA | DC2OH | 6.5 | 14.3 | 1.6 | 7.1 |
| 011021-A6 FA | DC2OH | 3.0 | 14.3 | 1.5 | 6.1 |
| 002050-A1 FA | ECH | 8.3 | 14.4 | 1.7 | 7.0 |
| 002050-A2 FA | ECH | 8.8 | 14.2 | 1.6 | 7.1 |

Step 1 polymers selected from Exemplary Synthesis B and D were subjected to Step 2 crosslinking according to the following general procedure. Dry preformed amine polymer beads were added to a reactor vessel equipped with a stir paddle and nitrogen gas inlet. To the beads was added 1,2-dichloroethane (DCE). The beads were dispersed in the DCE using mechanical agitation. Water was added directly to the dispersion, and stirring was continued. The flask was immersed into an oil bath held at a chosen temperature. The reaction was held in the oil bath and agitated using mechanical stirring under a nitrogen atmosphere for a chosen amount of time. Methanol was added to the reaction and, solvent was removed by decanting. The beads were then filtered, and then purified by washing. Swelling ratio, chloride binding capacity in SGF and chloride binding capacity (SIB-Cl) and phosphate binding capacity (SIB-P) in SIB are presented in Table S-3.

TABLE S-3

| | Preformed | | | Binding (mmol/g | | |
|---|---|---|---|---|---|---|
| | amine | Step 1 | | dry weight) | | |
| Unique ID | polymer | xlinker | Swelling | SGF | SIB-Cl | SIB-P |
| 018022-A2 FA | 018013-A1 FA | DCE | 1.7 | 14.9 | 4.0 | 4.6 |
| 015032-A1 FA | 015026-A1 FA | DCE | 1.4 | 13.2 | 6.1 | 1.5 |
| 015032-B2 FA | 015026-A1 FA | DCE | 1.2 | 13.0 | 6.1 | 1.5 |
| 002064-B4 FA | 002054-A3 FA | DC2OH | 3.1 | 12.1 | 1.7 | 5.6 |
| 002064-B5 FA | 002054-A3 FA | DC2OH | 2.7 | 12.3 | 1.7 | 5.5 |

### Exemplary Synthesis F

Step 2 Exemplary Synthesis F: Dry preformed amine polymer beads (3.00 g) (prepared as described in Step 1 of Exemplary Synthesis A) were added to a 100 mL round bottom flask equipped with a stir paddle and nitrogen gas inlet. To the beads was added DCP (4.30 mL) and DCE (13.70 mL), resulting in a 1:6 bead to DCE mass/volume ratio). The beads were dispersed in the DCE using mechanical agitation (˜150 rpm stirring). Water (3.00 mL, resulting in a 1:1 water to bead mass ratio) was added directly to the dispersion, and stirring was continued for 30 minutes. After 30 minutes, the flask was immersed into an oil bath held at 70° C. The reaction was held in the oil bath and agitated using mechanical stirring under a nitrogen atmosphere for 16 hours. Methanol (60 mL) was added to the reaction and, solvent was removed by decanting. The beads were then filtered, and then purified by washing (MeOH two times, H₂O once, 1N HCl two times, H₂O once, 1N NaOH three times, and then H₂O until the pH of solution after washing was 7). The purified beads were then dried by lyophilization for 48. Swelling ratio, chloride binding capacity in SGF and chloride binding capacity (SIB-Cl) and phosphate binding capacity (SIB-P) in SIB are presented in Table S-4.

TABLE S-4

| | | | Binding (mmol/g | | |
|---|---|---|---|---|---|
| | Vol % | | dry weight) | | |
| Unique ID | DCE | Swelling | SGF | SIB-Cl | SIB-P |
| 019031-B1 FA | 100 | 1.1 | 11.3 | 5.2 | 1.3 |
| 019031-B2 FA | 92 | 1.0 | 11.2 | 5.2 | 1.4 |
| 019031-B3 FA | 84 | 0.9 | 11.3 | 4.9 | 1.7 |
| 019031-B4 FA | 76 | 1.0 | 11.3 | 4.8 | 1.8 |
| 019031-B5 FA | 68 | 1.0 | 11.4 | 4.6 | 1.9 |
| 019031-B6 FA | 0 | 1.1 | 11.2 | 3.1 | 3.5 |

### Exemplary Synthesis G

Polyallylamine hydrochloride is dissolved in water. Sodium hydroxide is added to partially deprotonate the polyallylamine hydrochloride (preferably 50 mol %). The aqueous phase generated has a water content (by weight) 2.42 times the weight of the polyallylamine hydrochloride. A baffled 3 necked flask, equipped with an overhead mechanical stirrer, nitrogen inlet, Dean Stark apparatus with condenser is set up to conduct the suspension reaction. A dichloroethane heptane mixture is prepared, such that there is 3 times by weight dichloroethane to heptane. This dichloroethane, heptane mixed solvent is added to the baffled 3 neck flask. The aqueous solution is added to the flask, such that the ratio is 6.4 dichloroethane to one water by volume. The reaction mixture is stirred and heated to 70° C. for 16 hours. At this point beads are formed. The Dean Stark step is initiated to remove all the water from the beads, while returning the dichloromethane and heptane back to the reaction mixture. Once no more water is removed the reaction mixture is cooled. Water and sodium hydroxide is added back to the reaction mixture at a ratio of 0.25 water to polyallylamine and up to 1 equivalent of sodium hydroxide per chloride on allylamine added (both calculated from polyallylamine hydrochloride added at the beginning of the reaction). The reaction is heated for a further 16 hours at 70° C. The reaction is cooled to room temperature. The beads are purified using a filter frit with the following wash solvents; methanol, water, aqueous solution of HCl, water, aqueous solution of sodium hydroxide and 3 water washes or until the filtrate measures a pH of 7.

### Example 1

### Efficacy of TRC101 in the Treatment of Acidosis in an Adenine-Induced Model of Nephropathy in Rats

The drug substance, TRC101, is a non-absorbed free-flowing powder composed of low-swelling, spherical beads, approximately 100 micrometers in diameter; each bead is a single crosslinked, high molecular weight molecule. TRC101 is a highly crosslinked aliphatic amine polymer that is synthesized by first copolymerizing two monomers, allylamine hydrochloride and N,N′-diallyl-1,3-diaminopropane dihydrochloride, followed by crosslinking the polymer with 1,2-dichloroethane as described in Exemplary Synthesis A and in WO2016/094685 A1. TRC101 is the polymer with unique ID 019070-A3 FA in Table S-1 of Exemplary Synthesis A. TRC101 is also known by the USAN veverimer.

TRC101 is administered as a free-amine polymer and contains no counterion. TRC101 is insoluble in aqueous and non-aqueous solvents. TRC101 has both high proton and chloride binding capacity and chloride binding selectivity. The high amine content of the polymer is responsible for the high proton and chloride binding capacity of TRC101; the polymer's extensive crosslinking provides size exclusion properties and selectivity over other potential interfering anions, such as phosphate, citrate, bile acids, and short-chain and long-chain fatty acids.

TRC101 was evaluated in vivo in an adenine-induced rat model of chronic kidney disease (CKD) and metabolic acidosis. The study was designed in two parts. In both parts, male Sprague-Dawley rats weighing 260-280 g (10 per group) were first administered adenine (0.75 wt % in casein diet) for 2 weeks to induce nephropathy. Study Part 1 investigated the effect of early treatment with TRC101 administered in a casein diet with 0.25 wt % adenine for the 4 weeks following the 2-week nephropathy induction period. In contrast, study Part 2 assessed the effect of TRC101 administered after animals had been kept on casein diet with 0.25 wt % adenine for 5 weeks following the induction period, before the 4-week TRC101 treatment period was started. The dose levels of TRC101 were 0, 1.5, 3.0, and 4.5 wt % in the diet. Both study parts assessed the effect of withdrawing TRC101 after the end of the Treatment Phase with a 2-week Withdrawal Phase, in which TRC101 was discontinued in the low (1.5 wt %) and high (4.5 wt %) TRC101 dose groups, while dosing of TRC101 was continued in the mid dose group (3.0 wt %). All animals received casein diet with 0.25 wt % adenine during the Withdrawal Phase.

In both study parts, blood samples were taken from the tail vein of animals before treatments started and weekly during the Treatment and Withdrawal Periods for measurement of blood bicarbonate (SBC) using a HESKA Element POC™ blood gas analyzer. Animals were randomized based on SBC levels at baseline (i.e., following adenine induction of nephropathy and before initiation of the dosing period) so that mean baseline SBC levels were comparable across all dose groups. In addition, 24-h fecal collections were performed for the untreated and 4.5 wt % TRC101 groups. Collected fecal samples were stored at −20° C. before drying in a lyophilizer for 3 days followed by homogenization with a mortar and pestle. Anions (Cl, SO₄, and PO₄) were extracted from lyophilized, homogenized fecal samples by incubating the samples with NaOH for 18 hours. Sample supernatants were analyzed for by ion chromatography (IC).

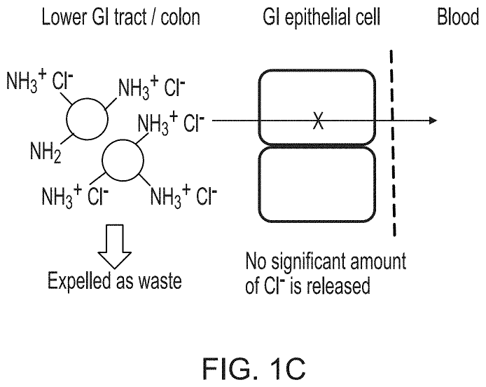

In Part 1, early treatment with TRC101 resulted in a significant, dose-dependent increase in SBC in all treated groups, relative to the untreated controls (FIG. 2; statistical analysis: 2-way ANOVA with Dunnett's multiple comparisons test vs. untreated group; horizontal dotted lines mark the normal SBC range for male Sprague-Dawley rates of the same age). In contrast to the control group, which had a progressive decline in mean SBC due to adenine-induced renal insufficiency over the 4-week treatment period, mean SBC levels increased and remained in the normal range for low, mid and high treatment groups. Upon withdrawal of TRC101, mean SBC levels fell below the normal range in the low and high treatment groups and were similar to the untreated controls at the end of the withdrawal period; whereas, continued treatment with TRC101 (3.0 wt %) maintained SBC levels within the normal range, with the mean value significantly higher than that of the untreated controls.

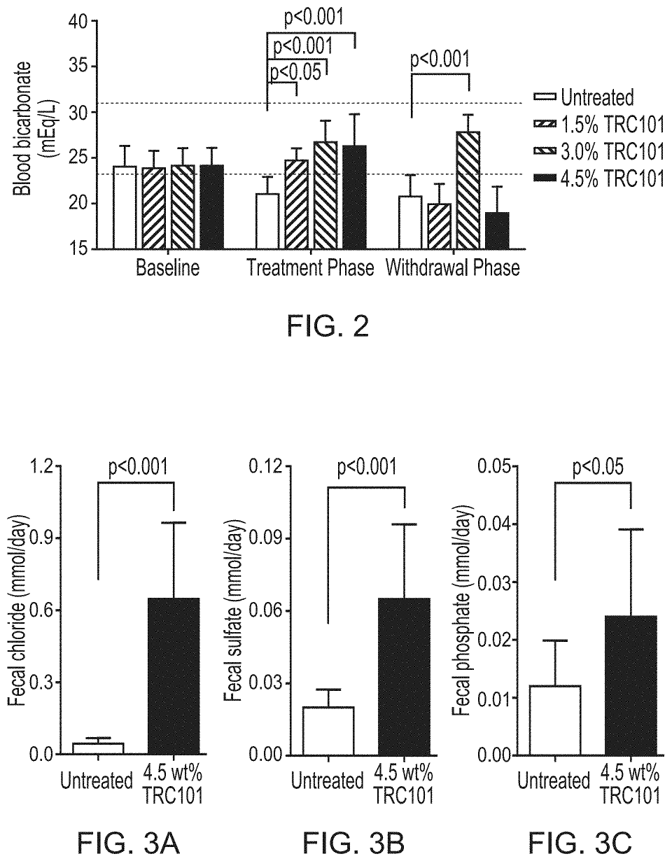

Consistent with the results observed on SBC, recovered fecal samples from animals treated with 4.5 wt % TRC101 in Part 1 of the study demonstrated a significant 15-fold increase in fecal Cl relative to untreated controls (FIG. 3). TRC101 also significantly increased fecal SO₄ and PO₄ excretion, but the effect was much less (3- and 2-fold increase, respectively, compared to untreated controls) than that observed for Cl.

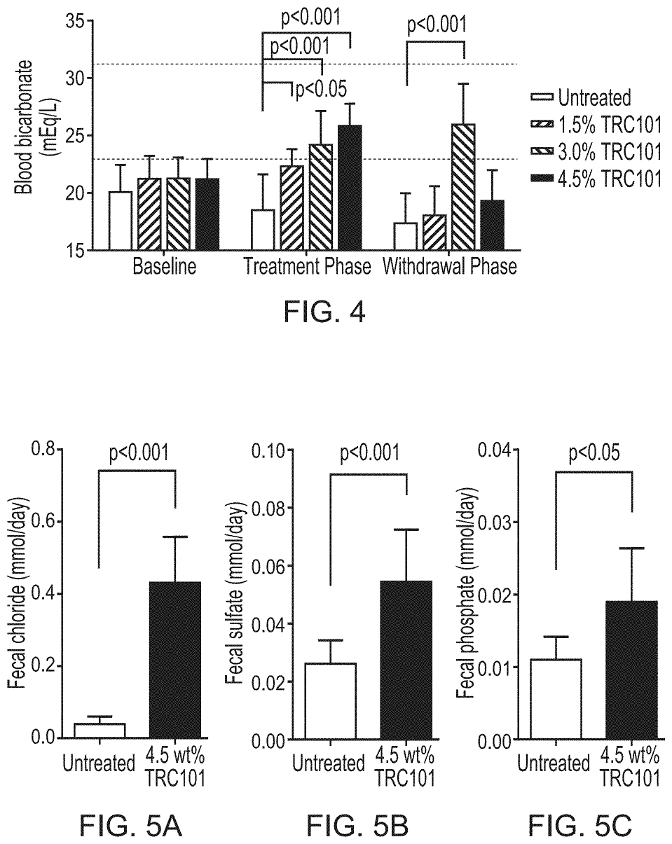

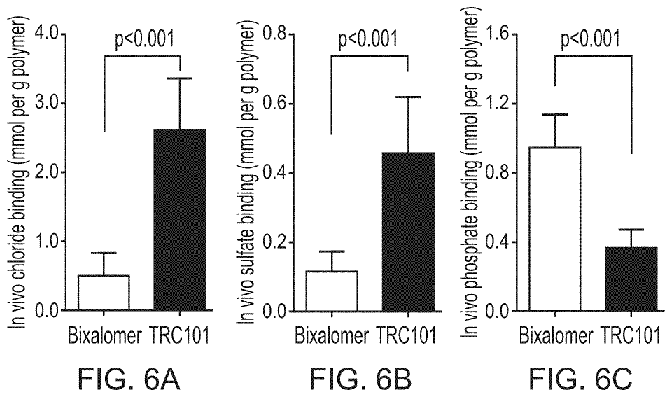

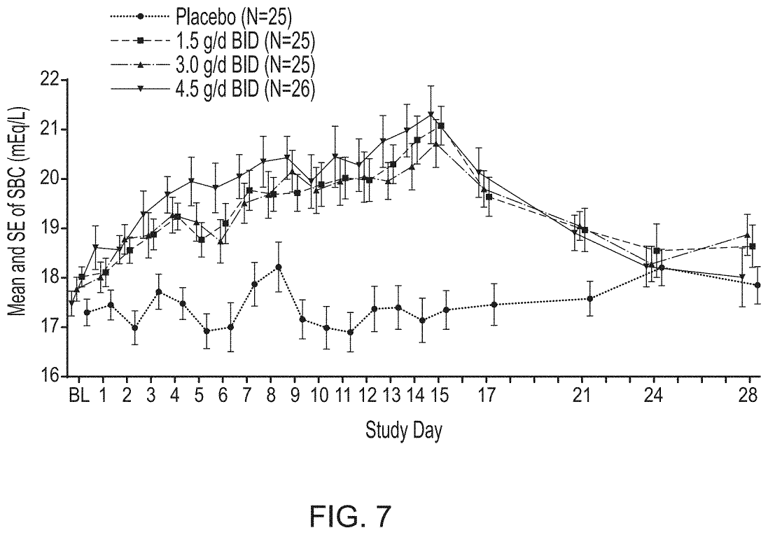

In Part 2 of the study, maintaining rats for a total of 7 weeks on adenine-containing diet prior to the start of the Treatment Phase resulted in mean baseline SBC values that were below the normal range in all treatment groups at a mean of approximately 20 to 21 mEq/L. Treatments with TRC101 resulted in a significant, dose-dependent increase in SBC in all treated groups, relative to the untreated controls. At the end of the 4-week treatment period, mean SBC levels in control animals remained below the normal range. The mean SBC level at the low dose (1.5 wt % TRC101) was only marginally below normal range. At the mid (3.0 wt %) and high (4.5 wt %) doses of TRC101, mean SBC values remained within the normal range (FIG. 4; 2-way ANOVA with Dunnett's multiple comparisons test vs. untreated group; horizontal dotted lines mark the normal SBC range for male Sprague-Dawley rates of the same age). Similar to the results observed in Part 1 of the study, withdrawal of TRC101 administration in Part 2 resulted in a decrease in mean SBC to below the normal range in the low and high doses treatment groups; whereas, continued treatment with 3.0 wt % TRC101 maintained mean SBC levels within the normal range (FIG. 5). The mean SBC level in the 3.0 wt % TRC101 group remained significantly higher than that of the untreated control group throughout the study.

Consistent with the results observed on SBC, recovered fecal samples from animals treated with 4.5 wt % TRC101 in Part 2 of the study demonstrated a significant 10-fold increase in fecal Cl relative to controls, but only a 2-fold increase in fecal SO₄ and PO₄ excretion (FIG. 5).

### Example 2

### In Vivo Anion Binding of Polymers in a Pig with Normal Renal Function

The anion binding capacities of TRC101 (as described in Example 1) was evaluated in vivo in a female Yorkshire pig with normal renal function. A comparative experiment was conducted using the free amine form of bixalomer (approved in Japan), an anion-binding resin designed to bind phosphate and available commercially to treat hyperphosphatemia. TRC101 and the free amine form of bixalomer were each individually sealed in nylon sachets (with a 64 micrometer mesh size and differentiated for each polymer by sachet shape), fed to a single pig at a total dose of 2 g for each polymer (i.e., 10 sachets each), and then the polymers were recovered from the sachets collected in the feces over a 10-day period (seven and six sachets were recovered from feces for bixalomer and TRC101, respectively). Bound anions were extracted from the polymers by incubating with NaOH for 18 hours. The anion concentrations in the samples were determined in supernatant by IC.

Analysis of the anions bound to the polymers after recovery from the feces revealed in vivo average binding of 2.62 and 0.50 mEq of chloride, 0.46 and 0.11 mmol of sulfate, and 0.37 and 0.95 mmol of phosphate per gram of TRC101 and bixalomer, respectively (FIG. 61 statistical analysis unpaired T test; Mean±standard deviation; N=7 and 6 sachets for Bixalomer and TRC101, respectively). Therefore, TRC101 removed 5- and 4-fold more chloride and sulfate, respectively, than bixalomer removed from the GI tract of the pig. On the other hand, bixalomer, a phosphate binder, removed 2.5-fold more phosphate than TRC101 removed from the GI tract of the pig.

### Example 3

### Efficacy of TRC101 in Subjects with Chronic Kidney Disease and Low Serum Bicarbonate Levels

Part 1

TRC101 (as described in Example 1) was studied in a double-blind, placebo-controlled, parallel-design, 4-arm, fixed dose study to evaluate the ability of TRC101 to control serum bicarbonate (SBC) in human subjects with marked metabolic acidosis. A total of 101 subjects with chronic kidney disease (CKD) and low SBC values were randomized into one of the four arms in an approximately 1:1:1:1 ratio (total daily doses of 3, 6 or 9 g/day TRC101 or 3 g/day placebo [microcrystalline cellulose], administered twice daily [BID]).

Subjects were eligible for inclusion in the study if they were 18 to 80 years of age, had Stage 3 or 4 CKD (estimated glomerular filtration rate [eGFR], 20 to <60 mL/min/1.73 m² of body surface area) and SBC levels of 12 to 20 mEq/L (inclusive) at both Screening and study Day −1, had systolic blood pressure (SBP) at Screening<170 mmHg, had a hemoglobin A1c (HbA1c) value of ≤9.0% and a fasting serum glucose (FSG) value of ≤250 mg/dL (13.9 mmol/L) at Screening. Key exclusion criteria were history of anuria, dialysis, acute kidney injury, acute renal insufficiency or >30% increase in serum creatinine or 30% decrease in eGFR in the past 3 month, severe comorbid conditions (other than CKD) such as congestive heart failure with maximum New York Heart Association (NYHA) Class III or IV symptoms, unstable angina or acute coronary syndrome, dementia, hypertensive urgency or emergency, transient ischemic attack, stroke, or use of home oxygen during the 6 months prior to Screening. Other exclusion criteria were serum potassium values of <3.8 mEq/L or >5.9 mEq/L at Screening, Type 1 diabetes or chronic obstructive pulmonary disease, history or current diagnosis of heart or kidney transplant, clinically significant diabetic gastroparesis, bariatric surgery, bowel obstruction, swallowing disorders, severe gastrointestinal disorders, severe recurrent diarrhea or severe recurrent constipation.

At the time of Screening, subjects who met all the entry criteria were admitted to the Clinical Research Unit (CRU) on Day −1 and placed on a study diet controlled for protein, caloric content, anions, cations and fiber, in accordance with dietary recommendations for patients with CKD (KDOQI, 2003). The potential renal acid load (i.e., PRAL value) (Scialla, 2013) was calculated for the daily meal plans to ensure that the study diet was neither acidic nor basic; PRAL values for the four daily meal plans ranged from −1.71 to +1.92 and averaged 0.82. The PRAL is calculated as follows:

PRAL(mEq/d)=(0.49*protein [g/d])+(0.037*phosphorus [mg/d])−(0.21*potassium [mg/d])−(0.26*magnesium [mg/d])—0.013*(calcium [mg/d])

Four detailed meal plans were developed that specified the foods (including measured quantities) provided at breakfast, lunch, dinner and two light snacks each day (Table S-5). Care was taken to ensure the diet closely approximated the subjects' typical diet so that perturbations in serum bicarbonate related to a sudden change in diet would be minimized. The dietary sources of protein were predominantly plant-based. Meat (i.e., pork, fish) was served once per day on two of the four meal plans. The sites rotated among the four daily meal plans over the course of the treatment period. The mean (±standard deviation) serum bicarbonate level in the placebo group was 17.6 (±1.43) mEq/L at baseline and remained constant during the 14-day treatment period (17.5 [±1.87] mEq/L at Day 15), demonstrating that the study diet did not change the level of serum bicarbonate.

TABLE S-5
Composition of Study Treatment Period Diet

| Parameter | Calories | Protein (g) | Ca (mg) | Mg (mg) | P (mg) | K (mg) | Na (mg) | Fiber (g) | PRAL |
|---|---|---|---|---|---|---|---|---|---|
| Mean | 2209.25 | 52.32 | 810 | 232.5 | 1008.125 | 2171.375 | 2249.5 | 27.022 | 0.82 |
| Range | 2129-2246 | 50.6-53.4 | 778-849 | 210-235 | 991-1060 | 2048-2277 | 2076-2370 | 22.9-32.1 | −1.71-+1.92 |

Ca = calcium;
K = potassium;
Mg = magnesium;
Na = sodium;
P = phosphate

Enrolled subjects were randomized to one of three TRC101 doses or placebo on Day −1 and dosing was initiated in the morning on Day 1 (next day) in accordance with the randomization assignment. 101 subjects were randomized in an approximately 1:1:1:1 ratio to one of the following groups: Group 1. 3 g/day of placebo administered in equally divided doses BID (twice daily) for 14 days (n=25); Group 2. 3 g/day of TRC101 administered in equally divided doses BID for 14 days (n=25); Group 3. 6 g/day of TRC101 administered in equally divided doses BID for 14 days (n=25); Group 4. 9 g/day of TRC101 administered in equally divided doses BID for 14 days (n=26). TRC101 or placebo were administered orally as an aqueous suspension BID, with breakfast and dinner. The first dose of study drug was taken with breakfast. One hour prior to the administration of the study drug, venous blood was drawn for a pre-dose SBC (contributing to the baseline SBC value) and safety laboratory measurements. Subjects remained in the CRU and continued BID dosing with study drug (at breakfast and dinner) for 14 days. On Day 15, subjects were discharged from the CRU. All subjects who completed the study had a discharge assessment on Day 15 and returned to the CRU on Day 17 and Day 21 for AE collection, blood draws and safety assessments. A subset of patients (n=41) also returned to the CRU on Day 28 for AE collection, blood draws and safety assessments.

No subject was withdrawn early from the study for any reason. The majority of subjects were male (65%), all subjects were white, and the median age was 61 years (range: 30 to 79 years).

Subjects in the study had Stage 3-4 CKD (39% with Stage 4) with a mean baseline eGFR of 36.4 mL/min/1.73 m² (range 19.0 to 66.0 mL/min/1.73 m²) and metabolic acidosis characterized by a mean SBC level of 17.6 mEq/L (range 14.1-20.4 mEq/L). At baseline, 60% of subjects had an SBC value of 12-18 mEq/L and 40% had an SBC value of >18-20 mEq/L.

Subjects had baseline comorbidities common in CKD patients including hypertension (93%), diabetes (73%), left ventricular hypertrophy (30%), and congestive heart failure (21%). As would be expected in a CKD Stage 3-4 population, nearly all study subjects had indications for sodium restriction: hypertension (93%), congestive heart failure (21%), peripheral edema (15%) and use of diuretics (41%).

Over a 2-week treatment period, TRC101 significantly increased SBC levels in the study population of CKD patients with baseline SBC levels ranging from 14 to 20 mEq/L. At Day 15, all three doses tested (3, 6 and 9 g/day TRC101 BID) significantly (p<0.0001) increased mean SBC levels from baseline and each dose increased SBC levels to a significantly (p<0.0001) greater extent than placebo.

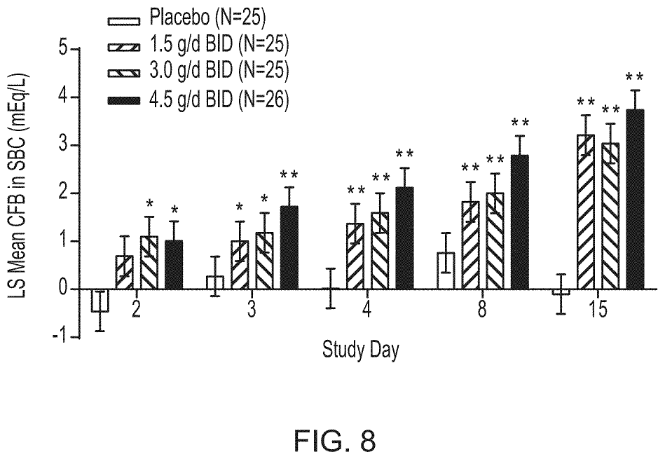

FIG. 7 illustrates the steady increase in mean SBC observed in all three TRC101 dose groups during the 14-day treatment period with a mean increase at the end of treatment of approximately 3-4 mEq/L across all three active dose groups. Serum bicarbonate levels in the placebo group remained essentially unchanged throughout the study, suggesting that the diet with a controlled protein and cation/anion content administered in the clinical research unit matched well with what the subjects ate at home and, therefore, had no significant impact on their SBC values.

TRC101 had a rapid onset of action (i.e., statistically significant increase in mean within group change from baseline in SBC; p<0.0001) within the first 24-48 hours following the initiation of treatment for all three TRC101 dose groups combined. The onset of action for between-group differences (active vs. placebo) appear to occur between 48-72 hours after the initiation of treatment with TRC101. At Day 4 (72 hours after the first dose of TRC101), the mean increase in SBC from baseline for each TRC101 group was 1-2 mEq/L: 3 g/day (p=0.0011); 6 g/day (p=0.0001); 9 g/day (p<0.0001).

Each of the TRC101 dose groups showed a statistically significant (p<0.0001) increase from baseline in SBC of approximately 3-4 mEq/L after 2 weeks of treatment (see Table 1).

TABLE 1
Change from Baseline in SBC at Day 15

| | | TRC101 | TRC101 | TRC101 | TRC101 |
|---|---|---|---|---|---|
| | Placebo | 3 g/d BID | 6 g/d BID | 9 g/d BID | Combined |
| | (N = 25) | (N = 25) | (N = 25) | (N = 26) | (N = 76) |
| Baseline | | | | | |
| N | 25 | 25 | 25 | 26 | 76 |
| Mean (SD) | 17.30 (1.338) | 18.02 (1.009) | 17.77 (1.212) | 17.48 (1.282) | 17.75 (1.180) |
| Median | 17.40 | 17.90 | 17.80 | 17.73 | 17.83 |
| Min, Max | 14.1, 19.6 | 15.6, 20.4 | 15.4, 19.9 | 14.5, 19.2 | 14.5, 20.4 |
| Day 15 | | | | | |
| N | 25 | 25 | 25 | 26 | 76 |
| Mean (SD) | 17.35 (1.958) | 21.08 (1.960) | 20.72 (2.423) | 21.30 (2.977) | 21.04 (2.475) |
| Median | 17.00 | 21.30 | 20.50 | 21.45 | 21.20 |
| Min, Max | 14.1, 21.7 | 17.3, 24.8 | 15.4, 25.9 | 15.1, 27.0 | 15.1, 27.0 |
| Day 15 Change from Baseline (CFB) | | | | | |
| N | 25 | 25 | 25 | 26 | 76 |
| Mean (SD) | 0.05 (1.955) | 3.06 (2.209) | 2.95 (2.625) | 3.83 (2.372) | 3.29 (2.408) |
| Median | −0.10 | 3.55 | 2.40 | 3.23 | 3.07 |
| Min, Max | −3.5, 4.6 | −1.6, 7.5 | −1.5, 8.6 | −0.4, 9.2 | −1.6, 9.2 |
| Within Group CFB | | | | | |
| LS Mean (SEM) | −0.10 (0.414) | 3.21 (0.415) | 3.04 (0.414) | 3.74 (0.406) | 3.33 (0.237) |
| 95% CI of LS Mean | −0.91, 0.71 | 2.39, 4.02 | 2.23, 3.85 | 2.95, 4.54 | 2.86, 3.80 |
| p-value | 0.8109 | <.0001 | <.0001 | <.0001 | <.0001 |
| Between Group CFB Difference (TRC101 − | | | | | |
| Placebo) | | | | | |
| LS Mean (SEM) | NA | 3.31 (0.588) | 3.14 (0.587) | 3.84 (0.579) | 3.43 (0.478) |
| 95% CI of LS Mean | NA | 2.15, 4.46 | 1.99, 4.29 | 2.70, 4.98 | 2.49, 4.37 |
| p-value | NA | <.0001 | <.0001 | <.0001 | <.0001 |

Note:
baseline serum bicarbonate (SBC) is defined as an average of two SBC values from samples collected on Day −1 and at Day 1 pre-dose. Change from baseline (CFB) is defined as post-baseline value minus baseline value.
Note:
Least squares (LS) mean, standard error of LS mean (SEM), 95% CI of LS mean, and p-values are based on the mixed-effect repeated measures model with the CFB in SBC value as the dependent variable; treatment (placebo, 3 g/d BID, 6 g/d BID, and 9 g/d BID), time point (Days 2 through 15), and treatment by time point as fixed effects; subject as a random effect; and baseline estimated glomerular filtration rate (eGFR) and baseline SBC as continuous covariates. Within-subject correlations are modeled assuming a first-order autoregressive covariance structure.

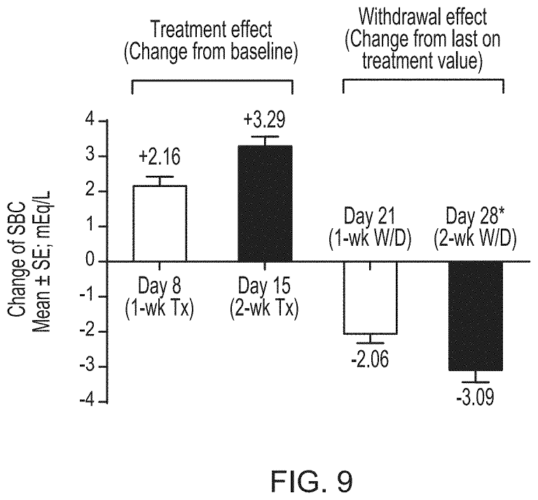

There appeared to be little difference in efficacy between the 3 g/day and 6 g/day TRC101 doses; however, subjects in the 9 g/day TRC101 dose group demonstrated a more rapid and larger increase in SBC. For example, the mean increases in SBC at Day 8 were 1.82, 2.00, and 2.79 mEq/L in the 3, 6 and 9 g/day TRC101 dose groups respectively (i.e., −0.8-1.0 mEq/L difference between the 9 g/day dose group and the other two TRC101 dose groups). At Day 15, the comparable SBC increases were 3.21, 3.04, and 3.74 mEq/L, respectively (i.e., −0.5-0.7 mEq/L difference between the 9 g/day dose group and the other two TRC101 dose groups) (FIG. 8)).

Statistically significant between-group (active vs. placebo) differences in SBC change from baseline to Day 15 ranged from 3.14 to 3.84 mEq/L across the TRC101 treatment groups, with a combined mean difference of 3.43 mEq/L between TRC101 and placebo (p<0.0001) (see Table 1).

As shown in Table 2, after 2 weeks of treatment, SBC levels increased by ≥3 mEq/L in over half of subjects (52.6%) in the combined TRC101 group compared to 8.0% of subjects in the placebo group (p<0.0001). In addition, 22.4% of all TRC101-treated subjects had increases in SBC mEq/L compared to 0 subjects in the placebo group.

TABLE 2
Change in SBC by Category over Time

| | | | | | |
|---|---|---|---|---|---|
| | | TRC101 | TRC101 | TRC101 | TRC101 |
| Subjects with Post | Placebo | 3 g/d | 6 g/d | 9 g/d | Combined |
| Baseline SBC | N = 25 | N = 25 | N = 25 | N = 26 | N = 76 |

Day 15 Increase from Baseline

| | | | | | |
|---|---|---|---|---|---|
| ≥2 mEq/L | 4 (16.0%) | 18 (72.0%) | 14 (56.0%) | 19 (73.1%) | 1 (67.1%) |
| ≥3 mEq/L | 2 (8.0%) | 14 (56.0%) | 10 (40.0%) | 16 (61.5%) | 40 (52.6%) |
| ≥4 mEq/L | 1 (4.0%) | 8 (32.0%) | 10 (40.0%) | 11 (42.3%) | 29 (38.2%) |
| ≥5 mEq/L | 0 | 3 (12.0%) | 6 (24.0%) | 8 (30.8%) | 17 (22.4%) |
| ≥6 mEq/L | 0 | 3 (12.0%) | 3 (12.0%) | 4 (15.4%) | 10 (13.2%) |
| ≥7 mEq/L | 0 | 1 (4.0%) | 2 (8.0%) | 2 (7.7%) | 5 (6.6%) |

In the combined TRC101 treatment group, 35.5% of subjects had their SBC corrected into the normal range (22-29 mEq/L) after 2 weeks of treatment, and at the end of the treatment period, 64.5% of TRC101-treated subjects had SBC levels that were above the upper limit of the baseline range (>20 mEq/L) (Table 3). The proportion of subjects achieving an SBC>22 mEq/L was similar in the 3, 6 and 9 g/day TRC101 dose groups (40.0%, 28.0%, and 38.5%, respectively). At Day 8 of the treatment period, only about half of the treatment effect was seen, again suggesting that the SBC increase has not yet plateaued by the end of the 2-week treatment period.

TABLE 3
Change in SBC by Category over Time

| | | | | | |
|---|---|---|---|---|---|
| | | TRC101 | TRC101 | TRC101 | TRC101 |
| Subjects with Post | Placebo | 3 g/d | 6 g/d | 9 g/d | Combined |
| Baseline SBC | N = 25 | N = 25 | N = 25 | N = 26 | N = 76 |

Day 8 SBC Values

| | | | | | |
|---|---|---|---|---|---|
| >20 mEq/L | 3 (12.0%) | 9 (36.0%) | 7 (28.0%) | 12 (46.2%) | 28 (36.8%) |
| >22 mEq/L | 2 (8.0%) | 2 (8.0%) | 5 (20.0%) | 6 (23.1%) | 13 (17.1%) |
| >27 mEq/L | 0 | 0 | 0 | 0 | 0 |
| >29 mEq/L | 0 | 0 | 0 | 0 | 0 |

Day 15 SBC Values

| | | | | | |
|---|---|---|---|---|---|
| >20 mEq/L | 2 (8.0%) | 16 (64.0%) | 14 (56.0%) | 19 (73.1%) | 49 (64.5%) |
| >22 mEq/L | 0 | 10 (40.0%) | 7 (28.0%) | 10 (38.5%) | 27 (35.5%) |
| >27 mEq/L | 0 | 0 | 0 | 0 | 0 |
| >29 mEq/L | 0 | 0 | 0 | 0 | 0 |

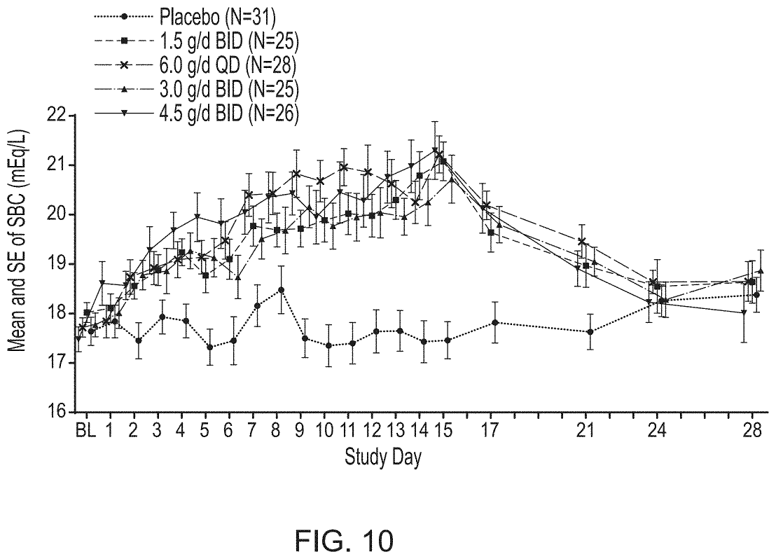

The 2-week treatment period in the study was followed by a 2-week follow-up period in which subjects were off treatment. At the end of the 2-week follow-up period, a withdrawal effect of approximately 3 mEq/L was observed in the combined TRC101 group, with SBC levels returning nearly to baseline (FIG. 9). These results underscore the chronic nature of the underlying metabolic acidosis in these CKD patients, and suggest that continued treatment with TRC101 is needed to maintain elevated SBC levels.

There were no mean changes in serum parameters (sodium, calcium, potassium, phosphate, magnesium, low density lipoprotein) observed in the study that would indicate off-target effects of TRC101; there were also no mean changes in serum chloride.

Part 2

The double-blind, placebo-controlled, parallel-design, fixed dose study of Part 1 was extended by the introduction of two additional arms: a total of 34 subjects with chronic (CKD) and low SBC values were randomized into one of two additional arms: total daily dose of 6 g/day TRC101 (28 subjects) or 3 g/day placebo (6 subjects) [microcrystalline cellulose], administered once daily [QD]). All subjects who completed Part 2 of the study had a discharge assessment on Day 15 and returned to the CRU on Day 17, Day 21, and Day 28 for AE collection, blood draws and safety assessments. Part 2 of the study was otherwise unchanged from Part 1.

Discussion of Part 1 and Part 2 Study Results

There were no significant differences between the TRC101 and placebo treatment groups with respect to demographics, baseline eGFR or serum bicarbonate, or comorbidities (Table 4). Patients had a mean baseline eGFR of 34.8 mL/min/1.73 m2 and a mean baseline serum bicarbonate level of 17.7 mEq/L. Study participants had conditions common to CKD patients, including patients with hypertension (93.3%), diabetes (69.6%), left ventricular hypertrophy (28.9%), congestive heart failure (21.5%), peripheral edema (14.1%) and stable diuretic use (42.2%).

Analysis of the mean serum bicarbonate level in the placebo group over the course of the in-unit treatment period and out-patient follow-up period demonstrated that the study diet did not change the level of serum bicarbonate. The mean (±standard deviation) serum bicarbonate level in the placebo group was 17.6 (±1.43) mEq/L at baseline and remained constant during the 14-day treatment period (17.5 [±1.87] mEq/L at Day 15).

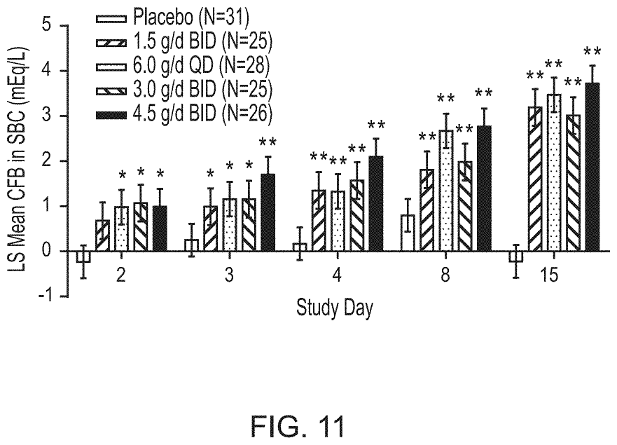

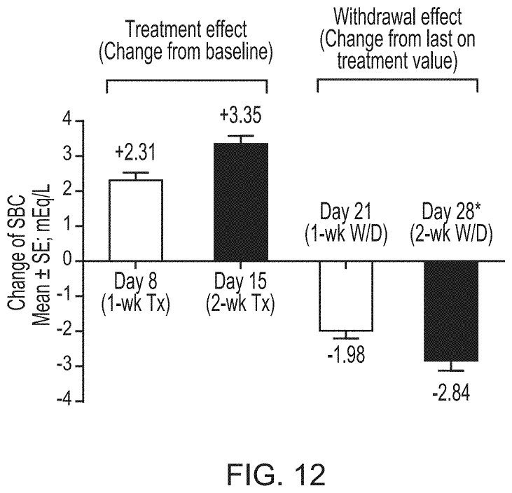

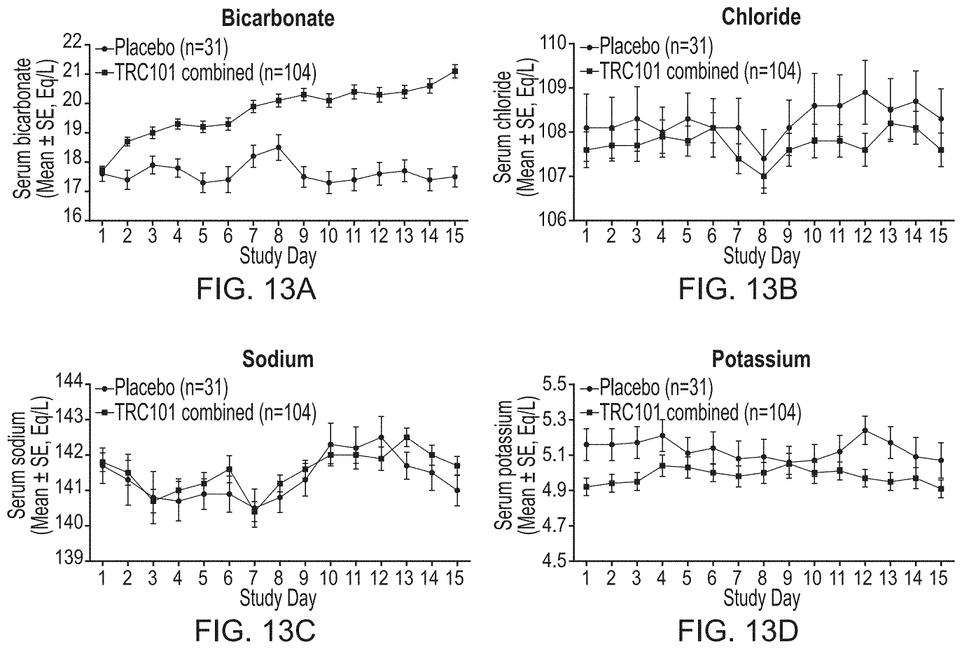

There was a significant increase in mean serum bicarbonate in all groups treated with TRC101 within the first 24-48 hours compared to placebo (FIGS. 10 & 11). Within 72 hours after the first dose of TRC101, the mean increase in serum bicarbonate from baseline for each TRC101 group was 1-2 mEq/L

Over the 2-week treatment period, TRC101 increased serum bicarbonate values over the respective baseline values for each group, while placebo-treated patients had no change in serum bicarbonate (FIGS. 10 & 11). At day 15, the between group difference of serum bicarbonate versus placebo was 3.31 mEq/L (95% CI of LS mean 2.15 to 4.46; p<0.0001), 3.14 mEq/L (95% Cl of LS mean 1.99 to 4.29; p<0.0001), 3.84 mEq/L (95% Cl of LS mean 2.70 to 4.98; p<0.0001), and 3.72 mEq/L (95% Cl of LS mean 2.70 to 4.74; p<0.0001), for TRC101 dose groups 1.5 g, 3.0 g, 4.5 g BID and 6 g QD, respectively. By comparison, the placebo within group change from baseline to day 15 was −0.21 mEq/L (95% Cl of LS mean −0.91 to 0.49; p=0.56). The mean increase in the combined TRC101 dose groups was 3.57 mEq/L higher than in the placebo group at the end of the 14-day treatment period (95% Cl of LS mean 2.75 to 4.38; p<0.0001). At day 15 there was no significant difference in the mean serum bicarbonate increase when TRC101 was given as a dose of 6.0 g once daily versus 3.0 g twice daily (−0.53 mEq/L; 95% Cl of LS mean −1.61 to 0.56; p=0.34).

Treatment with TRC101 caused a steady increase in mean serum bicarbonate in all TRC101 dose groups during the 14-day treatment period. The slope of serum bicarbonate increase remained constant, with no evidence of a plateau at the end of treatment, indicating that the maximal increase in serum bicarbonate using the study doses of TRC101 was not established. The change in serum bicarbonate was similar in all groups treated with TRC101 at the end of the treatment period (FIGS. 10 & 11).

After 2 weeks of treatment with TRC101, serum bicarbonate increased by ≥3 mEq/L in over half of the patients (51.9%) in the combined TRC101 dose group, compared to 6.5% of patients in the placebo group (Table 5). In addition, 38.5% and 22.1% of all TRC101-treated patients, compared to 3.2% and 0% of placebo-treated patients, had increases in serum bicarbonate of ≥4 mEq/L and mEq/L, respectively.

At the end of TRC101 treatment, 34.6% of patients in the combined TRC101 group had a serum bicarbonate in the normal range (22-29 mEq/L) compared to no patients in the placebo group. At the end of TRC101 dosing, the proportion of patients with a normal serum bicarbonate was similar in the four TRC101 dose groups (40.0%, 28.0%, and 38.5%, 32.1% for 1.5 g BID, 3.0 g BID, 4.5 g BID, and 6.0 g QD, respectively) while none of the patients in the placebo group had a normal serum bicarbonate (Table 6).

At the end of the 2-week, off-treatment, follow-up period, a decrease in serum bicarbonate of approximately 3.0-3.5 mEq/L from the end-of-treatment value was observed in all TRC101 dose groups, with serum bicarbonate levels returning nearly to baseline value in each respective group (FIGS. 10 & 11).

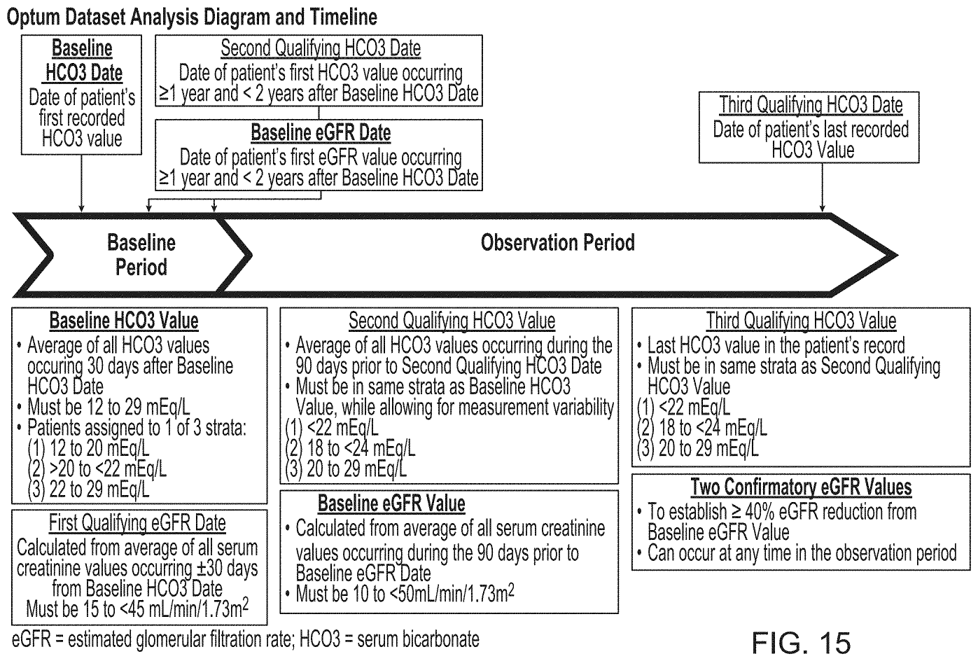

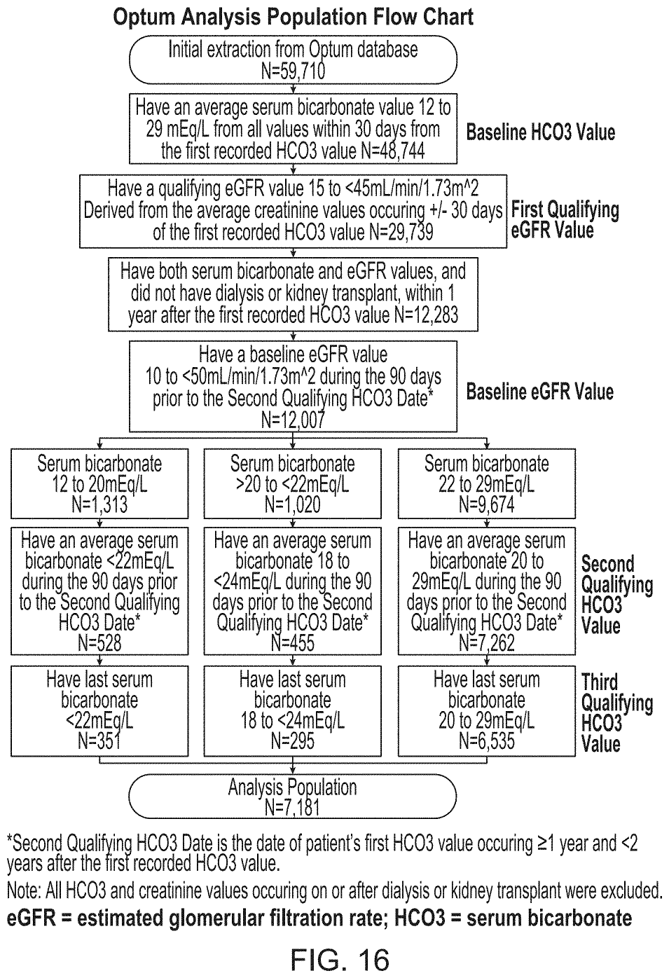

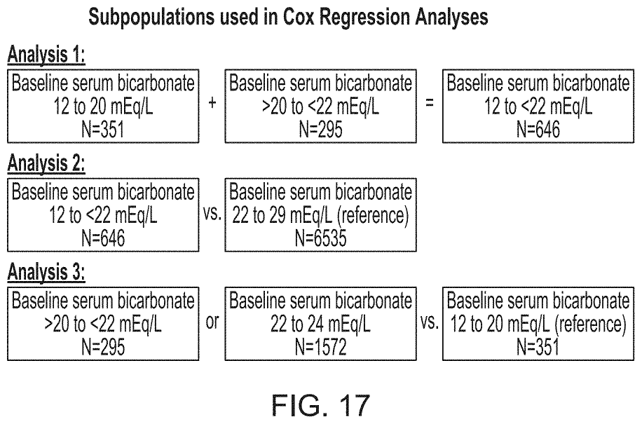

In contrast to serum bicarbonate, serum potassium, serum sodium and serum chloride levels did not significantly change over the course of the study (FIGS. 13A-13D), yielding a change in the serum anion gap in excess of 2 mEq/l (FIG. 14) over the course of the study.

All 135 randomized patients received TRC101 or placebo daily for 14 consecutive days and were included in the safety analysis population. No patients died during the study, or had any adverse events resulting in treatment discontinuation, and no patients suffered serious or severe adverse events. Gastrointestinal adverse events were the most commonly reported events in TRC101-treated patients, and all events were mild or moderate in severity (Table 7). Diarrhea was the most common adverse event; all diarrhea events were mild, self-limited, of short duration, and none required treatment. There were no trends suggesting an off-target effect of TRC101 on electrolytes (i.e., sodium, potassium, magnesium, calcium or phosphate). There were also no trends suggesting an effect of TRC101 on vital signs or ECG intervals. No subject experienced increases in serum bicarbonate that resulted in metabolic alkalosis (i.e., serum bicarbonate>29 mEq/L).

This two-part, double-blind, placebo-controlled, parallel-design, 6-arm, fixed dose clinical study demonstrates that ingestion of TRC101 highly significantly increases serum bicarbonate level in patients with Stage 3 or 4 CKD and low SBC as assessed both by change from baseline within group and by comparisons between active and placebo groups. The rapid onset of action (within 24-72 hours) and efficacy (>3.0 mEq/L increase in SBC) observed in the study suggests that TRC101 is an effective agent in controlling SBC level in the target patient population. Unlike sodium bicarbonate, TRC101 does not introduce cations, such as sodium or potassium, which are deleterious to sodium-sensitive patients with common CKD comorbidities (e.g. hypertension, edema and heart failure). Therefore, TRC101 is expected to provide a safe treatment to control SBC in CKD patients with low SBC, including those who are sodium-sensitive.

TABLE 4
Baseline demographics, dietary intake, renal function, serum bicarbonate and co-morbidities

| | Placebo | TRC101 | TRC101 | TRC101 | TRC101 | TRC101 | |
|---|---|---|---|---|---|---|---|
| | Combined | 1.5 g BID | 3.0 g BID | 6 g QD | 4.5 g BID | Combined | Total |
| | N = 31 | N = 25 | N = 25 | N = 28 | N = 26 | N = 104 | N = 135 |
| Age^{a} (years) | 65.0 | 59.0 | 61.0 | 65.0 | 66.0 | 62.5 | 63.0 |
| Gender | 19 (61.3%)/ | 19 (76.0%)/ | 17 (68.0%)/ | 16 (57.1%)/ | 15 (57.7%)/ | 68 (65.4%)/ | 87 (64.4%)/ |
| (Male/Female) | 12 (38.7%) | 6 (24.6%) | 8 (32.0%) | 12 (42.9%) | 11 (42.3%) | 36 (34.6%) | 48 (35.6%) |
| Weight^{a}, kg | 81.0 | 80.0 | 84.70 | 84.2 | 81.2 | 83.0 | 82.0 |
| Average Daily | 0.64 | 0.65 | 0.61 | 0.62 | 0.64 | 0.63 | 0.63 |
| Protein Intake^{a}, | | | | | | | |
| g/kg/d | | | | | | | |
| Diabetes | 20 (64.5%)/ | 18 (72.0%)/ | 20 (80.0%)/ | 17 (60.7%)/ | 19 (73.1%)/ | 74 (71.2%)/ | 94 (69.6%)/ |
| Mellitus | 11 (35.5%) | 7 (28.0%) | 5 (20.0%) | 11 (39.3%) | 7 (26.9%) | 30 (28.8%) | 41 (30.4%) |
| (Yes/No) | | | | | | | |
| Hypertension | 30 (96.8%)/ | 24 (96.0%)/ | 23 (92.0%)/ | 26 (92.9%)/ | 23 (88.5%)/ | 96 (92.3%)/ | 126 (93.3%)/ |
| (Yes/No) | 1 (3.2%) | 1 (4.0%) | 2 (8.0%) | 2 (7.1%) | 3 (11.5%) | 8 (7.7%) | 9 (6.7%) |
| Heart Failure | 7 (22.6%)/ | 5 (20.0%)/ | 7 (28.0%)/ | 5 (17.9%)/ | 5 (19.2%)/ | 22 (21.1%)/ | 29 (21.5%)/ |
| (Yes/No) | 24 (77.4%) | 20 (80.0%) | 18 (72.0%) | 23 (82.1%) | 21 (80.8%) | 82 (78.9%) | 106 (78.5%) |
| Left Ventricular | 8 (25.8%)/ | 7 (28.0%)/ | 7 (28.0%)/ | 8 (28.6%)/ | 9 (34.6%)/ | 31 (29.8%)/ | 39 (28.9%)/ |
| Hypertrophy | 23 (74.2%) | 18 (72.0%) | 18 (72.0%) | 20 (71.4%) | 17 (65.4%) | 73 (70.2%) | 96 (71.1%) |
| (Yes/No) | | | | | | | |
| Peripheral | 4 (12.9%)/ | 3 (12.0%)/ | 4 (16.0%)/ | 4 (14.3%)/ | 4 (15.4%)/ | 15 (14.4%)/ | 19 (14.1%)/ |
| Edema | 27 (87.1%) | 22 (88.0%) | 21 (84.0%) | 24 (85.7%) | 22 (84.6%) | 89 (85.6%) | 116 (85.9%) |
| (Yes/No) | | | | | | | |
| SBP^{a}, mmHg | 128.00 | 132.00 | 133.00 | 130.00 | 128.50 | 131.50 | 130.00 |
| eGFR^{a}, | 29.0 | 34.0 | 35.0 | 28.0 | 34.0 | 33.0 | 32.0 |
| m>/min/1.73 m² | | | | | | | |
| SBC^{a}, mEq/L | 17.6 | 17.9 | 17.8 | 17.7 | 17.7 | 17.8 | 17.7 |

(^{a}median values)

TABLE 5
Proportion of Patients by Serum Bicarbonate Increase Category at Day 15

| Patients with | | | | | | | | | | | | |
|---|---|---|---|---|---|---|---|---|---|---|---|---|
| Post-baseline | Pooled | | TRC101 | | TRC101 | | TRC101 | | TRC101 | | TRC101 | |
| Serum | Placebo | | 1.5 g BID | | 6 g QD | | 3.0 g BID | | 4.5 g BID | | Combined | |
| Bicarbonate | N = 31 | | N = 25 | | N = 28 | | N = 25 | | N = 26 | | N = 104 | |
| ≥2 mEq/L | 4 | (12.9%) | 18 | (72.0%) | 23 | (82.1%) | 14 | (56.0%) | 19 | (73.1%) | 74 | (71.2%) |
| ≥3 mEq/L | 2 | (6.5%) | 14 | (56.0%) | 14 | (50.0%) | 10 | (40.0%) | 16 | (61.5%) | 54 | (51.9%) |
| ≥4 mEq/L | 1 | (3.2%) | 8 | (32.0%) | 11 | (39.3%) | 10 | (40.0%) | 11 | (42.3%) | 40 | (38.5%) |

| | | | | | | | | | | | |
|---|---|---|---|---|---|---|---|---|---|---|---|
| ≥5 mEq/L | 0 | 3 | (12.0%) | 6 | (21.4%) | 6 | (24.0%) | 8 | (30.8%) | 23 | (22.1%) |
| ≥6 mEq/L | 0 | 3 | (12.0%) | 5 | (17.9%) | 3 | (12.0%) | 4 | (15.4%) | 15 | (14.4%) |
| ≥7 mEq/L | 0 | 1 | (4.0%) | 1 | (3.6%) | 2 | (8.0%) | 2 | (7.7%) | 6 | (5.8%) |

TABLE 6
Proportion of Patients by Serum Bicarbonate Category (Days 8 and 15)

| | | | | | | |
|---|---|---|---|---|---|---|
| Patients with | | | | | | |
| Post-baseline | Pooled | TRC101 | TRC101 | TRC101 | TRC101 | TRC101 |
| Serum | Placebo | 1.5 g BID | 6 g QD | 3.0 g BID | 4.5 g BID | Combined |
| Bicarbonate | N = 31 | N = 25 | N = 28 | N = 25 | N = 26 | N = 104 |

Day 8 Serum Bicarbonate Values

| | | | | | | |
|---|---|---|---|---|---|---|
| >20 mEq/L | 5 (16.1%) | 9 (36.0%) | 16 (57.1%) | 7 (28.0%) | 12 (46.2%) | 44 (42.3%) |
| >22 mEq/L | 2 (6.5%) | 2 (8.0%) | 5 (17.9%) | 5 (20.0%) | 6 (23.1%) | 18 (17.3%) |
| >27 mEq/L | 0 | 0 | 0 | 0 | 0 | 0 |
| >29 mEq/L | 0 | 0 | 0 | 0 | 0 | 0 |

Day 15 Serum Bicarbonate Values

| | | | | | | |
|---|---|---|---|---|---|---|
| >20 mEq/L | 2 (6.5%) | 16 (64.0%) | 17 (60.7%) | 14 (56.0%) | 19 (73.1%) | 69 (66.3%) |
| >22 mEq/L | 0 | 10 (40.0%) | 9 (32.1%) | 7 (28.0%) | 10 (38.5%) | 36 (34.6%) |
| >27 mEq/L | 0 | 0 | 0 | 0 | 0 | 0 |
| >29 mEq/L | 0 | 0 | 0 | 0 | 0 | 0 |

TABLE 7
Treatment-Emergent Adverse Events Occurring in >5%
of Patients in any Treatment Group (Safety Analysis Set)

| | | TRC101 | | | | | |
|---|---|---|---|---|---|---|---|
| | Pooled | | | | | TRC101 | Study |
| | Placebo | 1.5 g BID | 6 g QD | 3.0 g BID | 4.5 g BID | Combined | Total |
| | (N = 31) | (N = 25) | (N = 28) | (N = 25) | (N = 26) | (N = 104) | (N = 135) |
| Preferred Term | n (%) | n (%) | n (%) | n (%) | n (%) | n (%) | n (%) |
| Patients | 14 (45.2) | 13 (52.0) | 17 (60.7) | 9 (36.0) | 17 (65.4) | 56 (53.8) | 70 (51.9) |
| reporting any | | | | | | | |
| TEAE | | | | | | | |
| Diarrhea | 4 (12.9) | 9 (36.0) | 3 (10.7) | 3 (12.0) | 6 (23.1) | 21 (20.2) | 25 (18.5) |
| Headache | 1 (3.2) | 4 (16.0) | 1 (3.6) | 1 (4.0) | 2 (7.7) | 8 (7.7) | 9 (6.7) |
| Constipation | 0 | 1 (4.0) | 3 (10.7) | 1 (4.0) | 2 (7.7) | 7 (6.7) | 7 (5.2) |
| Hyperglycemia | 0 | 0 | 3 (10.7) | 2 (8.0) | 2 (7.7) | 7 (6.7) | 7 (5.2) |
| Hypoglycemia | 2 (6.5) | 2 (8.0) | 0 | 1 (4.0) | 2 (7.7) | 5 (4.8) | 7 (5.2) |
| Hypertension | 1 (3.2) | 1 (4.0) | 2 (7.1) | 0 | 2 (7.7) | 5 (4.8) | 6 (4.4) |
| Glomerular | 2 (6.5) | 2 (8.0) | 0 | 1 (4.0) | 1 (3.8) | 4 (3.8) | 6 (4.4) |
| filtration rate | | | | | | | |
| decreased | | | | | | | |
| Blood glucose | 2 (6.5) | 1 (4.0) | 1 (3.6) | 0 | 0 | 2 (1.9) | 4 (3.0) |
| increased | | | | | | | |

BID = twice daily; GFR = glomerular filtration rate; QD = once daily; TEAE = treatment-emergent adverse event.

### Example 4

Retrospective Analysis of the Benefit of Elevated Serum Bicarbonate Levels

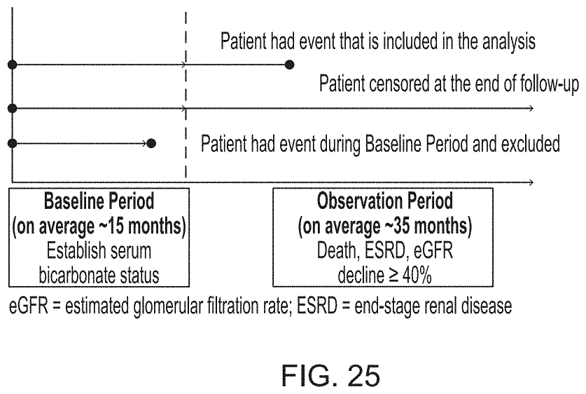

The relationship between decreasing serum bicarbonate levels and worsening clinical outcomes is believed to be a continuum (i.e., as bicarbonate decreases from normal levels the risk of developing adverse outcomes associated with metabolic acidosis, such as death or progression of CKD, progressively increases). To develop a quantitative, predictive model of this relationship and to assess the influence of various CKD comorbidities, a longitudinal analysis using a population from the Optum, Inc. database including CKD patients with serum bicarbonate levels in the range of 12 to 29 mEq/L and eGFR 15 to 45 mL/min/1.73 m² was performed The key advantage of a large longitudinal dataset is the ability to designate a baseline period of significant duration (1 to 2 years) that establishes the presence (or absence) of metabolic acidosis and only then evaluates kidney disease progression. See. e.g. FIG. 25.

Since it can be difficult with retrospective database analyses to distinguish cause and effect (i.e., decreases in eGFR can cause decreases in serum bicarbonate and in turn metabolic acidosis can further cause decline in kidney function), all patients included in the analysis dataset were required to have at least two eGFR and serum bicarbonate measurements over a 1- to 2-year baseline period. Any renal progression events during this period were not counted and patients who died or progressed to dialysis/kidney transplant during the baseline period were excluded. Only after the end of the baseline period did we count the primary outcome event (i.e., death, progression to dialysis or kidney transplant, or 40% reduction from baseline in eGFR; hereafter, “DD40”). In addition, we mandated at least one additional confirmatory serum bicarbonate value in the observation period (i.e., the last available value in the patient's history).

To understand the quantitative relationship of increases in serum bicarbonate to the outcome of interest (i.e., DD40), three separate analyses were performed. These analyses evaluated: 1) the hazard ratio of DD40 in patients with metabolic acidosis (12 to <22 mEq/L); 2) the hazard ratio of DD40 in patients with metabolic acidosis compared to those with normal serum bicarbonate levels (22 to 29 mEq/L); and 3) the benefit (i.e., reduction in hazard ratio of DD40) associated with different magnitudes of serum bicarbonate increase in the population of patients with serum bicarbonate 12 to 20 mEq/L (i.e., the population we intend to enroll in the TRCA-301 and TRCA-303 studies). These analyses are described in more detail below:
Analysis 1: In the subpopulation of acidotic patients with serum bicarbonate values between 12 and <22 mEq/L (N=646), we evaluated the Cox proportional hazards model using baseline serum bicarbonate as a covariate to determine the effect of incremental changes in bicarbonate on the hazard ratio of DD40.
Analysis 2: The subpopulation of acidotic patients with serum bicarbonate values between 12 and <22 mEq/L (N=646) was compared to the subpopulation of patients with normal serum bicarbonate (22 to 29 mEq/L; N=6,535) to quantify the hazard associated with having low serum bicarbonate.
Analysis 3: The subpopulation of patients with serum bicarbonate 12 to 20 mEq/L (i.e., the population to be enrolled in the TRCA-301 and TRCA-303 studies; N=351) was used as the reference group to evaluate the hazard reduction of the DD40 clinical outcome associated with an average 3 or 5 mEq/L increase in serum bicarbonate value. This analysis was performed to quantify the benefit of increases in serum bicarbonate of 2 to <4 mEq/L or ≥4 mEq/L in the population to be enrolled in the TRCA-301 and TRCA-303 studies.

The dataset used for this analysis is an extract of the Optum de-identified Electronic Health Record dataset (2007-2013), which contained longitudinal electronic health records for >22 million unique patient lives. The source of the information in the database was approximately three dozen US health systems in 43 states covering approximately 200 hospitals and 1,800 outpatient clinics. The extract included patients with a documented diagnosis code or clinical evidence of Stage 3, 4, or 5 CKD (based on the Ninth Revision, International Classification of Diseases [ICD-9] code 585.4, 585.5) or an estimated glomerular filtration rate (eGFR)<30 mL/min/1.73 m² at the start of the data period and with at least one serum bicarbonate (HCO₃) test result. The data period of the electronic health records is from January 2007 to July 2013. To exclude erroneous values, only serum bicarbonate values in the range 10 to 40 mEq/L and serum creatinine values in the range 0 to 20 mg/dL were included in the analysis dataset.

To assess the quantitative relationship between a death, dialysis or eGFR decline of at least 40% (a “DD40” endpoint) and serum bicarbonate level, an analysis population was first defined that included only CKD patients with an eGFR value in the range of 15 to <45 mL/min/1.73 m² and a serum bicarbonate level in the range of 12 to 29 mEq/L. Evidence of a Baseline Period of 1 to 2 years duration during which the patient had stable serum bicarbonate and eGFR values, prior to an up to 6.5-year Observation Period during which renal outcomes were assessed.

For inclusion in the analysis population, patients were required to have consistent evidence of the status of their acidosis by remaining in the same serum bicarbonate stratum (i.e., low [12 to 20 mEq/L], borderline [>20 to <22 mEq/L] or normal [22 to 29 mEq/L]) at the following three time points (with slightly wider ranges allowed for the second and third values to account for measurement variability):
1. Baseline serum bicarbonate value, defined as the average of serum bicarbonate results collected within 30 days of the first date of collection from the records with serum bicarbonate results between 10 and 40 mEq/L;
2. First recorded serum bicarbonate value occurring at least 1 year but not more than 2 years after the Baseline HCO3 Date; and
3. Last recorded serum bicarbonate value.

Patients were required to have remained in the same serum bicarbonate stratum to which they were assigned at the beginning of the Baseline Period both at the end of the 1- to 2-year Baseline Period as well as at the end of the Observation Period. This approach was chosen to ensure that DD40 endpoints recorded during the Observation Period could be reliably associated with a particular serum bicarbonate stratum. One drawback of this approach is that the analysis population is likely to be somewhat healthier than the population that will be enrolled in the post-marketing study, TRCA-303. Since this is not expected to exaggerate differences in DD40 event rates observed between strata, the approach is considered reasonable.

Requirements for inclusion in the analysis population also mandated that the patient's eGFR must have remained in the target range of 15 to <45 mL/min/1.73 m² at the beginning, and a target range of 10 to <50 mL/min/1.73 m² at the end, of the 1- to 2-year Baseline Period. The eGFR at the end of the Baseline Period was used as the eGFR Baseline Value from which reductions in eGFR were calculated for the purposes of DD40 endpoint assessment. For a reduction in eGFR from the eGFR Baseline Value to have counted toward the DD40 endpoint, it must have been supported by a confirmatory eGFR value also occurring during the Observation Period that also represented at least a 40% reduction from Baseline.

The requirements for inclusion in the analysis population, and the resulting size of the analysis population after each requirement was implemented, are summarized in 15 and 16, respectively.

Baseline Characteristics for the Analysis Population

The analysis population contained 7,181 CKD patients, which were divided into three strata based on their baseline serum bicarbonate level: 351 patients with low bicarbonate levels (12 to 20 mEq/L), 295 patients with borderline acidosis (>20 to <22 mEq/L) and 6,535 patients with normal serum bicarbonate (22 to 29 mEq/L). Demographic and baseline information for the analysis population by serum bicarbonate stratum is provided in Table 400.

The analysis population was approximately 60% female, with an average age of approximately 74 years. Two-thirds (67%) of patients had a diagnosis of hypertension and 32% had a diagnosis of diabetes. A smaller proportion of patients (12%) had a history of cerebrovascular disease. Renin angiotensin aldosterone system (RAAS) inhibitor use prior to the beginning of the Observation Period was common in the analysis population (˜42% of patients).

Demographic and baseline characteristics were similar among the three serum bicarbonate strata, with the following exceptions: patients in the low serum bicarbonate stratum (12 to 20 mEq/L) were younger, had lower baseline eGFR, and were more often male than patients in the normal serum bicarbonate stratum (22 to 29 mEq/L).

At the time of the first qualifying eGFR value (i.e., at the beginning of the Baseline Period), the majority (79%) of patients in the analysis population had CKD stage 3b, with the remainder having more severe disease (CKD stage 4). The lowest serum bicarbonate stratum had a greater proportion of patients with CKD stage 4 (44%) than did the borderline acidotic and normal serum bicarbonate strata (26% and 20%, respectively).

TABLE 400
Demographics and Baseline Characteristics of the Analysis Population

| | Serum Bicarbonate Stratum | | | | | | | | |
|---|---|---|---|---|---|---|---|---|---|
| | 12 to 20 mEq/L | | >20 to <22 mEq/L | | 22 to 29 mEq/L | | Total | | |
| | (N = 351) | | (N = 295) | | (N = 6,535) | | (N = 7,181) | | p-value |
| Age (years)^{a} | | | | | | | | | <0.0001 |
| N | 351 | | 295 | | 6535 | | 7181 | | |
| Mean (SD) | 69.4 | (11.88) | 72.9 | (9.31) | 74.1 | (8.01) | 73.9 | (8.36) | |
| Median | 73.0 | | 77.0 | | 78.0 | | 78.0 | | |
| Min, Max | 13, 83 | | 22, 83 | | 13, 83 | | 13, 83 | | |
| <18 years | 1 | (0.3%) | 0 | | 2 | (<0.1%) | 3 | (<0.1%) | |
| 18 to <65 years | 94 | (26.8%) | 49 | (16.6%) | 774 | (11.8%) | 917 | (12.8%) | |
| ≥65 years | 256 | (72.9%) | 246 | (83.4%) | 5759 | (88.1%) | 6261 | (87.2%) | |
| Sex | | | | | | | | | 0.0015 |
| Male | 155 | (44.2%) | 145 | (49.2%) | 2590 | (39.6%) | 2890 | (40.2%) | |
| Female | 196 | (55.8%) | 150 | (50.8%) | 3945 | (60.4%) | 4291 | (59.8%) | |
| Hypertension | | | | | | | | | 0.3615 |
| Yes | 225 | (64.1%) | 190 | (64.4%) | 4379 | (67.0%) | 4794 | (66.8%) | |
| No | 126 | (35.9%) | 105 | (35.6%) | 2156 | (33.0%) | 2387 | (33.2%) | |
| Cerebrovascular disease | | | | | | | | | 0.1622 |
| Yes | 40 | (11.4%) | 25 | (8.5%) | 791 | (12.1%) | 856 | (11.9%) | |
| No | 311 | (88.6%) | 270 | (91.5%) | 5744 | (87.9%) | 6325 | (88.1%) | |
| Diabetes | | | | | | | | | 0.0655 |
| Yes | 118 | (33.6%) | 110 | (37.3%) | 2042 | (31.2%) | 2270 | (31.6%) | |
| No | 233 | (66.4%) | 185 | (62.7%) | 4493 | (68.8%) | 4911 | (68.4%) | |
| Baseline ACEi or ARB Use | | | | | | | | | 0.1582 |
| Yes | 162 | (46.2%) | 135 | (45.8%) | 2704 | (41.4%) | 3001 | (41.8%) | |
| No | 150 | (42.7%) | 118 | (40.0%) | 2961 | (45.3%) | 3229 | (45.0%) | |
| Missing | 39 | (11.1%) | 42 | (14.2%) | 870 | (13.3%) | 951 | (13.2%) | |
| Baseline Serum Bicarbonate (HCO3) (mEq/L)^{b} | | | | | | | | | <0.0001 |
| N | 351 | | 295 | | 6535 | | 7181 | | |
| Mean (SD) | 18.3 | (1.65) | 21.0 | (0.26) | 25.3 | (2.02) | 24.8 | (2.59) | |
| Median | 19.0 | | 21.0 | | 25.0 | | 25.0 | | |
| Min, Max | 13, 20 | | 20, 22 | | 22, 29 | | 13, 29 | | |
| First Qualifying eGFR (mL/min/1.73 m²) | | | | | | | | | <0.0001 |
| N | 351 | | 295 | | 6535 | | 7181 | | |
| Mean (SD) | 30.7 | (7.86) | 33.7 | (7.28) | 35.3 | (6.56) | 35.0 | (6.74) | |
| Median | 31.0 | | 35.0 | | 36.0 | | 36.0 | | |
| Min, Max | 15, 44 | | 15, 44 | | 15, 44 | | 15, 44 | | |
| Stage 3b Moderate CKD | 197 | (56.1%) | 217 | (73.6%) | 5260 | (80.5%) | 5674 | (79.0%) | |
| (30 to 44 | | | | | | | | | |
| mL/min/1.73 m²) | | | | | | | | | |
| Stage 4 Severe CKD | 154 | (43.9%) | 78 | (26.4%) | 1275 | (19.5%) | 1507 | (21.0%) | |
| (15 to 29 | | | | | | | | | |
| mL/min/1.73 m²) | | | | | | | | | |

ACEi = angiotensin converting enzyme inhibitor;
ARB = angiotensin receptor blocker;
CKD = chrome kidney disease;
eGFR = estimated glomerular filtration rate;
HCO3 = serum bicarbonate;
SD = standard deviation
Note:
Race and urine albumin-to-creatinine ratio not included because >95% and >85% of patients, respectively, did not have this information recorded.
^{a}Age was calculated as the difference between date of birth and date of baseline bicarbonate, in years. All patients had their birth month and day set to June 1. Patients with no available birth date (i.e., no birth year) had their birth year set to the same year as the baseline serum bicarbonate measurement collection year (i.e., age = 0 years). Patients with birth year of 1928 or earlier had their birth year set to 1928 (i.e., age = 85 years).
^{b}Baseline serum bicarbonate calculated as average of all HCO₃ values within 30 days of first HCO₃ value.

Qualifying Serum Bicarbonate and eGFR Values for the Analysis Population

Because patients were required to remain in the same serum bicarbonate stratum to which they were assigned at the beginning of the Baseline Period, there was little difference in the mean serum bicarbonate values in a particular stratum over the course of the Baseline and Observation Periods. For example, patients in the low serum bicarbonate stratum had an average serum bicarbonate level of 18.3 mEq/L at the beginning of the Baseline Period, 18.5 mEq/L at the end of the Baseline Period (which is also the beginning of the Observation Period), and 18.5 mEq/L at the end of the Observation Period. Similarly, patients in the borderline and normal serum bicarbonate strata had serum bicarbonate values across the analysis periods that did not vary significantly (Table 500). The length of the Baseline Period varied among patients because it was determined by the time of the first serum bicarbonate record occurring at least 1 year but not more than 2 years after the patient's first recorded serum bicarbonate value. The average duration of the Baseline Period, determined by the second qualifying serum bicarbonate value, was 15.2 months in the analysis population overall, and it was similar among the three serum bicarbonate strata (Table 500).

To ensure that we were assessing the effects of various levels of acidosis in CKD patients who represent our intended TRCA-303 population, patients in the analysis population were required to have an eGFR value at least 1 year but not more than 2 years after the patient's first recorded serum bicarbonate value that was in the range between 10 and 50 mL/min/1.73 m². The patient's Baseline eGFR Value was calculated from the average of all serum creatinine values recorded in the 90 days prior to this second qualifying eGFR value. The mean Baseline eGFR Values were 29.6, 32.8 and 35.4 mL/min/1.73 m² in the low, borderline and normal serum bicarbonate groups, respectively. The duration of the Baseline Period, as defined by the time between the first and second qualifying eGFR values, was 15.06 months in the analysis population overall, and it was similar among the three serum bicarbonate strata (Table 500). A patient's Baseline eGFR Value was used for assessment of all DD40 endpoint events occurring during the Observation Period. Reductions in eGFR sufficiently large to contribute to the DD40 event rate (i.e., 40%) were calculated as reductions from this eGFR value, not the eGFR at the beginning of the Baseline Period. All reductions in eGFR contributing to the DD40 event rate were also confirmed by a second eGFR value that confirmed magnitude of the reduction.

The average duration of the Observation Period, as defined by the time from the Baseline eGFR Date to the last recorded patient contact, was 34.9 months in the analysis population overall, and ranged from 32.9 to 35.0 months among the three serum bicarbonate strata.

TABLE 500
Serum Bicarbonate and eGFR Values of the Analysis Population

| | Serum Bicarbonate Stratum | | | |
|---|---|---|---|---|
| | 12 to 20 mEq/L | >20 to <22 mEq/L | 22 to 29 mEq/L | Total |
| All Patients | (N = 351) | (N = 295) | (N = 6,535) | (N = 7,181) |

Baseline Serum Bicarbonate (mEq/L; First HCO3 Value)

| | | | | | | | | |
|---|---|---|---|---|---|---|---|---|
| Mean (SD) | 18.3 | (1.65) | 21.0 | (0.26) | 25.3 | (2.02) | 24.8 | (2.59) |
| Median (Min, Max) | 19.0 | (13, 20) | 21.0 | (20, 22) | 25.0 | (22, 29) | 25.0 | (13, 29) |

First Qualifying eGFR (mL/min/1.73 m²)

| | | | | | | | | |
|---|---|---|---|---|---|---|---|---|
| Mean (SD) | 30.7 | (7.86) | 33.7 | (7.28) | 35.3 | (6.56) | 35.0 | (6.74) |
| Median (Min, Max) | 31.0 | (15, 44) | 35.0 | (15, 44) | 36.0 | (15, 44) | 36.0 | (15, 44) |

Second Qualifying Serum Bicarbonate (mEq/L; i.e., End of 1- to 2-year Baseline Period)

| | | | | | | | | |
|---|---|---|---|---|---|---|---|---|
| Mean (SD) | 18.5 | (2.37) | 21.2 | (1.50) | 25.0 | (2.29) | 24.5 | (2.75) |
| Median (Min, Max) | 19.0 | (10, 22) | 21.0 | (18, 24) | 25.0 | (20, 29) | 25.0 | (10, 29) |

Baseline eGFR Value (mL/min/1.73 m²; i.e., End of 1- to 2-year Baseline Period)

| | | | | | | | | |
|---|---|---|---|---|---|---|---|---|
| Mean (SD) | 29.6 | (9.25) | 32.8 | (8.93) | 35.4 | (7.98) | 35.0 | (8.20) |
| Median (Min, Max) | 30.0 | (10, 49) | 34.0 | (10, 49) | 36.0 | (10, 49) | 36.0 | (10, 49) |

Last Serum Bicarbonate (mEq/L)

| | | | | | | | | |
|---|---|---|---|---|---|---|---|---|
| Mean (SD) | 18.5 | (2.51) | 21.0 | (1.61) | 24.9 | (2.40) | 24.4 | (2.85) |
| Median (Min, Max) | 19.0 | (10, 22) | 21.0 | (18, 24) | 25.0 | (20, 29) | 25.0 | (10, 29) |

Time (months) from First HCO3 to Second Qualifying Serum Bicarbonate Value (i.e., length of Baseline Period)

| | | | | | | | | |
|---|---|---|---|---|---|---|---|---|
| Mean (SD) | 15.35 | (3.283) | 14.95 | (2.851) | 15.21 | (3.149) | 15.20 | (3.144) |
| Median (Min, Max) | 14.10 | (12.0, 24.0) | 13.90 | (12.0, 23.9) | 14.10 | (12.0, 24.0) | 14.10 | (12.0, 24.0) |

Time (months) from First Qualifying eGFR to Baseline eGFR Date (i.e., length of Baseline Period)

| | | | | | | | | |
|---|---|---|---|---|---|---|---|---|
| Mean (SD) | 14.90 | (3.016) | 14.83 | (2.847) | 15.08 | (3.081) | 15.06 | (3.069) |
| Median (Min, Max) | 13.80 | (12.0, 23.8) | 13.80 | (12.0, 23.9) | 14.00 | (12.0, 24.0) | 13.90 | (12.0, 24.0) |

Time (months) from Baseline eGFR Date to Last Contact Date (i.e., length of Observation Period)

| | | | | | | | | |
|---|---|---|---|---|---|---|---|---|
| Mean (SD) | 32.92 | (19.80) | 34.74 | (20.61) | 35.02 | (19.47) | 34.90 | (19.53) |
| Median (Min, Max) | 32.30 | (0.0, 73.0) | 33.60 | (0.4, 72.2) | 35.30 | (0.0, 77.1) | 35.20 | (0.0, 77.1) |

eGFR = estimated glomerular filtration rate;
HCO₃ = serum bicarbonate;
SD = standard deviation

Analysis

Frequency of Endpoint Events by Serum Bicarbonate Stratum

During the almost 6.5-year follow up of the 7,181 patients in the analysis population, 572 patients experienced the DD40 endpoint (Table 600). Of these patients, 50 died, 60 initiated dialysis or received renal transplantation and 462 had a 40% decline from baseline in eGFR. The incidence rate of each of the individual endpoint events, as well as of the DD40 composite endpoint, was 2.4- to 6.7-fold higher in the group of patients with low baseline serum bicarbonate than in the patients with normal serum bicarbonate.

TABLE 600
Frequency and Incidence Rates of Endpoint Events by Serum Bicarbonate Stratum

| | 12 to 20 | | >20 to <22 | | 12 to <22 | | 22 to 24 | | 22 to 29 | | | |
|---|---|---|---|---|---|---|---|---|---|---|---|---|
| Endpoint | mEq/L | | mEq/L | | mEq/L | | mEq/L | | mEq/L | | Total | |
| Event | (N = 351) | | (N = 295) | | (N = 646) | | (N = 1,572) | | (N = 6,535) | | (N = 7,181) | |
| DD40 | 71 | (20.2%) | 35 | (11.9%) | 106 | (16.4%) | 110 | (7.0%) | 466 | (7.1%) | 572 | (8.0%) |
| Death | 7 | (2.0%) | 5 | (1.7%) | 12 | (1.9%) | 9 | (0.6%) | 38 | (0.6%) | 50 | (0.7%) |
| Dialysis or | 14 | (4.0%) | 6 | (2.0%) | 20 | (3.1%) | 9 | (0.6%) | 40 | (0.6%) | 60 | (0.8%) |
| kidney | | | | | | | | | | | | |
| transplant | | | | | | | | | | | | |
| ≥40% decline | 50 | (14.2%) | 24 | (8.1%) | 74 | 11.5%) | 92 | 5.9%) | 388 | (5.9%) | 462 | (6.4%) |
| from baseline | | | | | | | | | | | | |
| in eGFR | | | | | | | | | | | | |

Number (%) of patients are reported.
DD40 = the composite of death, dialysis or kidney transplant, and ≥40% decline from baseline in eGFR;
eGFR = estimated glomerular filtration rate

Cox Regression Analyses

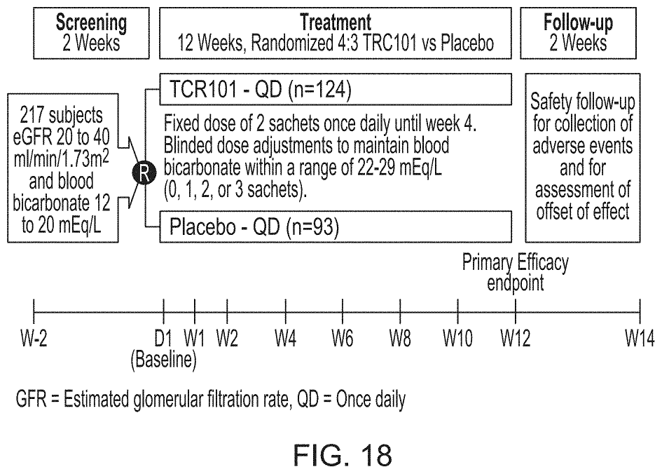

To understand the impact of each 1 mEq/L increase in serum bicarbonate on the hazard reduction of DD40, three separate analyses were performed (17). These analyses evaluated: 1) the hazard ratio of DD40 in patients with metabolic acidosis (12 to 22 mEq/L); 2) the hazard ratio of DD40 in patients with metabolic acidosis compared to those with normal serum bicarbonate levels (22 to 29 mEq/L); and 3) the benefit (i.e., reduction in hazard ratio of DD40) associated with different magnitudes of serum bicarbonate increase compared with the population of patients with serum bicarbonate 12 to 20 mEq/L (FIG. 17).

Analysis 1: Effect of Incremental Changes in Serum Bicarbonate on the Hazard of Dd40

For the subpopulation of patients with below-normal serum bicarbonate, we evaluated the Cox proportional hazards model for the hazard ratio of the DD40 clinical endpoint adjusted for the following covariates: baseline bicarbonate, age, sex, hypertension, cerebrovascular disease, diabetes, baseline angiotensin converting enzyme (ACE) inhibitor or angiotensin receptor blocker (ARB) use, and initial eGFR. Stepwise model selection using the backward-forward method was applied. A variable had to be significant at the 0.3 level before it could be entered into the model, and the variable had to be significant at the 0.21 level for it to remain in the final Cox proportional-hazards regression model. The significance level for entry into and staying in the model was intentionally conservative to allow variables to be included in the final model, even if all were not significant at the 0.05 level.

As shown in Table 401, each 1 mEq/L increase in baseline serum bicarbonate was associated with a reduction in the DD40 hazard of approximately 12%.

TABLE 401
Cox Regression Model for DD40 (Patients
with Serum Bicarbonate 12 to 22 mEq/L)

| | Hazard Ratio (95% CI) | |
|---|---|---|
| Covariate^{a} | (N = 646) | p-value |
| Baseline HCO₃ increases | 0.88 (0.80, 0.97) | 0.0084 |
| of 1 mEq/L^{b} | | |
| Sex (Male/Female) | 1.33 (0.90, 1.94) | 0.1492 |
| Diabetes (Yes/No) | 1.63 (1.11, 2.40) | 0.0125 |
| Cerebrovascular disease | 1.55 (0.88, 2.74) | 0.1307 |
| (Yes/No) | | |
| Initial qualifying eGFR | 1.04 (1.02, 1.07) | 0.0013 |
| decreases of 1 (mL/min/1.73 m²) | | |

ACEi = angiotensin converting enzyme inhibitor;
ARB = angiotensin receptor blocker;
CI = confidence interval;
eGFR = estimated glomerular filtration rate;
HCO₃ = serum bicarbonate
^{c} Cox proportional hazard model used stepwise selection for baseline HCO₃ value, age (<65 or ≥65), sex (Male or Female), hypertension (Yes, No), cerebrovascular disease (Yes, No), diabetes (Yes, No), initial qualifying eGFR value, and baseline ACEi/ARB use (Yes, No).
^{d} Baseline serum bicarbonate was calculated as the average of measurements within 30 days of first measurement of HCO₃.

Analysis 2: Effect of Low Serum Bicarbonate on DD40

A Cox regression analysis adjusted for multiple covariates (i.e., age, sex, hypertension, cerebrovascular disease, diabetes, baseline ACE inhibitor or ARB use, and initial eGFR) was performed with two serum bicarbonate strata: patients with baseline serum bicarbonate in the ranges of 12 to <22 mEq/L and 22 to 29 mEq/L.

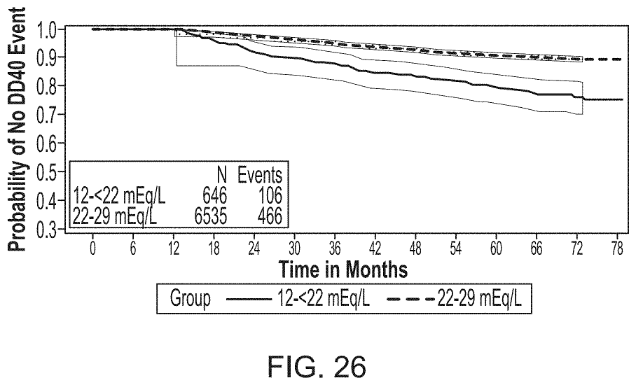

The results demonstrate that the hazard ratio of the DD40 endpoint is higher in the group of patients with low serum bicarbonate compared with those with normal serum bicarbonate. The adjusted hazard ratio was 1.79 (95% confidence interval [CI]: 1.31, 2.43; p=0.0002; Table 501; FIG. 26).

We also explored the potential impact of factors other than serum bicarbonate level on the DD40 hazard ratio in our analysis population. The factors investigated included demographic factors (age, sex), comorbidities common in the CKD population (hypertension, diabetes, cerebrovascular disease), use of an ACE inhibitor or ARB at baseline and the patient's initial eGFR value as a measure of the severity of their renal disease. The hazard ratios (95% CI) for each factor, while adjusting for the other factors, are provided in Table 501.

TABLE 501
Cox Model Hazard Ratios (95% CI) for DD40 by Serum Bicarbonate Stratum

| | | | |
|---|---|---|---|
| 12 to <22 mEq/L | 22 to 29 mEq/L | | |
| (N = 646) | (N = 6,535) | Covariate | p-value |

Baseline serum bicarbonate (mEq/L)^{a}

| | | | | |
|---|---|---|---|---|
| Mean (SD) | 19.6 | (1.82) | 25.3 | (2.02) |

Number (%) patients

| | | | | |
|---|---|---|---|---|
| With | 106 | (16.4%) | 466 | (7.1%) |
| Event during | | | | |
| Observation | | | | |
| Period | | | | |

Model without adjustment^{a}

| | | | | |
|---|---|---|---|---|
| Hazard Ratio | 2.50 | (2.02, 3.09) | Reference | None |
| (95% CI) | | | | |

| | | |
|---|---|---|
| p-value | <0.0001 | Reference |

Model with adjustment^{b}

| | | | |
|---|---|---|---|
| Hazard Ratio | 1.79 | (1.31, 2.43) | Reference |
| (95% CI) | | | |

| | | |
|---|---|---|
| p-value | 0.0002 | Reference |

| | | |
|---|---|---|
| Age (≥65 years/<65 years) | 0.68 (0.51, 0.91) | 0.0091 |
| Diabetes (Yes/No) | 1.57 (1.23, 2.00) | 0.0002 |
| Hypertension (Yes/No) | 0.77 (0.56, 1.07) | 0.1163 |
| Cerebrovascular disease (Yes/No) | 1.83 (1.38, 2.43) | <0.0001 |
| Initial qualifying eGFR value | 1.06 (1.04, 1.08) | <0.0001 |
| by decreases of 1 mL/min/1.73 m² | | |

ACEi = angiotensin converting enzyme inhibitor;
ARB = angiotensin receptor blocker;
CI = confidence interval;
eGFR = estimated glomerular filtration rate;
SD = standard deviation
^{e} Cox proportional hazard model without adjustment for covariates.
^{f} Cox proportional hazard model stepwise selection for age (<65 or ≥65), sex (male or female), hypertension (Yes, No), cerebrovascular disease (Yes, No), diabetes (Yes, No), initial qualifying eGFR value, and baseline ACEi/ARB use (Yes, No).
The p-value for these covariates must be <0.3 to be included in the model.

Analysis 3: Effect of Incrementally Higher Serum Bicarbonate Levels on Dd40

As shown in Table 700, the hazard ratio of the DD40 endpoint is reduced with both moderate and large increases in serum bicarbonate. This analysis used the low serum bicarbonate (12 to 20 mEq/L) stratum as Reference for comparison with two strata with higher average serum bicarbonate levels. The average baseline serum bicarbonate level in the Reference stratum was 18.3 mEq/L. The Test strata had average baseline serum bicarbonate levels of 21.0 and 23.1 mEq/L, representing increases in serum bicarbonate of approximately 3 and 5 mEq/L. A 3 mEq/L higher average serum bicarbonate level resulted in an adjusted hazard ratio of 0.60 (95% CI: 0.40, 0.91; p=0.0153), indicating that moderately higher serum bicarbonate levels significantly reduce the hazard of the DD40 endpoint. A 5 mEq/L higher average serum bicarbonate level resulted in an adjusted hazard ratio of 0.39 (95% CI: 0.29, 0.53; p<0.0001).

TABLE 700
Hazard Ratios and 95% Confidence Intervals for DD40 from Cox Model
(10 <= Baseline eGFR < 50 mL/min/1.73 m²)
Patients^{a} with Baseline SBC >= 12 mEq/L and <= 29 mEq/L and First Qualifying eGFR >= 15 and < 45 mL/min/1.73 m²

| Baseline Serum Bicarbonate | | | | | | |
|---|---|---|---|---|---|---|
| >27-29 mEq/L | >24-27 mEq/L | 22-24 mEq/L | >20-<22 mEq/L | 12-20 mEq/L | | |
| (N = 612) | (N = 2413) | (N = 1572) | (N = 295) | (N = 351) | Covariate | p-value^{e} |

Baseline serum bicarbonate (mEq/L)

| n | 612 | | 2413 | | 1572 | | 295 | | 351 | |
|---|---|---|---|---|---|---|---|---|---|---|
| Mean (SD) | 28.4 | (0.54) | 25.9 | (0.82) | 23.1 | (0.79) | 21.0 | (0.26) | 18.3 | (1.65) |

| | | | | | |
|---|---|---|---|---|---|
| Median | 28.0 | 26.0 | 23.0 | 21.0 | 19.0 |
| Min, Max | 27, 29 | 24, 27 | 22, 24 | 20, 22 | 13, 20 |

1st qualifying estimated glomerular filtration rate (eGFR) (mL/min/1.73 m²)

| n | 612 | | 2413 | | 1572 | | 295 | | 351 | |
|---|---|---|---|---|---|---|---|---|---|---|
| Mean (SD) | 36.7 | (5.91) | 35.5 | (6.44) | 34.9 | (6.81) | 33.7 | (7.28) | 30.7 | (7.86) |

| | | | | | |
|---|---|---|---|---|---|
| Median | 38.0 | 36.0 | 36.0 | 35.0 | 31.0 |
| Min, Max | 17, 44 | 15, 44 | 15, 44 | 15, 44 | 15, 44 |

Length of observation period (months)

| n | 612 | | 2413 | | 1572 | | 295 | | 351 | |
|---|---|---|---|---|---|---|---|---|---|---|
| Mean (SD) | 33.25 | (19.62) | 34.15 | (19.67) | 34.54 | (19.86) | 34.74 | (20.61) | 32.92 | (19.80) |

| | | | | | |
|---|---|---|---|---|---|
| Median | 33.02 | 33.91 | 34.30 | 33.58 | 32.30 |
| Min, Max | 0.1, 71.3 | 0.0, 77.1 | 0.1, 71.0 | 0.4, 72.2 | 0.0, 73.0 |

Number (%) patients

| | | | | | | | | | | |
|---|---|---|---|---|---|---|---|---|---|---|
| Had Event | 23 | (3.8%) | 154 | (6.4%) | 110 | (7.0%) | 35 | (11.9%) | 71 | (20.2%) |
| (New DD40 >1 | | | | | | | | | | |
| Year (baseline | | | | | | | | | | |
| eGFR date 1 | | | | | | | | | | |
| to 2 years)) | | | | | | | | | | |
| Censored | 589 | (96.2%) | 2259 | (93.6%) | 1462 | (93.0%) | 260 | (88.1%) | 280 | (79.8%) |

Model without adjustment^{b}

| | | | | | | | | | | |
|---|---|---|---|---|---|---|---|---|---|---|
| Hazard Ratio | 0.17 | (0.10, 0.27) | 0.28 | (0.21, 0.37) | 0.31 | (0.23, 0.42) | 0.54 | (0.36, 0.81) | Reference | None |
| (95% CI) | | | | | | | | | | |

| | | | | | |
|---|---|---|---|---|---|
| p-value^{c} | <.0001 | <.0001 | <.0001 | 0.0027 | Reference |

Model with adjustment^{d}

| | | | | | | | | | |
|---|---|---|---|---|---|---|---|---|---|
| Hazard Ratio | 0.25 | (0.16, 0.41) | 0.41 | (0.31, 0.55) | 0.43 | (0.32, 0.59) | 0.66 | (0.44, 0.99) | Reference |
| (95% CI) | | | | | | | | | |

| p-value^{c} | <.0001 | <.0001 | <.0001 | 0.0452 | Reference | | |
|---|---|---|---|---|---|---|---|
| Age (>=65 years/ | | | | | | 0.69 (0.54, 0.89) | 0.0046 |
| <65 years) | | | | | | | |
| Sex | | | | | | 1.24 (1.01, 1.51) | 0.0367 |
| (Male/Female) | | | | | | | |
| Diabetes | | | | | | 1.68 (1.36, 2.06) | <.0001 |
| (Yes/No) | | | | | | | |
| Cerebrovascular | | | | | | 1.76 (1.35, 2.28) | <.0001 |
| disease (Yes/No) | | | | | | | |
| Initial | | | | | | 1.06 (1.04, 1.07) | <.0001 |
| qualifying eGFR | | | | | | | |
| decreases of 1 | | | | | | | |
| (mL/min/1.73 m²) | | | | | | | |
| Baseline ACEi | | | | | | 1.24 (1.01, 1.53) | 0.0366 |
| or ARB use | | | | | | | |

Note:
Events are counted when occurred >=365 days after the first recorded HCO3 value.
^{a}Baseline serum bicarbonate was calculated as the average of measurements within 30 days of first measurement of HCO3 (between 10 and 40 mEq/L).
^{b}Cox proportional hazard model stepwise selection for age (<65 or >=65), sex (Male or Female), hypertension (Yes, No), cerebrovascular disease (Yes, No), diabetes (Yes, No), initial qualifying eGFR value, and baseline ACEi/ARB use (Yes, No). The p-value for these covariates must be <0.3 to be included in the model.
^{c}p-value is testing for the hazard ratio for eGFR decline >=40% in patients with a baseline SBC category over the reference category.
^{d}Cox proportional hazard model stepwise selection for age (<65 or >=65), sex (Male or Female), hypertension (Yes, No), cerebrovascular disease (Yes, No), diabetes (Yes, No), initial qualifying eGFR value, baseline proteinuria (Moderate or Severe), baseline proteinuria (Severe), and baseline ACEi/ARB use (Yes, No). The p-value for these covariates must be <0.3 to be included in the model.
^{e}The p-value is testing effect of covariates.

Conclusion

The subpopulations of patients that represent increases in serum bicarbonate of 2 to <4 mEq/L or ≥4 mEq/L in the acidotic (serum bicarbonate 12 to 20 mEq/L) population have a 40% and 61% reduced hazard of DD40, respectively. This result suggests that each 1 mEq/L increase of serum bicarbonate reduces hazard of DD40 by ˜12 to 13%.

### Example 5

### Description and Analysis of Results from Clinical Trial

A double blind, randomized, placebo-controlled study that enrolled 217 subjects with Stage 3b or 4 CKD (an estimated glomerular filtration rate [eGFR] of 20 to 40 mL/min/1.73 m²) and low blood bicarbonate levels (between 12 mEq/L and 20 mEq/L) was conducted. At the beginning of the 12-week treatment period, subjects were randomized in a 4:3 ratio to receive once-daily, or QD, a pharmaceutical composition according to the present invention, e.g., TRC101, or placebo. Subjects in the active group initially received a QD dose of 6 grams of TRC101 (2 sachets). After week 4, bi-directional blinded dose adjustments to 3 grams/day (1 sachet) or 9 grams/day (3 sachets) were allowed in order to maintain blood bicarbonate in the normal range. Subjects in the placebo group initially received 2 sachets of placebo, with the same ability for bi-directional dose adjustments after 4 weeks. The dose titration algorithm required down-titration at blood bicarbonate values of 27 to 30 mEq/L. Subjects with a blood bicarbonate level greater than 30 underwent an interruption of the study drug in accordance with the titration algorithm. Subjects were permitted to continue their existing oral alkali supplement during the trial, provided that dosing remained stable. The trial was conducted at 47 sites in the United States and Europe.

Eligible patients were aged 18 to 85 years and had systolic blood pressure<170 mmHg and hemoglobin A1c≤9%. During the up to 2-week Screening Period, three qualifying fasting serum bicarbonate values over 14 days were required to establish eligibility; the first two values and the average of all three were required to be within the range 12-20 mmol/L. Two qualifying eGFR values not different by >20% and in the range 20-40 mL/min/1.73 m² were required during screening. Patients were excluded if their serum bicarbonate level was low enough to need emergency intervention or evaluation for an acute acidotic process, or if in the 3 months prior to the first Screening Visit they had anuria, dialysis, or acute or chronic worsening renal function (e.g., ≥30% decline in eGFR). Patients with recent history of chronic obstructive pulmonary disease, heart failure with New York Heart Association Class IV symptoms, stroke, transient ischemic attack, cancer, cardiac event, diabetic gastroparesis, bariatric surgery, bowel obstruction, swallowing disorders, severe gastrointestinal disorders, or hospitalization other than for pre-planned diagnostic or minor invasive procedures, those who had a heart or kidney transplant, and those who planned initiation of renal replacement therapy within 12 weeks were also excluded. Eligible patients did not have liver enzyme levels>3 times the upper limit of normal, serum calcium levels≤2 mmol/L or serum potassium levels<3.8 mmol/L or >5.9 mmol/L. Concomitant medication requirements for study participation precluded use of any other investigational medication as well as other binder drugs (except for short-term use of potassium binders for treatment of hyperkalemia) and required stable doses (whenever possible) of the following if they were used: calcium-containing supplements; antacids; H2-blockers; proton pump inhibitors; oral alkali; diuretics; renin-angiotensin-aldosterone system inhibitors; and non-ophthalmic carbonic anhydrase inhibitors. Dosing of oral concomitant medications and study drug was separated by ≥4 hours.

The starting study drug dose was 6 grams/day TRC101 (2 packets/day) or placebo (2 packets/day) administered orally as a suspension in water with lunch. The first dose was administered in the clinic on the day of randomisation, after which, patients self-administered the study drug for 12 weeks and recorded the dose in a diary, which was reviewed, together with used and unused study drug returned at each visit. Beginning at Week 4, the study drug dose was algorithmically titrated by the interactive response technology system in the range from 0-9 grams/day (or equivalent number of packets of placebo) to a target bicarbonate of 22-29 mmol/L based on the bicarbonate measurement at each visit. The dose was down-titrated if bicarbonate was high-normal (27-30 mmol/L) and interrupted if it was >30 mmol/L (Table 800).

TABLE 800
Dose Titration Algorithm

| Serum | | |
|---|---|---|
| Bicarbonate | | |
| (mmol/L) | Before Week 4 Visit | Week 4 through Week 11 |
| <12* | Evaluate for new acute | Evaluate for new acute |
| | acidotic process, contact | acidotic process, contact |
| | Medical Monitor. Maintain | Medical Monitor. Maintain |
| | dose pending discussion | dose pending discussion |
| | with Medical Monitor | with Medical Monitor. |
| 12 to <22 | Maintain dose until next | Increase the study drug |
| | scheduled visit. | dose by 1 packet/day |
| | | (maximum dose is 3 packets/ |
| | | day). Only increase the |
| | | dose if NO dose changes |
| | | have been made during the |
| | | previous 14 days. Retest |
| | | serum bicarbonate at next |
| | | scheduled visit. |

| | |
|---|---|
| 22 to <27 | Maintain dose until next scheduled visit. |
| 27 to 30 | Decrease the study drug dose by 1 packet/day (minimum |
| | dose is 0 packets/day). Invite subject for a visit in |
| | approximately 1 week to retest serum bicarbonate. Only |
| | decrease the dose if NO dose changes have been made |
| | during the previous 14 days. |
| >30* | Interrupt (hold) study drug. |
| | Invite subject for a visit in approximately 1 week to |
| | retest serum bicarbonate. If serum bicarbonate at that |
| | visit is: |
| | <27 mmol/L, restart study drug at a lower dose |
| | (1 packet/day less than before dose interruption). |
| | ≥27 |
| | mmol/L, continue to hold the dose and retest again in |
| | approximately 1 week. |

*Serum bicarbonate value of <12 mmol/L or >30 mmol/L confirmed by a repeated measurement from a separate blood draw.

Comorbid conditions between treated and placebo subjects entering the trial were equally balanced and included: 97% with hypertension, 65% with type 2 diabetes, 44% with left ventricular hypertrophy, and 31% with congestive heart failure; during the three months prior to baseline, 12% of subjects had shortness of breath with exertion and 9% had recurrent or continuous signs/symptoms of edema or fluid overload. Nine percent of the total patient population in the trial reported the use of oral alkali therapy at baseline.

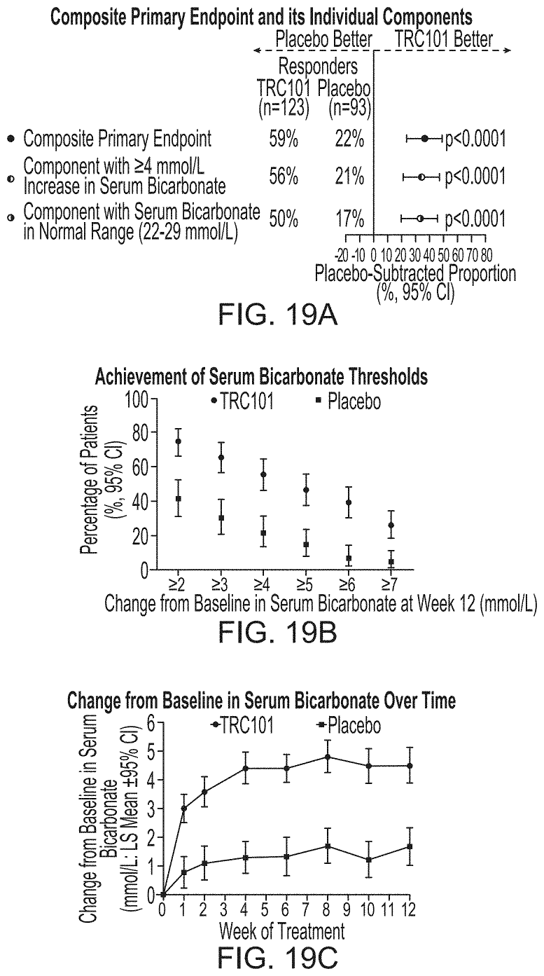

The blood bicarbonate levels of subjects were measured on day 1, week 1, week 2, and bi-weekly thereafter, up to and including week 14 (see, e.g., FIG. 18). The primary efficacy endpoint of the trial was an increase in blood bicarbonate level of at least 4 mEq/L or achieving a blood bicarbonate level in the normal range of 22 to 29 mEq/L at the end of the 12-week treatment period. The secondary efficacy endpoint of the trial was the change from baseline in blood bicarbonate at the end of treatment.

The study was conducted according to the principles of the Declaration of Helsinki and according to Good Clinical Practice guidelines. The study protocol was approved by each site's relevant institutional review board or ethics committee and appropriate competent authorities in accordance with applicable laws and regulations. Prior to enrollment, all patients provided written informed consent. An unblinded, independent Data Monitoring Committee performed scheduled reviews of safety data during the study.

Procedures

During the Screening Period, the Screening 1 and Screening 2 Visits were ≥5 days apart, and Screening 1 and Baseline Visits were ≤1.4 days apart. Following randomisation, patients attended scheduled visits at Weeks 1, 2, 4, 6, 8, 10, and 12 during which serum bicarbonate was measured using an i-STAT® Handheld Blood Analyzer (Abbott Point of Care) and safety assessments were conducted (FIG. 18: eGFR, estimated glomerular filtration rate; n, number of patients in each treatment group; QD, once daily; R, randomization; W, week).

Patients fasted for hours (other than water) prior to measurements of bicarbonate levels to reduce the indirect effect of food-induced secretion of bicarbonate into the bloodstream. Venous blood for bicarbonate measurement was drawn into a 2 mL lithium heparin tube and transferred with a mini-pipette as soon as possible (within 10 minutes) into an i-STAT G3+cartridge for assessment of bicarbonate with the i-STAT device. Tubes were capped until blood was transferred into the cartridge, and strict adherence to blood drawing and transfer techniques were required. The i-STAT devices were calibrated prior to and during the study according to the manufacturer's recommendations. The Kidney Disease and Quality of Life (KDQOL) Short Form-36, Question 3 (Physical Functioning Domain) (FIG. 21) and standardized repeated chair stand test (FIGS. 22A & 22B) were administered at baseline and Week 12. The KDQOL was forward and backwards translated, linguistically validated, culturally adapted, reviewed by clinicians, and cognitively debriefed in CKD patients. Following completion of study treatment at Week 12, patients either rolled over into a 40-week extension study or underwent two follow-up visits (Week 13 and Week 14) after the last dose of study drug.

Serum Bicarbonate

A total of 71 of 120 (59%) TRC101-treated patients and 20 of 89 (22%) placebo-treated patients met the primary endpoint responder definition (p<0.0001 for the comparison), with a treatment difference (TRC101—placebo) of 37% (95% CI, 23%-49%). A similar placebo-subtracted treatment difference was observed for each of the two components of the primary endpoint. Compared with the placebo group, a higher percentage of patients in the TRC101 group had increases in serum bicarbonate at all pre-defined thresholds (≥2 through ≥7 mmol/L).

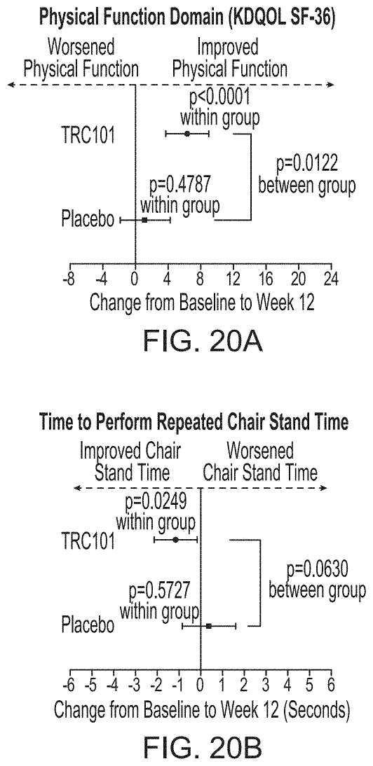

The serum bicarbonate curves for the TRC101 and placebo groups separated over time starting at Treatment Week 1 and maintained separation through the end of treatment (FIG. 19C). At Week 12, the mean change from baseline in the TRC101 and placebo groups was 4.5 (95% CI, 3.9 to 5.1) mmol/L and 1.7 (95% CI, 1.0 to 2.3) mmol/L, respectively (p<0.0001). The LS mean (SEM) change from baseline to Week 12, the secondary endpoint, was 4.4 (3.5) mmol/L and 1.8 (3.1) mmol/L in the TRC101 and placebo groups, respectively (p<0.0001). (FIG. 19A-C: Change in Serum Bicarbonate—FIG. 19A: The composite primary endpoint, the placebo-subtracted percentage of patients achieving a ≥4 mmol/L increase from baseline in serum bicarbonate or a serum bicarbonate in the normal range (22-29 mmol/L) at Treatment Week 12 (37%, 95% CI: 23%, 49%), is depicted as the top line. The two lower lines depict each component of the primary endpoint. The individual primary endpoint component analyses were pre-specified but were not adjusted for multiple comparisons. P-values are for the difference in proportions between TRC101 and placebo groups (Fisher's exact test). FIG. 19B: The percentage of patients in the TRC101 (circles) and placebo (squares) groups whose serum bicarbonate level increased from baseline to Week 12 by pre-specified thresholds. Achieving a ≥4 mmol/L increase was a component of the primary endpoint. FIG. 19C: The baseline bicarbonate (Treatment Week 0), the mean of the Screening 1, Screening 2, and Baseline Day 1 values, was 17.3 mmol/L in both treatment groups. Values depicted are the means (±95% CI) change from baseline in serum bicarbonate (mmol/L). At Week 12, the mean serum bicarbonate increase was 4.5 (95% CI, 3.9 to 5.1) mmol/L in the TRC101 group (circles) vs. 1.7 (95% CI, 1.0 to 2.3) mmol/L in the placebo group (squares).

Results from post-hoc analyses using a rank-based model were consistent with those from the pre-specified MMRM model (p<0.0001 for treatment effect).

Other than in subgroups with <8 patients, the lower-bound of the 95% confidence interval for the treatment difference exceeded 0 within all pre-specified subgroups, including age, gender, geographical region, baseline bicarbonate, screening eGFR, and baseline alkali use. Other than in subgroups with <8 patients, the lower-bound of the 95% confidence interval for the treatment difference exceeded 0 within all pre-specified subgroups, including age, gender, geographical region, baseline alkali use, baseline bicarbonate and screening eGFR. P-values for the interaction between treatment and each subgroup were obtained from logistic regression models, where treatment, subgroup, and interaction of treatment×subgroup were included as predictors. However, these should be interpreted with caution given the post-hoc nature of the analysis and multiple comparisons.)

Physical Functioning

Metabolic acidosis has been implicated as an important factor contributing to reduced muscle mass, manifested in decreases in lean body mass and muscle strength as well as increased protein catabolic rate. Prior to a measurable decrease in blood bicarbonate, the body adapts, in part, to the increasing acid load by using intracellular buffers in muscle (primarily proteins and organic phosphates).

The two exploratory endpoints in this study were included to assess whether improvement in muscle function and patient quality of life could be demonstrated in the patient population through the treatment of metabolic acidosis. The first exploratory endpoint examined the effect of treatment with TRC101 on self-reported responses to the physical functioning subpart of the Kidney Disease and Quality of Life Short Form, or the KDQOL-SF, survey. The KDQOL-SF survey is a validated questionnaire designed to assess health-related quality of life, or HRQOL, in kidney disease patients. Subjects in the trial responded to 10 questions related to physical function during daily activities, or KDQOL-SF Physical Function Survey (Question 3) (see, e.g., FIG. 21). The score conversion for the Survey is as follows: 1 (limited a lot)=0; 2 (limited a little)=50; 3 (not limited)=100. Total score=sum of all 10, divided by 10. The second exploratory endpoint objectively measured physical function derived from a repeated chair stand test, or Repeated Chair Stand Test. In the Repeated Chair Stand Test, subjects were asked to fold their arms across their chests and to stand up from a sitting position once; if they successfully rose from the chair, they were asked to stand up and sit down five times as quickly as possible, and the time for these five repetitions was recorded (see, e.g., FIG. 22). The KDQOL-SF Physical Function Survey and Repeated Chair Stand Test were administered and scored in a blinded fashion, and a change in Physical Function Survey score and Repeated Chair Stand Test time from baseline at week 12 were pre-defined as exploratory endpoints.

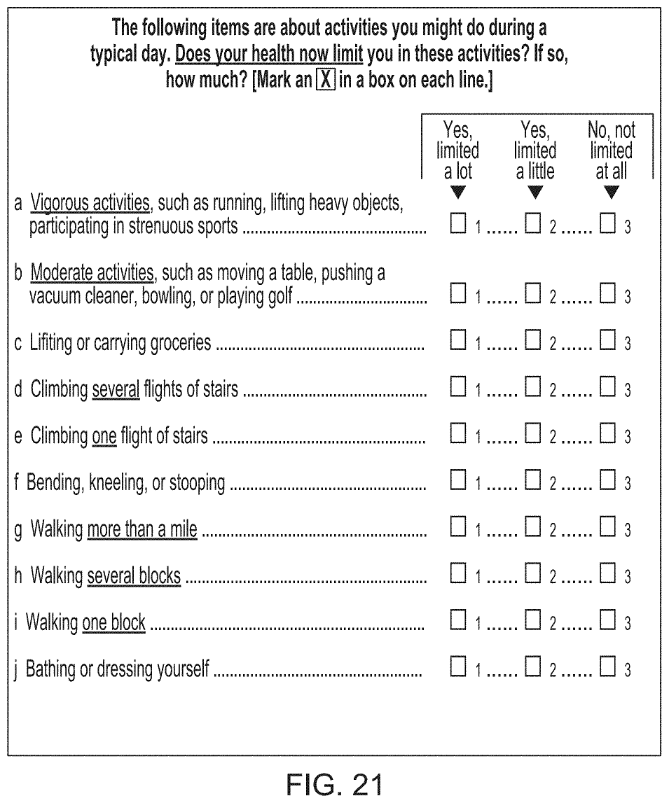

At the end of 12 weeks of treatment, physical functioning, as measured by the KDQOL Physical Function Domain, which quantifies patients' self-reported degree of limitation in performing daily activities such as climbing stairs and walking (FIG. 21), increased significantly in TRC101-treated patients compared to placebo-treated patients (p=0.0122) (FIG. 20A). The LS mean (95% CI) change within the TRC101 group (6.3 [3.7, 8.9]) and the placebo-subtracted treatment effect (5.2 [1.1, 9.2]) both exceeded the minimal clinically important difference in KDQOL subscales as reported in the literature (Clement, F M et al., 2009, The Impact of Selecting a High Hemoglobin Target Level on Health-Related Quality of Life for Patients with Chronic Kidney Disease: A Systematic Review and Meta-Analysis, Arch. Intern. Med. 169 (12): 1104-1112; Collister, D et al., 2016, The Effect of Erythropoietin-Stimulating Agents on Health-Related Quality of Lide in Anemia of Chronic Kidney Disease: A Systematic Review and Meta-Analysis, Ann. Intern. Med. 164(7): 472-478; Leaf, D E et al., 2009, Interpretation and Review of Health-Related Quality of Life Data in CKD Patients Receiving Treatment for Anemia, Kidney Int. 75(1): 15-24; Samsa, G et al., Determining Clinically Important Differences in Health Status Measures: A General Approach with Illustration to the Health Utilities Index Mark II, Pharmacoeconomics, 15(2): 141-155). Physical function, as measured by the repeated chair stand test, numerically improved in the TRC101 group (p=0.0249) and numerically worsened (p=0.5727) in the placebo group: on average (LS mean [95% CI]), the chair stand time increased by 0.35 (−0.9, 1.6) seconds in the placebo group and declined by 1.17 (0.2, 2.2) seconds in the TRC101 group (FIG. 20B). The between-group difference was not statistically significant (p=0.0630). (FIGS. 20A-20B—Changes in Physical Functioning.) (FIG. 20A: Patients reported how limited they were on the 10 items of the Physical Functioning Domain of the Kidney Disease and Quality of Life (KDQOL) at Baseline and at Treatment Week 12 (see FIG. 21). The least squares mean and 95% Cl of the change from baseline to Week 12 in total score is presented for each group. The range for the minimal clinically important differences reported for the KDQOL subscales is 3-5 points.) (FIG. 20B: Patients were timed on the speed with which they could repeatedly stand from a chair five times at baseline and at Treatment Week 12. Least squares mean and 95% Cl of the change from baseline to Week 12 in the time to perform the repeated chair stand test is presented for each group. Not all patients were able to perform the test. Data are presented for patients who performed the test at both baseline and Week 12. (TRC101, n=109; Placebo, n=76).)

Post-hoc rank-based analyses of physical function showed consistent results for patient-reported physical function (p=0.0117) and a stronger association for the between-group difference in the time to complete the repeated chair stand test (p=0.0027), both favoring TRC101.

Safety

TRC101 was well-tolerated. In total, over 95% of subjects in each of the groups completed the trial. Overall treatment-related adverse events occurred in 9.7% of subjects in the placebo group and 13.7% of TRC101-treated subjects. The most common treatment-related adverse events were mild to moderate GI disorders, which occurred in 5.4% of subjects in the placebo group and 12.9% of TRC101-treated subjects. The GI adverse events that occurred in more than one subject in the trial included diarrhea, flatulence, nausea and constipation. The only other treatment-related adverse event that occurred in more than one subject was paresthesia (1.1% of subjects in the placebo group and 0.8% of TRC101-treated subjects). There were no apparent effects of TRC101 on serum parameters, such as sodium, calcium, potassium, phosphate, magnesium, or low-density lipoprotein observed in the trial that would indicate off-target effects of TRC101. A high blood bicarbonate level, defined as greater than 30 mEq/L, was observed transiently in 2 subjects, or 0.9%. Discontinuation of TRC101 per the protocol-defined dosing algorithm resulted in normalization of blood bicarbonate in these subjects.

There were no apparent effects of TRC101 on vital signs, ECG intervals, renal function, hematology parameters, liver function tests, lipids, or urinalyses (Table 806).

TABLE 806
Change from Baseline in Laboratory Parameters
and Blood Pressure after 12 Weeks of Treatment

| | Placebo | | TRC101 | |
|---|---|---|---|---|
| | (N = 93) | | (N = 124) | |
| Blood urea nitrogen - mmol/L | | | | |
| no. | 89 | | 120 | |
| Mean (SD) | 0.65 | (3.96) | −0.05 | (4.02) |
| Median (IQR) | 0.36 | (3.21) | 0.00 | (3.75) |
| Serum creatinine - μmol/L | | | | |
| no. | 89 | | 120 | |
| Mean (SD) | 13.3 | (49.4) | 11.1 | (44.9) |
| Median (IQR) | 6.2 | (37.1) | 6.6 | (40.2) |
| Serum sodium - mmol/L | | | | |
| no. | 89 | | 120 | |
| Mean (SD) | 0.0 | (2.9) | 0.3 | (2.9) |
| Median (IQR) | 0.0 | (4.0) | 0.5 | (3.0) |
| Serum potassium - mmol/L | | | | |
| no. | 89 | | 118 | |
| Mean (SD) | 0.05 | (0.63) | 0.03 | (0.60) |
| Median (IQR) | 0.00 | (0.80) | 0.00 | (0.80) |
| Serum chloride - mmol/L | | | | |
| no. | 89 | | 120 | |
| Mean (SD) | −0.1 | (3.4) | −0.2 | (3.3) |
| Median (IQR) | 0.0 | (4.0) | 0.0 | (5.0) |
| Serum calcium - mmol/L | | | | |
| no. | 89 | | 120 | |
| Mean (SD) | −0.02 | (0.13) | −0.02 | (0.12) |
| Median (IQR) | −0.03 | (0.13) | −0.01 | (0.15) |
| Serum phosphate - mmol/L | | | | |
| no. | 89 | | 120 | |
| Mean (SD) | 0.03 | (0.22) | 0.02 | (0.19) |
| Median (IQR) | 0.03 | (0.23) | 0.03 | (0.19) |
| Serum magnesium - mmol/L | | | | |
| no. | 89 | | 120 | |
| Mean (SD) | 0.02 | (0.09) | 0.02 | (0.09) |
| Median (IQR) | 0.04 | (0.12) | 0.00 | (0.12) |
| Estimated glomerular filtration rate - | | | | |
| mL/min/1.73 m² | | | | |
| no. | 89 | | 120 | |
| Mean (SD) | −0.8 | (5.1) | −0.8 | (6.0) |
| Median (IQR) | −1.0 | (6.0) | −1.0 | (5.0) |
| Venous blood pH | | | | |
| no. | 89 | | 120 | |
| Mean (SD) | 0.03 | (0.12) | 0.05 | (0.10) |
| Median (IQR) | 0.03 | (0.10) | 0.05 | (0.11) |
| Venous blood base excess - mmol/L | | | | |
| no. | 89 | | 120 | |
| Mean (SD) | 2.1 | (4.2) | 5.3 | (4.3) |
| Median (IQR) | 2.0 | (6.0) | 5.0 | (7.0) |
| Cholesterol (total) - mmol/L | | | | |
| no. | 89 | | 120 | |
| Mean (SD) | −0.16 | (0.93) | 0.05 | (0.99) |
| Median (IQR) | −0.08 | (0.88) | 0.03 | (1.03) |
| Low-density lipoprotein cholesterol- | | | | |
| mmol/L | | | | |
| no. | 87 | | 115 | |
| Mean (SD) | −0.06 | (0.81) | 0.02 | (0.84) |
| Median (IQR) | −0.05 | (0.85) | −0.03 | (0.93) |
| High-density lipoprotein cholesterol - | | | | |
| mmol/L | | | | |
| no. | 89 | | 120 | |
| Mean (SD) | 0.02 | (0.29) | 0.04 | (0.34) |
| Median (IQR) | 0.03 | (0.28) | 0.08 | (0.25) |
| Systolic blood pressure- mmHg | | | | |
| no. | 89 | | 120 | |
| Mean (SD) | −1.1 | (8.7) | −2.4 | (7.6) |
| Median (IQR) | −1.0 | (6.0) | −2.0 | (7.5) |
| Diastolic blood pressure- mmHg | | | | |
| no. | 89 | | 120 | |
| Mean (SD) | −1.4 | (7.4) | −1.5 | (6.6) |
| Median (IQR) | −1.0 | (7.0) | −10 | (8.5) |

A high (>30 mmol/L) serum bicarbonate level was observed transiently in two patients but normalized following interruption of study drug per the protocol titration algorithm. There were no apparent effects on serum electrolytes that would indicate off-target effects of TRC101 (Table 806). The incidence of serum potassium≥5.0 or ≥6.0 mmol/L (Table 807), and mean serum potassium over time, were similar in both groups.

TABLE 807
Proportion of Patients with Serum Potassium
Exceeding Predefined Thresholds

| | |
|---|---|
| Placebo | TRC101 |
| (N = 93) | (N = 124) |

Baseline

| | | |
|---|---|---|
| no. | 92 | 124 |

| | | | | |
|---|---|---|---|---|
| >5 mmol/L - no. (%) | 31 | (34) | 41 | (33) |
| >6 mmol/L - no. (%) | 2 | (2) | 4 | (3) |

| | | |
|---|---|---|
| Week 1 | | |
| no. | 88 | 117 |

| | | | | |
|---|---|---|---|---|
| >5 mmol/L - no. (%) | 39 | (44) | 51 | (44) |
| >6 mmol/L - no. (%) | 7 | (8) | 5 | (4) |

| | | |
|---|---|---|
| Week 2 | | |
| no. | 87 | 115 |

| | | | | |
|---|---|---|---|---|
| >5 mmol/L - no. (%) | 39 | (45) | 46 | (40) |
| >6 mmol/L - no. (%) | 3 | (3) | 2 | (2) |

| | | |
|---|---|---|
| Week 4 | | |
| no. | 87 | 118 |

| | | | | |
|---|---|---|---|---|
| >5 mmol/L - no. (%) | 46 | (53) | 53 | (45) |
| >6 mmol/L - no. (%) | 5 | (6) | 7 | (6) |

| | | |
|---|---|---|
| Week 6 | | |
| no. | 91 | 120 |

| | | | | |
|---|---|---|---|---|
| >5 mmol/L - no. (%) | 46 | (51) | 52 | (43) |
| >6 mmol/L - no. (%) | 6 | (7) | 8 | (7) |

| | | |
|---|---|---|
| Week 8 | | |
| no. | 89 | 119 |

| | | | | |
|---|---|---|---|---|
| >5 mmol/L - no. (%) | 40 | (45) | 54 | (45) |
| >6 mmol/L - no. (%) | 6 | (7) | 8 | (7) |

| | | |
|---|---|---|
| Week 10 | | |
| no. | 87 | 119 |

| | | | | |
|---|---|---|---|---|
| >5 mmol/L - no. (%) | 40 | (46) | 45 | (38) |
| >6 mmol/L - no. (%) | 2 | (2) | 7 | (6) |

| | | |
|---|---|---|
| Week 12 | | |
| no. | 89 | 118 |

| | | | | |
|---|---|---|---|---|
| >5 mmol/L - no. (%) | 38 | (43) | 43 | (36) |
| >6 mmol/L - no. (%) | 5 | (6) | 5 | (4) |

Discussion

In non-dialysis-dependent patients with CKD and chronic metabolic acidosis (mean serum bicarbonate 17.3 mmol/L), 12 weeks of treatment with TRC101 significantly increased serum bicarbonate, with 50% of patients achieving normalization, 56% achieving a ≥4 mmol/L increase, and 59% meeting the composite primary endpoint definition. The mean increase in serum bicarbonate at Week 12 in the TRC101 group was 4.5 mmol/L, and 39% and 26% of TRC101-treated patients had an increase in serum bicarbonate≥6 and ≥7 mmol/L, respectively. The effect of TRC101 on serum bicarbonate was both rapid and sustained over 12 weeks in these outpatients whose dietary protein intake was not governed by the study protocol.

Accumulation of metabolically produced acid stimulates increases kidney production of endothelin, angiotensin II and aldosterone, substances that provide the short-term benefit of enhancing renal tubule acid excretion but are detrimental in the long term by promoting inflammation and fibrosis in the kidney interstitium that contributes to a progressive decline of kidney function. Similarly, in response to acid retention the kidney increases ammonia production per functioning nephron to facilitate acid excretion; however, the increased ammonia levels promote inflammation and activation of complement that also contributes to kidney fibrosis.

Metabolic acidosis in patients with CKD has traditionally been treated with sodium-based alkali supplements (sodium bicarbonate, sodium citrate) that enter the systemic circulation and neutralize accumulated acid. Potassium-based alkali therapies (e.g., potassium bicarbonate) are rarely used in patients with CKD because of the risk of life-threatening hyperkalemia. Alternative treatments for metabolic acidosis include vegetarian diets, but these limit patient choice and have low long-term adherence. An alternative treatment would remove, rather than neutralize, acid, without administering a sodium or potassium load. Removal of acid by binding to a non-absorbed polymer that is then excreted is a potential new mechanism for treating metabolic acidosis in patients with CKD.

The study described in this Example demonstrates that TRC101, a non-absorbed, counterion-free, polymeric drug that selectively binds and removes hydrochloric acid from the gastrointestinal tract, thus increasing systemic bicarbonate concentration, is effective in treating metabolic acidosis. These findings demonstrate that the effect of TRC101 on serum bicarbonate reaches a plateau after 4 to 8 weeks of treatment and the effect is sustained over 12 weeks in an outpatient CKD population eating a free choice diet.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes can be made by those skilled in the art without departing from the spirit of this disclosure. Those skilled in the art will appreciate that the inventive principles can be practiced in other than the described embodiments, which are presented for purposes of illustration and not limitation.

### Example 6

A Blinded, Placebo-Controlled Extension to Study TRCA-301 to Evaluate the Long-term Safety and Durability of Effect of TRC101 in Subjects with Chronic Kidney Disease and Metabolic Acidosis

The study described in example 5 was continued in an additional 40-week, double-blinded, placebo-controlled extension of that study, taking the total study time to 52 weeks. The study of example 5 is referred to as TRCA-301 and the study of example 6 is referred to as TRCA-301E. A total of 196 subjects with chronic kidney disease (CKD) and blood bicarbonate value of ≤12 mEq/L who completed the 12-week Treatment Period in TRCA-301 participated in this extension study.

Subjects were eligible for inclusion in the study if they completed the 12-week treatment period of TRCA-301 and attended the Week 12 Visit in Study TRCA-301, had a blood bicarbonate value of ≤12 mEq/L at the Week 12 Visit in Study TRCA-301, based on onsite measurement using the i-STAT point-of-care device, and had adequate peripheral venous access for blood draws. Women who were of childbearing potential were included if they had negative pregnancy test at the Week 12 Visit and were willing to use an acceptable method of birth control until 1 month after study completion.

Subjects were excluded if they had low blood bicarbonate at the Week 12 Visit in Study TRCA-301 requiring emergency intervention or evaluation for an acute acidotic process, required dialysis for acute kidney injury or worsening CKD during Study TRCA-301, or planned initiation of renal replacement therapy (dialysis or transplantation) within 6 months following study entry. Other key exclusion criteria were diabetic gastroparesis or a history of bariatric surgery, a history or current diagnosis of bowel obstruction, swallowing disorders, severe gastrointestinal (GI) disorders, inflammatory bowel disease, major GI surgery, or active gastric/duodenal ulcers, history of bariatric surgery, a serum calcium 8.0 mg/dL at the Week 10 Visit in Study TRCA-301, active cancer during the 1 year prior to the Week 12 Visit in Study TRCA-301, or inability to consume the study drug.

After the subject provided informed consent, the subject's eligibility were evaluated on the basis of laboratory values, vital signs, renal status and pregnancy test (if applicable) during the 1-day screening visit (Week 12 Visit of Study TRCA-301). Eligible subjects were treated with TRC101 or placebo once daily (QD) on an out-patient basis for the subsequent 40 weeks (Treatment Period); subjects continued to receive the same blinded treatment (TRC101 or placebo) that they received in Study TRCA-301. As in Study TRCA-301, the study drug dose was algorithmically titrated by the interactive response technology system in the range from 0-9 g/day (or equivalent number of packets of placebo) to a target bicarbonate concentration of 22-29 mmol/L based on the bicarbonate measurement at each visit. Subjects with a confirmed blood bicarbonate level>30 mEq/L underwent an interruption of the study drug dose in accordance with the titration algorithm. Subjects with a blood bicarbonate level below the normal range (<22 mEq/L) might have a blinded adjustment of the study drug dose in accordance with the titration algorithm. Dosing of oral concomitant medications and study drug were separated by at least 4 hours. No addition or dose changes of any other concomitant therapy to raise blood bicarbonate were allowed. Subjects who entered the study at the Week 12 Visit in Study TRCA-301 on an oral alkali supplement were taken off the supplement if their blood bicarbonate was within the normal range (22 to 29 mEq/L) or above it (>29 mEq/L).

Subjects returned to the study center for study visits at Weeks 2, 4, 8, 12, 16, 22, 28, 34, and 40 of the TRCA-301E study (equivalent to weeks 14, 16, 20, 24, 28, 34, 40, 46 and 52 weeks when added to the duration of the TRCA-301 study) for assessments of safety and durability of effect. Subjects who completed the Treatment Period entered the 2-week Follow-up Period and returned to the study site for two visits: Follow-up 1 (Week 41) and Follow-up 2 (Week 42) of the TRCA-301E trial, for adverse event (AE) collection, fasting blood draws and safety assessments. Subjects who withdrew from the study prematurely (i.e., prior to the Week 40 Visit) underwent an Early Termination (ET) Visit, during which all Week 40 Visit assessments were performed, and were asked to attend Follow-up 1 and 2 Visits in 1 and 2 weeks, respectively. All subjects who discontinued study drug prior to the Week 40 Visit were contacted by telephone 40 weeks after their 1-day screening visit to ascertain vital status and renal status (i.e., receiving renal replacement therapy or not).

Blood draws for bicarbonate measurements were performed when subjects were in a fasted state (at least 4 hours) and at approximately the same time of day for each subject. Safety assessments included AEs, vital signs, physical examination, safety laboratory measurements, coagulation, pregnancy test, and electrocardiograms (ECGs). The Primary Endpoint was incidence of AEs, serious AEs and AEs leading to withdrawal. The Secondary Endpoints were 1) having a change from baseline (CFB) in blood bicarbonate 4 mEq/L or having blood bicarbonate in the normal range (22 to 29 mEq/L) at the end of treatment; 2) CFB in blood bicarbonate at the end of treatment; 3) CFB in the total score of the Kidney Disease and Quality of Life (KDQOL) Question 3 items (daily activities) at the end of treatment; 4) CFB in repeated chair stand test duration at the end of treatment.

Results

The TRCA-301E study demonstrates that TRC101 is consistently highly effective over the course of a 52 week trial for the treatment of metabolic acidosis in non-dialysis-dependent patients with CKD and chronic metabolic acidosis (mean serum bicarbonate 17.3 mmol/L).

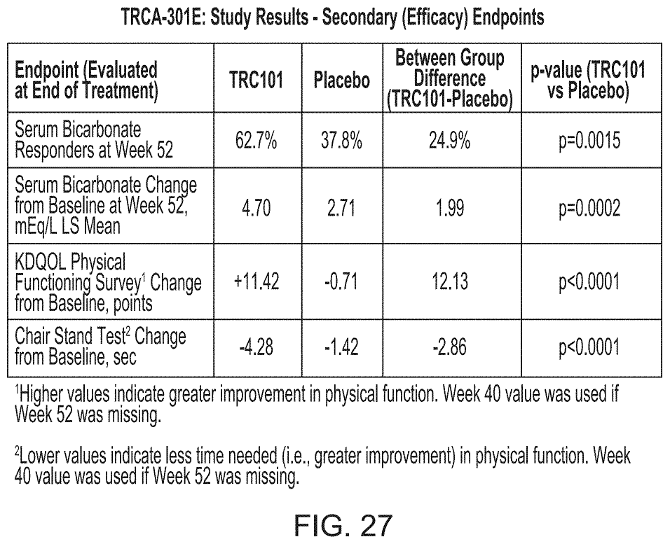

FIG. 27 summarises the key markers which demonstrate the effectiveness of TRC101 for the long term management and/or treatment of metabolic acidosis in patients with CKD. The effect of TRC101 on serum bicarbonate, as observed in the TRCA-301 trial is rapid, but the extension trial demonstrates how the improvements in serum bicarbonate levels are maintained upon continued use of the drug for 52 weeks. On average, at the end of 52 weeks of treatment, TRC101-treated patients experienced a 4.70 mEq/L increase in serum bicarbonate level when compared to baseline, whilst for placebo-treated patients this value improved only by an average of 2.71 mEq/L, with a difference between these groups of 1.99 mEq/L. These values were statistically significant (p=0.0002).

At the end of 52 weeks of treatment, 62.7% of TRC101-treated patients experienced an increase in SBC level, compared to only 37.8% of placebo-treated patients. The difference between these 2 groups was 24.9%, and this was a statistically significant result (p=0.0015).

As for the TRCA-301 study, the two exploratory endpoints in this extension study were included to assess whether improvement in muscle function and patient quality of life could be demonstrated in the patient population through the extended treatment of metabolic acidosis. The first exploratory endpoint examined the effect of treatment with TRC101 on self-reported responses to the physical functioning subpart of the Kidney Disease and Quality of Life Short Form, or the KDQOL-SF, survey. The second exploratory endpoint objectively measured physical function derived from a repeated chair stand test, or Repeated Chair Stand Test. The KDQOL-SF physical function survey and the repeated chair stand test are described in more detail in Example 5.

At the end of 52 weeks of treatment, the KDQOL of TRC101-treated patients was increased by 11.42 from baseline. Placebo-treated patients saw a decrease in KDQOL of 0.71, generating an overall difference of 12.13 points after 52 weeks of treatment, and this difference was statistically significant (p<0.0001). An average reduction of 4.28 seconds for the time taken to complete the repeated chair stand test was also seen for TRC101-treated patients at the end of the 52 week study, whilst placebo-treated patients experienced a decrease of only 1.42 seconds, generating a difference of −2.86 seconds and this difference was statistically significant (p<0.0001).

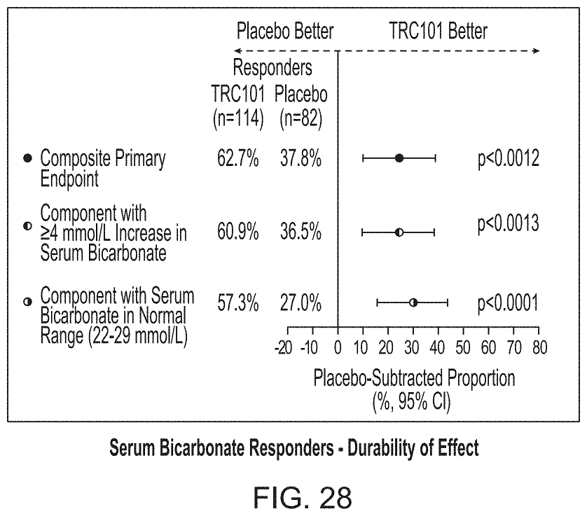

The SBC durability effect for TRC101-treated patients against placebo treated patients at the end of the TRCA-301 and TRCA-301E 52 week study is shown in FIG. 28. For the composite primary endpoint out to Treatment Week 52, the placebo-subtracted percentage of patients achieving a ≥4 mmol/L increase from baseline in serum bicarbonate or a serum bicarbonate in the normal range (22-29 mmol/L), is depicted as the top line (24.9%, 95% CI). The two lower lines depict each component of the primary endpoint (24.4%, 95% Cl and 30.3%, 95% CI). The values were statistically significant, and demonstrate the durability of the serum bicarbonate response out to 52 weeks.

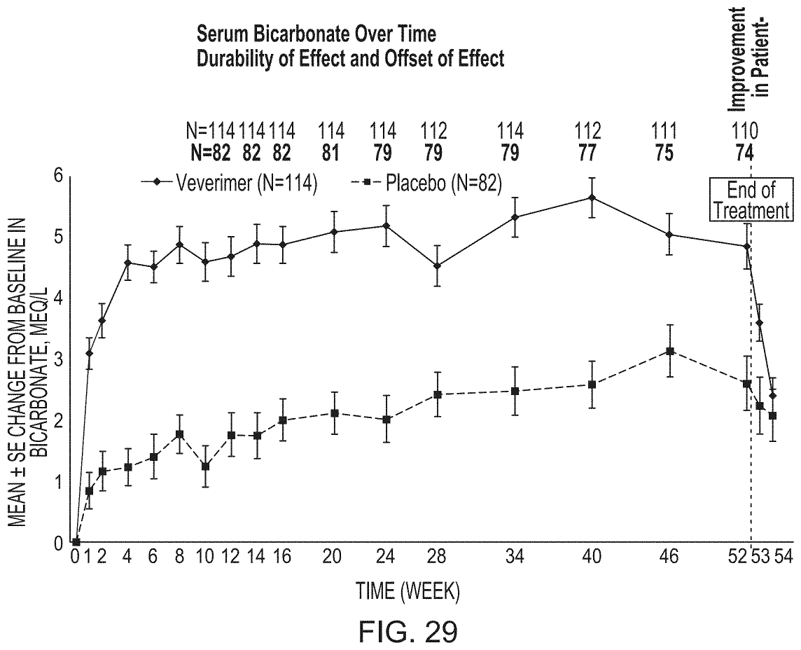

The average change in serum bicarbonate curves for the TRC101 and placebo groups separated over time starting at Treatment Week 1 and maintained a significant degree of separation throughout the 52 week treatment period, as illustrated in FIG. 29. At week 52 the mean change from baseline in the TRC101-treated group was 4.70 mEq/L. A rapid and immediate decrease in serum bicarbonate level was seen at weeks 53 and 54 following the cessation of treatment with TRC101 after 52 weeks. FIG. 29 demonstrates how TRC101 can significantly raise the serum bicarbonate levels of CKD patients with metabolic acidosis, and this improvement is maintained upon continued use of the drug.

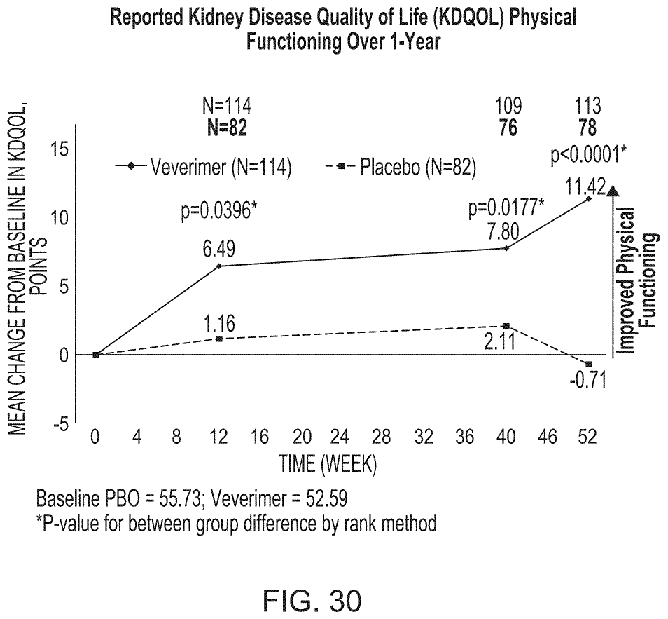

The change in KDQOL-Physical Functioning domain was observed from week 1 of treatment with TRC101 through to week 52, as shown in FIG. 30. TRC101 was instantly effective and continued to improve patient's KDQOL-physical functioning domain scores throughout the 52 week trial period. Notably, between weeks 40 and 52, the KDQOL-physical functioning domain of the placebo-treated patients deteriorated, whilst the KDQOL-physical functioning domain of TRC101-treated patients continued to increase. Following 52 weeks of treatment, TRC101-treated patients had an average increase in physical functioning domain of 11.42 compared to baseline, whilst placebo-treated patients experienced a decrease in physical functioning domain of 0.71 compared to baseline. The difference between the TRC101-treated group and the placebo-treated group was 12.13, and this result was statistically significant (p<0.0001).

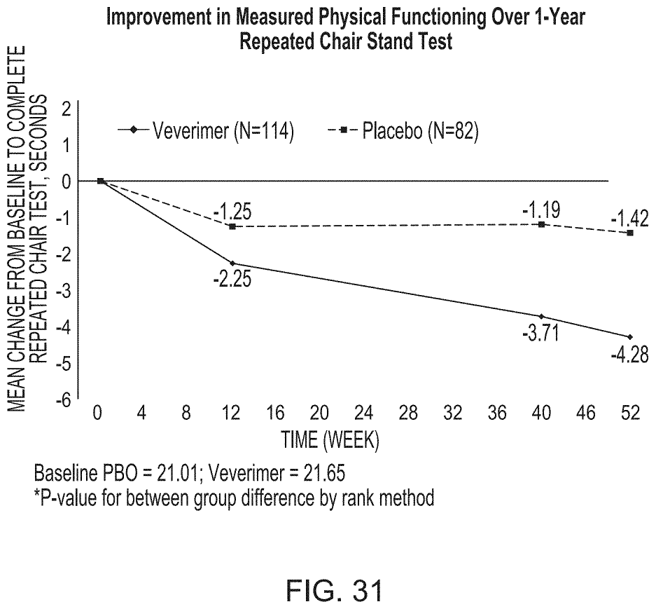

The time taken to complete the repeated chair stand test decreased continuously from week 1 to week 52 for TRC101-treated patients, as shown in FIG. 31. At week 12 there was a mean decrease of 2.25 seconds relative to baseline, by week 40 there was a mean decrease of 3.71 seconds relative to baseline and by week 52 there was a mean decrease of 4.28 seconds relative to baseline. Placebo-treated patients also saw a decrease in the average time taken to complete the repeated chair stand test, but this was of a much smaller degree and mostly plateaued after 12 weeks. N represents the number of participants who contributed to each data point.

Safety

TRC101 was well-tolerated throughout the 52 week trial period. In total, 93.5% of patients in the TRC101 group completed the trial. Only 87.1% of the placebo group completed the 52 week trial. The most common treatment-related adverse effects were mild to moderate GI disorders. The GI adverse effects that occurred in more than one subject in the trial included flatulence and diarrhea.

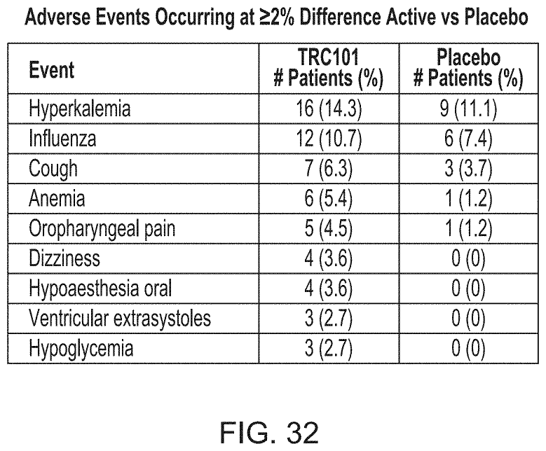

FIG. 32 shows the adverse events occurring at ≥2% difference between the TRC101-treated patients and the placebo-treated patients. 14.3% of TRC101-treated patients suffered from hyperkalemia, whilst only 11.1% of placebo-treated patients suffered from the same. 10.7% of TRC101-treated patients contracted influenza, whilst only 7.4% of placebo-treated patients contracted the same. 6.3% of TRC101-treated patients experienced a cough, whilst only 3.7% of placebo-treated patients experienced the same. 5.4% of TRC101-treated patients suffered from anemia, whilst only 1.2% of placebo-treated patients suffered the same. 4.5% of TRC101-treated patients suffered oropharyngeal pain, whilst only 1.2% of placebo-treated patients suffered the same.

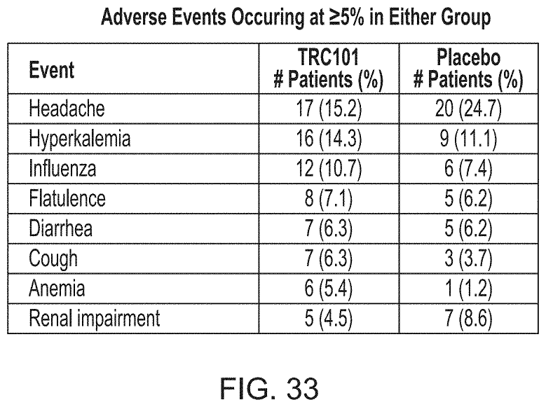

FIG. 33 lists the adverse effects which occurred at ≥5% percentage of both TRC101-treated patients and placebo-treated patients. Instances of headache were most common between the 2 groups, with 24.7% of placebo-treated patients experiencing headache and 15.2% of TRC101 treated patients experiencing headache. The most commonly experienced adverse effects between the 2 groups were headache, hyperkalemia and influenza.

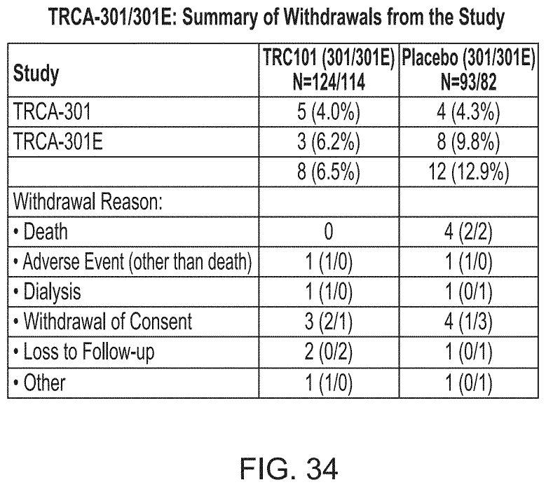

TRC101 was well tolerated, with only 8 subjects withdrawing from the combined TRCA-301 and TRCA-301 E extension studies. Notably, none of these withdrawals were the result of death. 4 subjects were withdrawn from the placebo trial as a result of death. In both the TRC101-treated patients and the placebo-treated patients, 1 patient from each group was withdrawn to begin dialysis. This data is shown in FIG. 34.

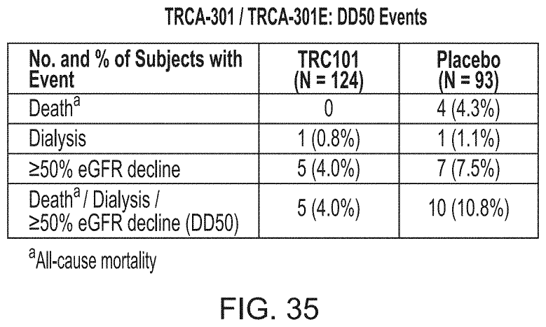

FIG. 35 shows that across the TRCA-301 and the TRCA-301E study, only 4% of TRC101-treated patients experienced a ≥50% decline in eGFR. 7.5% of placebo-treated patients experienced a ≥50% reduction in eGFR. It is important to note that placebo-treated patients were permitted to continue their existing oral alkali supplement throughout the TRCA-301 and TRCA-301 E trials.

FIG. 35 also shows the number of patients who experienced death (all-cause mortality), dialysis/kidney transplant (renal replacement therapy) or a ≥50% decline in eGFR, (which combined are equal to DD50) over the combined TRCA-301 and TRCA-301 E 52-week treatment period, relative to the placebo group. This data has not been annualized. Of the 124 subjects randomized to the TRC101-treated group, 5 (4.0%) subjects had a DD50 event, whereas 10 (10.8%) of subjects receiving placebo had a DD50 event. There were no deaths in the TRC101-treated group and only one TRC101-treated subject initiated dialysis during the 52-week treatment period. Of the 93 subjects randomized to the placebo group, 4 (4.3%) subjects died and 1 (1.1%) initiated dialysis during the 52-week treatment period.

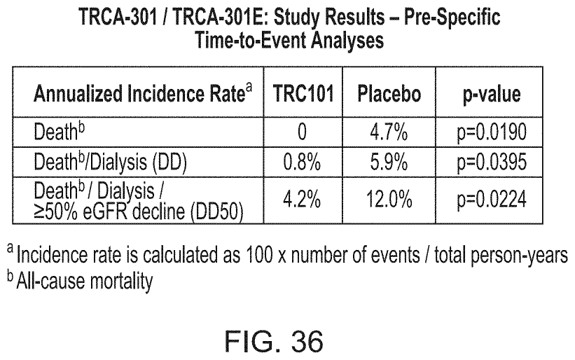

FIG. 36 shows that the time to DD50 was prolonged in the TRC101-treated group compared to the placebo group, with an annualized DD50 incidence rate, calculated as 100×number of events/total person-years, of 4.2% in the TRC101-treated group vs 12.0% in the placebo-treated group (p=0.0224). The annualized incident rate for TRC101-treated patients time to death was 0%, compared to 4.7% for the placebo-treated group (p=0.0190). The annualized incident rate for TRC101-treated patients time to death/dialysis was 0.8% compared to 5.9% in the placebo-treated group (p=0.0224).

Over the course of 52 weeks of treatment with Veverimer, as shown in Table 900 below, there were no mean changes on serum parameters/elecrtolytes or on blood pressure.

TABLE 900
Change from Baseline in Laboratory Parameters
and Blood Pressure after 52 Weeks of Treatment

| | Placebo | | Veverimer | |
|---|---|---|---|---|
| | (N = 81) | | (N = 112) | |
| Blood urea nitrogen - mg/dL | | | | |
| no. | 72 | | 109 | |
| Mean (SD) | 4.1 | (10.85) | 3.2 | (12.86) |
| Serum creatinine - mg/dL | | | | |
| no. | 72 | | 109 | |
| Mean (SD) | 0.288 | (0.6934) | 0.248 | (0.5905) |
| Serum sodium - mEq/L | | | | |
| no. | 72 | | 109 | |
| Mean (SD) | −0.2 | (3.13) | −0.4 | (2.82) |
| Serum potassium - mEq/L | | | | |
| no. | 68 | | 101 | |
| Mean (SD) | −0.03 | (0.657) | 0.02 | (0.680) |
| Serum chloride - mEq/L | | | | |
| no. | 72 | | 109 | |
| Mean (SD) | −1.0 | (4.23) | −0.4 | (3.75) |
| Serum calcium - mg/dL | | | | |
| no. | 72 | | 109 | |
| Mean (SD) | −0.09 | (0.582) | −0.25 | (0.581) |
| Serum phosphate - mg/dL | | | | |
| no. | 72 | | 107 | |
| Mean (SD) | 0.19 | (0.678) | 0.16 | (0.770) |
| Serum magnesium - mg/dL | | | | |
| no. | 72 | | 109 | |
| Mean (SD) | 0.14 | (0.316) | 0.11 | (0.241) |
| Estimated glomerular filtration | | | | |
| rate - mL/min/1.73 m² | | | | |
| no. | 72 | | 109 | |
| Mean (SD) | −1.5 | (10.70) | −2.0 | (6.45) |
| Cholesterol (total) - mg/dL | | | | |
| no. | 73 | | 109 | |
| Mean (SD) | −7.9 | (42.57) | 1.3 | (47.86) |
| Low-density lipoprotein | | | | |
| cholesterol- mg/dL | | | | |
| no. | 70 | | 106 | |
| Mean (SD) | −6.6 | (32.36) | 2.6 | (40.24) |
| High-density lipoprotein | | | | |
| cholesterol - mg/dL | | | | |
| no. | 73 | | 109 | |
| Mean (SD) | 0.7 | (14.30) | −0.9 | (12.99) |
| Systolic blood pressure- mmHg | | | | |
| no. | 73 | | 109 | |
| Mean (SD) | −2.0 | (7.06) | −2.0 | (7.60) |
| Diastolic blood pressure- mmHg | | | | |
| no. | 73 | | 109 | |
| Mean (SD) | −2.9 | (5.78) | −2.6 | (8.04) |

In summary, TRC101 is highly is a highly effective and safe treatment for metabolic acidosis when administered to patients for 52 weeks. TRC101 consistently, and statistically significantly raises serum bicarbonate levels over 52 weeks and consistently statistically significantly improves both physical function and quality of life of CKD patients with metabolic acidosis. These data also show that it is possible to consistently improve clinical measures and symptoms of patients having CKD and/or MA using a nonabsorbable composition and those improvements are sustainable over a significant period of time, e.g. 12, 40 and/or 52 weeks from first taking the composition.

What is claimed is:

1. A method of improving the quality of life of a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

2. A method of improving the quality of life of a patient afflicted with chronic kidney disease and an acid-base disorder, the method comprising oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in quality of life is statistically significant compared to a placebo control group for a period of at least twelve weeks as assessed by a Quality of Life (QoL) questionnaire.

3. A method of improving quality of life of a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to statistically significantly increase the patient's quality of life compared to a placebo control.

4. A method of improving quality of life of a patient afflicted with metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the patient's quality of life compared to a placebo control group over the period, wherein the improvement in quality of life is statistically significant.

5. A pharmaceutical composition for improving the quality of life of a human patient afflicted with chronic kidney disease and an acid-base disorder, the patient having a baseline serum bicarbonate level of ≤22 mEq/L prior to treatment, the composition being a nonabsorbable composition having the capacity to: (a) remove a target species from the patient selected from the group consisting of protons, strong acids, and conjugate bases of strong acids; and (b) improve the patient's quality of life compared to a placebo control in a statistically significant manner over at least a twelve-week period.

6. A pharmaceutical composition for improving the quality of life of a human patient suffering from a disease or disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to improve the patient's quality of life compared to a placebo control in a statistically significant manner over at least the twelve-week period.

7. A pharmaceutical composition for improving the quality of life of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in quality of life compared to a placebo control is statistically significant over the twelve-week period.

8. A method of improving the physical function of a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

9. A method of improving the physical function of a patient afflicted with chronic kidney disease and an acid-base disorder, the method comprising oral administration of a pharmaceutical composition having: (a) the capacity to selectively bind a target species selected from the group consisting of protons, strong acids, and conjugate bases of strong acids; and (b) a target species binding capacity of at least 3 mEq/g in a Simulated Small Intestine Inorganic Buffer (SIB) assay, wherein the improvement in physical function is statistically significant compared to a placebo control group at least twelve weeks after initiation of treatment as assessed by the patient's answers to question 3 of the Kidney Disease Quality of Life Short Form (KDQOL-SF).

10. A method of improving the physical function of a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to statistically significantly increase the patient's physical function score based on answers to question 3 of the Kidney Disease Quality of Life Short Form (KDQOL-SF) compared to the patient's baseline physical function score.

11. A method of improving the physical function of a patient afflicted with metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to improve the physical function score of the patient compared to a placebo control group at the end of the period, wherein the improvement in the physical function score is statistically significant.

12. A pharmaceutical composition for improving the physical function score of a human patient afflicted with chronic kidney disease and an acid-base disorder, the patient having a baseline serum bicarbonate level of ≤22 mEq/L prior to treatment, the composition being a nonabsorbable composition having the capacity to: (a) remove a target species from the patient selected from the group consisting of protons, strong acids, and conjugate bases of strong acids; and (b) improve the patient's physical function score based on answers to question 3 of the Kidney Disease Quality of Life Short Form (KDQOL-SF) compared to a placebo control in a statistically significant manner at the end of at least a twelve-week period.

13. A pharmaceutical composition for improving the physical function score of a human patient suffering from a disease or disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to improve the patient's physical function score based on answers to question 3 of the Kidney Disease Quality of Life Short Form (KDQOL-SF) compared to a placebo control in a statistically significant manner at the end of an at least the twelve-week period.

14. A pharmaceutical composition for improving the physical function score of a human patient suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the improvement in physical function score is a statistically significant improvement over a baseline physical function score based on answers to question 3 of the Kidney Disease Quality of Life Short Form (KDQOL-SF) compared to a placebo control at the end of the at least twelve-week period.

15. A method of slowing the progression of kidney disease in a patient afflicted with chronic kidney disease and an acid-base disorder characterized by a baseline serum bicarbonate value of ≤22 mEq/L, the method comprising oral administration of a pharmaceutical composition capable of increasing and maintaining the patient's serum bicarbonate above 20 mEq/L for a period of at least twelve weeks, the pharmaceutical composition having the capacity to bind a target species selected from the group consisting of protons, strong acids, and conjugate bases of strong acids.

16. A method of slowing the progression of kidney disease in a patient afflicted with chronic kidney disease and an acid-base disorder, wherein the patient has a baseline serum bicarbonate value of ≤22 mEq/L, comprising orally administering to the patient an effective amount of TRC101 once daily for a period of time sufficient to increase the patient's serum bicarbonate by at least 1 mEq/L.

17. A method of slowing the progression of kidney disease in a patient afflicted with chronic kidney disease and metabolic acidosis disease, the method comprising administering to the patient a daily dose of a nonabsorbed crosslinked amine polymer, which daily dose: (a) is sufficient to increase the patient's serum bicarbonate concentration by at least 1 mEq/L; (b) results in a sustained serum bicarbonate increase of at least 1 mEq/L over a period of at least twelve weeks; and (c) is sufficient to slow the progression of kidney disease.

18. A pharmaceutical composition for slowing the progression of kidney disease in a human patient afflicted with chronic kidney disease and an acid-base disorder, the patient having a baseline serum bicarbonate level of ≤22 mEq/L prior to treatment, the composition being a nonabsorbable composition having the capacity to: (a) remove a target species from the patient selected from the group consisting of protons, strong acids, and conjugate bases of strong acids; and (b) slow the progression of kidney disease in a human patient over at least a twelve-week period.

19. A pharmaceutical composition for slowing the progression of kidney disease in a human patient afflicted with chronic kidney disease and an acid-base disorder by increasing that patient's serum bicarbonate value by at least 1 mEq/L over at least twelve weeks of treatment, the composition: (a) being a nonabsorbable composition having the capacity to remove a target species from the patient selected from the group consisting of protons, strong acids, and conjugate bases of strong acids; (b) characterized by a target species binding capacity of at least 3 mEq/g in a Simulated Small Intestine Inorganic Buffer (SIB) assay; and (c) having the capacity to slow the progression of kidney disease over at least the twelve-week period.

20. A pharmaceutical composition for slowing the progression of kidney disease in a human patient also suffering from metabolic acidosis disease, wherein: (a) an effective amount of the pharmaceutical composition is administered to the patient per day over at least a twelve-week period; (b) the pharmaceutical composition is nonabsorbable with the capacity to remove from the patient a target species selected from the group consisting of protons, strong acids, and conjugate bases of strong acids; (c) the pharmaceutical composition is characterized by a chloride ion binding capacity of at least 3 mEq/g in a Simulated Small Intestine Inorganic Buffer (SIB) assay; and (d) the progression of kidney disease in the patient is slowed over the twelve-week period compared to a placebo control group not receiving the pharmaceutical composition.

21. A pharmaceutical composition for use in a method of treating an acid-base disorder in a patient, wherein the method of treatment improves the quality of life of the patient.

22. A pharmaceutical composition for use in a method of treating an acid-base disorder in a patient, wherein the method of treatment improves the physical function of the patient.

23. The method/composition according to any one of the preceding claims, wherein the pharmaceutical composition increases the serum bicarbonate level by at least 1 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

24. The method/composition according to any one of the preceding claims, wherein the pharmaceutical composition increases the serum bicarbonate level by at least 2 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

25. The method/composition according to any one of the preceding claims, wherein the pharmaceutical composition increases the serum bicarbonate level by at least 2.5 mEq/L in a placebo controlled study, said increase being the difference between the cohort average serum bicarbonate level in a first cohort at the end of the study, relative to the cohort average serum bicarbonate level in a second cohort at the end of the study, wherein the first cohort's subjects receive the pharmaceutical composition and the second cohort's subjects receive a placebo, wherein the first and second cohorts each comprise a patient population sufficient in size to evaluate statistically significant serum bicarbonate level differences between the cohorts over the period.

26. The method/composition according to any one of the preceding claims, wherein the pharmaceutical composition has the capacity to change the patient's baseline serum bicarbonate level by at least 2 mEq/L in at the end of an at least twelve week placebo controlled study.

27. The method/composition according to any one of the preceding claims, wherein the pharmaceutical composition has the capacity to change the patient's baseline serum bicarbonate level by at least 3 mEq/L in at the end of an at least twelve week placebo controlled study.

28. The method/composition according to any one of the preceding claims, wherein the pharmaceutical composition has the capacity to change the patient's baseline serum bicarbonate level by at least 4 mEq/L in at the end of an at least twelve week placebo controlled study.

29. The method/composition according to any one of the preceding claims, wherein the patient population for each cohort is at least 25 patients.

30. The method/composition according to any one of the preceding claims, wherein the patient population for each cohort is at least 50 patients.

31. The method/composition according to any one of the preceding claims, wherein the patient population for each cohort is at least 100 patients.

32. The method/composition according to any one of the preceding claims, wherein the patient population for each cohort is at least 150 patients.

33. The method/composition according to any one of the preceding claims, wherein the patient population for each cohort is at least 200 patients.

34. The method/composition according to any one of the preceding claims, wherein improvement in quality of life or physical function is assessed by a questionnaire answered by a first cohort at the end of the period, relative to a second cohort who answered the same questionnaire at the end of the period, wherein the first cohort's subjects receive the pharmaceutical composition and the second cohort's subjects receive a placebo.

35. The method/composition according to any one of the preceding claims, wherein improvement in quality of life or physical function is assessed by a questionnaire, which is a clinically validated assessment for evaluating a patient's physical and mental health.

36. The method/composition according to any one of the preceding claims, wherein the questionnaire comprises questions concerning parameters selected from the group consisting of symptoms/problems related to the disease/condition, effects of the disease/condition, burden of the disease/condition, work status, cognitive function, quality of social interaction, sleep, social support, physical functioning, pain, general health, emotional well-being, social function, energy/fatigue, and combinations thereof.

37. The method/composition according to any one of the preceding claims, wherein the questionnaire comprises questions concerning how the patient's health limits the patient's ability to engage in physical activities selected from the group: vigorous activities; moderate activities; lifting or carrying groceries; climbing several flights of stairs; climbing one flight of stairs; bending, kneeling or stooping; walking more than one mile; walking several blocks; walking one block; and bathing or dressing.

38. The method/composition according to any one of the preceding claims, wherein the patient achieves at least about a 10% improvement on the quality of life scale relative to the placebo control.

39. The method/composition according to any one of the preceding claims, wherein the patient achieves at least about a 25% improvement on the quality of life scale relative to the placebo control.

40. The method/composition according to any one of the preceding claims, wherein the patient achieves at least about a 50% improvement on the quality of life scale relative to the placebo control.

41. The method/composition according to any one of the preceding claims, wherein the patient achieves at least about a 75% improvement on the quality of life scale relative to the placebo control.

42. The method/composition according to any one of the preceding claims, wherein the improvement of the physical function comprises: (a) an improvement in the patient's baseline physical function score of at least 1.5 points based on the patient's answers to question 3 of the Kidney Disease Quality of Life Short Form (KDQOL-SF); (b) an improvement in the patient's baseline repeated chair stand times of at least −1.5 seconds; or (c) an improvement in the patient's baseline physical function score of at least 1.5 points based on the patient's answers to question 3 of the KDQOL-SF and an improvement in the patient's baseline repeated chair stand times of at least −1.5 seconds.

43. The method/composition according to any one of the preceding claims, wherein the improvement in the patient's baseline physical function score is based on the patient's performance in the Single Chair Stand and/or Repeated Chair Stand protocols as depicted in FIG. 22.

44. The method/composition according to any one of the preceding claims, wherein the improvement in the patient's baseline repeated chair stand times represents an improvement for at least one repetition.

45. The method/composition according to any one of the preceding claims, wherein the improvement in the patient's baseline repeated chair stand times represents an improvement for at least two repetitions.

46. The method/composition according to any one of the preceding claims, wherein the improvement in the patient's baseline repeated chair stand times represents an improvement for at least three repetitions.

47. The method/composition according to any one of the preceding claims, wherein the improvement in the patient's baseline repeated chair stand times represents an improvement for at least four repetitions.

48. The method/composition according to any one of the preceding claims, wherein the improvement in the patient's baseline repeated chair stand times represents an improvement for at least five repetitions.

49. The method/composition according to any one of the preceding claims, wherein the improvement in the patient's baseline physical function score is based on the patient's answers to at least one question to Question 3 the of KDQOL-SF as depicted in FIG. 21.

50. The method/composition according to any one of the preceding claims, wherein the improvement in the patient's baseline physical function score is based on the patient's answers to at least five questions to Question 3 the of KDQOL-SF as depicted in FIG. 21.

51. The method/composition according to any one of the preceding claims, wherein the improvement in the patient's baseline physical function score is based on the patient's answers to at least seven questions to Question 3 of the KDQOL-SF as depicted in FIG. 21.

52. The method/composition according to any one of the preceding claims, wherein the improvement in the patient's baseline physical function score is based on the patient's answers to all questions to Question 3 of the KDQOL-SF as depicted in FIG. 21.

53. The method/composition according to any one of the preceding claims, wherein the improvement in the quality of life of the patient comprises a decrease or prevention of further bone loss and/or a decrease or prevention of further muscle loss in the patient.

54. The method according to any one of the preceding claims, wherein the improvement in physical function score further includes an improvement in the patient's baseline repeated chair stand times compared to a placebo control of at least −1.5 seconds over the period.

55. The method/composition according to any one of the preceding claims, wherein the patient achieves at least about a 1.5 point improvement on the KDQOL-SF scale relative to the placebo control.

56. The method/composition according to any one of the preceding claims, wherein the patient achieves at least about a 3.0 point improvement on the KDQOL-SF scale relative to the placebo control.

57. The method/composition according to any one of the preceding claims, wherein the patient achieves at least about a 4.5 point improvement on the KDQOL-SF scale relative to the placebo control.

58. The method/composition according to any one of the preceding claims, wherein the patient achieves at least about a 6.0 point improvement on the KDQOL-SF scale relative to the placebo control.

59. The method/composition according to any one of the preceding claims, wherein the patient's KDQOL-SF score is calculated as follows: 1 (limited a lot)=0; 2 (limited a little)=50; 3 (not limited)=100. Total score=sum of all 10, divided by 10.

60. The method/composition according to any one of the preceding claims, wherein the disease or disorder is characterized by a baseline serum bicarbonate value of less than 18 mEq/L.

61. The method/composition according to any one of the preceding claims, wherein the disease or disorder is characterized by a baseline serum bicarbonate value of at least 12 mEq/L.

62. The method/composition according to any one of the preceding claims, wherein the disease or disorder is characterized by a baseline serum bicarbonate value of at least 15 mEq/L.

63. The method/composition according to any one of the preceding claims, wherein the patient's baseline serum bicarbonate value increases by at least 1 mEq/L during the period.

64. The method/composition according to any one of the preceding claims, wherein the patient's baseline serum bicarbonate value increases by at least 2 mEq/L during the period.

65. The method/composition according to any one of the preceding claims, wherein the patient's baseline serum bicarbonate value increases by at least 3 mEq/L during the period.

66. The method/composition according to any one of the preceding claims, wherein a daily dose of the pharmaceutical composition is administered to the patient and the daily dose has the capacity to remove at least about 10 mEq/day, at least about 15 mEq/day, at least about 20 mEq/day, at least about 25 mEq/day, or at least about 30 mEq/day of the target species.

67. The method/composition according to any one of the preceding claims, wherein a daily dose of the pharmaceutical composition is administered to the patient and the daily dose has the capacity to remove less than 50 mEq/day or less than 35 mEq/day of the target species.

68. The method/composition according to any one of the preceding claims, wherein the period is at least three weeks, at least one month, at least two months, at least six months, at least 12 months, at least 18 months, or at least 24 months.

69. The method/composition according to any one of the preceding claims, wherein the pharmaceutical composition has the capacity to bind a target species selected from the group consisting of protons, strong acids, and conjugate bases of strong acids.

70. The method/composition according to any one of the preceding claims, wherein the conjugate base of a strong acid is selected from the group consisting of chloride, bisulfate and sulfate ions.

71. The method/composition according to any one of the preceding claims, wherein the target species comprises chloride ions.

72. The method/composition according to any one of the preceding claims, wherein the target species comprises hydrochloric acid.

73. The method/composition according to any one of the preceding claims, wherein the pharmaceutical composition is characterized by a chloride ion binding capacity of at least 1 mEq/g in a SIB assay.

74. The method/composition according to any one of the preceding claims, wherein the pharmaceutical composition is characterized by a chloride ion binding capacity of at least 1.5 mEq/g in a SIB assay.

75. The method/composition according to any one of the preceding claims, wherein the pharmaceutical composition is characterized by a chloride ion binding capacity of at least 2 mEq/g in a SIB assay.

76. The method/composition according to any one of the preceding claims, wherein the pharmaceutical composition has the capacity to bind chloride and phosphate ions in a SIB assay at a ratio that is at least 0.25:1, respectively.

77. The method/composition according to any one of the preceding claims, wherein the pharmaceutical composition has the capacity to bind chloride and phosphate ions in a SIB assay at a ratio that is at least 0.5:1, respectively.

78. The method/composition according to any one of the preceding claims, wherein the pharmaceutical composition has the capacity to bind chloride and phosphate ions in a SIB assay at a ratio that is at least 1:1, respectively.

79. The method/composition according to any one of the preceding claims, wherein the effective amount of the pharmaceutical composition or TRC101 comprises at least about 1 gm/day.

80. The method/composition according to any one of the preceding claims, wherein the effective amount of the pharmaceutical composition or TRC101 comprises from about 1-9 gm/day.

81. The method/composition according to any one of the preceding claims, wherein the effective amount of the pharmaceutical composition or TRC101 comprises about 4-6 gm/day.

82. The method/composition according to any one of the preceding claims, wherein the effective amount of the pharmaceutical composition or TRC101 comprises about 6 gm/day.

83. The method/composition according to any one of the preceding claims, wherein the effective amount of the pharmaceutical composition or TRC101 is administered to the patient in an oral dosage form once-a-day.

84. The method/composition according to any one of the preceding claims, wherein the effective amount of the pharmaceutical composition or TRC101 is adjusted to maintain the patient's serum bicarbonate level in a range between 22-29 mEq/L.

85. The method/composition according to any one of the preceding claims wherein the pharmaceutical composition comprises a polymer comprising a structure corresponding to Formula 4: 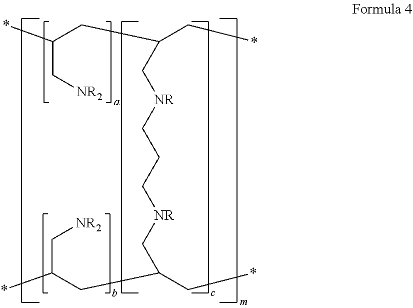
wherein each R is independently hydrogen or an ethylene crosslink between two nitrogen atoms of the crosslinked amine polymer 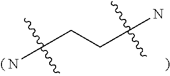
and a, b, c, and m are integers.

86. The method/composition according to any one of the preceding claims wherein the polymer comprises, 2-Propen-1-ylamine, 1,3-Bis(allylamino)propane and dichloroethane.
